(19) United States
(12) Patent Application Publication
(10) Pub. No.: US 2021/0101883 A1
(43) Pub. Date: Apr. 8, 2021

(54) ORGANIC COMPOUND AND ORGANIC ELECTROLUMINESCENCE DEVICE USING THE SAME

(71) Applicants: FENG-WEN YEN, Taipei (TW); LI-HSIN HSU, New Taipei (TW)

(72) Inventors: FENG-WEN YEN, Taipei (TW); LI-HSIN HSU, New Taipei (TW)

(73) Assignee: LUMINESCENCE TECHNOLOGY CORPORATION, HSINCHU (TW)

(21) Appl. No.: 16/592,769

(22) Filed: Oct. 4, 2019

(51) Int. Cl. C07D 403/04; C07D 519/00; C07D 491/048; C07D 495/04; C07D 403/10; C07D 401/14; C07D 403/14; C07D 407/14; C07D 409/14; H01L 51/00

(52) U.S. Cl. CPC C07D 403/04; H01L 51/5072; C07D 491/048; C07D 495/04; C07D 403/10; C07D 401/14; C07D 403/14; C07D 407/14; C07D 409/14; H01L 51/0072; H01L 51/0067; H01L 51/0061; H01L 51/006; H01L 51/0074; H01L 51/0073; H01L 51/0058; H01L 51/0056; H01L 51/0054; C07D 519/00

(57) ABSTRACT

An organic compound is described. An organic electroluminescence device comprises the organic compound, as an electron transporting layer, or as a hole blocking layer. The organic compound of the following formula may lower a driving voltage or increase a current efficiency or a half-life of the organic electroluminescence device.

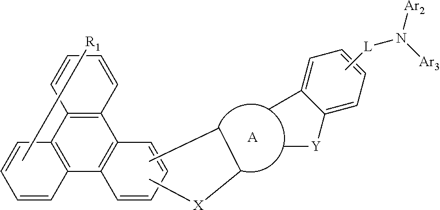

The same definition as described in the present invention.

### FIELD

The present invention relates to an organic compound and, more particularly, to an organic electroluminescence device using the organic compound.

### BACKGROUND

Organic electroluminescence (organic EL) devices, i.e., organic light-emitting diodes (OLEDs) that make use of organic compounds, are becoming increasingly desirable than before. The devices make use of thin organic films that emit light when voltage is applied across the device. They are becoming an interesting technology for use in applications such as flat panel displays, illumination, or backlighting.

One of the organic compounds, denoted ET2 hereinafter, has the following structure:

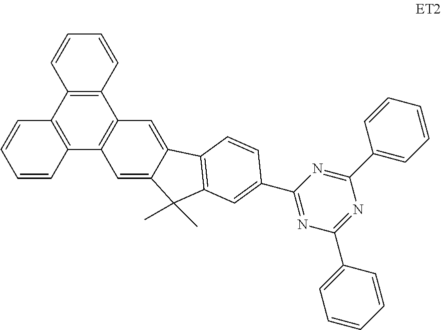 [SMILES: CC1(C)c2cc(-c3nc(-c4ccccc4)nc(-c4ccccc4)n3)ccc2-c2cc3c4ccccc4c4ccccc4c3cc21]

As used herein, “top” means furthest away from the substrate, while “bottom” means closest to the substrate. Where a second layer is described as formed on or onto a first layer, the second layer is formed further away from substrate. There may be other layers between the second layer and the first layer, unless it is specified that the second layer is “in contact with” the first layer. For example, a cathode may be described as formed onto an anode, even though there are various organic layers in between.

### SUMMARY

An organic compound is represented by the following formula:

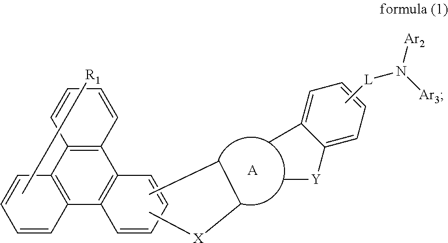

wherein X and Y independently represent a divalent bridge selected from the group consisting of O, S, NAr₁ and CR₂R₃;

wherein ring A represents a monocyclic aromatic group or a polycyclic aromatic group having at least two fused rings;

wherein L represents a single bond or an aromatic linker;

wherein R₁ to R₃ independently represent no substitution or mono to the maximum allowable substitution, each of the substituents is selected from the group consisting of alkyl, aryl, aralkyl, heteroaryl, and combinations thereof; and

wherein each of Ar₁, Ar₂ and Ar₃ is selected from the group consisting of aryl, heteroaryl, and combinations thereof.

The present invention further discloses an organic EL device. The organic EL device may comprise an anode, a cathode and one or more organic layers formed between the anode and the cathode. At least one of the organic layers comprises the organic compound of formula (1).

### BRIEF DESCRIPTION OF THE DRAWINGS

FIG. 1 is a cross-sectional view of a first organic EL device.

FIG. 2 is a cross-sectional view of an organic EL device without the electron transporting material ET2 of FIG. 1.

FIG. 3 is a cross-sectional view of a second organic EL device.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

It is to be understood that although particular phrases used herein, such as “first”, “second”, “third”, and so on, are used to describe different components, members, regions, layers, and/or sections, these components, members, regions, layers, and/or sections should not be limited by these terms. These phrases are used to distinguish one component, member, region, layer, or section from another component, member, region, layer, or section. In this way, a first component, member, region, layer, and/or section to be described below may be referred to as a second component, member, region, layer, and/or section, without departing from the spirit and scope of the present disclosure.

Spatially relative phrases, such as “onto”, “on”, “under”, “below”, “underlying”, “beneath”, “above”, and so on used herein, are used for facilitating description of a relation between one component or feature and another component or feature depicted in the drawings. Therefore, it can be understood that, in addition to directions depicted in the drawings, the spatially relative terms mean to include all different orientations during usage or operations of the device. For example, it is assumed that a device in a figure is reversed upside down, a component described as being “under”, “below”, or “beneath” another component or feature is oriented “onto” or “on” the other component or feature. Therefore, these exemplary terms “under” and “below” may include orientations above and below. The device may be otherwise oriented (e.g., turned by 90 degrees, or other orientations), and the spatially relative terms used herein should be explained accordingly.

Accordingly, it may be understood that when a component or a layer is referred to as being “onto”, “on”, “connected to”, or “coupled to” another component or another layer, it may be immediately on the other component or layer, or connected to or coupled to the other component or layer, or there may be one or more intermediate components or intermediate layers. Further, it can be understood that when a component or a layer is referred to as being “between” two components or two layers, it may be the only component or layer between the two components or layers, or there may be one or more intermediate components or intermediate layers.

Terminologies used herein are only for the purpose of describing particular embodiments, but not limiting the present disclosure. The singular form of “a” and “the” used herein may also include the plural form, unless otherwise indicated in the context. Accordingly, it can be understood that when there terms “include” or “comprise” are used in the specification, it clearly illustrates the existence of a specified feature, bulk, step, operation, component, and/or member, while not excluding the existence or addition of one or more features, bulks, steps, operations, components, members and/or groups thereof. “And/or” used herein includes any and all combinations of one or more related terms that are listed. When a leading word, such as “at least one of”, is added ahead of a component list, it is to describe the entire component list, but not individual components among the list.

The terms “substituted” and “substitution” refer to a substituent bonded to the relevant position, e.g., a carbon or nitrogen. When R₁ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms. A polycyclic aromatic group may have two or more rings possible for being substituted. In this case, a long straight line may be drawn to pass through each of the rings in a formula. The following formula may be an example:

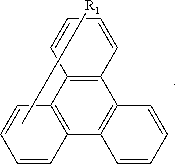

Generally, an organic EL device comprises at least one organic layer disposed between and electrically connected to an anode and a cathode. When an external voltage is applied across the organic EL device, electrons and holes are injected from the cathode and the anode, respectively. Electrons will be injected from a cathode into a LUMO (lowest unoccupied molecular orbital) and holes will be injected from an anode into a HOMO (highest occupied molecular orbital). Subsequently, the electrons recombine with holes in the light emitting layer to form excitons and then emit light. When luminescent molecules absorb energy to achieve an excited state, the exciton may either be in a singlet state or a triplet state, depending on how the spins of the electrons and holes have been combined.

The term “hydrogen” refers to a —H radical.

The terms “halogen” and “halide” are used interchangeably and refer to fluorine, chlorine, bromine, or iodine.

The term “trifluoromethyl” refers to a —CF₃ radical.

The term “cyano” refers to a —C═N radical.

The term “nitro” refers to a —NO₂ radical.

The term “silyl” refers to a —Si(Rₐ)₃ radical, wherein each Rₛ can be same or different. Rₐ can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combinations thereof. Preferred Rs is selected from the group consisting of alkyl, cycloalkyl, aryl, heteroaryl, and combinations thereof.

As used herein, “a first integer to a second integer” indicates a group comprising at least a first integer, a second integer, and all integers therebetween. For example, “1 to 4 atoms” indicates a group comprising 1, 2, 3 and 4 atoms; and “an integer of 0 to 3” indicates a group comprising 0, 1, 2, and 3.

As used herein, “combinations thereof” indicates that one or more members of the applicable list are combined to form a known or chemically stable arrangement that one of ordinary skill in the art can envision from the applicable list. For example, a monocyclic aromatic group and a polycyclic aromatic group can be combined by being joined through a direct bond, or can be combined to have two carbons common to two adjoining rings (the rings are “fused”); a halogen and alkyl can be combined to form a halogenated alkyl substituent; a halogen, alkyl, and aryl can be combined to form a halogenated arylalkyl; and an alkyl and deuterium can be combined to form a partial or fully deuterated alkyl group. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The term “alkyl” refers to and includes both straight and branched chain alkyl radicals. Preferred alkyl groups are those containing 30 or fewer carbon atoms, preferably 1 to 20 carbon atoms, more preferably 1 to 15 carbon atoms, and most preferably 1 to 12 carbon atoms. Suitable alkyl groups include methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “cycloalkyl” as used herein contemplates cyclic alkyl radicals. Preferred cycloalkyl groups are those containing 3 to 10 ring carbon atoms and includes cyclopropyl, cyclopentyl, cyclohexyl, adamantyl, and the like. Additionally, the cycloalkyl group may be optionally substituted.

The term “alkenyl” as used herein contemplates both straight and branched chain alkene radicals. Preferred alkenyl groups are those containing two to fifteen carbon atoms. Additionally, the alkenyl group may be optionally substituted.

The term “alkynyl” as used herein contemplates both straight and branched chain alkyne radicals. Preferred alkynyl groups are those containing two to fifteen carbon atoms. Additionally, the alkynyl group may be optionally substituted.

The term “heterocyclic group” as used herein contemplates aromatic and non-aromatic cyclic radicals. Hetero-aromatic cyclic radicals also means heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers, such as tetrahydrofuran, tetrahydropyran, and the like. Additionally, the heterocyclic group may be optionally substituted.

The term “aryl” or “aromatic group” as used herein contemplates a monocyclic aromatic group, a polycyclic aromatic group, and combinations thereof. The polycyclic aromatic group may have two, three, four or more rings in which two carbons are common to two adjoining rings (the rings are “fused”) wherein at least one of the fused rings is an aromatic hydrocarbyl, e.g., the other rings can be cycloalkyls, cycloalkenyls, aryl, heterocycles, and/or heteroaryls. Unless otherwise specified, preferred aryl groups are those containing 30 or fewer carbon atoms, preferably 6 to 30 carbon atoms, more preferably 6 to 20 carbon atoms, and most preferably 6 to 12 carbon atoms. Especially preferred is an aryl group having 6 carbons, 10 carbons or 12 carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group is optionally substituted.

The term “arylene” or “arenediyl” as used herein contemplates a substituent of an organic compound that is derived from an aromatic hydrocarbon (arene) that has had a hydrogen atom removed from two ring carbon atoms, such as phenylene. Unless otherwise specified, preferred arylene groups are those containing 30 or fewer carbon atoms, preferably 6 to 30 carbon atoms, more preferably 6 to 20 carbon atoms, and most preferably 6 to 12 carbon atoms. Especially preferred is an arylene group having 6 carbons, 10 carbons or 12 carbons. Additionally, the arylene group is optionally substituted.

The terms “aralkyl” or “arylalkyl” are used interchangeably and refer to an alkyl group that is substituted with an aryl group. Preferred aralkyl groups are those containing 30 or fewer carbon atoms, preferably 6 to 30 carbon atoms, more preferably 7 to 30 carbon atoms, and most preferably 7 to 13 carbon atoms. Additionally, the aralkyl group is optionally substituted.

The term “heteroaryl” as used herein contemplates a monocyclic aromatic group and a polycyclic aromatic group that both include at least one heteroatom. The heteroatoms include, but are not limited to O, S, N, P, B, Si, and Se. In many instances, O, S, Se, N or Si are the preferred heteroatoms. Hetero-monocyclic aromatic groups are preferably single rings with 5 or 6 ring atoms, and the ring can have from one to six heteroatoms. The hetero-polycyclic aromatic groups can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic groups can have from one to six heteroatoms per ring of the polycyclic aromatic group. Preferred heteroaryl groups are those containing 30 or fewer carbon atoms, preferably 3 to 30 carbon atoms, more preferably 3 to 20 carbon atoms, and most preferably 3 to 12 carbon atoms. Suitable heteroaryl groups include but not limited to dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

The terms alkyl, aralkyl, heteroaryl, aryl, cycloalkyl, heteroalkyl, heterocycloalkyl, alkenyl, cycloalkenyl, heteroalkenyl, alkynyl, alkoxy, and heterocyclic group, as used herein, are independently unsubstituted, or independently substituted, with one or more general substituents selected from the group consisting of deuterium, halogen, alkyl, cycloalkyl, heteroalkyl, arylalkyl, alkoxy, aryloxy, amino, cyclic amino, silyl, alkenyl, cycloalkenyl, heteroalkenyl, alkynyl, aryl, heteroaryl, acyl, carbonyl, carboxylic acid, ether, ester, nitrile, isonitrile, sulfanyl, sulfinyl, sulfonyl, phosphino, and combinations thereof.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

In some instances, the preferred general substituents are selected from the group consisting of hydrogen, halogen, trifluoromethyl, cyano, nitro, silyl, and combinations thereof.

In yet other instances, the more preferred general substituents are selected from the group consisting of hydrogen, alkyl, aralkyl, heteroaryl and combinations thereof.

The term “acyl” refers to a substituted carbonyl radical (C(O)—Rₛ).

The term “ester” refers to a substituted oxycarbonyl (—O—C(O)—Rₛ or —C(O)—O—Rₛ) radical.

The term “ether” refers to an —ORₛ radical.

The terms “sulfanyl” or “thio-ether” are used interchangeably and refer to a —SRₛ radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

It is to be understood that when a molecular fragment is described as being a substituent or otherwise attached to another moiety, its name may be written as if it were a fragment (e.g., phenyl, phenylene, naphthyl, dibenzofuryl, hydrocarbyl, aromatic linker, arylene) or as if it were the whole molecule (e.g., benzene, naphthalene, dibenzofuran, hydrocarbon, aromatic compound, aromatic hydrocarbon). As used herein, these different ways of designating a substituent or attached fragment are considered to be equivalent.

In one embodiment, an organic compound is represented by the following formula:

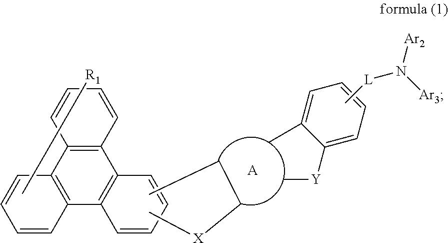

wherein X and Y independently represent a divalent bridge selected from the group consisting of O, S, NAr₁ and CR₂R₃;

wherein ring A represents a monocyclic aromatic group or a polycyclic aromatic group having at least two fused rings;

wherein L represents a single bond or an aromatic linker;

wherein R₁ to R₃ independently represent no substitution or mono to the maximum allowable substitution, each of the substituents is selected from the group consisting of alkyl, aryl, aralkyl, heteroaryl, and combinations thereof; and

wherein each of Ar₁, Ar₂ and Ar₃ is selected from the group consisting of aryl, heteroaryl, and combinations thereof.

In selected embodiments, the organic compound is represented by one of the following formulae:

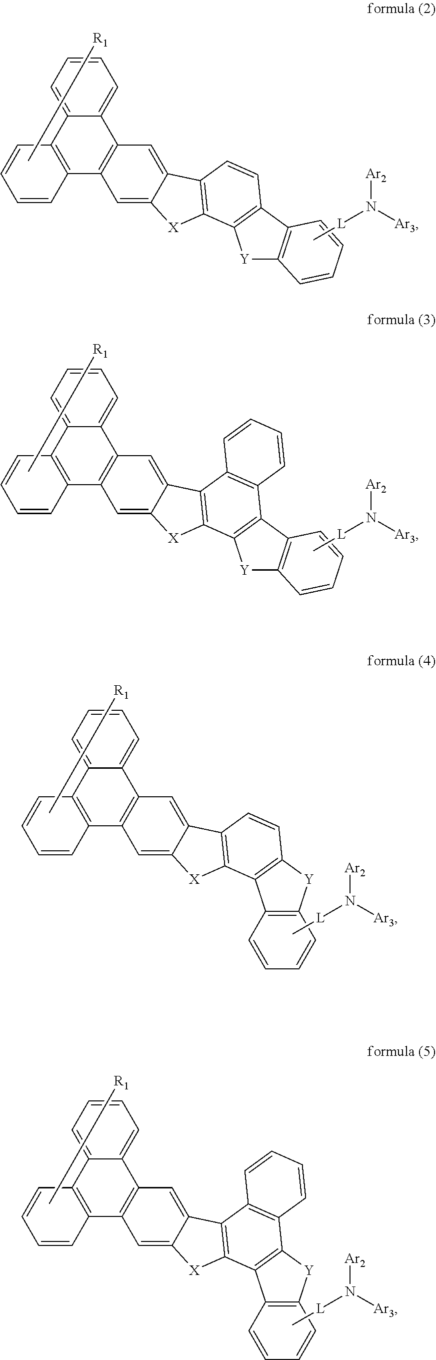

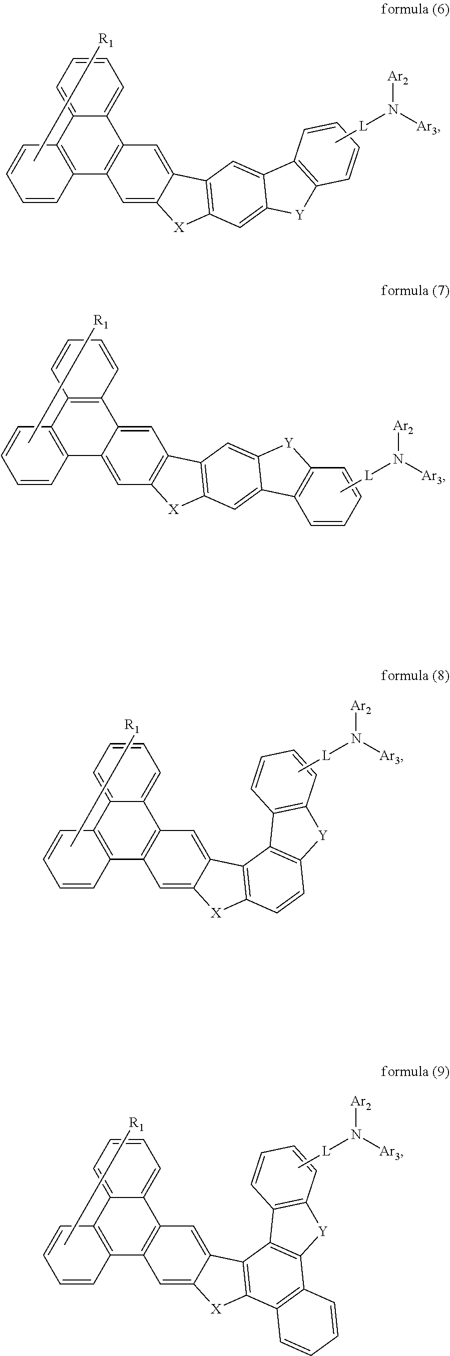

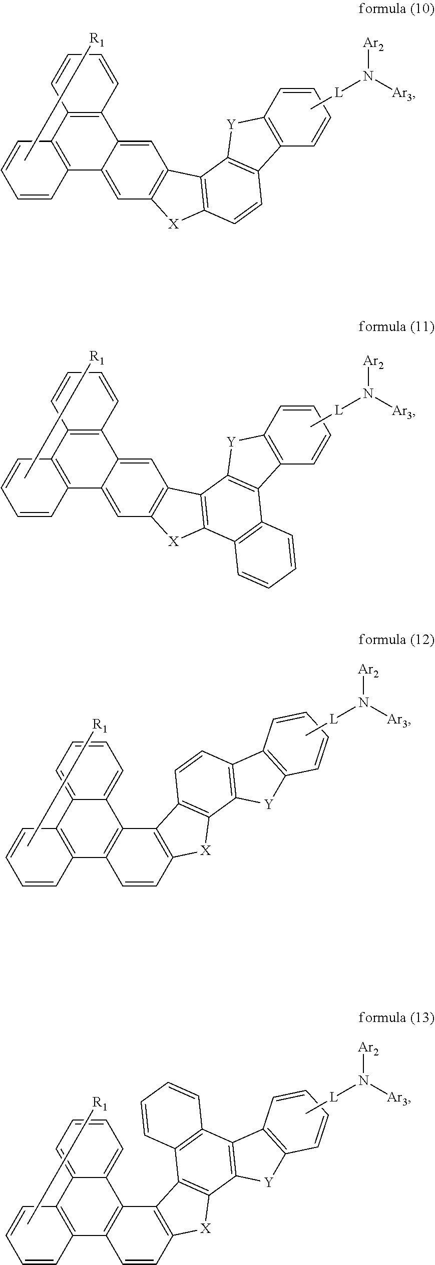

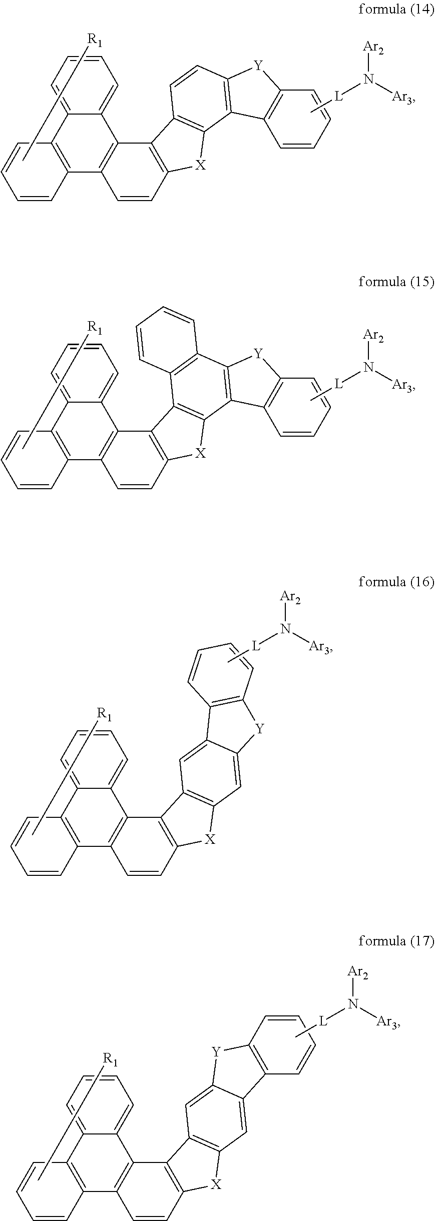

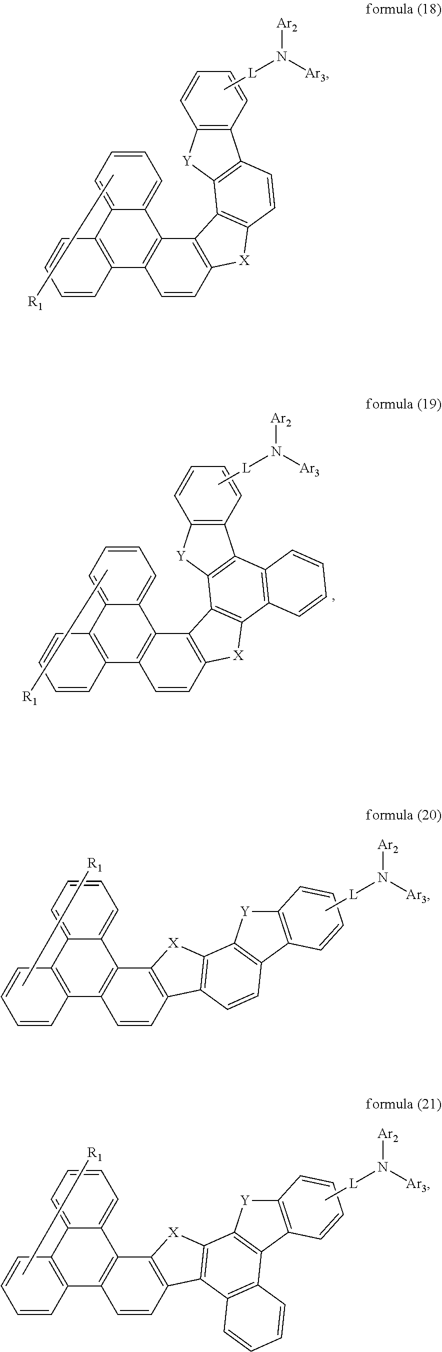

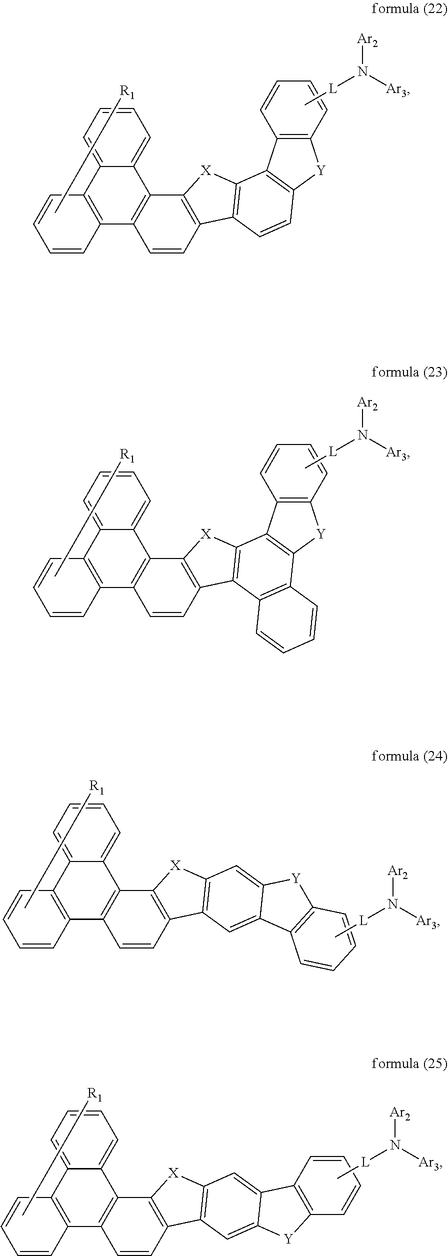

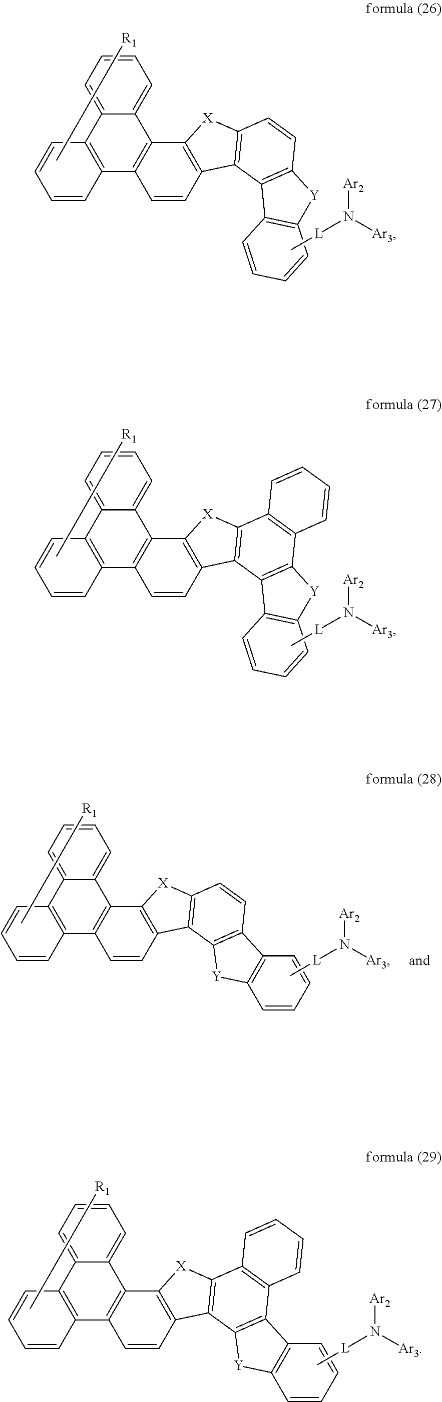

In some embodiments, Ar₂ and Ar₃ are independently selected from the group consisting of biphenyl, terphenyl, quaterphenyl, fluorenyl, carbazolyl, dibenzofuranyl, dibenzothiophenyl, naphtyl, anthracyl, phenanthryl, chrysenyl, triphenylenyl, pyrenyl, and combinations thereof.

In some embodiments, Ar₂ and Ar₃ are independently selected from the group consisting of:

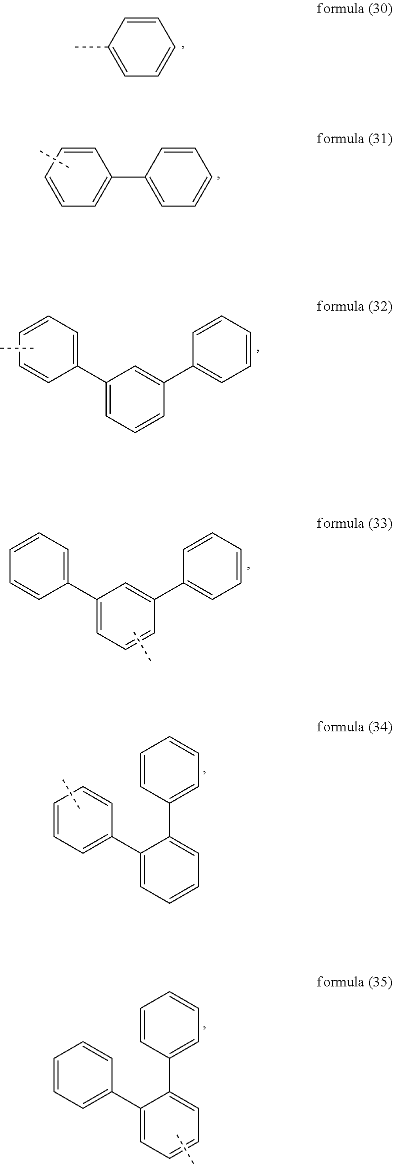

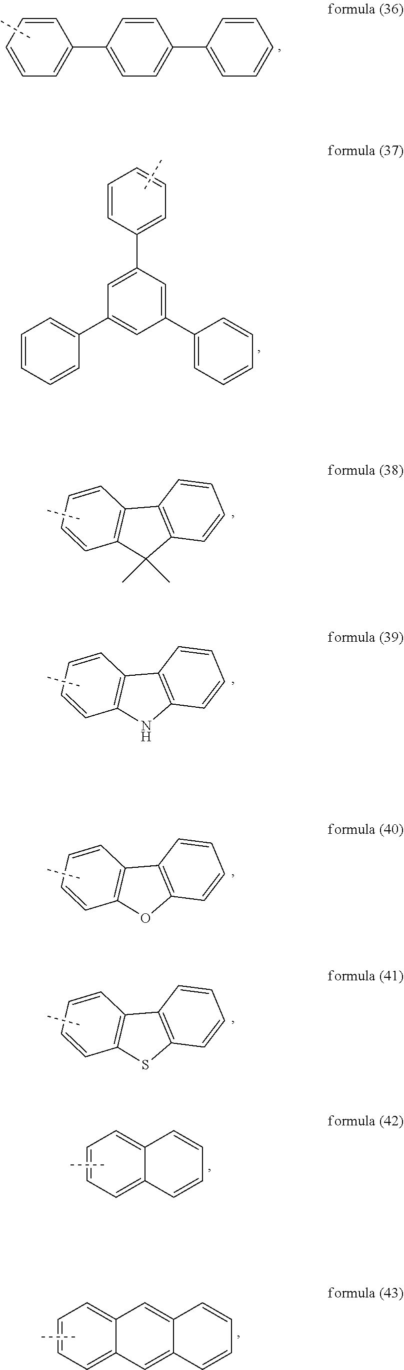

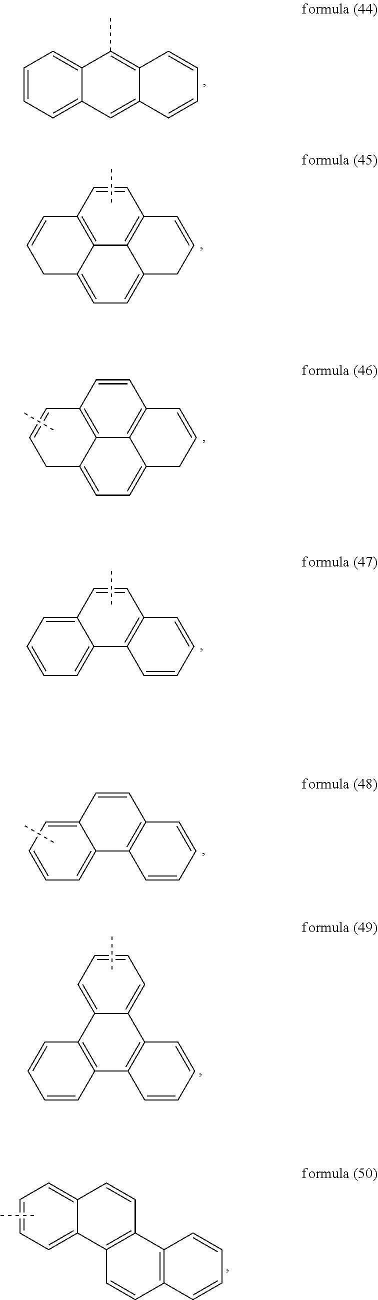

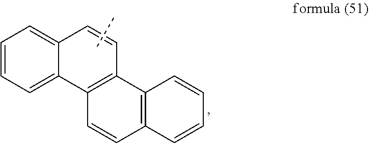

combinations thereof.

In some embodiments, each of the R₁ to R₃ represents no substitution or is a substituent selected from the group consisting of alkyl having 20 or fewer carbon atoms, aryl having 30 or fewer carbon atoms, aralkyl having 30 or fewer carbon atoms, heteroaryl having 30 or fewer carbon atoms, and combinations thereof.

In some embodiments, R₁ may represent no substitution or mono, di, tri, tetra, penta, hexa, hepta, or octa substitutions. L may represent a single bond or an aromatic linker selected from the group consisting of alkyl, aryl, aralkyl, heteroaryl, and combinations thereof. L may also represent an aromatic linker of arylene. Alternatively, L may represent a substituted or unsubstituted divalent arylene having 6 to 30 ring carbon atoms.

X and Y may independently represent a divalent bridge selected from the group consisting of O, S, NAr₁ and CR₂R₃; m represents an integer of 0 to 8. Ring A may represent a fused ring hydrocarbon unit with one, two rings. L may represent a single bond, a substituted or unsubstituted divalent arylene group having 6 to 30 ring carbon atoms. R₁ to R₃ may be independently selected from the group consisting of a hydrogen atom, a substituted or unsubstituted alkyl group having 1 to 20 carbon atoms, a substituted or unsubstituted aryl group having 6 to 30 carbon atoms, a substituted or unsubstituted aralkyl group having 6 to 30 carbon atoms and a substituted or unsubstituted heteroaryl group having 3 to 30 carbon atoms.

In formula (1), Ar₁ may represent a substituted or unsubstituted aryl group having 6 to 30 carbon atoms and a substituted or unsubstituted heteroaryl group having 3 to 30 carbon atoms. Ar₂ and Ar₃ may independently represent a substituted or unsubstituted aryl group having 6 to 30 carbon atoms, and a substituted or unsubstituted heteroaryl group having 6 to 30 carbon atoms.

Ar may represent a substituted or unsubstituted aryl group having 6 to 30 carbon atoms. R₁ to R₈ may be independently selected from the group consisting of a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 7 to 30 carbon atoms; a substituted or unsubstituted arylamine group having 6 to 30 ring carbon atoms, and a substituted or unsubstituted heteroarylamine group having 5 to 30 ring carbon atoms. The alkyl group, aralkyl group, aryl group, heteroaryl group, arylamine group, or heteroarylamine group is substituted by, for example, a halogen, an alkyl group, an aryl group, or a heteroaryl group.

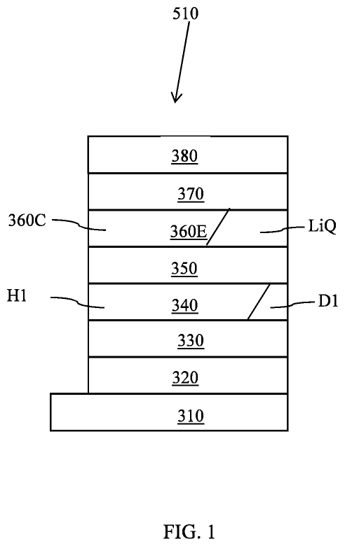

In one embodiment, a first organic EL device using the organic compound of formula (1) is disclosed. FIG. 1 is a cross-sectional view of the first organic EL device. Referring to FIG. 1, the first organic EL device 510 may comprise the organic compound of formula (1) as an electron transporting material 360C of an electron transport layer 360E.

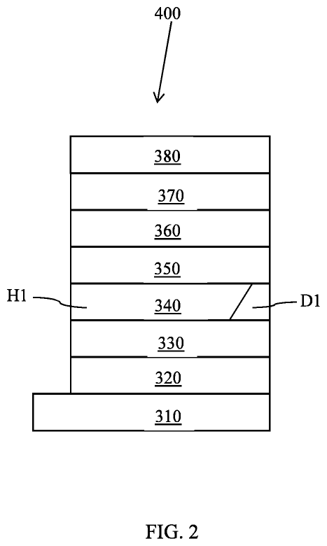

FIG. 2 is a cross-sectional view of an organic EL device without the organic compound of formula (1) (without 360C of FIG. 1). Referring to FIG. 2, the organic EL device 400 may have a driving voltage of about 4.5 V, a current efficiency of about 18 cd/A, or a half-life of about 420 hours.

Referring to FIG. 1, by comprising the organic compound of formula (1) as the electron transporting material 360C, the first organic EL device 510 may have a driving voltage lower than that of the organic EL device 400 (FIG. 2). Moreover, by comprising the organic compound of formula (1) as the electron transporting material 360C, the first organic EL device 510 of FIG. 1 may have a current efficiency higher than that of the organic EL device 400 (FIG. 2). Furthermore, by comprising the organic compound of formula (1) as the electron transporting material 360C, the first organic EL device 510 of FIG. 1 may have a half-life longer than that of the organic EL device 400 (FIG. 2).

As the electron transporting material 360C of the first organic EL device 510 of FIG. 1, the organic compound of formula (1) may lower the driving voltage to be about 3.5 V to about 4.5 V. Moreover, the organic compound of formula (1) may increase the current efficiency to be 18 cd/A to about 27 cd/A. Furthermore, the organic compound of formula (1) may increase the half-life to be about 430 hours to about 530 hours.

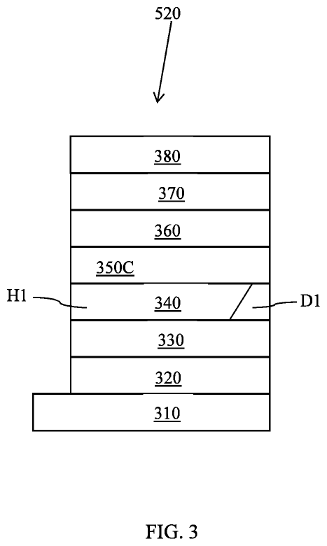

In one embodiment, a second organic EL device using the organic compound of formula (1) is disclosed. FIG. 3 is a cross-sectional view of the second organic EL device. Referring to FIG. 3, the second organic EL device 520 may comprise the organic compound of formula (1) as a hole blocking layer 350C.

FIG. 2 is a cross-sectional view of an organic EL device without the organic compound of formula (1) (without 350C of FIG. 3). Referring to FIG. 2, the organic EL device 400 may have a driving voltage of about 4.5 V, a current efficiency of about 18 cd/A, or a half-life of about 420 hours.

Referring to FIG. 3, by comprising the organic compound of formula (1) as the hole blocking layer 350C, the second organic EL device 520 may have a driving voltage lower than that of the organic EL device 400 (FIG. 2). Moreover, by comprising the organic compound of formula (1) as the hole blocking layer 350C, the second organic EL device 520 of FIG. 3 may have a current efficiency higher than that of the organic EL device 400 (FIG. 2). Furthermore, by comprising the organic compound of formula (1) as the hole blocking layer 350C, the second organic EL device 520 of FIG. 3 may have a half-life longer than that of the organic EL device 400 (FIG. 2).

Referring to FIG. 3, as the hole blocking layer 350C of the second organic EL device 520, the organic compound of formula (1) may lower the driving voltage to be about 3.9 V to about 4.9 V. Moreover, the organic compound of formula (1) may increase the current efficiency to be about 18 cd/A to about 26 cd/A. Furthermore, the organic compound of formula (1) may increase the half-life to be about 380 hours to about 510 hours.

In formula (1), Ar₁, Ar₂ or Ar₃ may represent a substituted or unsubstituted phenyl group, a substituted or unsubstituted biphenyl group, a substituted or unsubstituted fluorene group, a substituted or unsubstituted benzofluorene group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group.

The organic compound of the present invention may also be represented by one of the following formulae:

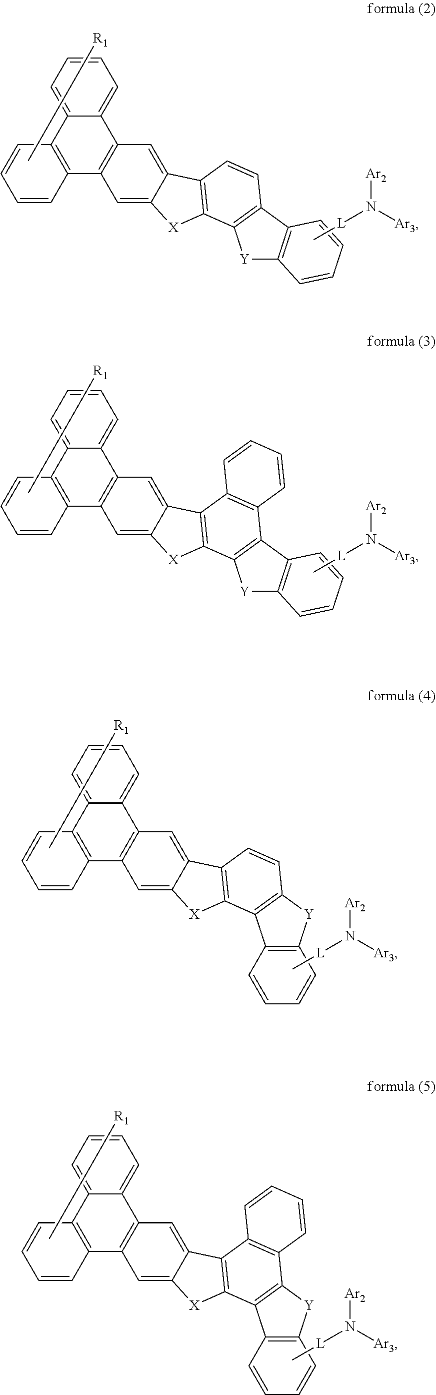

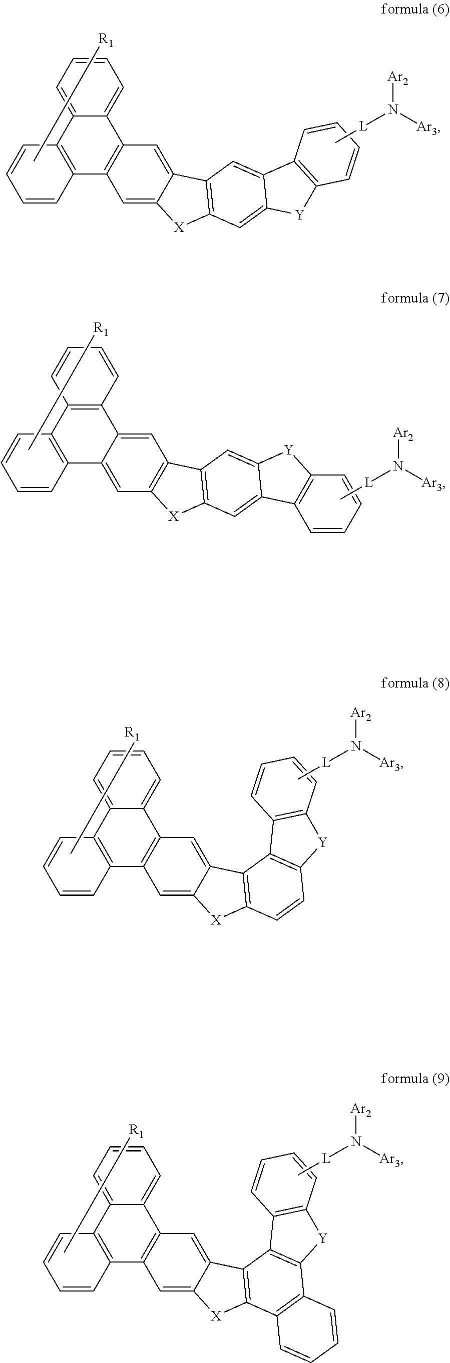

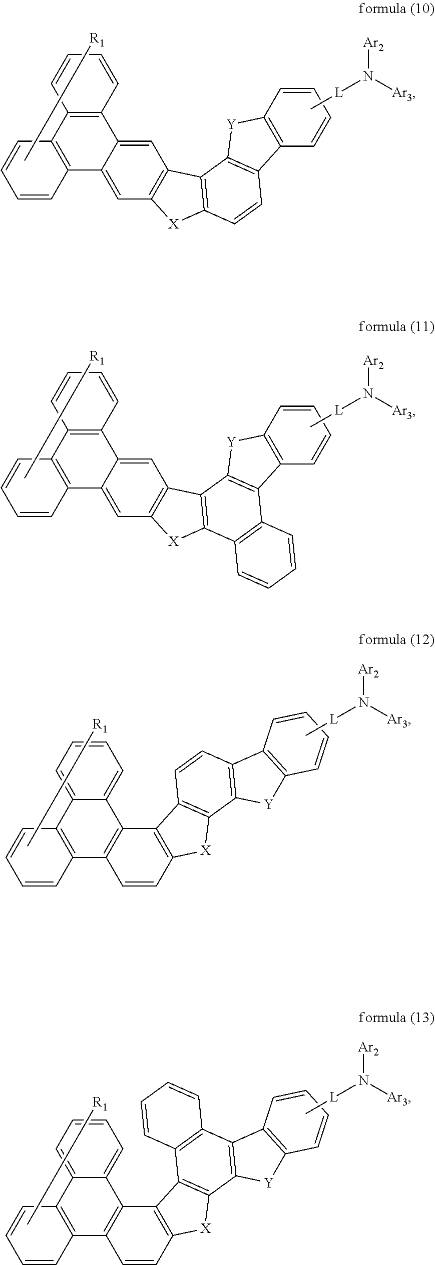

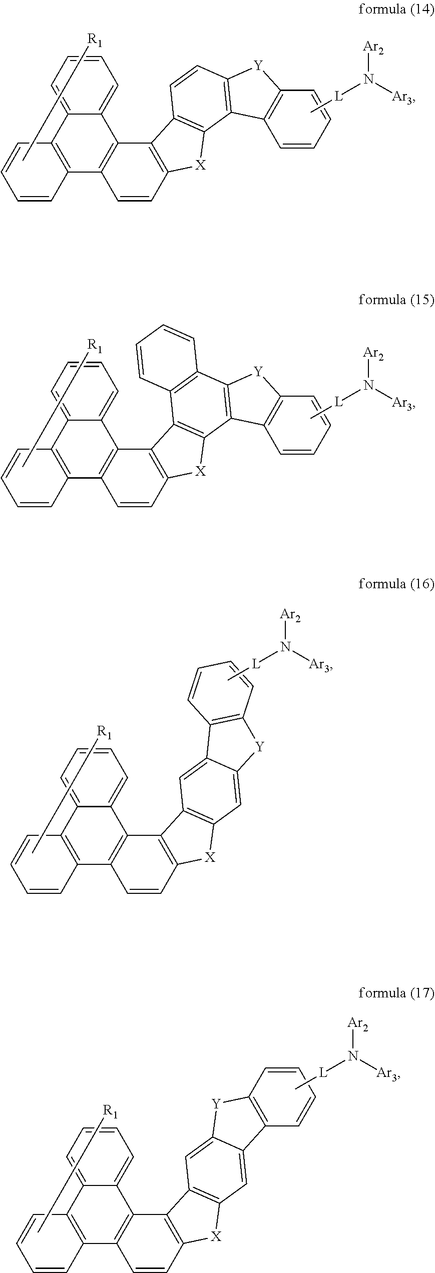

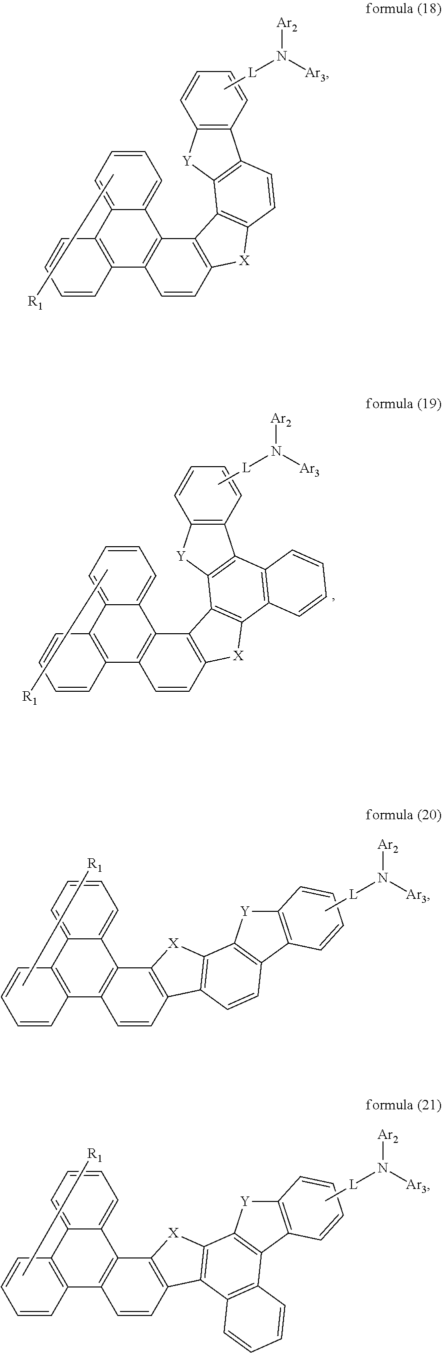

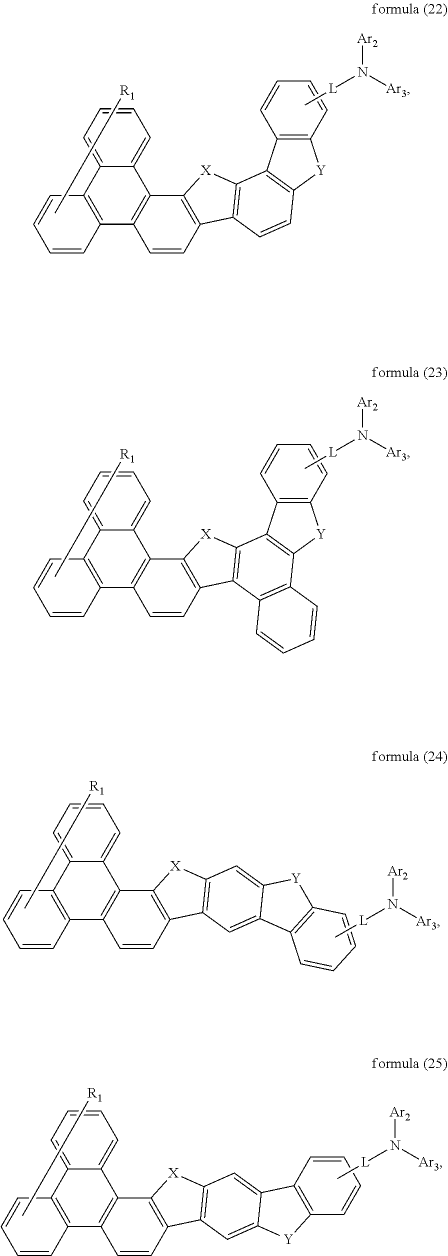

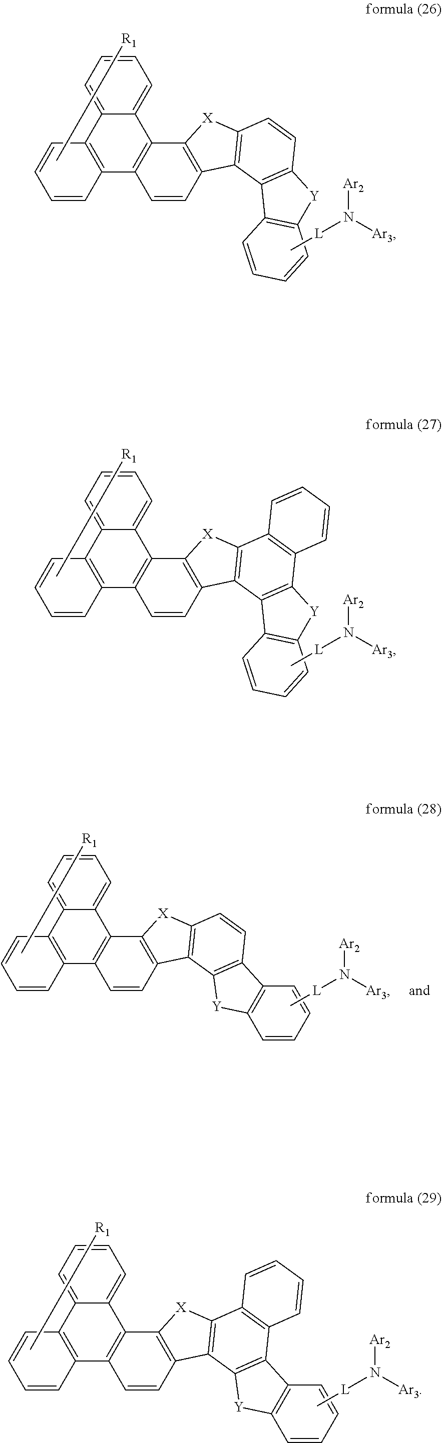

The same definition as described in the paragraph [0010] to paragraph [0065].

In selected embodiments, Ar₂ and Ar₃ are independently selected from the group consisting of biphenyl, terphenyl, quaterphenyl, fluorenyl, carbazolyl, dibenzofuranyl, dibenzothiophenyl, naphtyl, anthracyl, phenanthryl, chrysenyl, triphenylenyl, pyrenyl, and combinations thereof.

In selected embodiments, Ar₂ and Ar₃ are independently selected from the group consisting of:

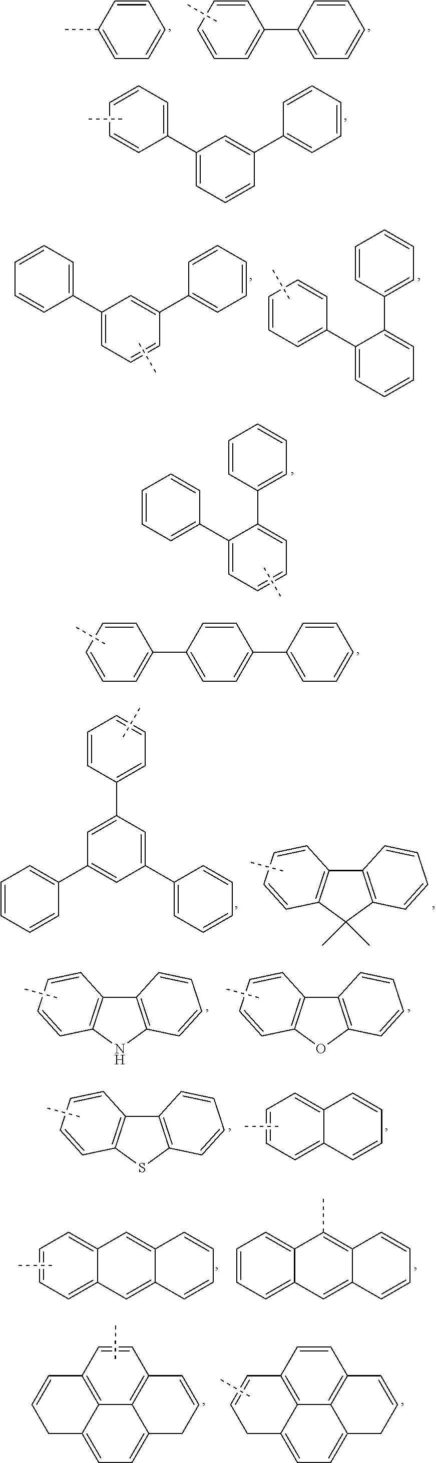

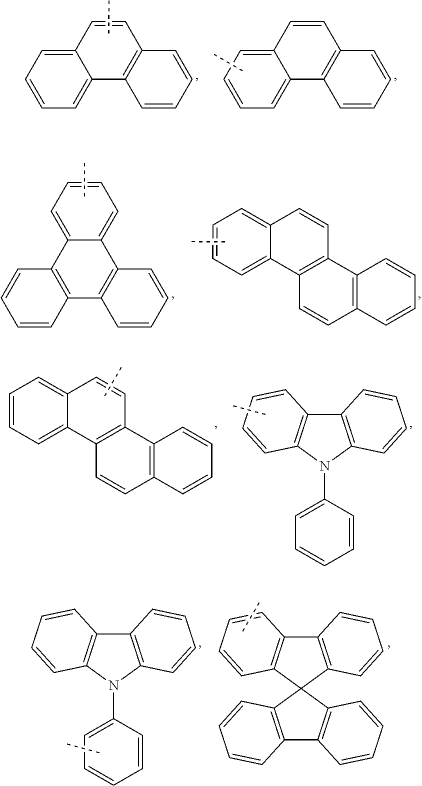

and combinations thereof.

The organic compound of the present invention may be one of the following compounds:

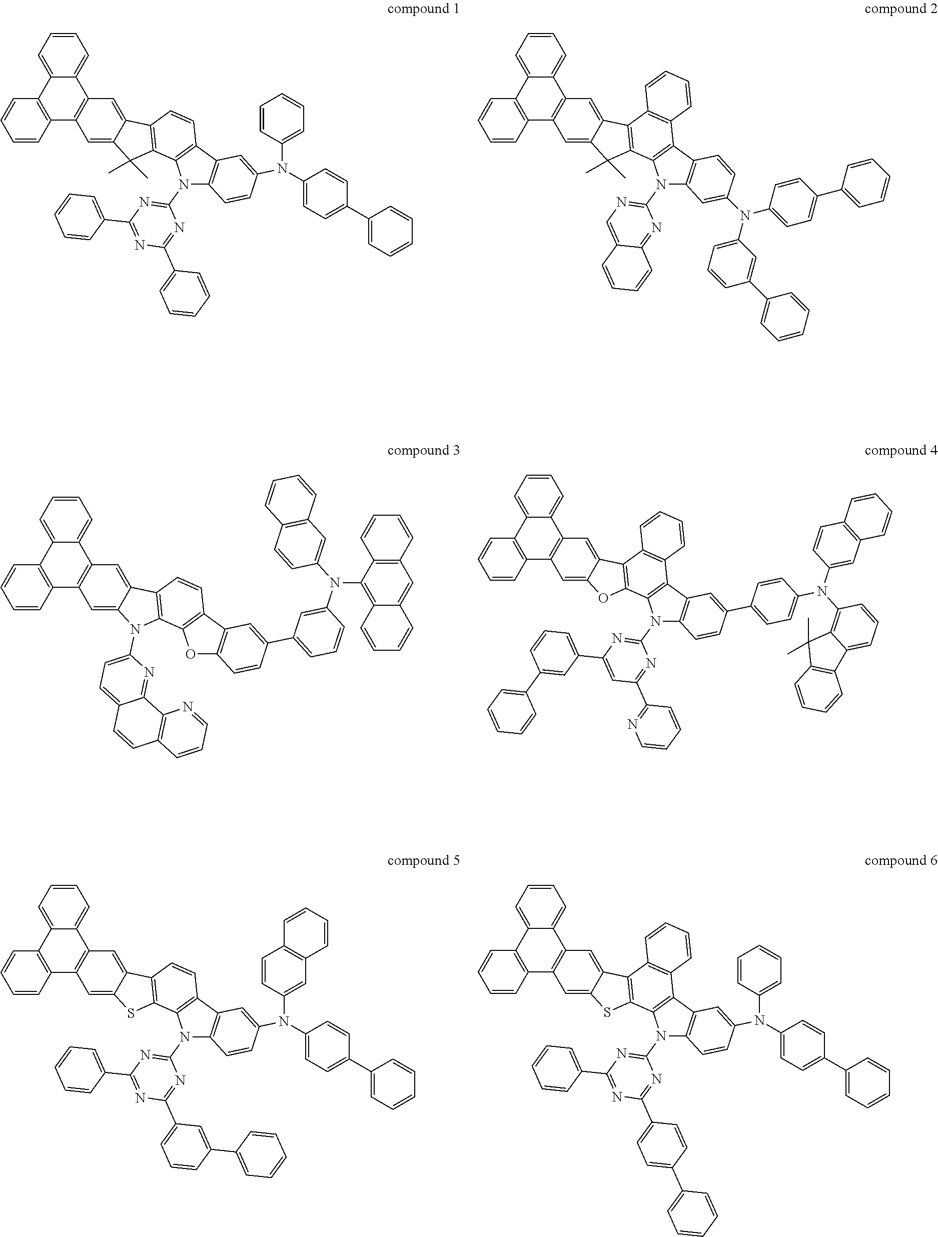

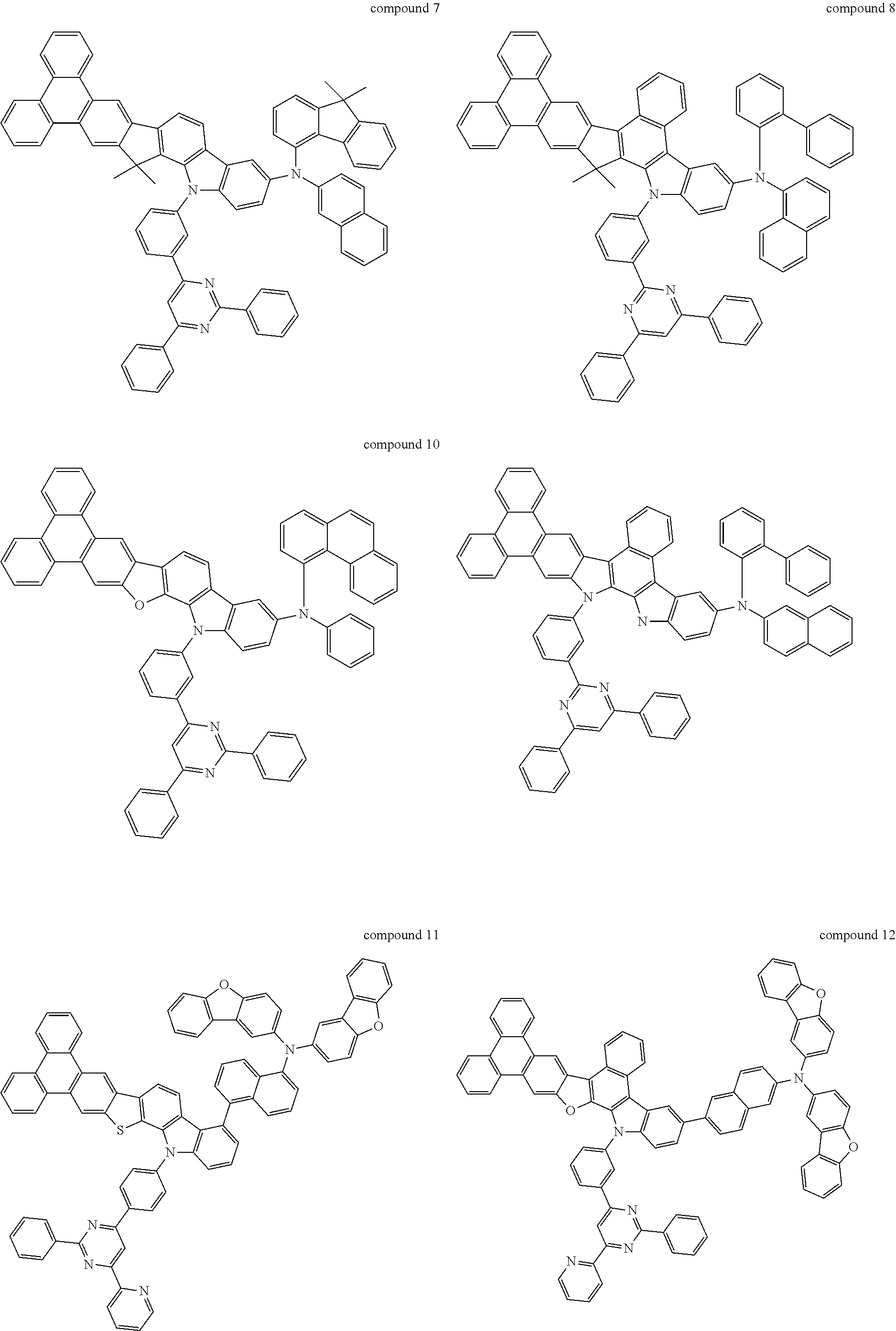

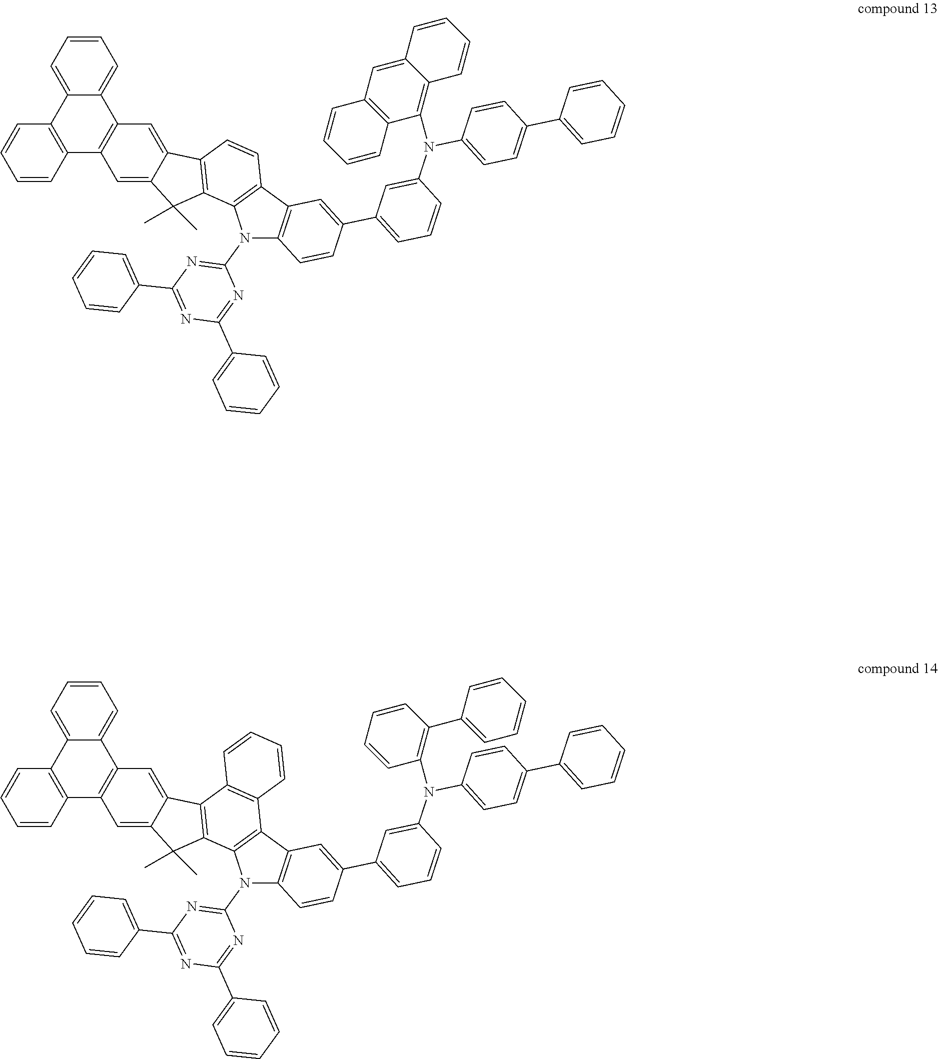 [SMILES: CC1(C)c2cc3c4ccccc4c4ccccc4c3cc2-c2ccc3c4cc(-c5cccc(N(c6ccc(-c7ccccc7)cc6)c6c7ccccc7cc7ccccc67)c5)ccc4n(-c4nc(-c5ccccc5)nc(-c5ccccc5)n4)c3c21 ; CC1(C)c2cc3c4ccccc4c4ccccc4c3cc2-c2c1c1c(c3ccccc23)c2cc(-c3cccc(N(c4ccc(-c5ccccc5)cc4)c4ccccc4-c4ccccc4)c3)ccc2n1-c1nc(-c2ccccc2)nc(-c2ccccc2)n1]

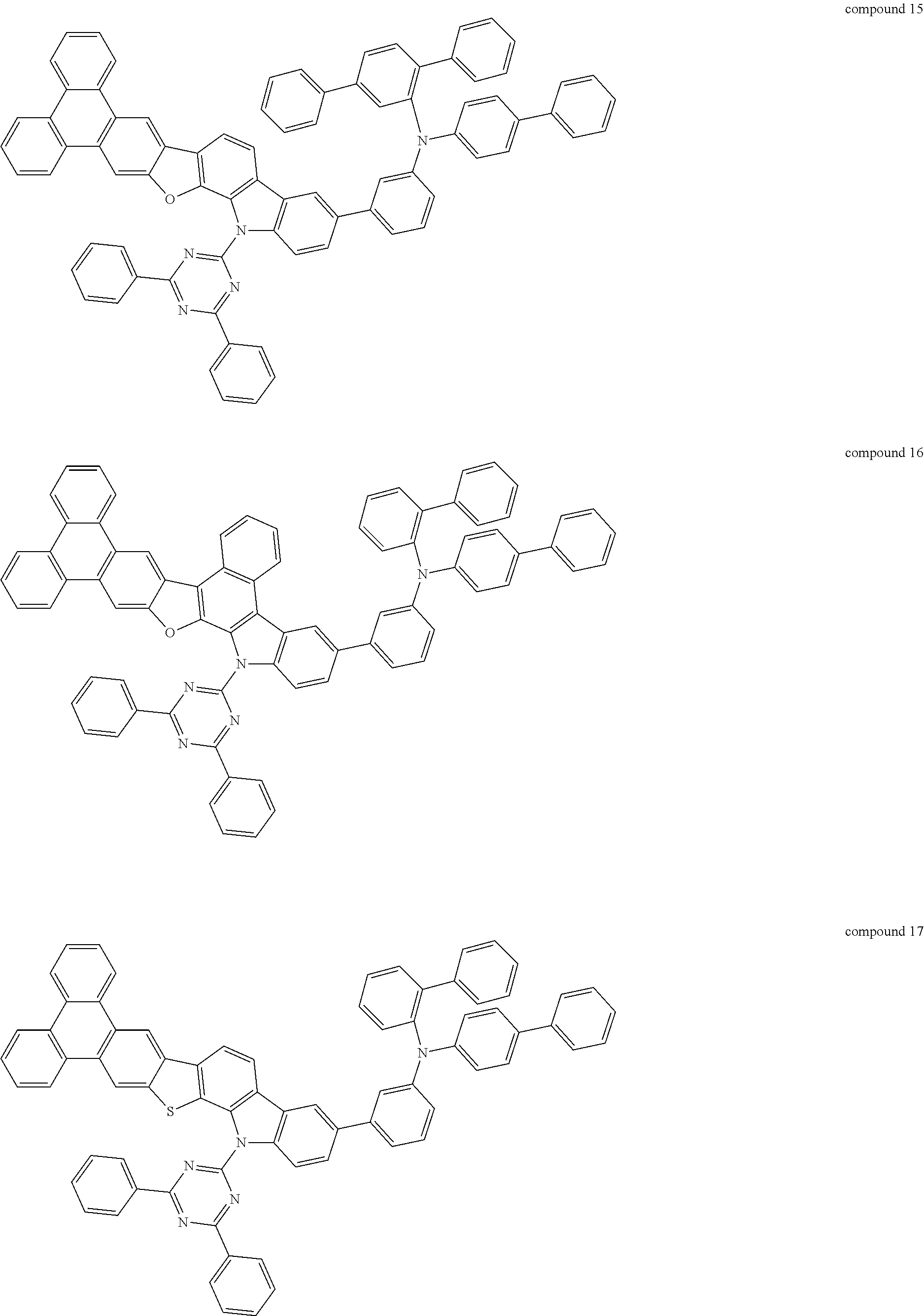 [SMILES: c1ccc(-c2ccc(N(c3cccc(-c4ccc5c(c4)c4ccc6c7cc8c9ccccc9c9ccccc9c8cc7oc6c4n5-c4nc(-c5ccccc5)nc(-c5ccccc5)n4)c3)c3cc(-c4ccccc4)ccc3-c3ccccc3)cc2)cc1 ; c1ccc(-c2ccc(N(c3cccc(-c4ccc5c(c4)c4c6ccccc6c6c7cc8c9ccccc9c9ccccc9c8cc7oc6c4n5-c4nc(-c5ccccc5)nc(-c5ccccc5)n4)c3)c3ccccc3-c3ccccc3)cc2)cc1 ; c1ccc(-c2ccc(N(c3cccc(-c4ccc5c(c4)c4ccc6c7cc8c9ccccc9c9ccccc9c8cc7sc6c4n5-c4nc(-c5ccccc5)nc(-c5ccccc5)n4)c3)c3ccccc3-c3ccccc3)cc2)cc1]

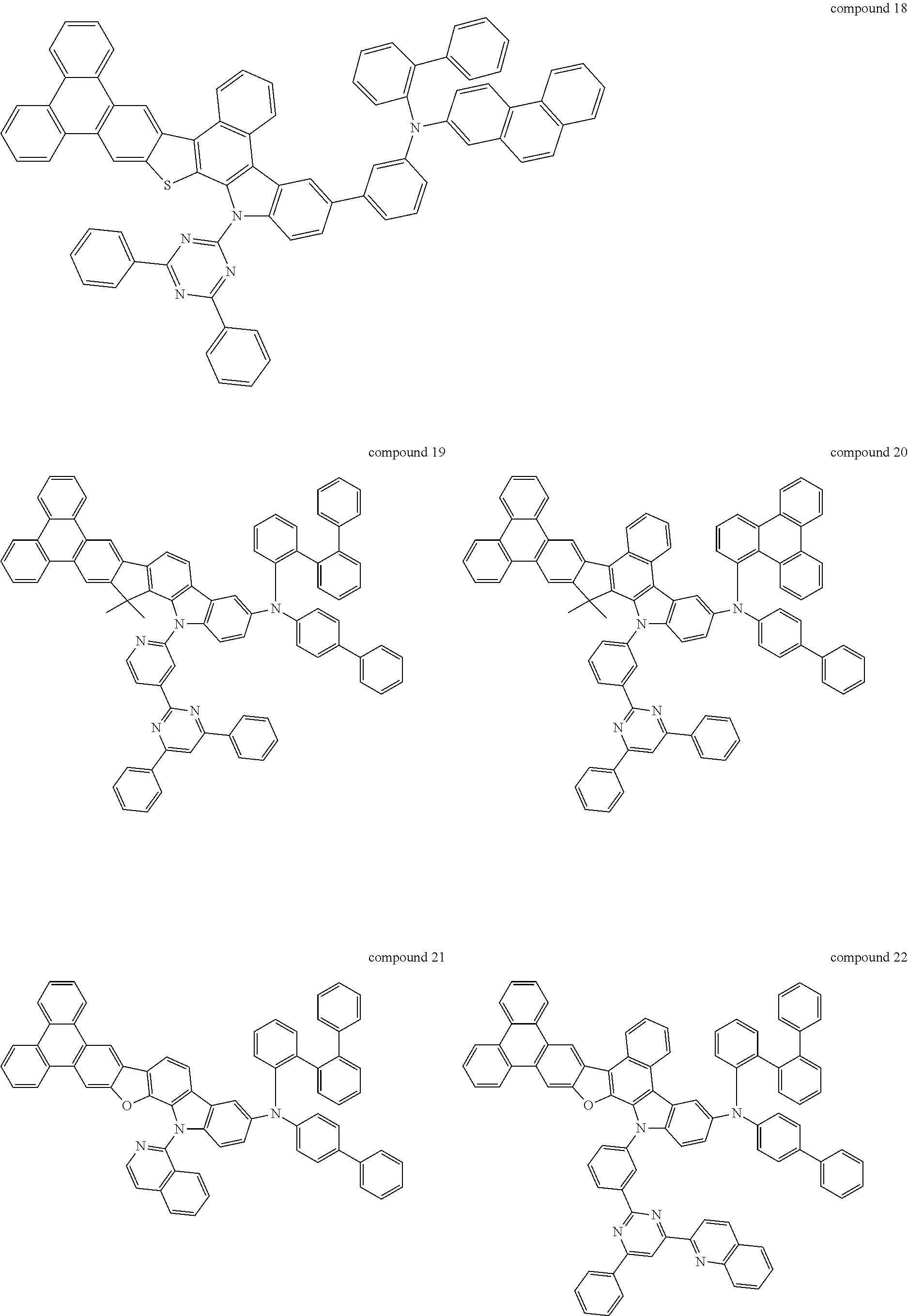

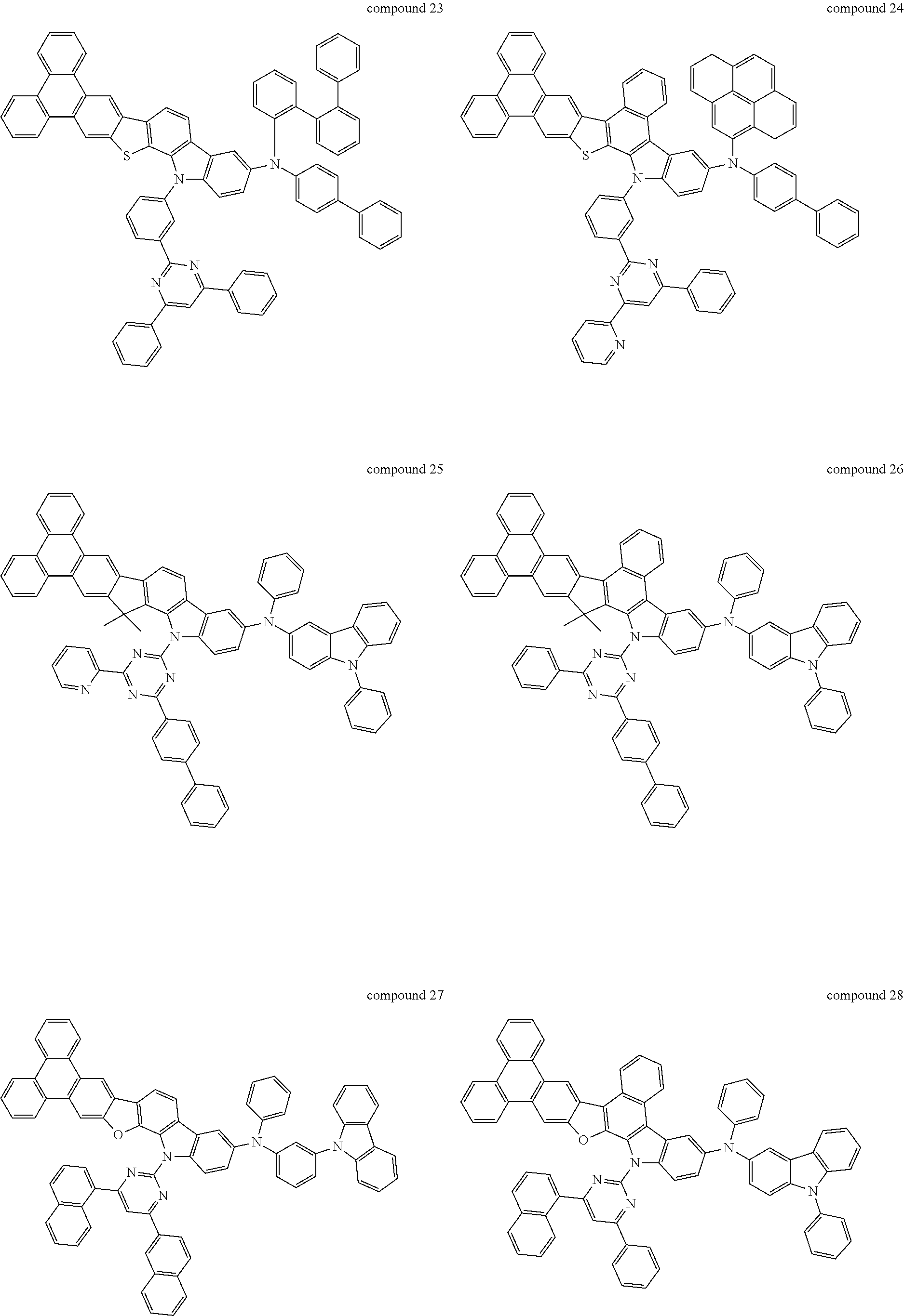

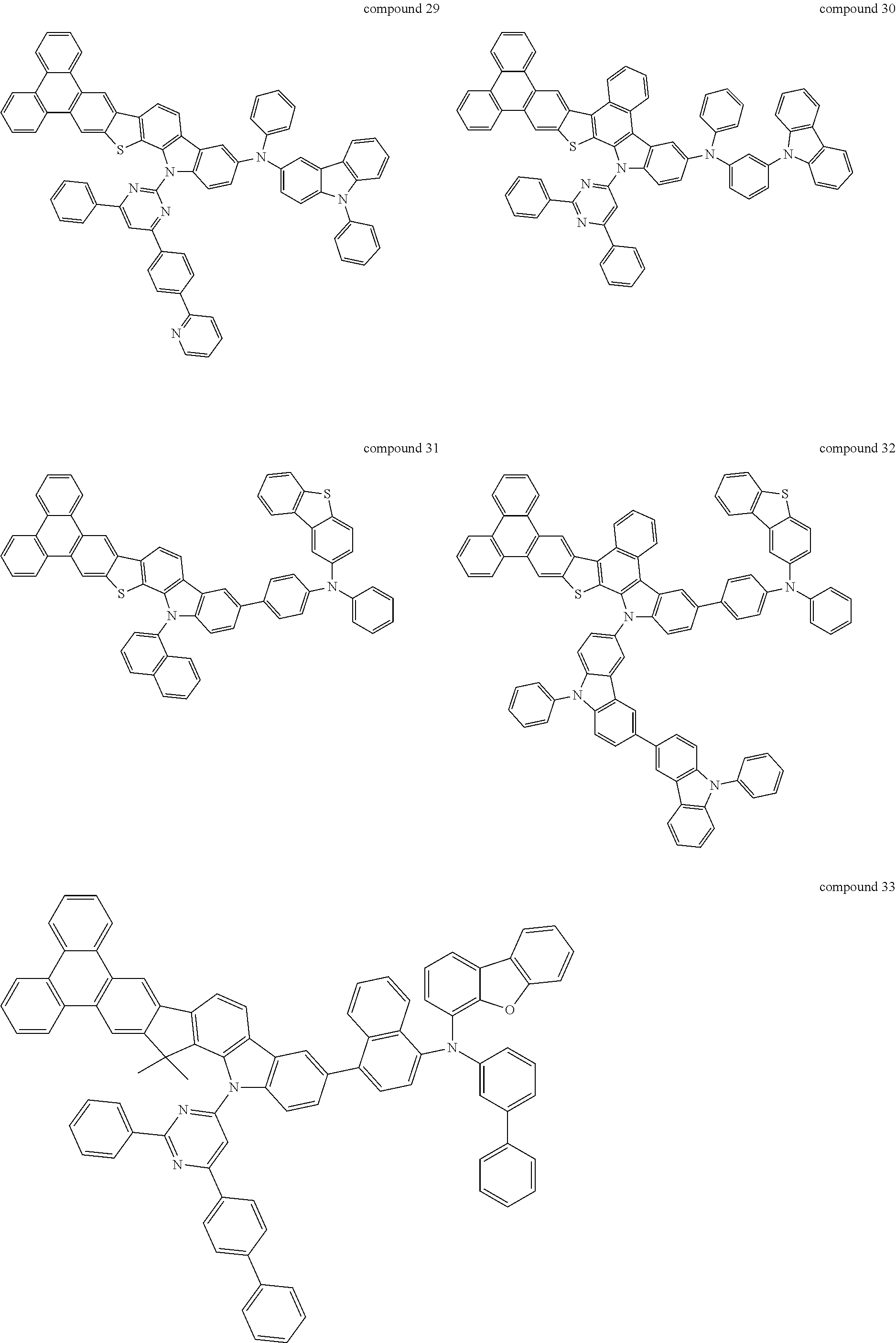

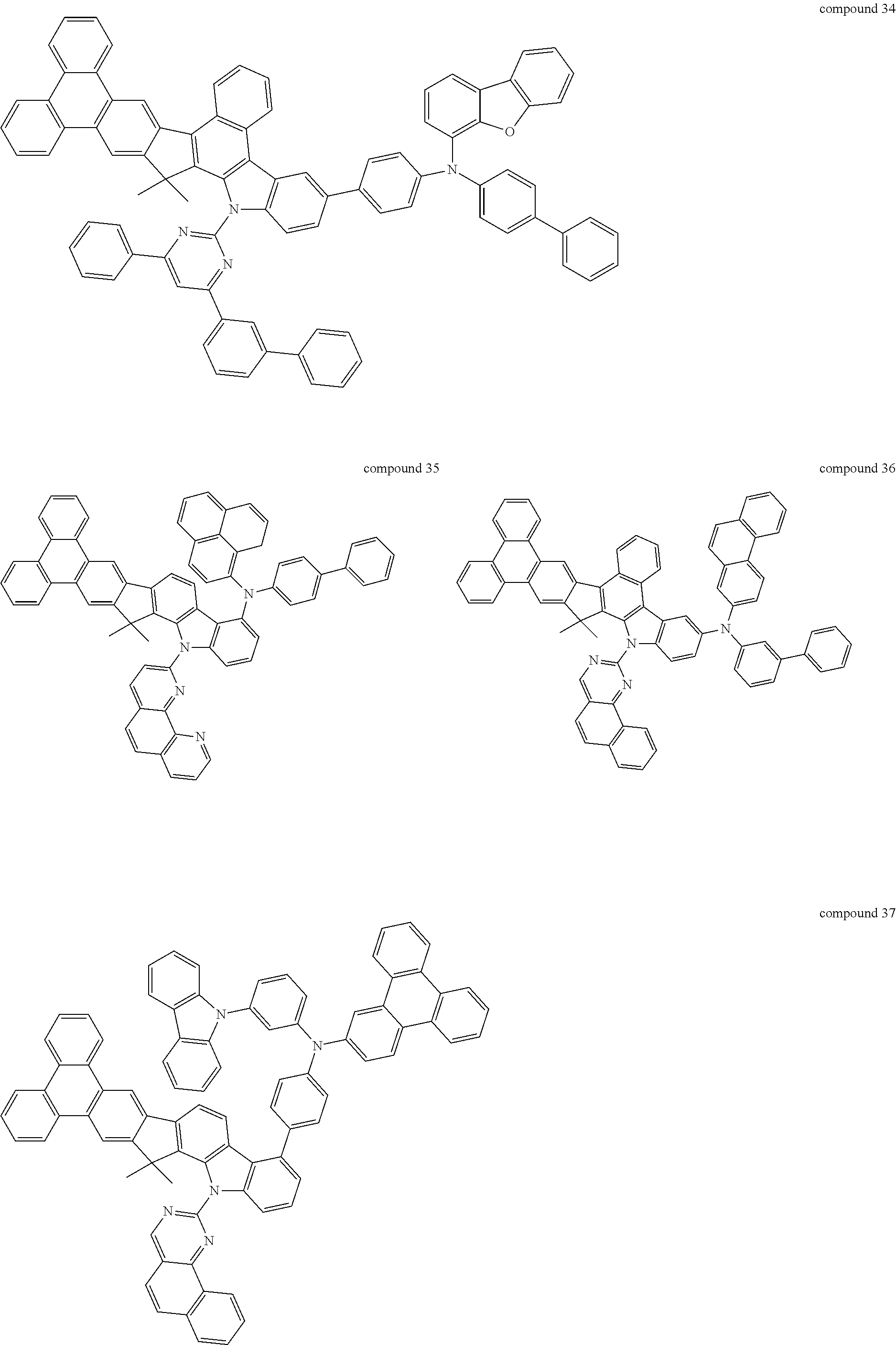

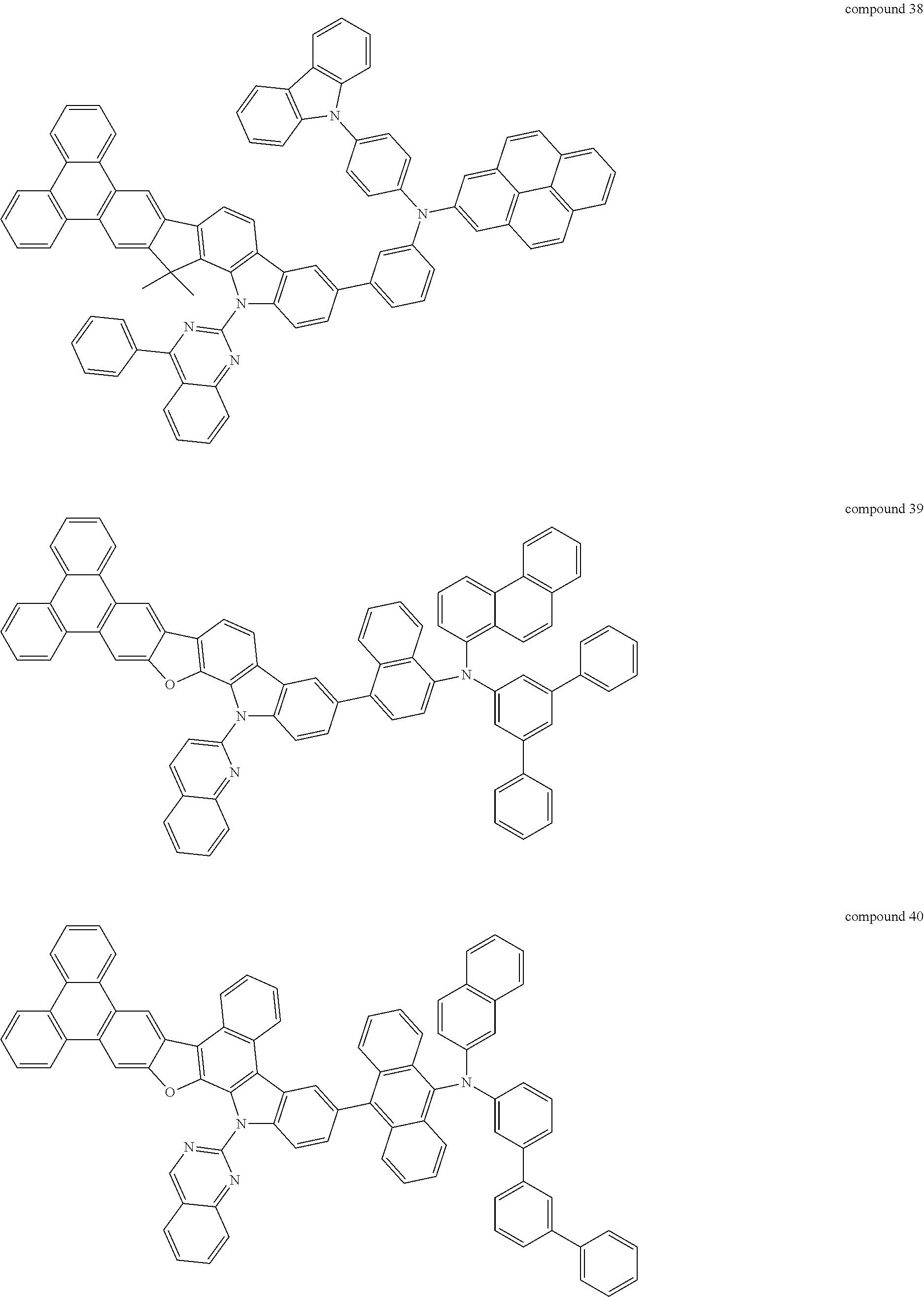 [SMILES: CC1(C)c2cc3c4ccccc4c4ccccc4c3cc2-c2ccc3c4cc(-c5cccc(N(c6ccc(-n7c8ccccc8c8ccccc87)cc6)c6cc7ccc8cccc9ccc(c6)c7c89)c5)ccc4n(-c4nc(-c5ccccc5)c5ccccc5n4)c3c21 ; c1ccc(-c2cc(-c3ccccc3)cc(N(c3ccc(-c4ccc5c(c4)c4ccc6c7cc8c9ccccc9c9ccccc9c8cc7oc6c4n5-c4ccc5ccccc5n4)c4ccccc34)c3cccc4c3ccc3ccccc34)c2)cc1 ; c1ccc(-c2cccc(-c3cccc(N(c4ccc5ccccc5c4)c4c5ccccc5c(-c5ccc6c(c5)c5c7ccccc7c7c8cc9c%10ccccc%10c%10ccccc%10c9cc8oc7c5n6-c5ncc6ccccc6n5)c5ccccc45)c3)c2)cc1]

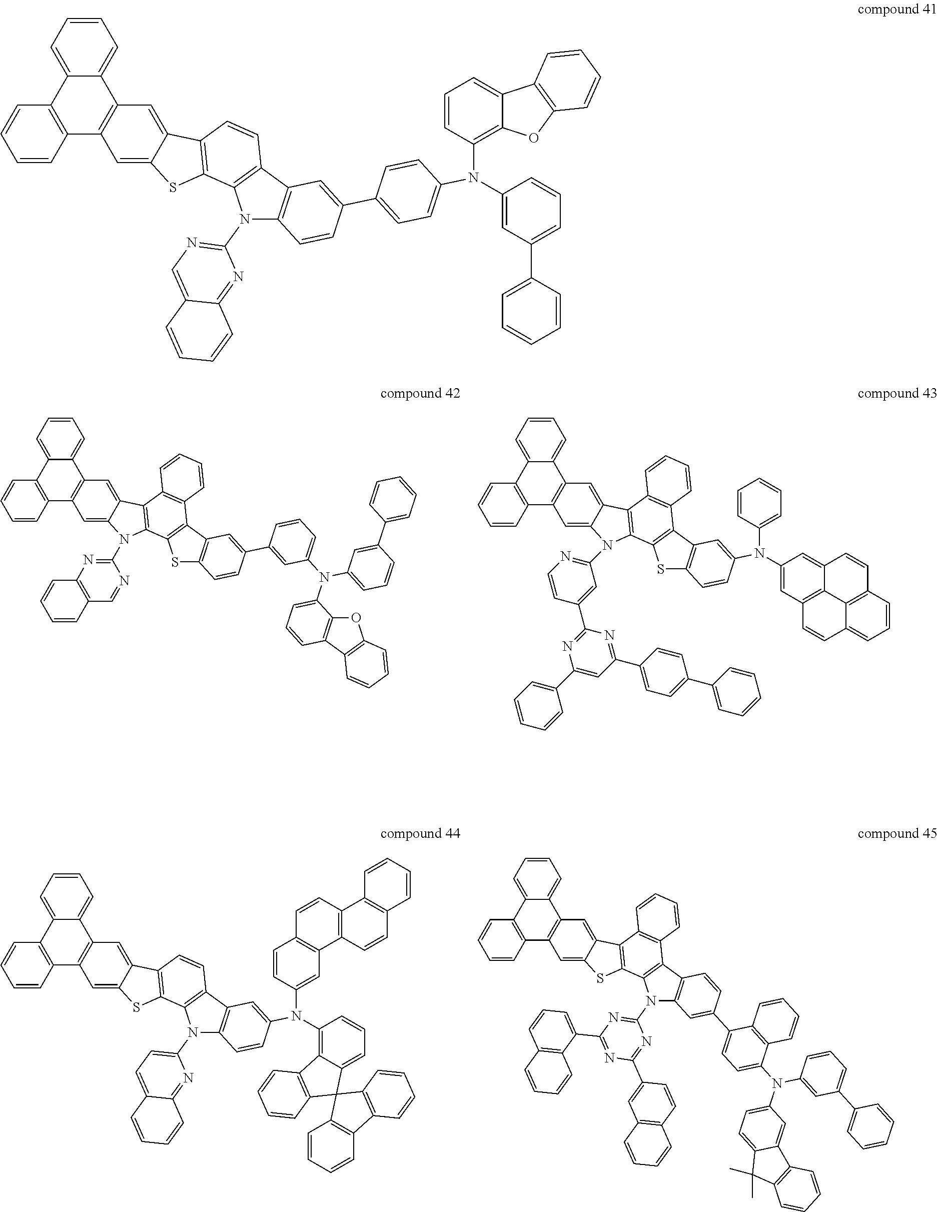

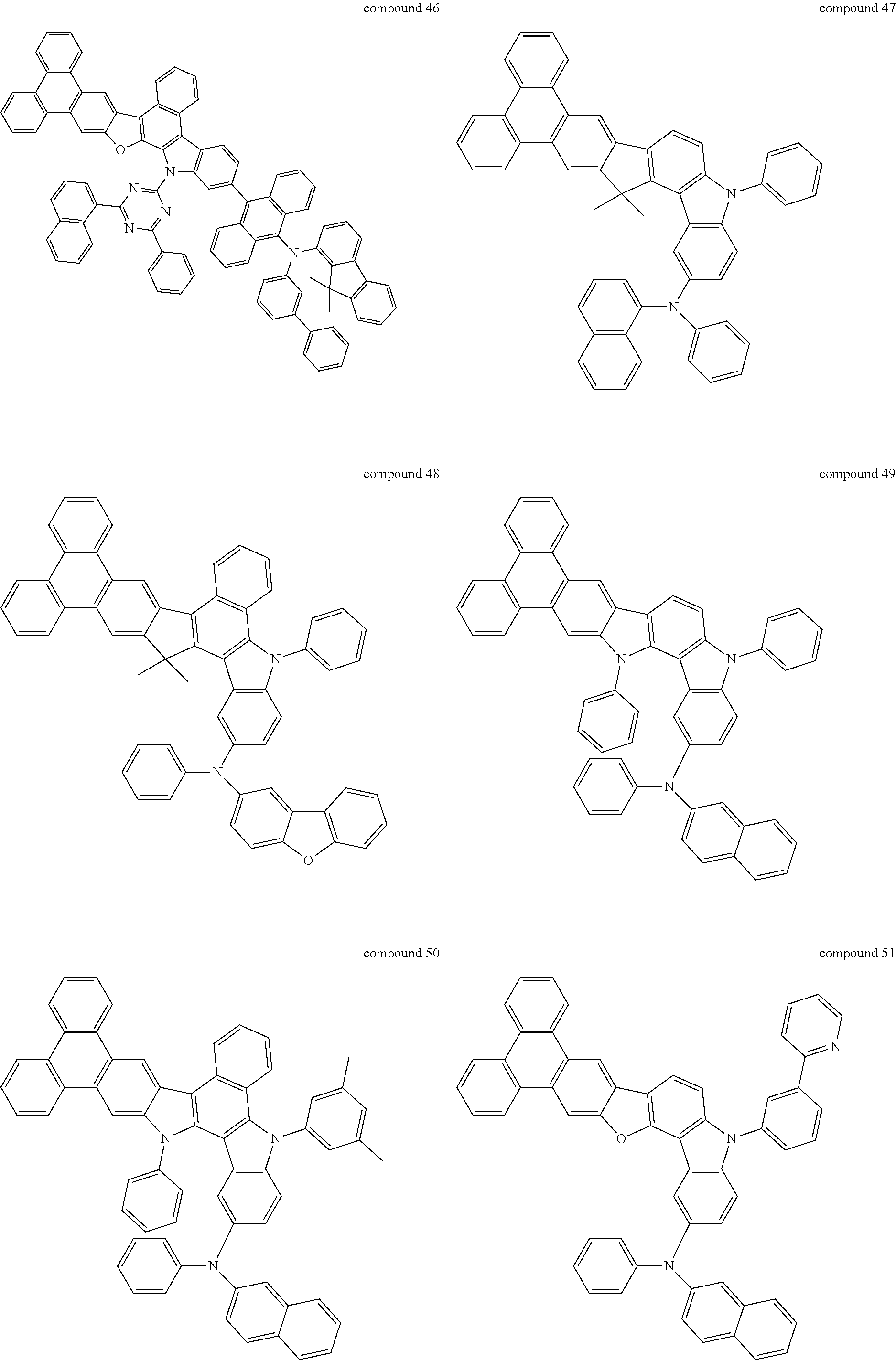

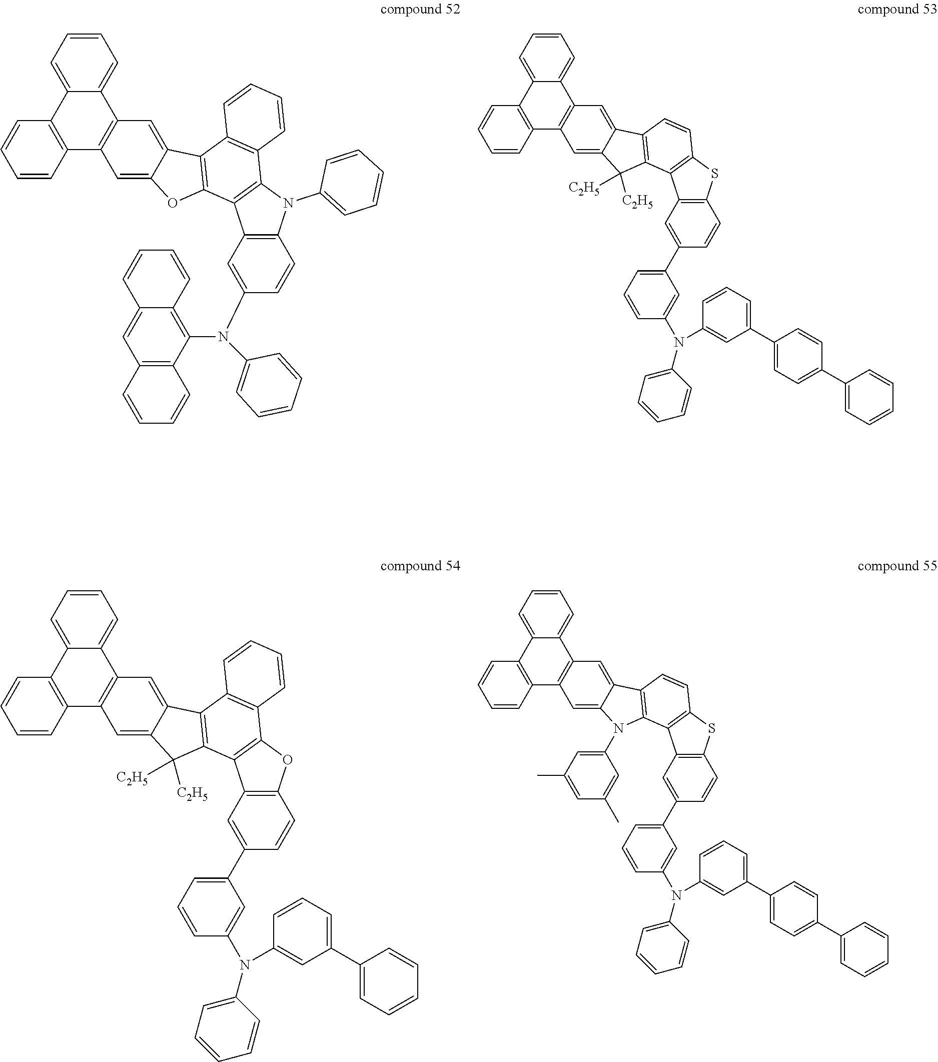 [SMILES: c1ccc(N(c2ccc3c(c2)c2c4oc5cc6c7ccccc7c7ccccc7c6cc5c4c4ccccc4c2n3-c2ccccc2)c2c3ccccc3cc3ccccc23)cc1 ; CCC1(CC)c2cc3c4ccccc4c4ccccc4c3cc2-c2ccc3sc4ccc(-c5cccc(N(c6ccccc6)c6cccc(-c7ccc(-c8ccccc8)cc7)c6)c5)cc4c3c21 ; CCC1(CC)c2cc3c4ccccc4c4ccccc4c3cc2-c2c1c1c3cc(-c4cccc(N(c5ccccc5)c5cccc(-c6ccccc6)c5)c4)ccc3oc1c1ccccc21 ; Cc1cc(C)cc(-n2c3cc4c5ccccc5c5ccccc5c4cc3c3ccc4sc5ccc(-c6cccc(N(c7ccccc7)c7cccc(-c8ccc(-c9ccccc9)cc8)c7)c6)cc5c4c32)c1]

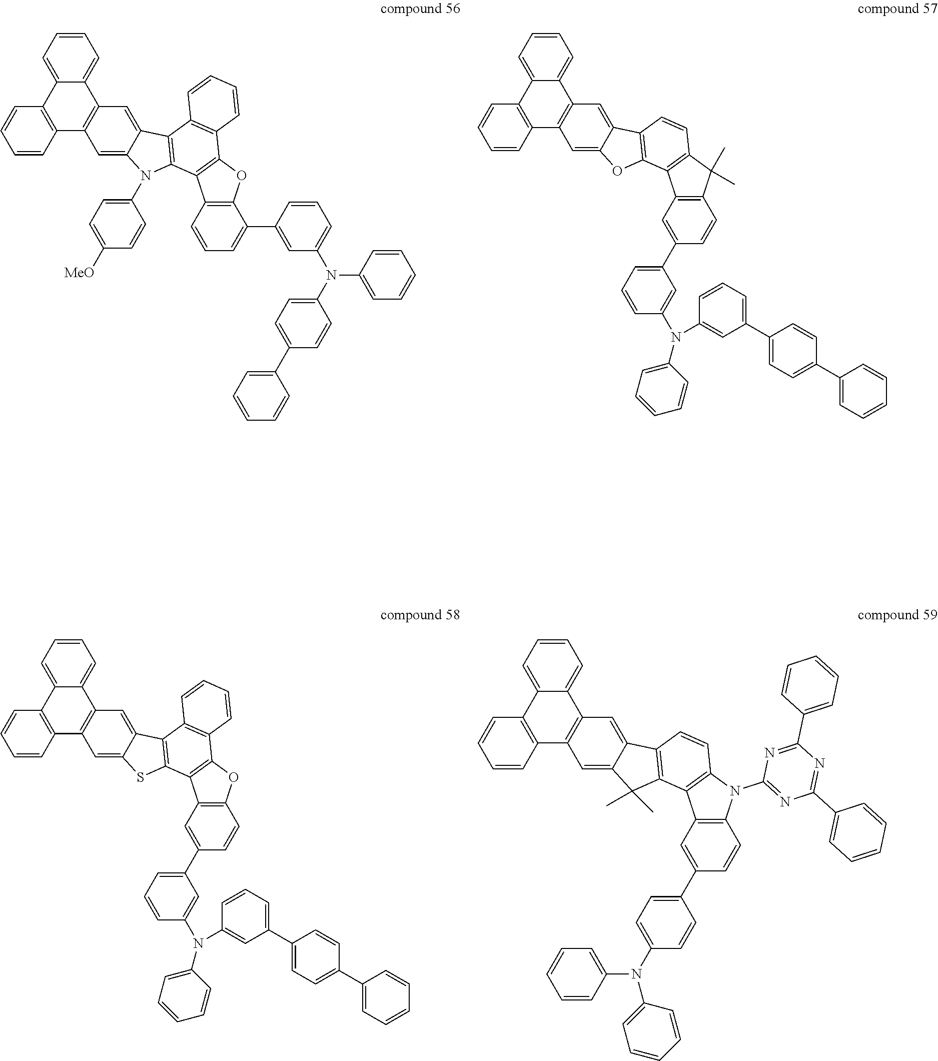 [SMILES: COc1ccc(-n2c3cc4c5ccccc5c5ccccc5c4cc3c3c4ccccc4c4oc5c(-c6cccc(N(c7ccccc7)c7ccc(-c8ccccc8)cc7)c6)cccc5c4c32)cc1 ; CC1(C)c2ccc(-c3cccc(N(c4ccccc4)c4cccc(-c5ccc(-c6ccccc6)cc5)c4)c3)cc2-c2c1ccc1c2oc2cc3c4ccccc4c4ccccc4c3cc21 ; c1ccc(-c2ccc(-c3cccc(N(c4ccccc4)c4cccc(-c5ccc6oc7c8ccccc8c8c9cc%10c%11ccccc%11c%11ccccc%11c%10cc9sc8c7c6c5)c4)c3)cc2)cc1 ; CC1(C)c2cc3c4ccccc4c4ccccc4c3cc2-c2ccc3c(c21)c1cc(-c2ccc(N(c4ccccc4)c4ccccc4)cc2)ccc1n3-c1nc(-c2ccccc2)nc(-c2ccccc2)n1]

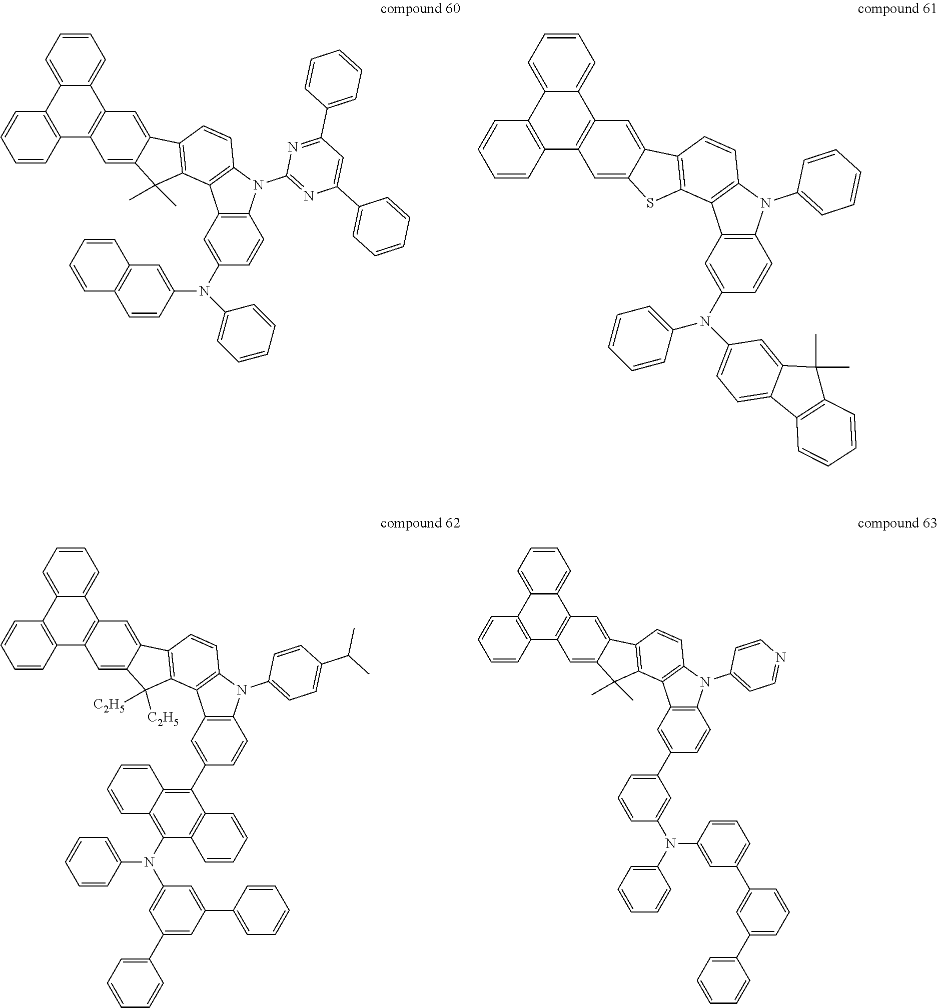 [SMILES: CC1(C)c2cc3c4ccccc4c4ccccc4c3cc2-c2ccc3c(c21)c1cc(N(c2ccccc2)c2ccc4ccccc4c2)ccc1n3-c1nc(-c2ccccc2)cc(-c2ccccc2)n1 ; CC1(C)c2ccccc2-c2ccc(N(c3ccccc3)c3ccc4c(c3)c3c5sc6cc7c8ccccc8c8ccccc8c7cc6c5ccc3n4-c3ccccc3)cc21 ; CCC1(CC)c2cc3c4ccccc4c4ccccc4c3cc2-c2ccc3c(c21)c1cc(-c2c4ccccc4c(N(c4ccccc4)c4cc(-c5ccccc5)cc(-c5ccccc5)c4)c4ccccc24)ccc1n3-c1ccc(C(C)C)cc1 ; CC1(C)c2cc3c4ccccc4c4ccccc4c3cc2-c2ccc3c(c21)c1cc(-c2cccc(N(c4ccccc4)c4cccc(-c5cccc(-c6ccccc6)c5)c4)c2)ccc1n3-c1ccncc1]

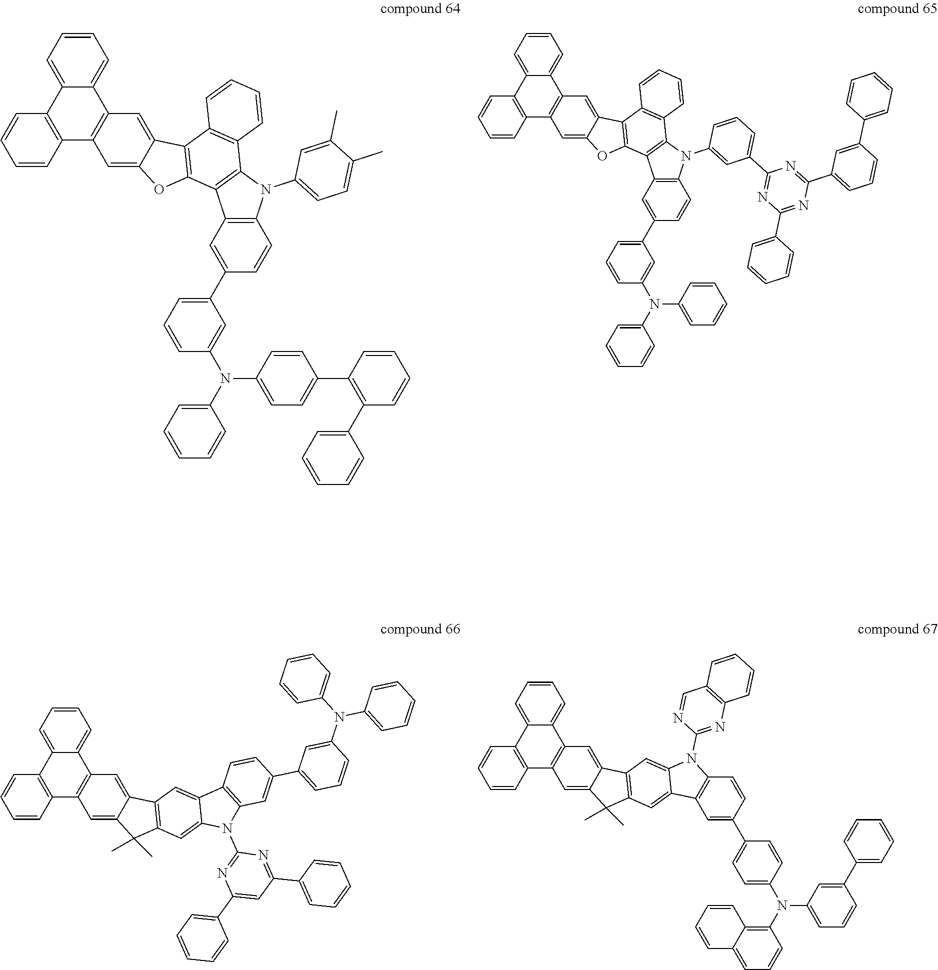

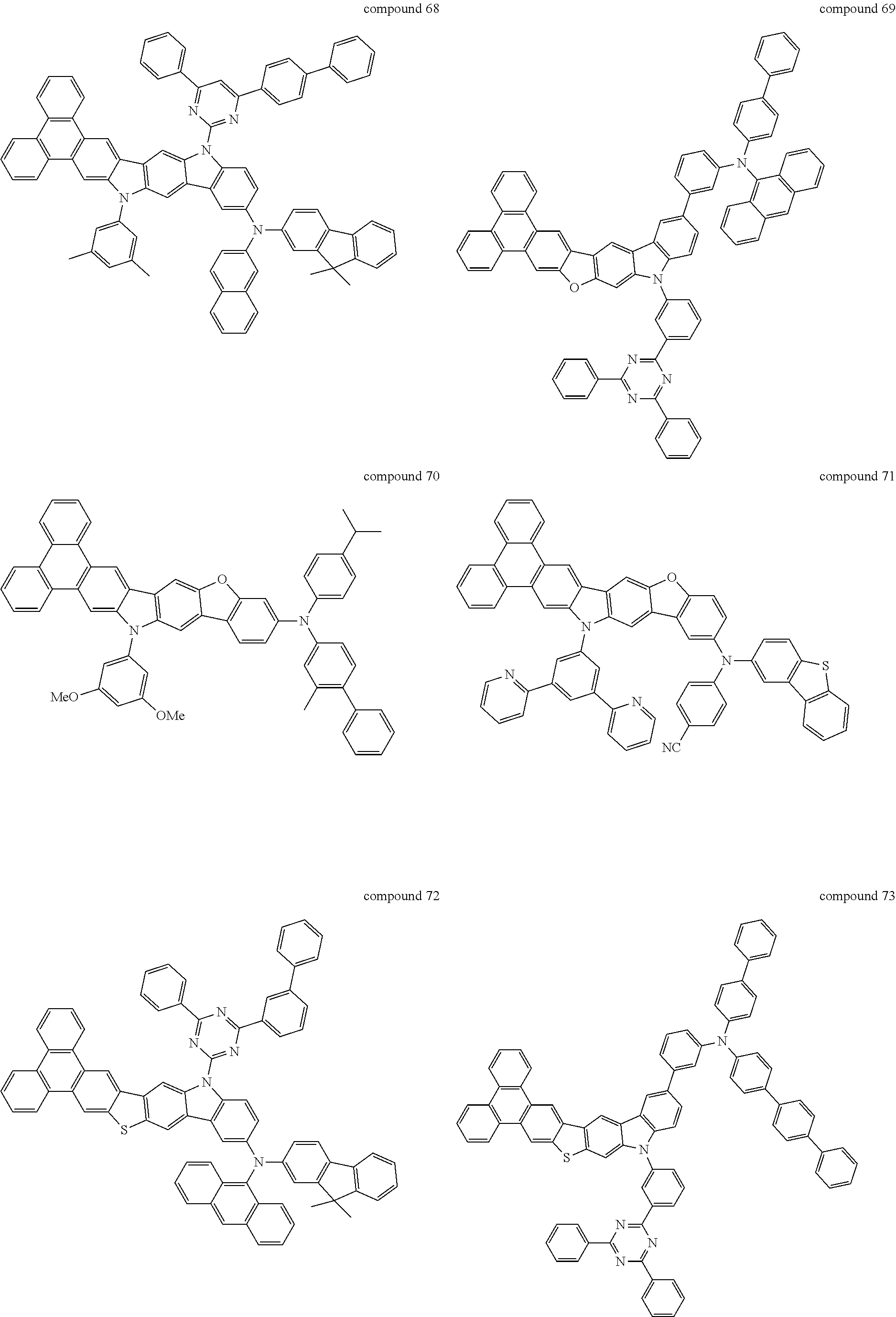

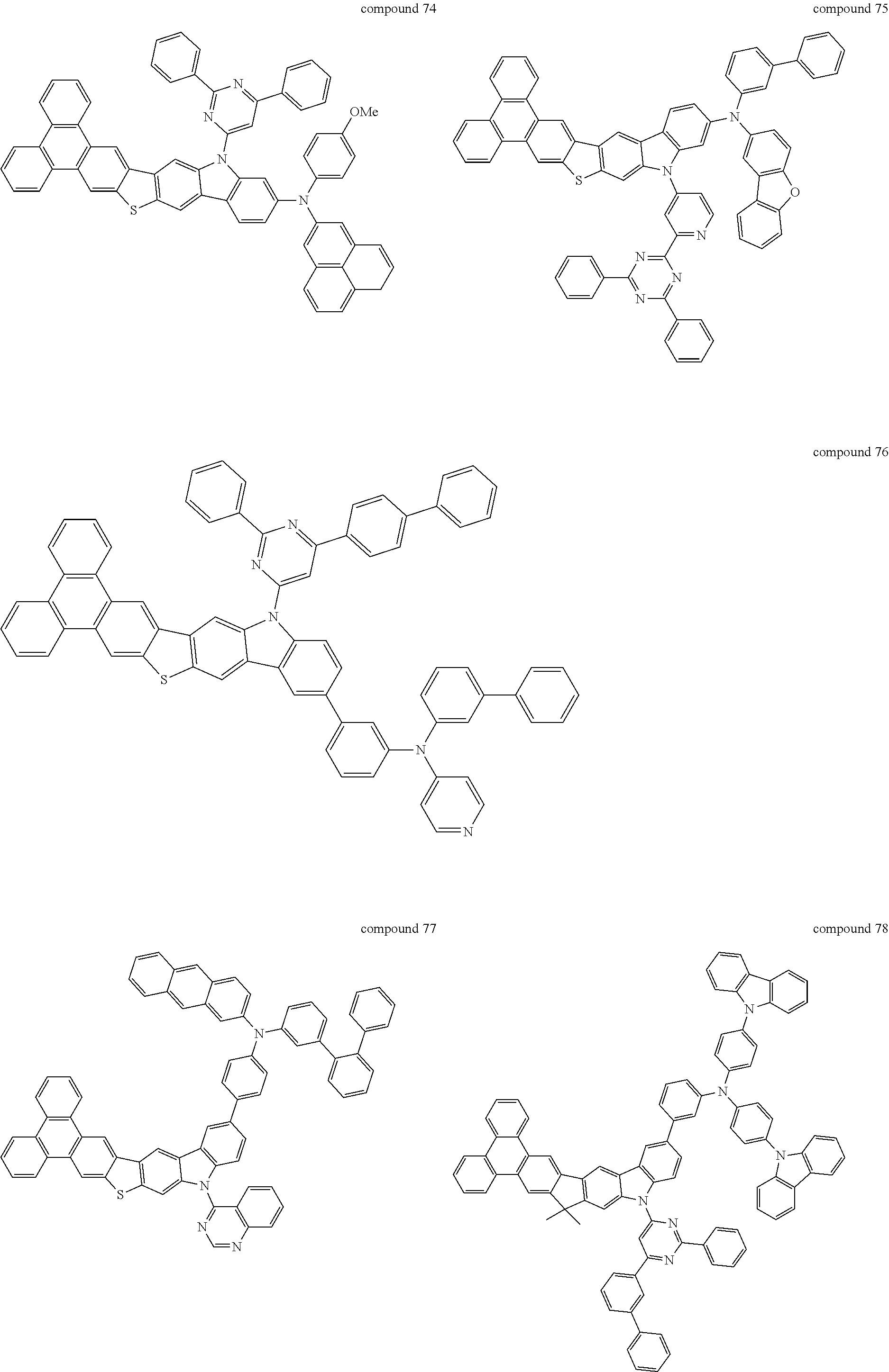

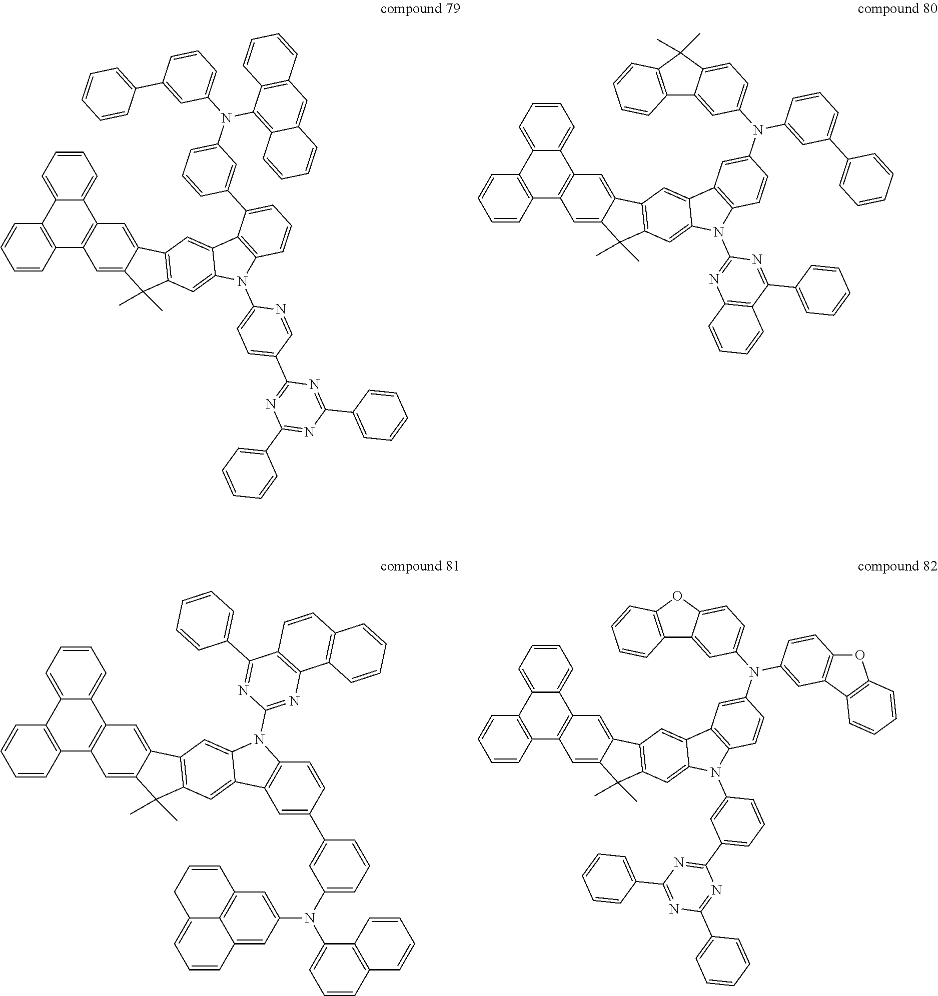

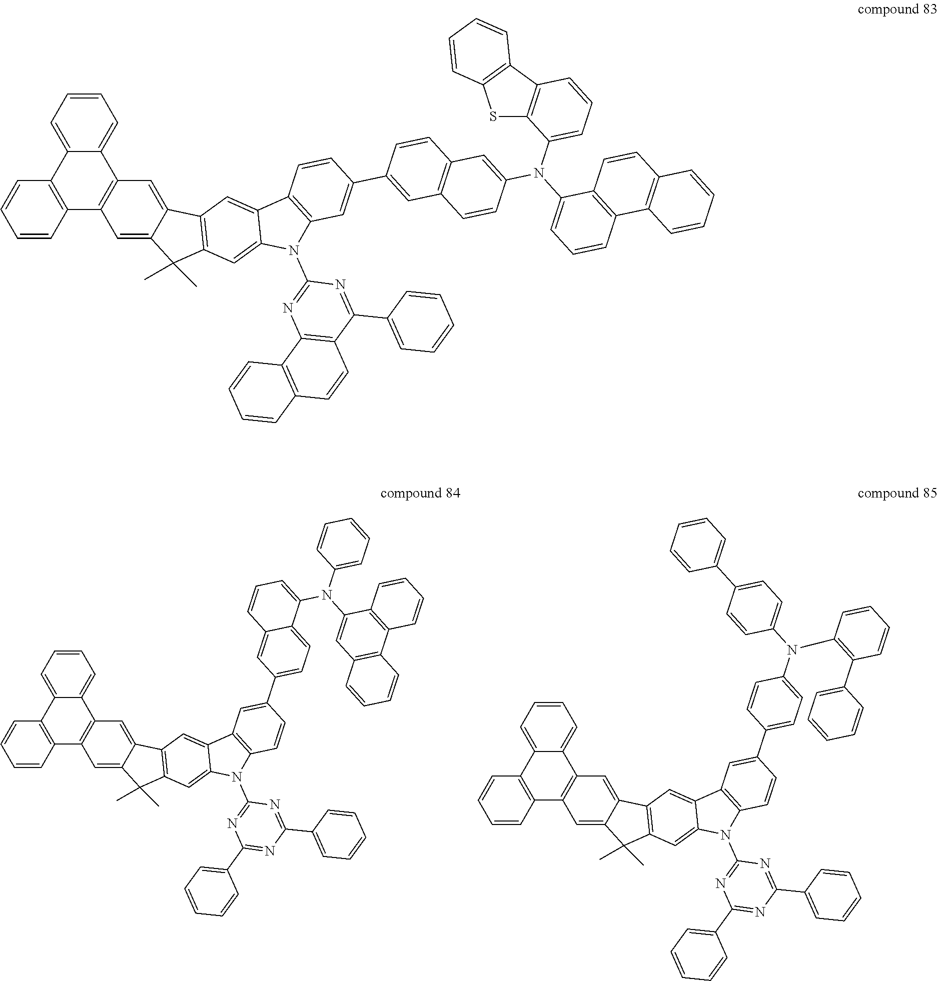 [SMILES: CC1(C)c2cc3c4ccccc4c4ccccc4c3cc2-c2cc3c4ccc(-c5ccc6cc(N(c7cccc8c7ccc7ccccc78)c7cccc8c7sc7ccccc78)ccc6c5)cc4n(-c4nc(-c5ccccc5)c5ccc6ccccc6c5n4)c3cc21 ; CC1(C)c2cc3c4ccccc4c4ccccc4c3cc2-c2cc3c4cc(-c5ccc6c(N(c7ccccc7)c7cc8ccccc8c8ccccc78)cccc6c5)ccc4n(-c4nc(-c5ccccc5)nc(-c5ccccc5)n4)c3cc21 ; CC1(C)c2cc3c4ccccc4c4ccccc4c3cc2-c2cc3c4cc(-c5ccc(N(c6ccc(-c7ccccc7)cc6)c6ccccc6-c6ccccc6)cc5)ccc4n(-c4nc(-c5ccccc5)nc(-c5ccccc5)n4)c3cc21]

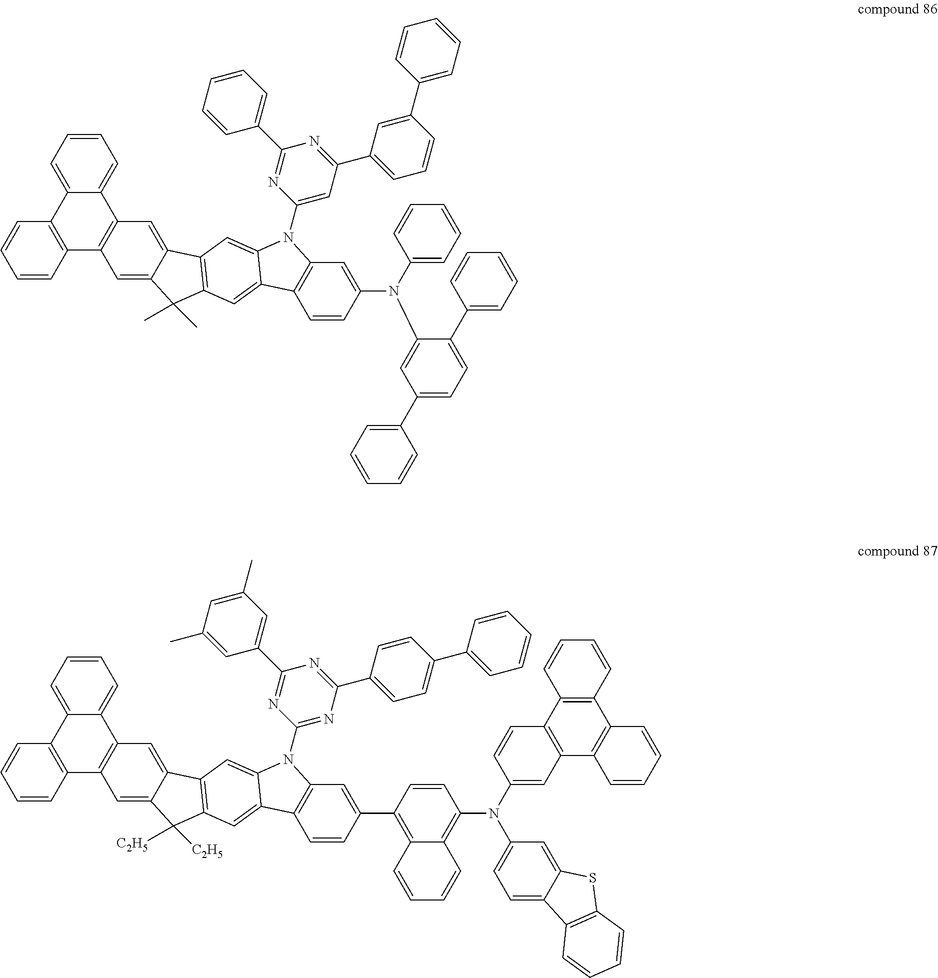 [SMILES: CC1(C)c2cc3c4ccccc4c4ccccc4c3cc2-c2cc3c(cc21)c1ccc(N(c2ccccc2)c2cc(-c4ccccc4)ccc2-c2ccccc2)cc1n3-c1cc(-c2cccc(-c3ccccc3)c2)nc(-c2ccccc2)n1 ; CCC1(CC)c2cc3c4ccccc4c4ccccc4c3cc2-c2cc3c(cc21)c1ccc(-c2ccc(N(c4ccc5c(c4)sc4ccccc45)c4ccc5c6ccccc6c6ccccc6c5c4)c4ccccc24)cc1n3-c1nc(-c2ccc(-c3ccccc3)cc2)nc(-c2cc(C)cc(C)c2)n1]

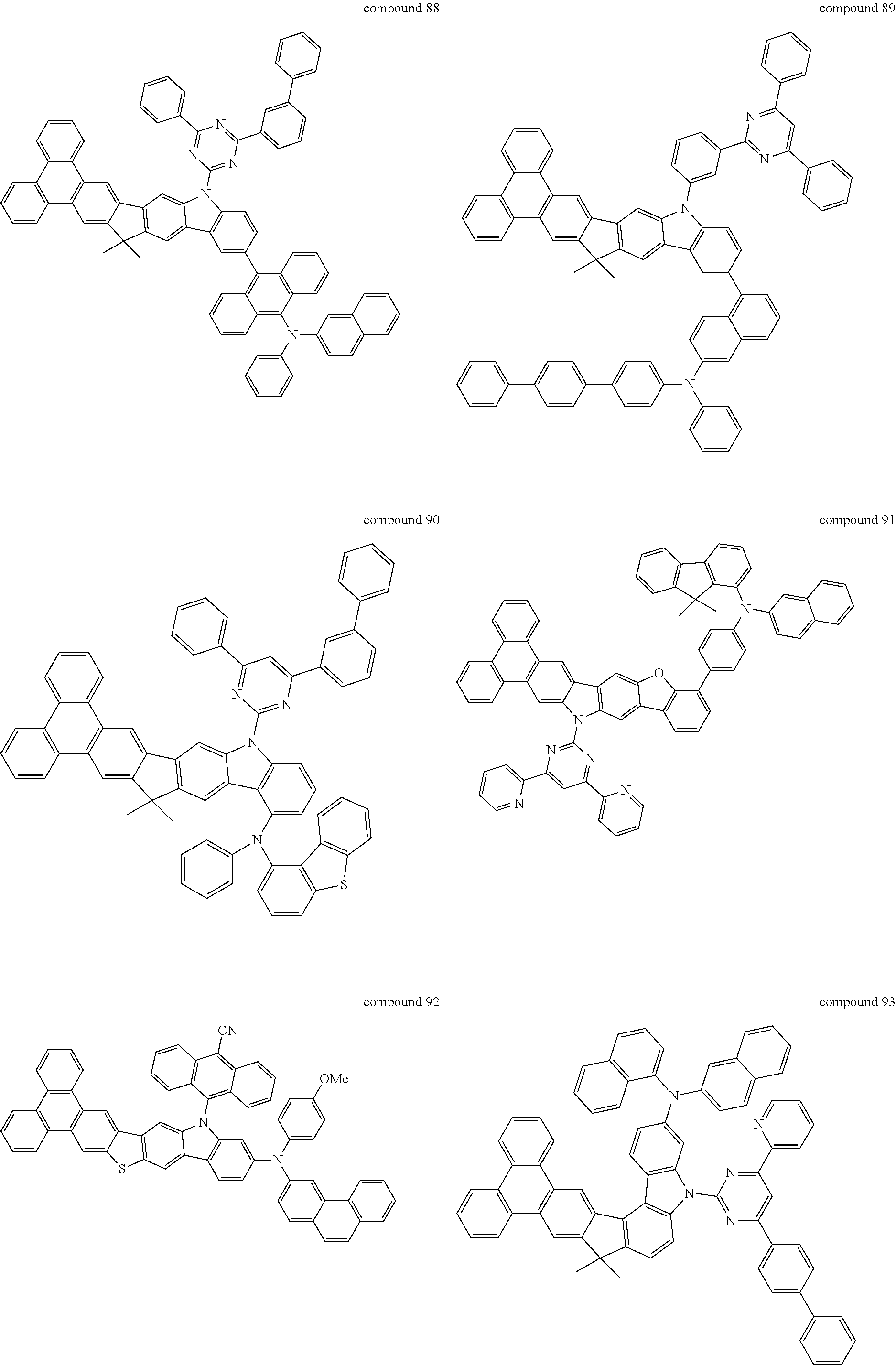

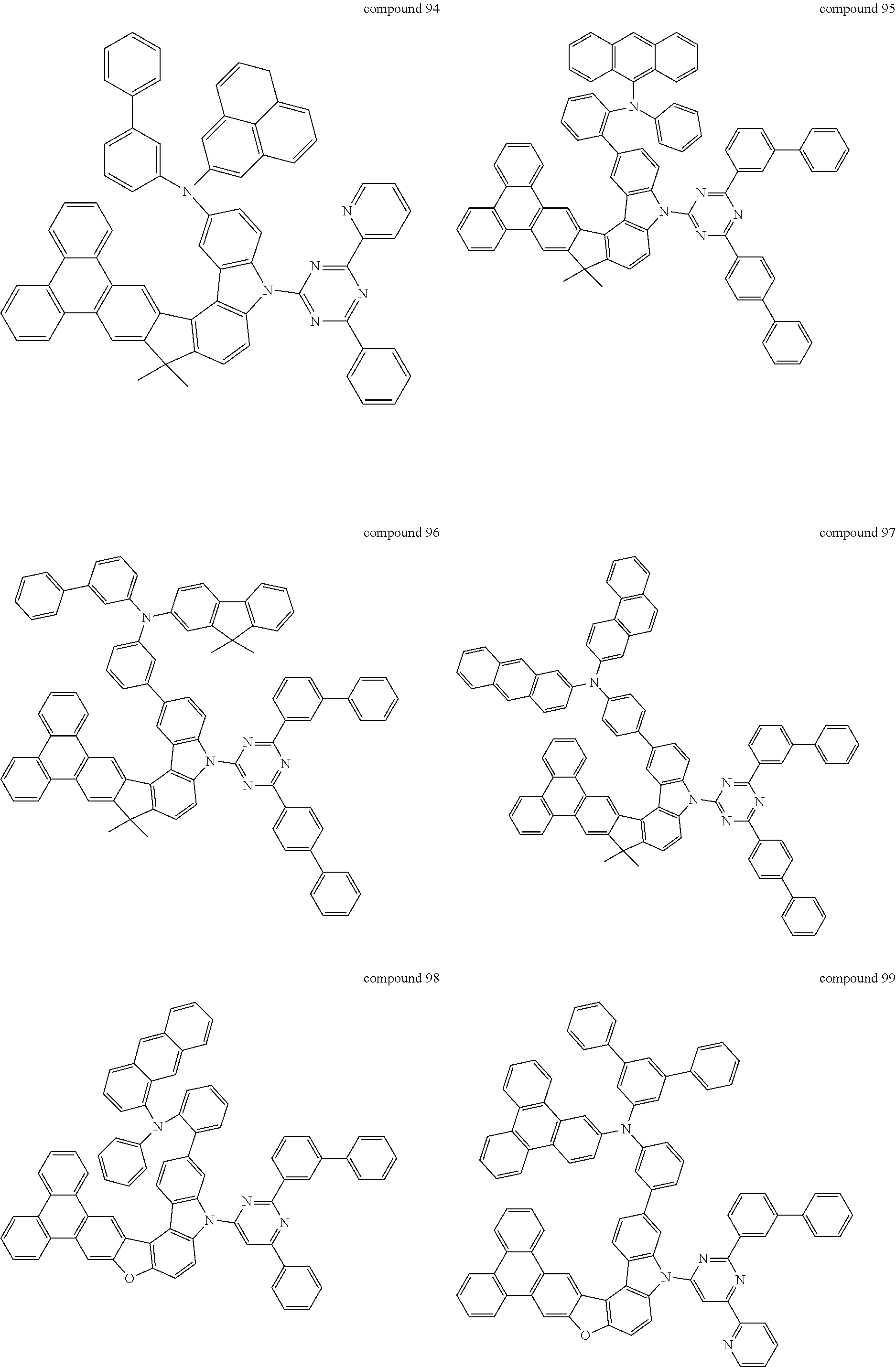

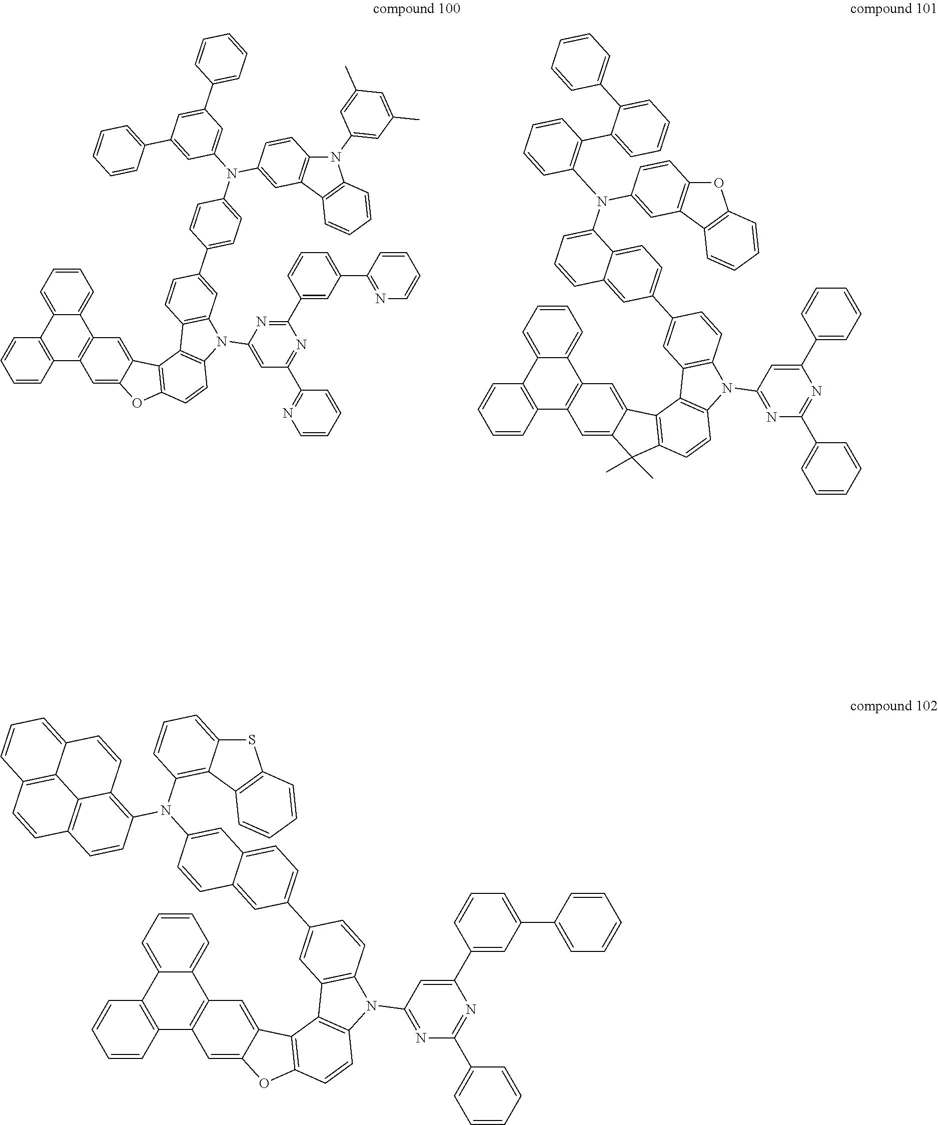

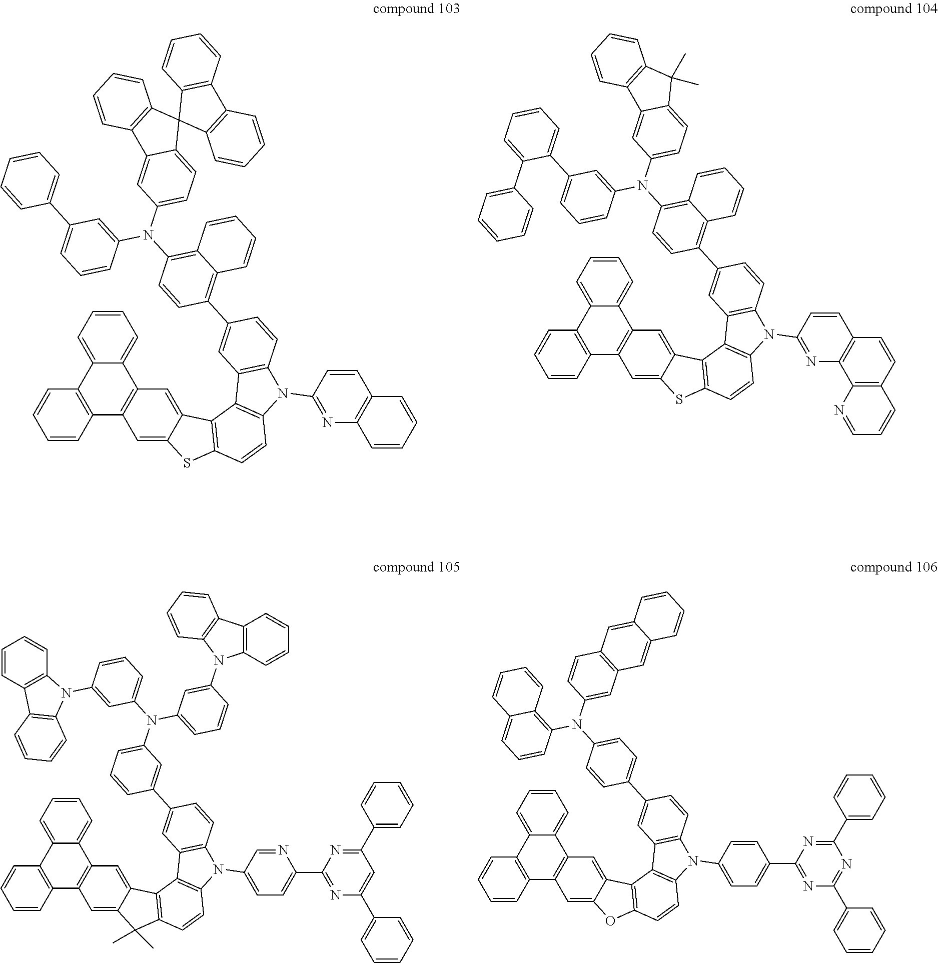

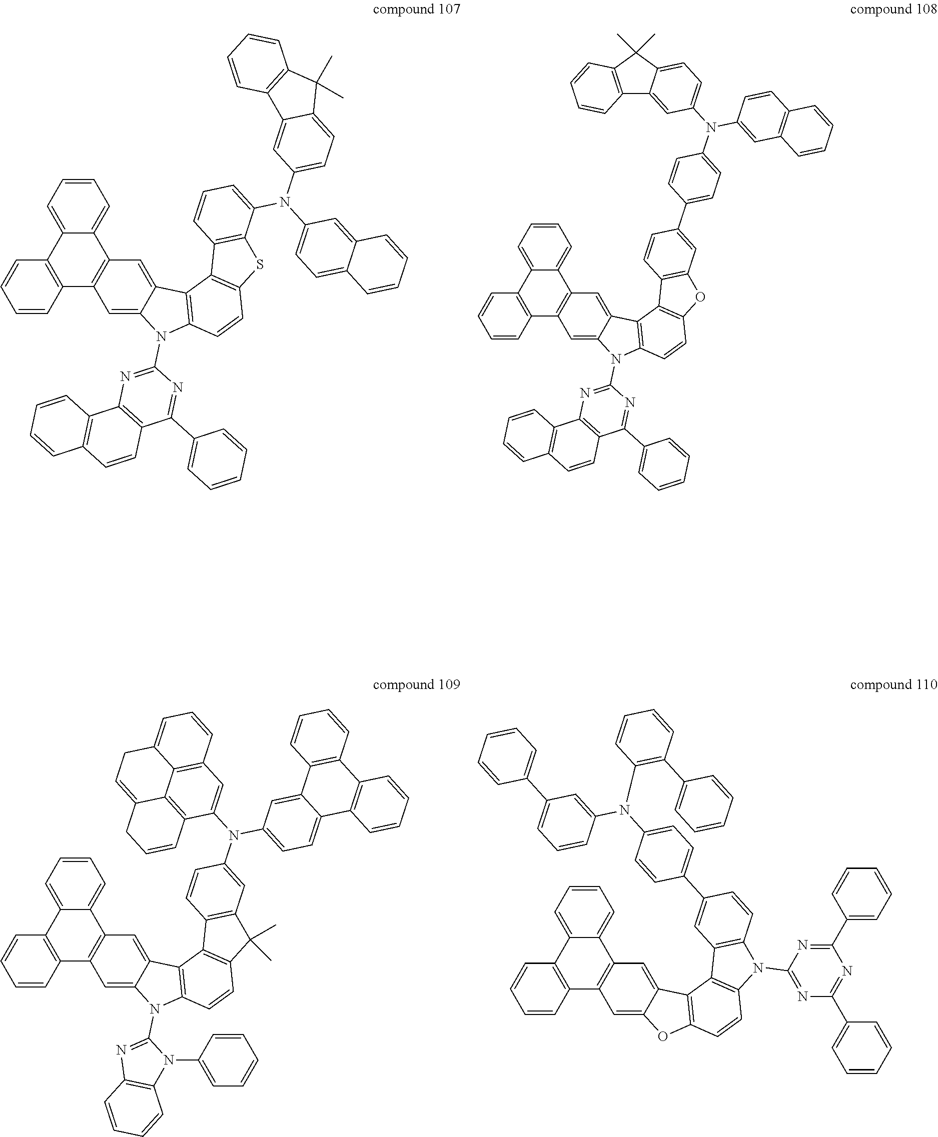

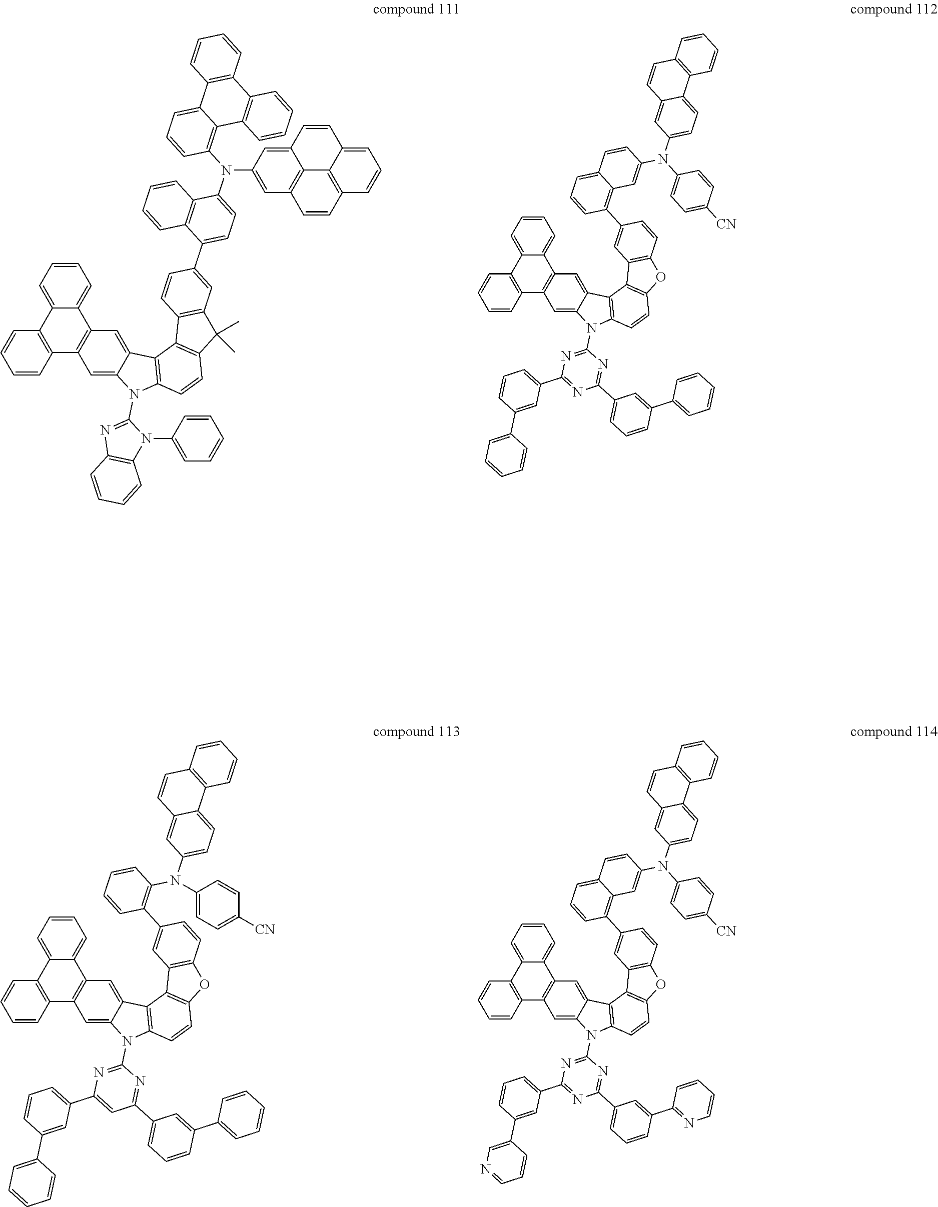

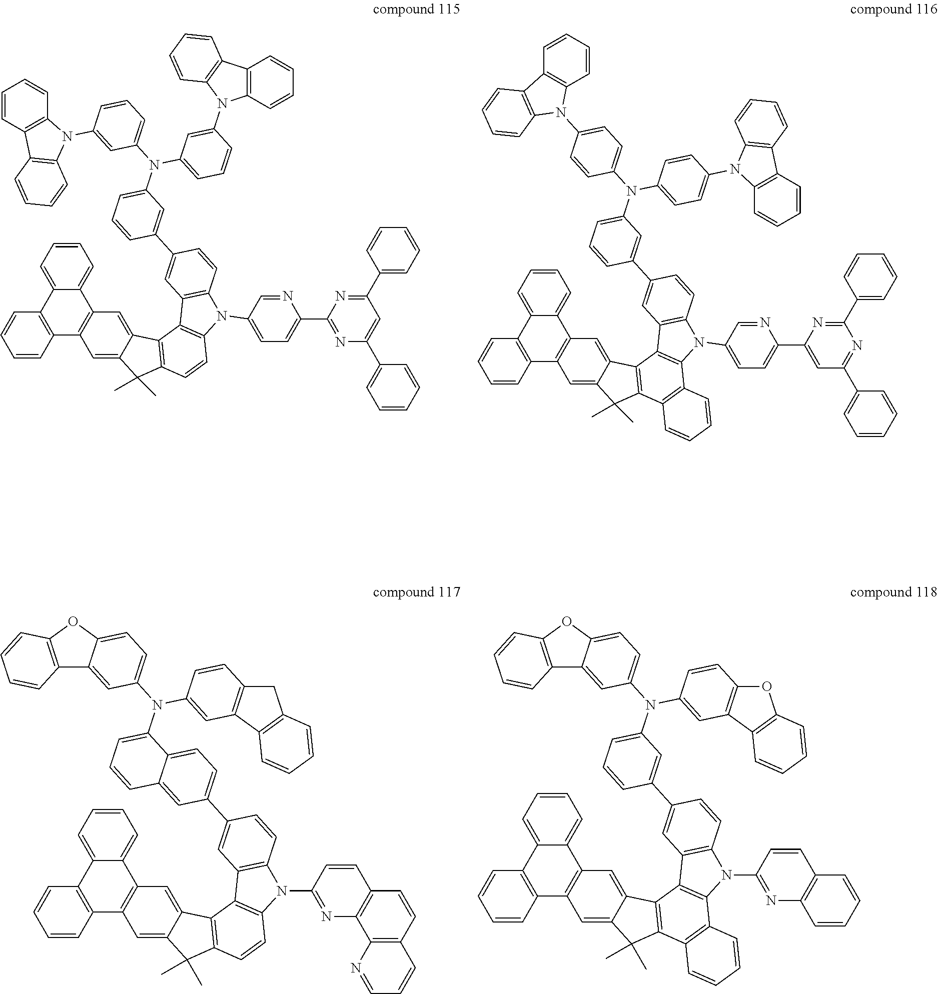

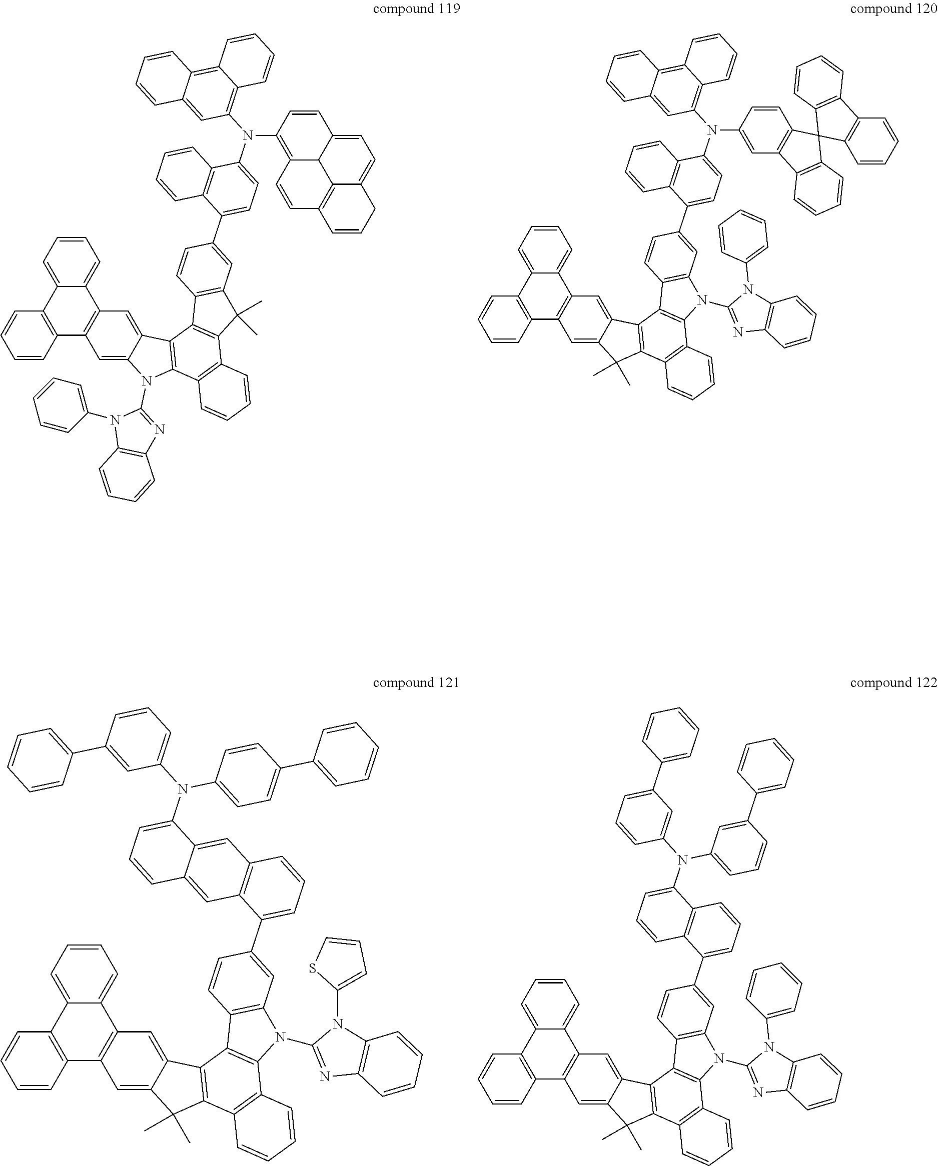

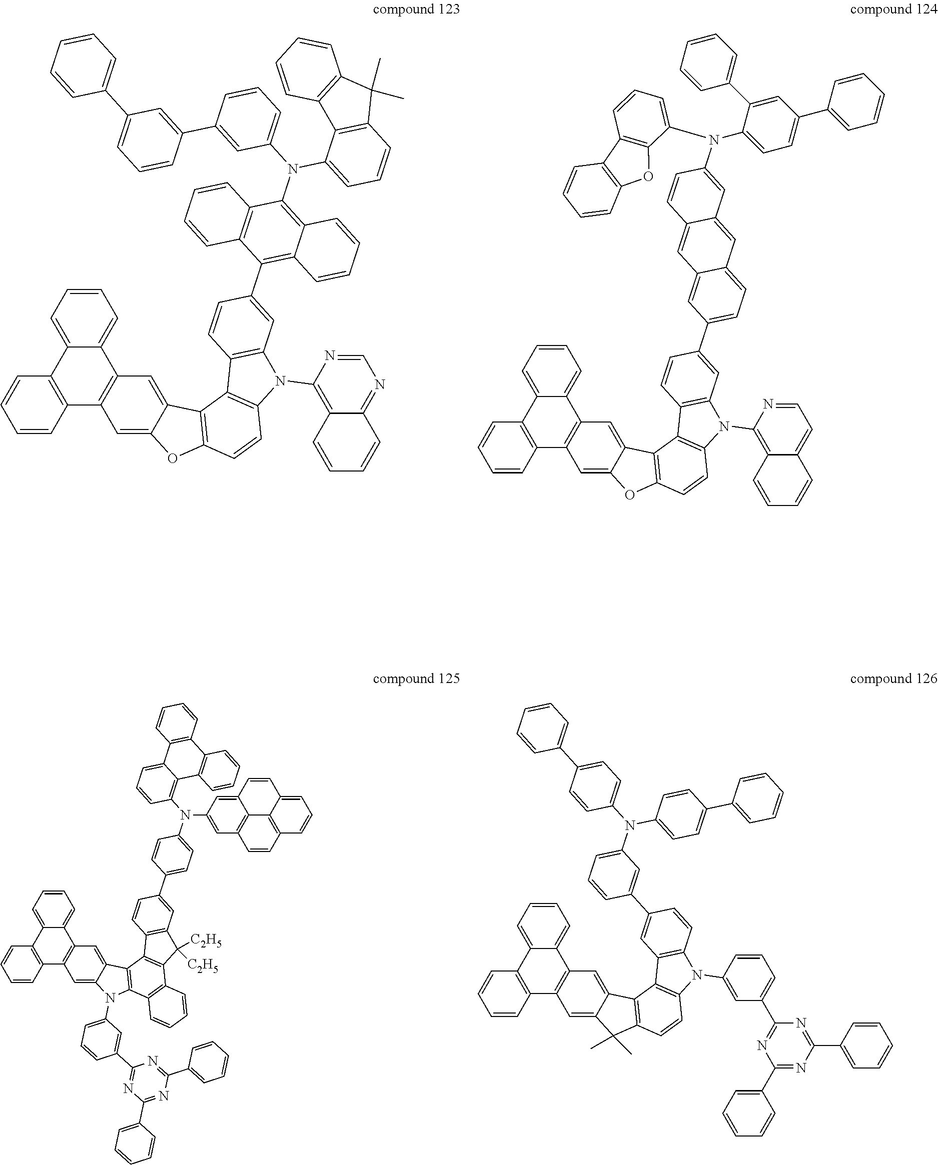

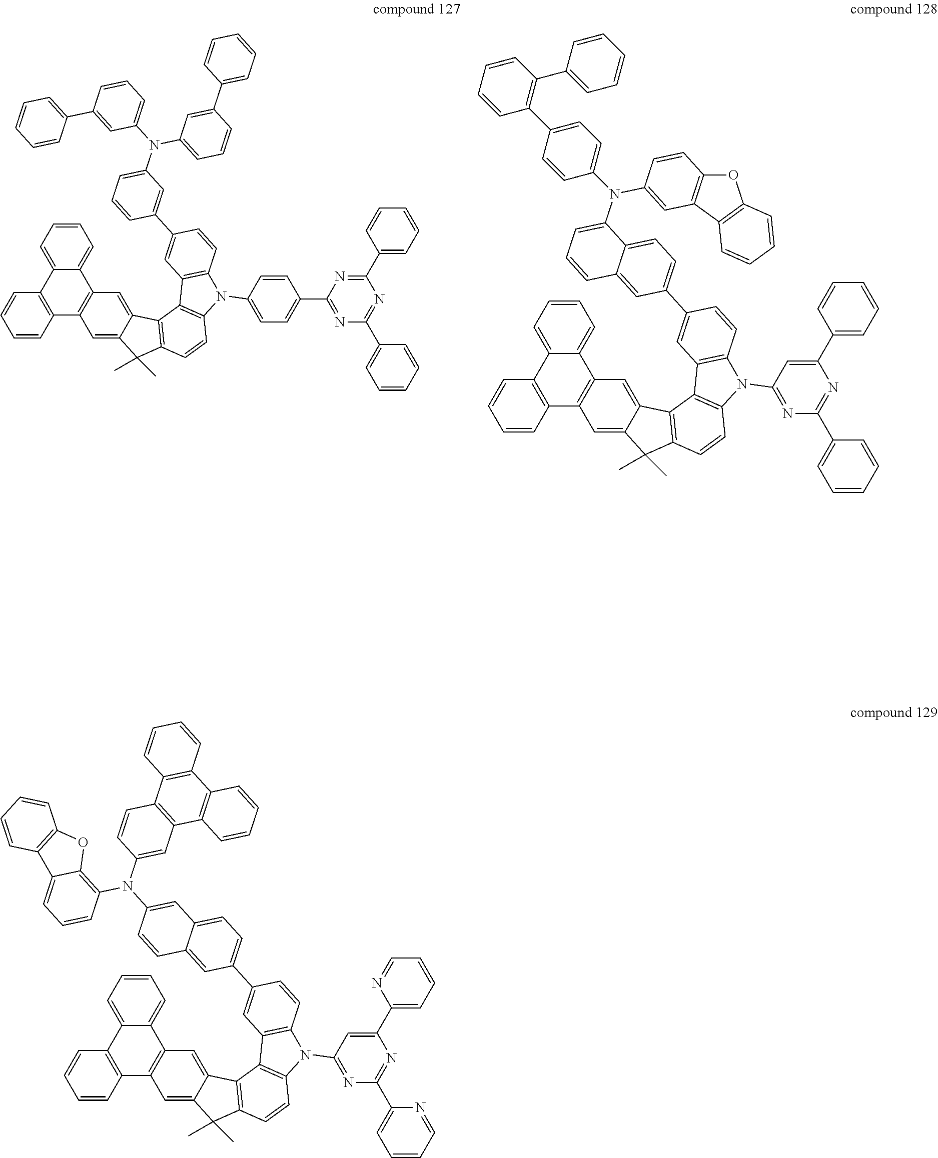 [SMILES: CC1(C)c2cc3c4ccccc4c4ccccc4c3cc2-c2c1ccc1c2c2cc(-c3cccc(N(c4cccc(-c5ccccc5)c4)c4cccc(-c5ccccc5)c4)c3)ccc2n1-c1ccc(-c2nc(-c3ccccc3)nc(-c3ccccc3)n2)cc1 ; CC1(C)c2cc3c4ccccc4c4ccccc4c3cc2-c2c1ccc1c2c2cc(-c3ccc4c(N(c5ccc(-c6ccccc6-c6ccccc6)cc5)c5ccc6oc7ccccc7c6c5)cccc4c3)ccc2n1-c1cc(-c2ccccc2)nc(-c2ccccc2)n1 ; CC1(C)c2cc3c4ccccc4c4ccccc4c3cc2-c2c1ccc1c2c2cc(-c3ccc4cc(N(c5ccc6c7ccccc7c7ccccc7c6c5)c5cccc6c5oc5ccccc56)ccc4c3)ccc2n1-c1cc(-c2ccccn2)nc(-c2ccccn2)n1]

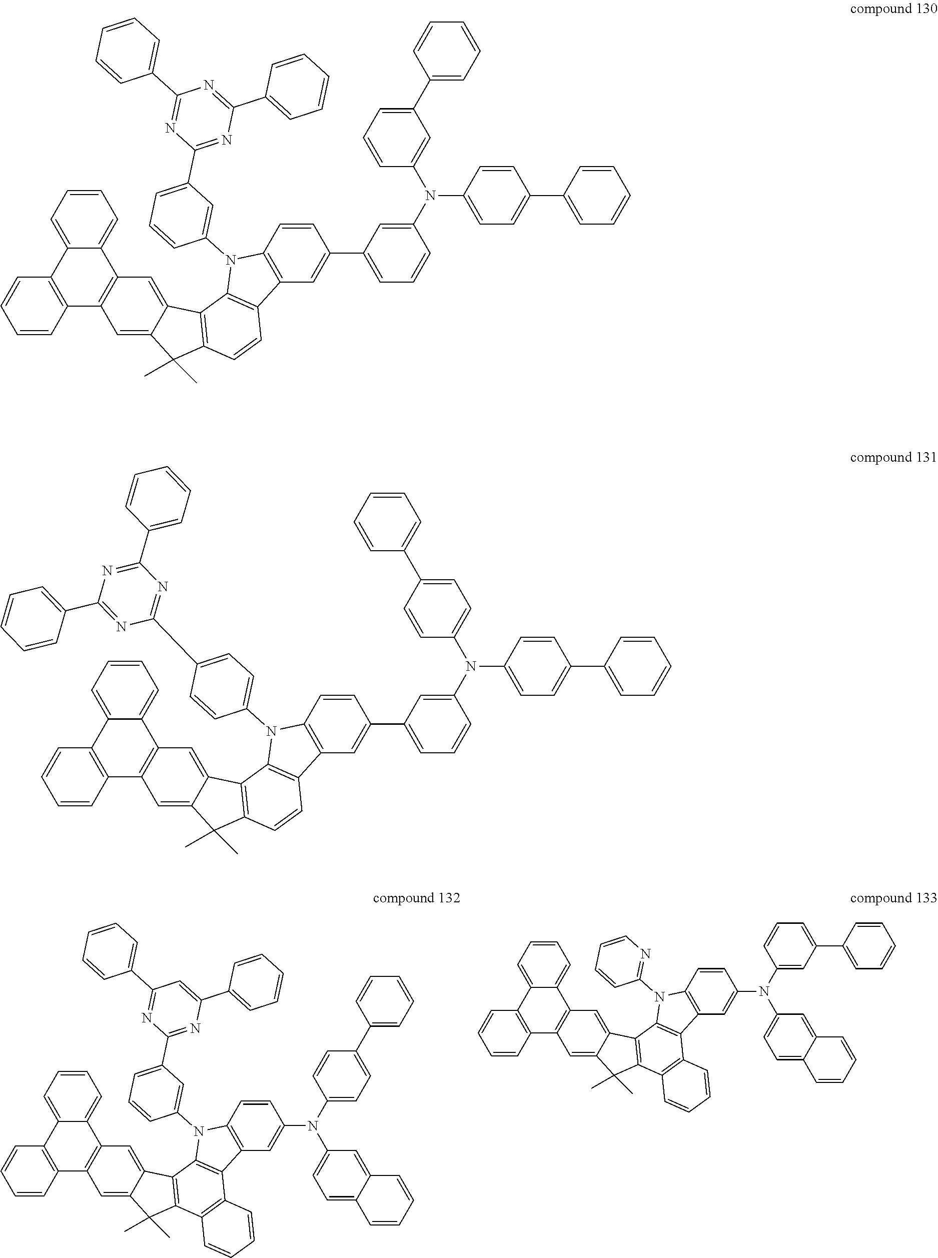

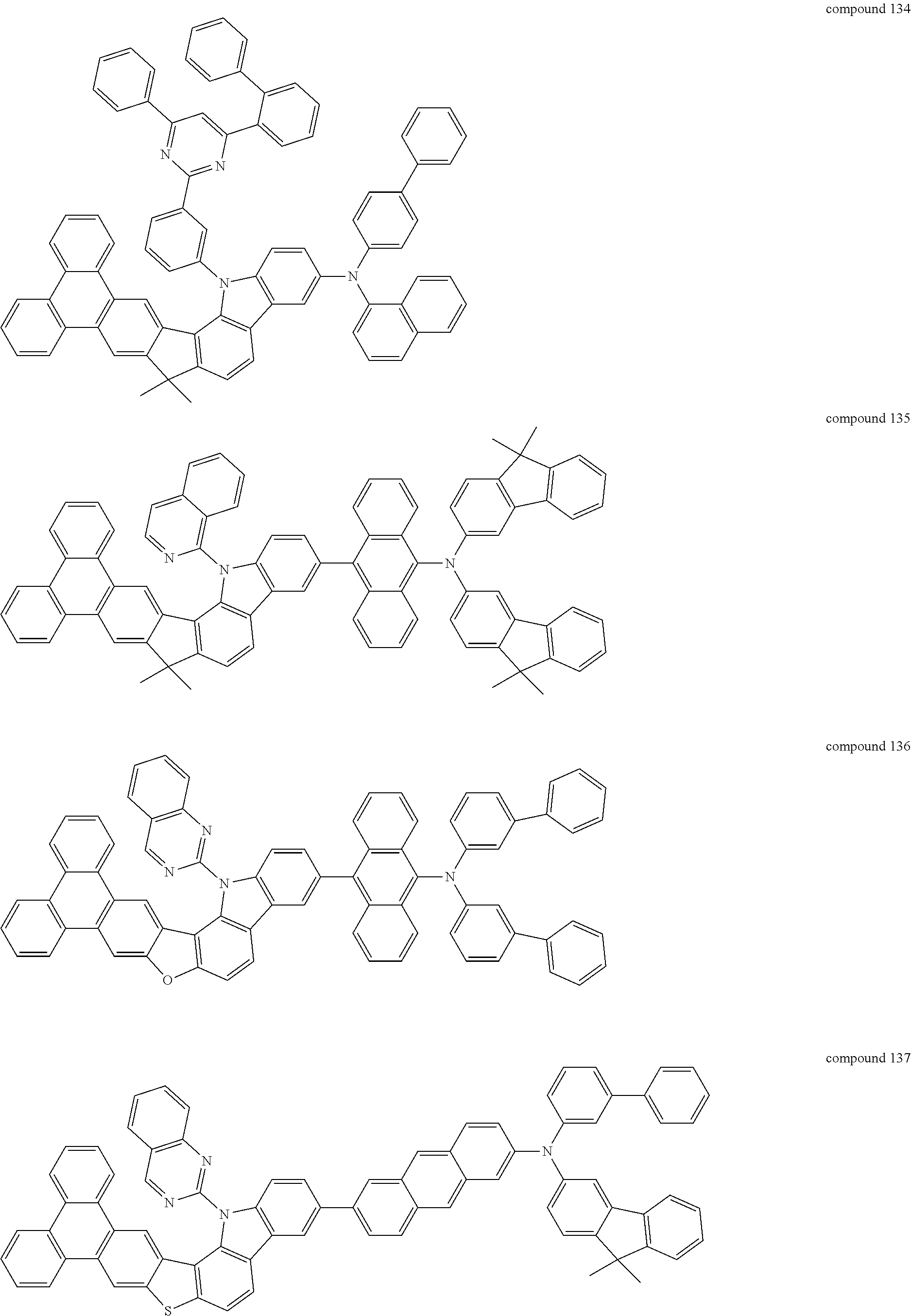

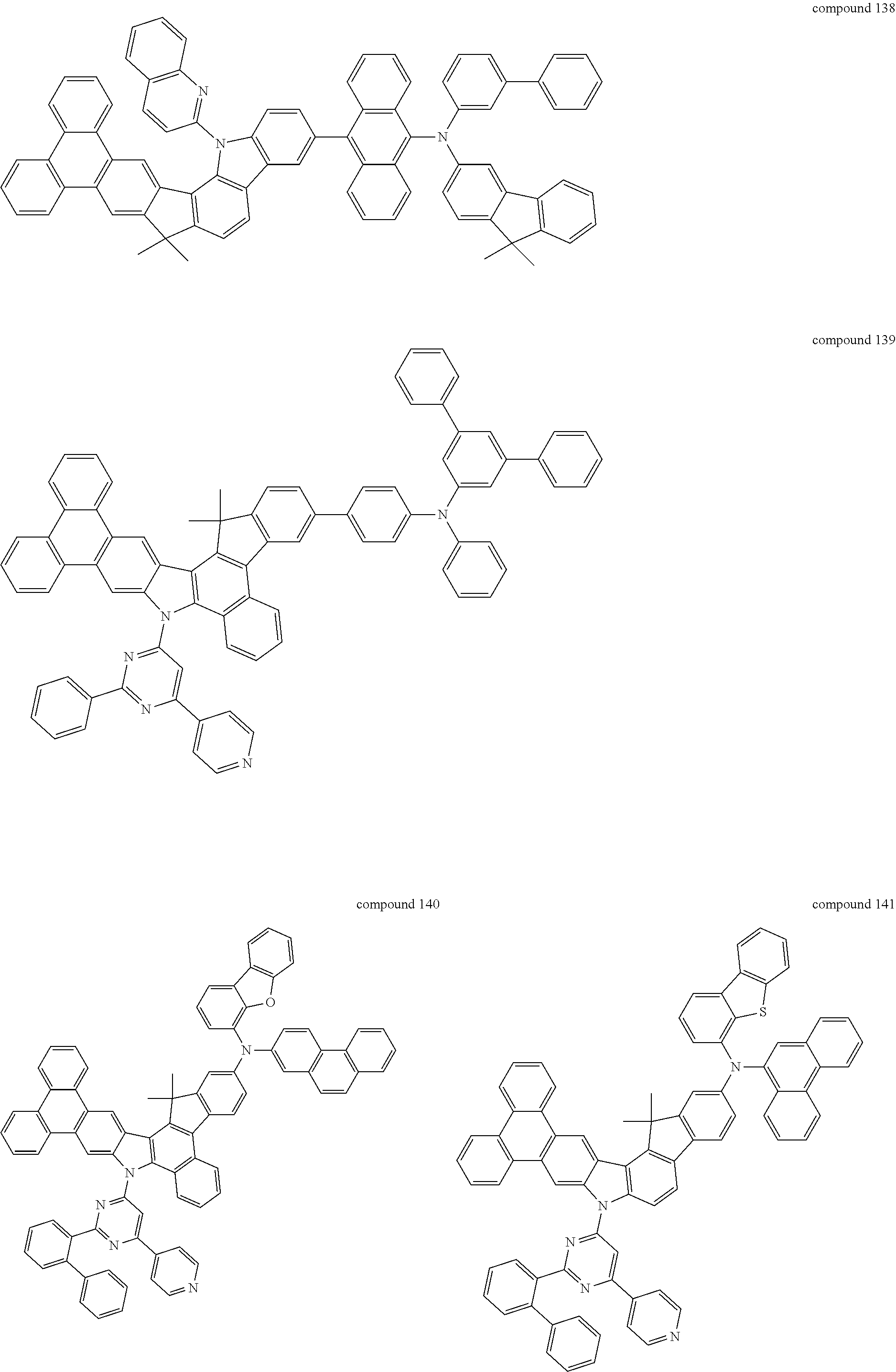

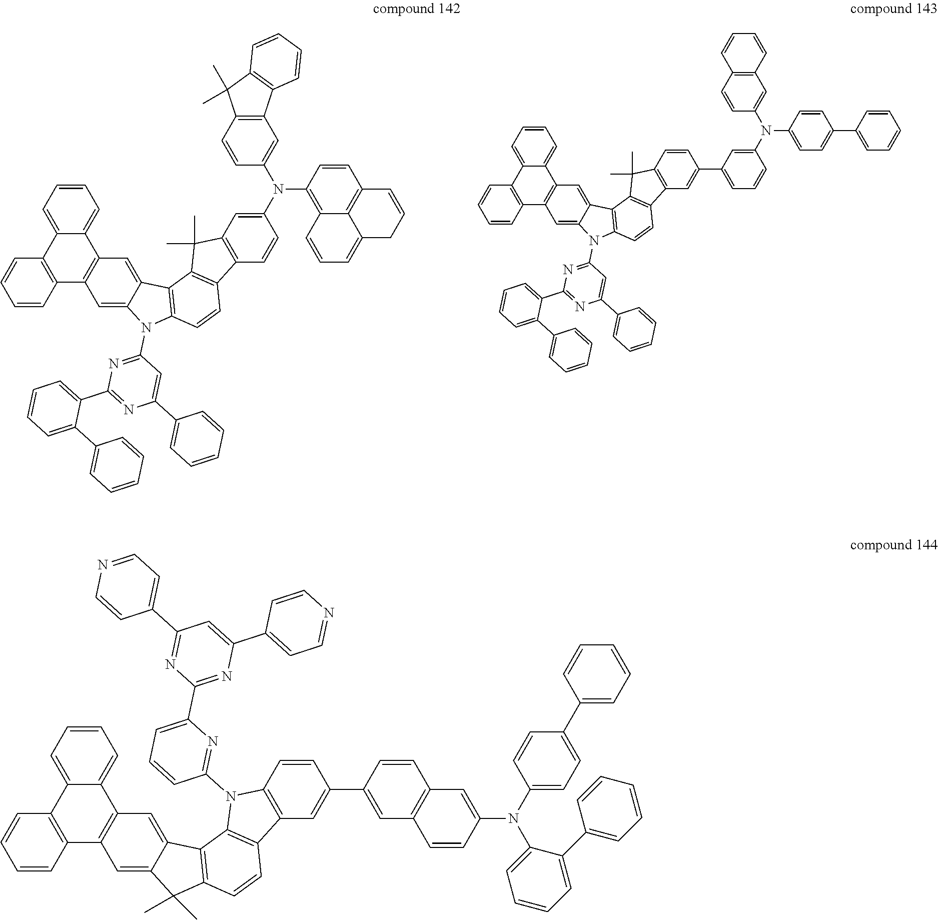 [SMILES: CC1(C)c2ccccc2-c2cc(N(c3ccc4c(c3)C(C)(C)c3c-4ccc4c3c3cc5c6ccccc6c6ccccc6c5cc3n4-c3cc(-c4ccccc4)nc(-c4ccccc4-c4ccccc4)n3)c3ccc4c5c(cccc35)CC=C4)ccc21 ; CC1(C)c2ccc(-c3cccc(N(c4ccc(-c5ccccc5)cc4)c4ccc5ccccc5c4)c3)cc2-c2ccc3c(c21)c1cc2c4ccccc4c4ccccc4c2cc1n3-c1cc(-c2ccccc2)nc(-c2ccccc2-c2ccccc2)n1 ; CC1(C)c2cc3c4ccccc4c4ccccc4c3cc2-c2c1ccc1c3cc(-c4ccc5cc(N(c6ccc(-c7ccccc7)cc6)c6ccccc6-c6ccccc6)ccc5c4)ccc3n(-c3cccc(-c4nc(-c5ccncc5)cc(-c5ccncc5)n4)n3)c21]

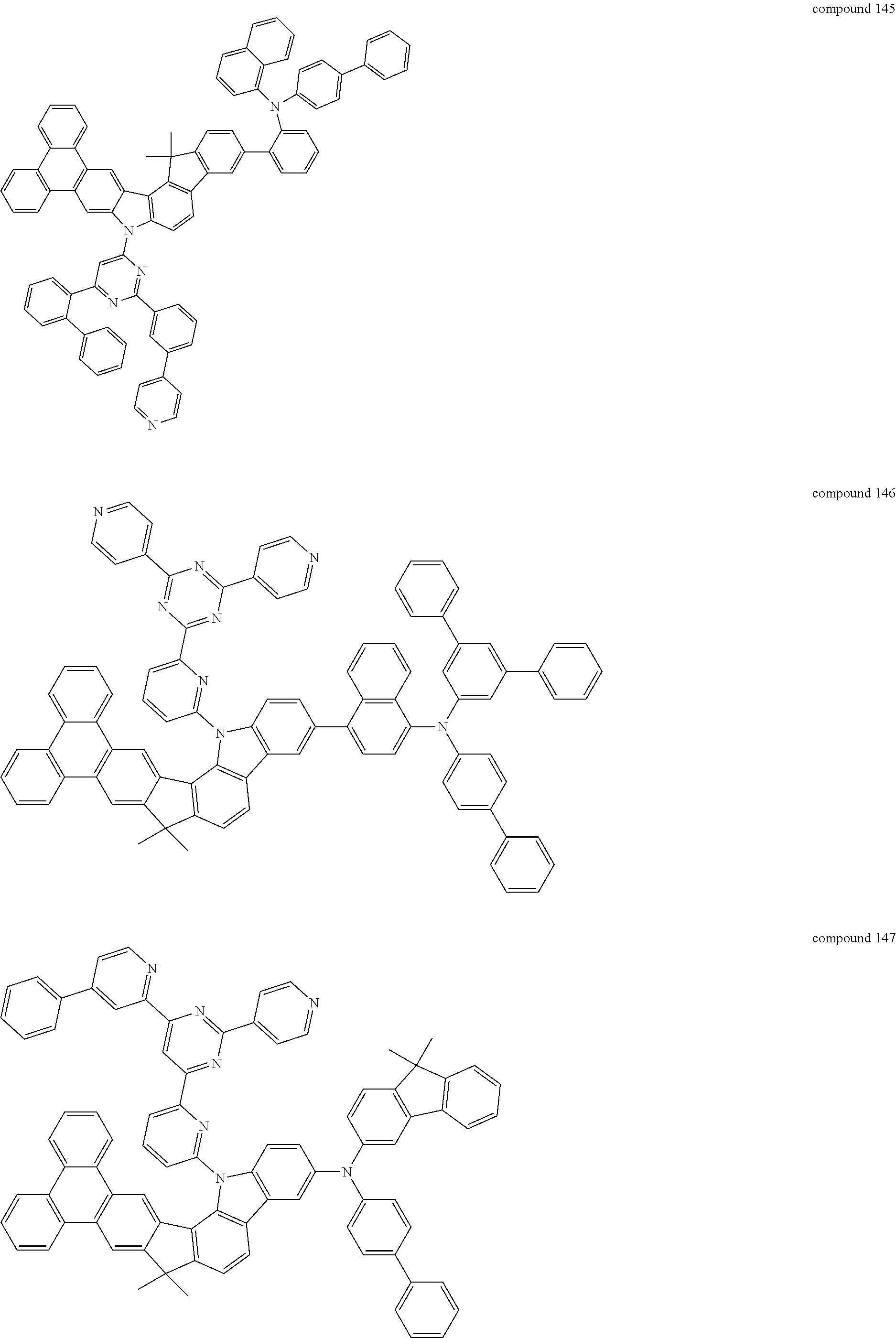 [SMILES: CC1(C)c2ccc(-c3ccccc3N(c3ccc(-c4ccccc4)cc3)c3cccc4ccccc34)cc2-c2ccc3c(c21)c1cc2c4ccccc4c4ccccc4c2cc1n3-c1cc(-c2ccccc2-c2ccccc2)nc(-c2cccc(-c3ccncc3)c2)n1 ; CC1(C)c2cc3c4ccccc4c4ccccc4c3cc2-c2c1ccc1c3cc(-c4ccc(N(c5ccc(-c6ccccc6)cc5)c5cc(-c6ccccc6)cc(-c6ccccc6)c5)c5ccccc45)ccc3n(-c3cccc(-c4nc(-c5ccncc5)nc(-c5ccncc5)n4)n3)c21 ; CC1(C)c2ccccc2-c2cc(N(c3ccc(-c4ccccc4)cc3)c3ccc4c(c3)c3ccc5c(c3n4-c3cccc(-c4cc(-c6cc(-c7ccccc7)ccn6)nc(-c6ccncc6)n4)n3)-c3cc4c6ccccc6c6ccccc6c4cc3C5(C)C)ccc21]

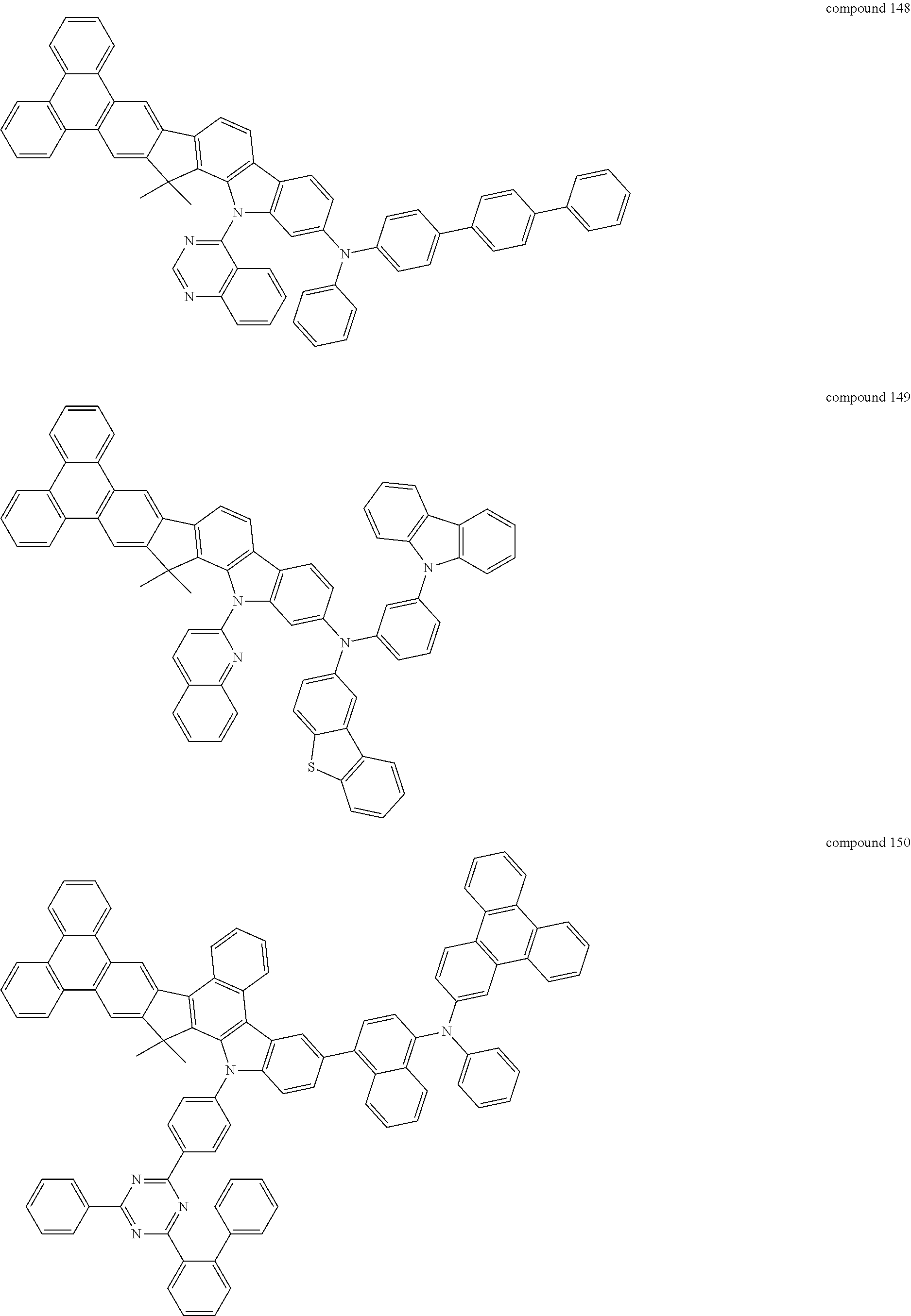 [SMILES: CC1(C)c2cc3c4ccccc4c4ccccc4c3cc2-c2ccc3c4ccc(N(c5ccccc5)c5ccc(-c6ccc(-c7ccccc7)cc6)cc5)cc4n(-c4ncnc5ccccc45)c3c21 ; CC1(C)c2cc3c4ccccc4c4ccccc4c3cc2-c2ccc3c4ccc(N(c5cccc(-n6c7ccccc7c7ccccc76)c5)c5ccc6sc7ccccc7c6c5)cc4n(-c4ccc5ccccc5n4)c3c21 ; CC1(C)c2cc3c4ccccc4c4ccccc4c3cc2-c2c1c1c(c3ccccc23)c2cc(-c3ccc(N(c4ccccc4)c4ccc5c6ccccc6c6ccccc6c5c4)c4ccccc34)ccc2n1-c1ccc(-c2nc(-c3ccccc3)nc(-c3ccccc3-c3ccccc3)n2)cc1]

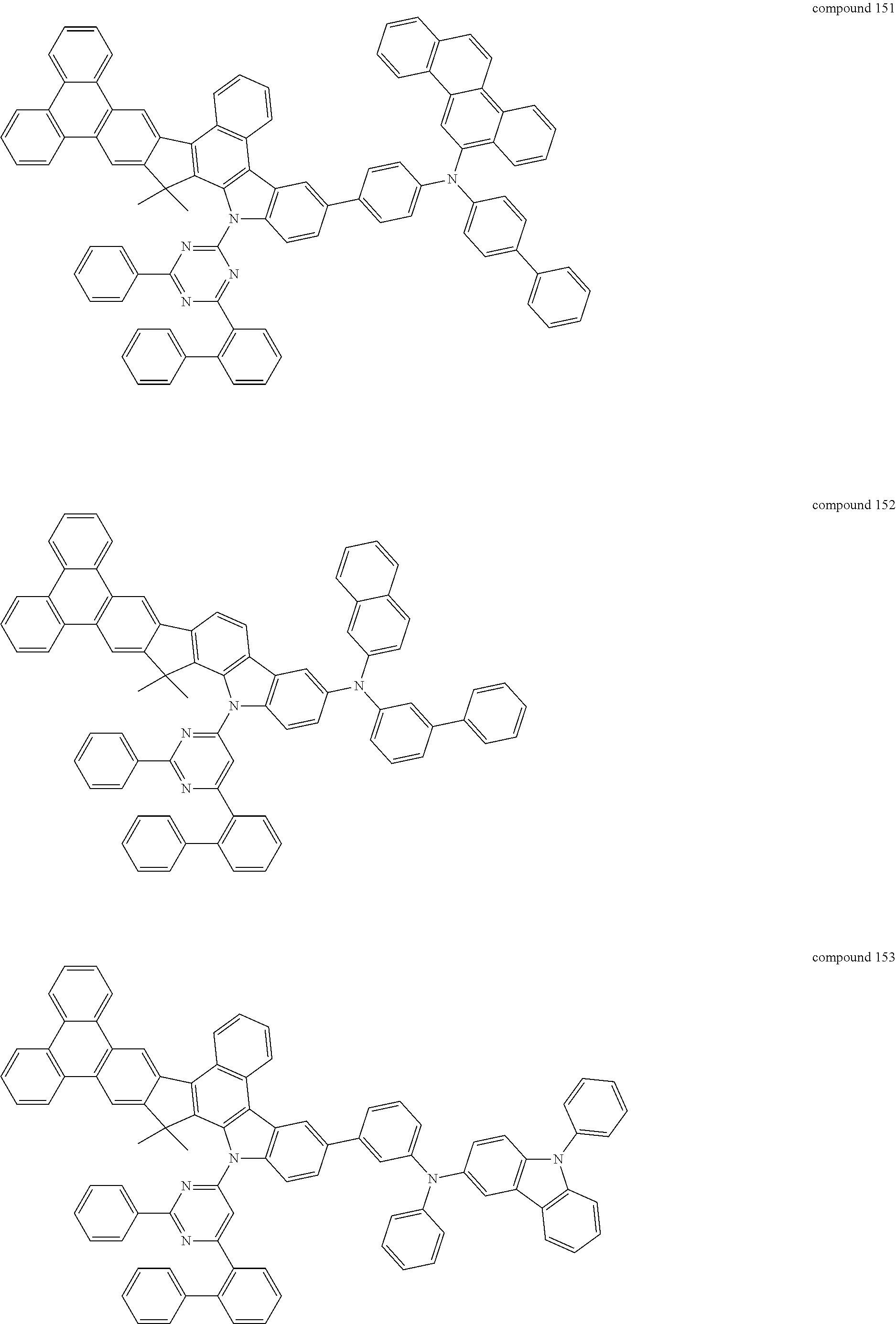 [SMILES: CC1(C)c2cc3c4ccccc4c4ccccc4c3cc2-c2c1c1c(c3ccccc23)c2cc(-c3ccc(N(c4ccc(-c5ccccc5)cc4)c4cc5c6ccccc6ccc5c5ccccc45)cc3)ccc2n1-c1nc(-c2ccccc2)nc(-c2ccccc2-c2ccccc2)n1 ; CC1(C)c2cc3c4ccccc4c4ccccc4c3cc2-c2ccc3c4cc(N(c5cccc(-c6ccccc6)c5)c5ccc6ccccc6c5)ccc4n(-c4cc(-c5ccccc5-c5ccccc5)nc(-c5ccccc5)n4)c3c21 ; CC1(C)c2cc3c4ccccc4c4ccccc4c3cc2-c2c1c1c(c3ccccc23)c2cc(-c3cccc(N(c4ccccc4)c4ccc5c(c4)c4ccccc4n5-c4ccccc4)c3)ccc2n1-c1cc(-c2ccccc2-c2ccccc2)nc(-c2ccccc2)n1]

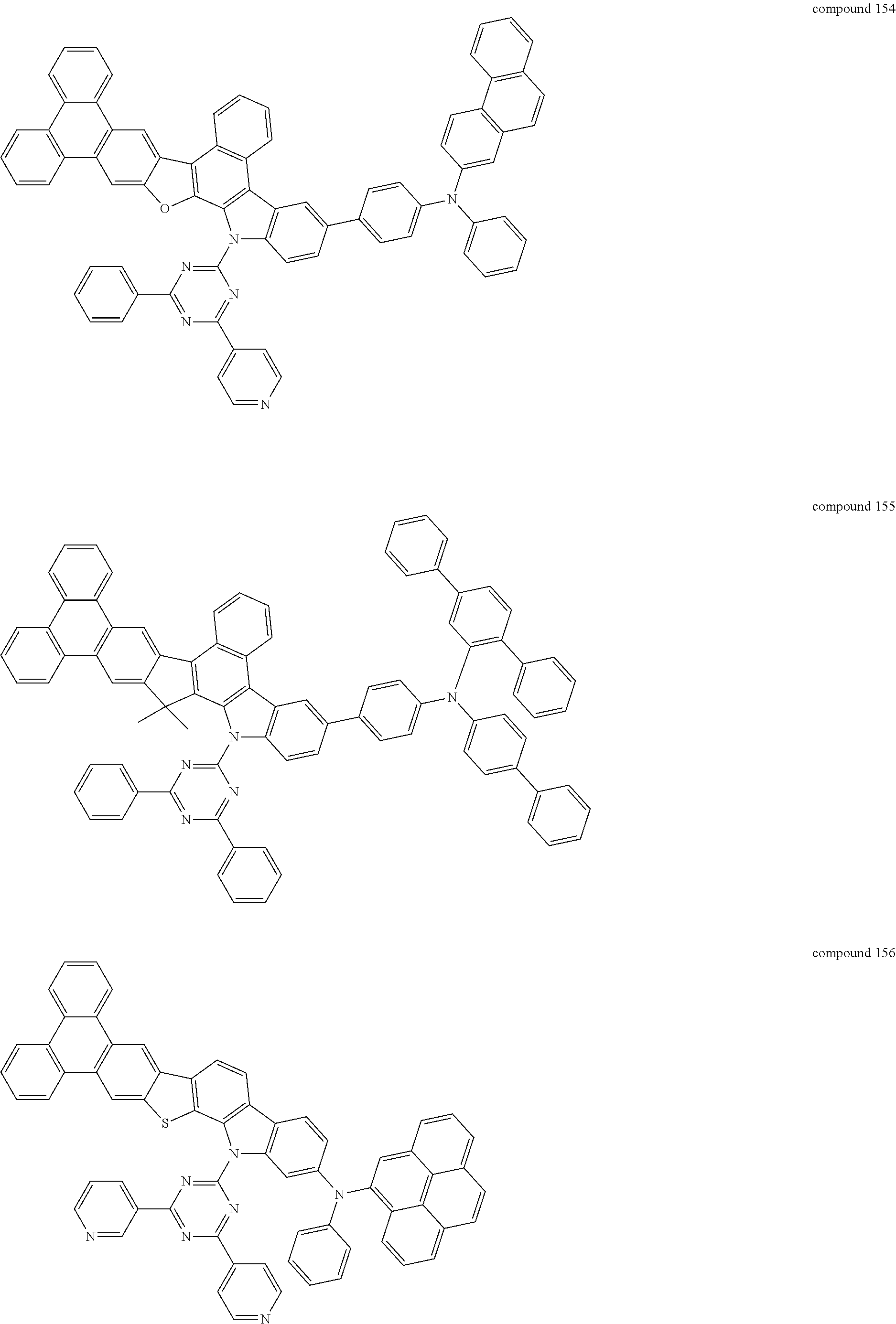 [SMILES: c1ccc(-c2nc(-c3ccncc3)nc(-n3c4ccc(-c5ccc(N(c6ccccc6)c6ccc7c(ccc8ccccc87)c6)cc5)cc4c4c5ccccc5c5c6cc7c8ccccc8c8ccccc8c7cc6oc5c43)n2)cc1 ; CC1(C)c2cc3c4ccccc4c4ccccc4c3cc2-c2c1c1c(c3ccccc23)c2cc(-c3ccc(N(c4ccc(-c5ccccc5)cc4)c4cc(-c5ccccc5)ccc4-c4ccccc4)cc3)ccc2n1-c1nc(-c2ccccc2)nc(-c2ccccc2)n1 ; c1ccc(N(c2ccc3c4ccc5c6cc7c8ccccc8c8ccccc8c7cc6sc5c4n(-c4nc(-c5ccncc5)nc(-c5cccnc5)n4)c3c2)c2cc3cccc4ccc5cccc2c5c43)cc1]

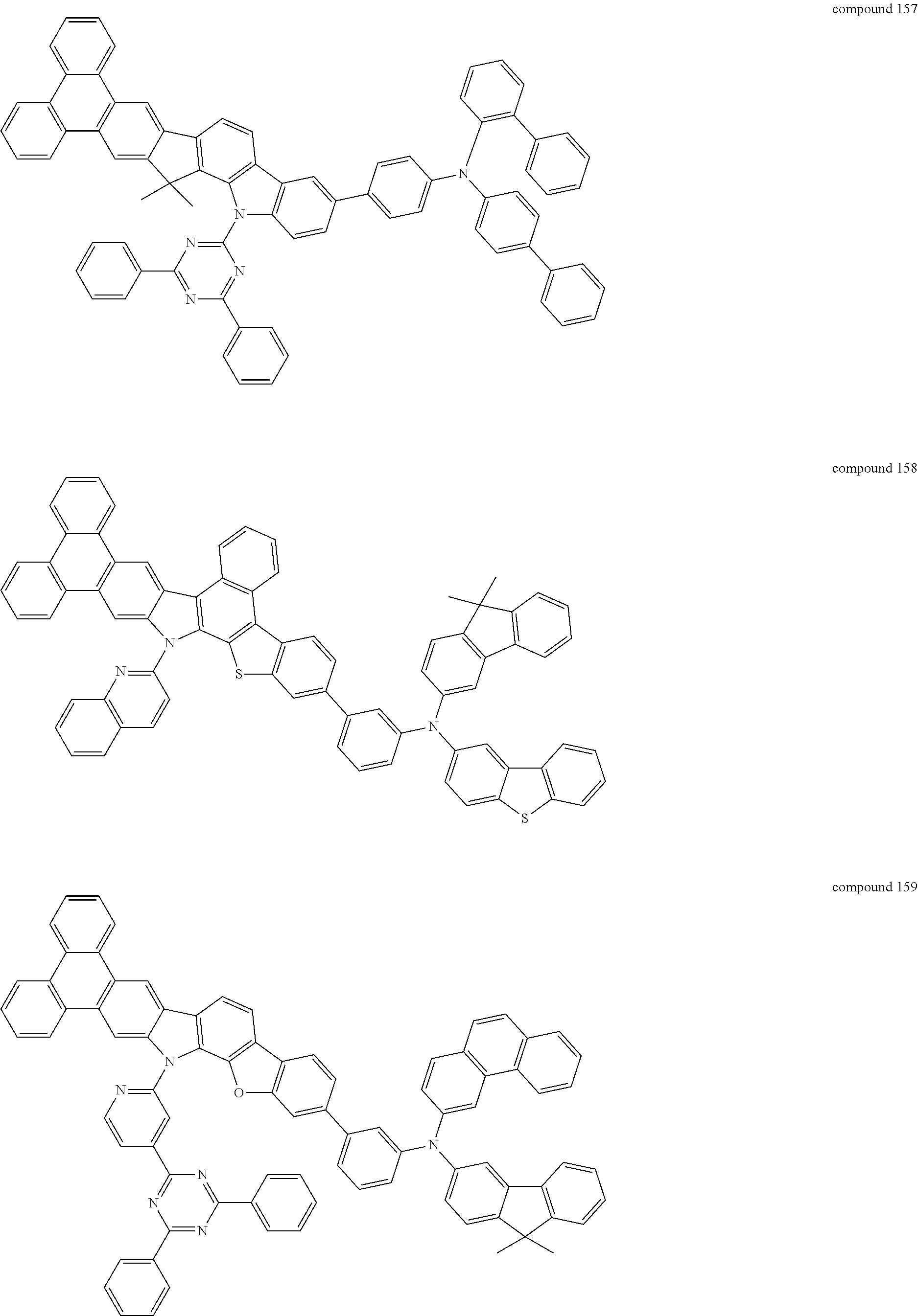 [SMILES: CC1(C)c2cc3c4ccccc4c4ccccc4c3cc2-c2ccc3c4cc(-c5ccc(N(c6ccc(-c7ccccc7)cc6)c6ccccc6-c6ccccc6)cc5)ccc4n(-c4nc(-c5ccccc5)nc(-c5ccccc5)n4)c3c21 ; CC1(C)c2ccccc2-c2cc(N(c3cccc(-c4ccc5c(c4)sc4c5c5ccccc5c5c6cc7c8ccccc8c8ccccc8c7cc6n(-c6ccc7ccccc7n6)c45)c3)c3ccc4sc5ccccc5c4c3)ccc21 ; CC1(C)c2ccccc2-c2cc(N(c3cccc(-c4ccc5c(c4)oc4c5ccc5c6cc7c8ccccc8c8ccccc8c7cc6n(-c6cc(-c7nc(-c8ccccc8)nc(-c8ccccc8)n7)ccn6)c54)c3)c3ccc4ccc5ccccc5c4c3)ccc21]

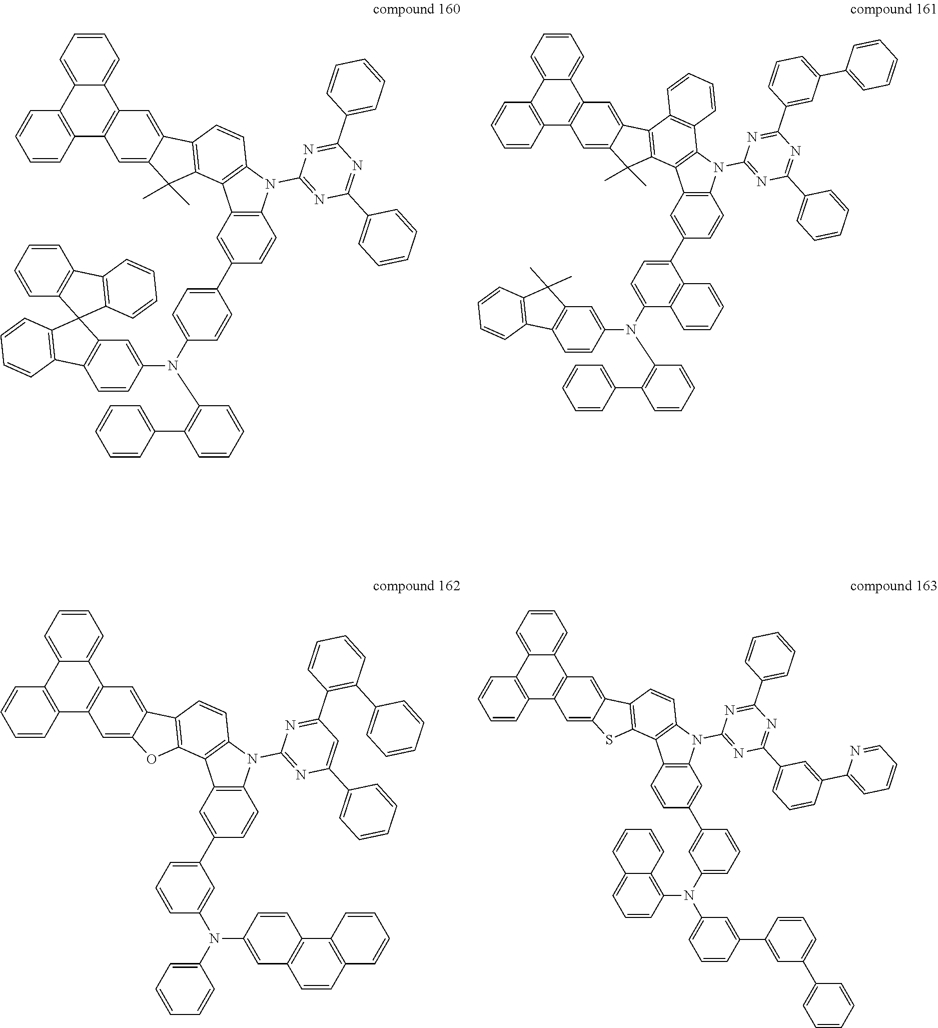

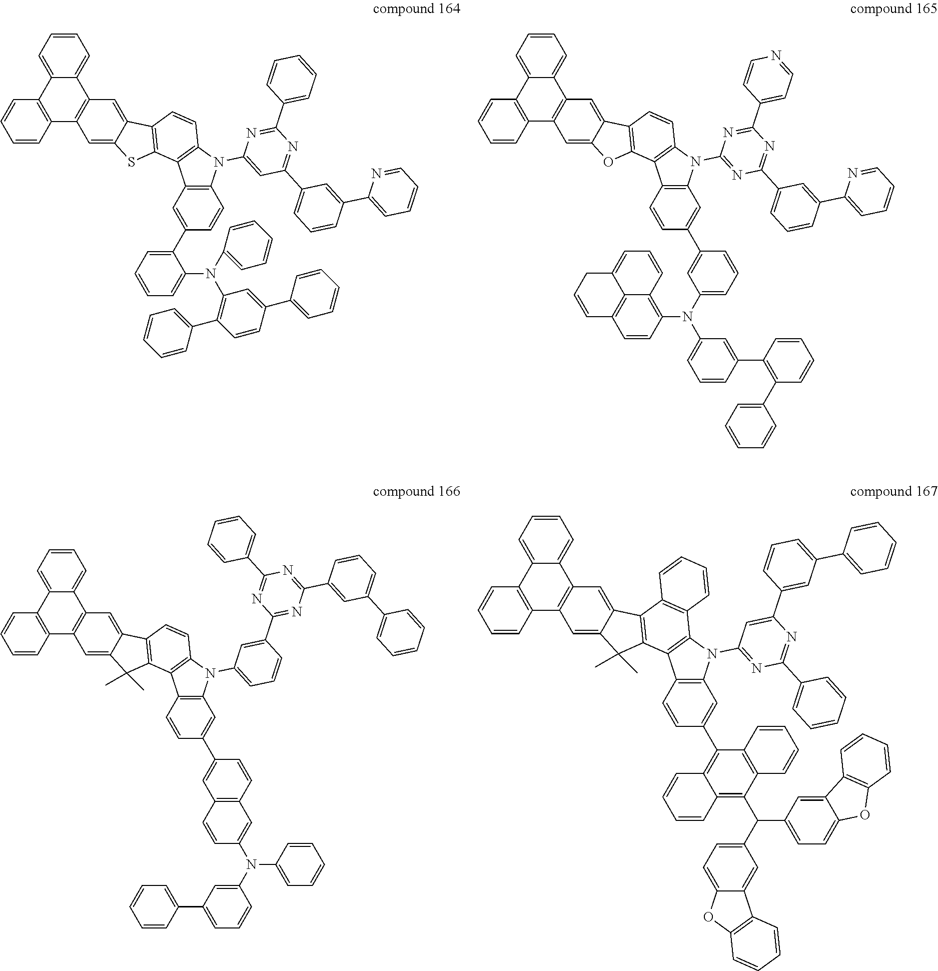

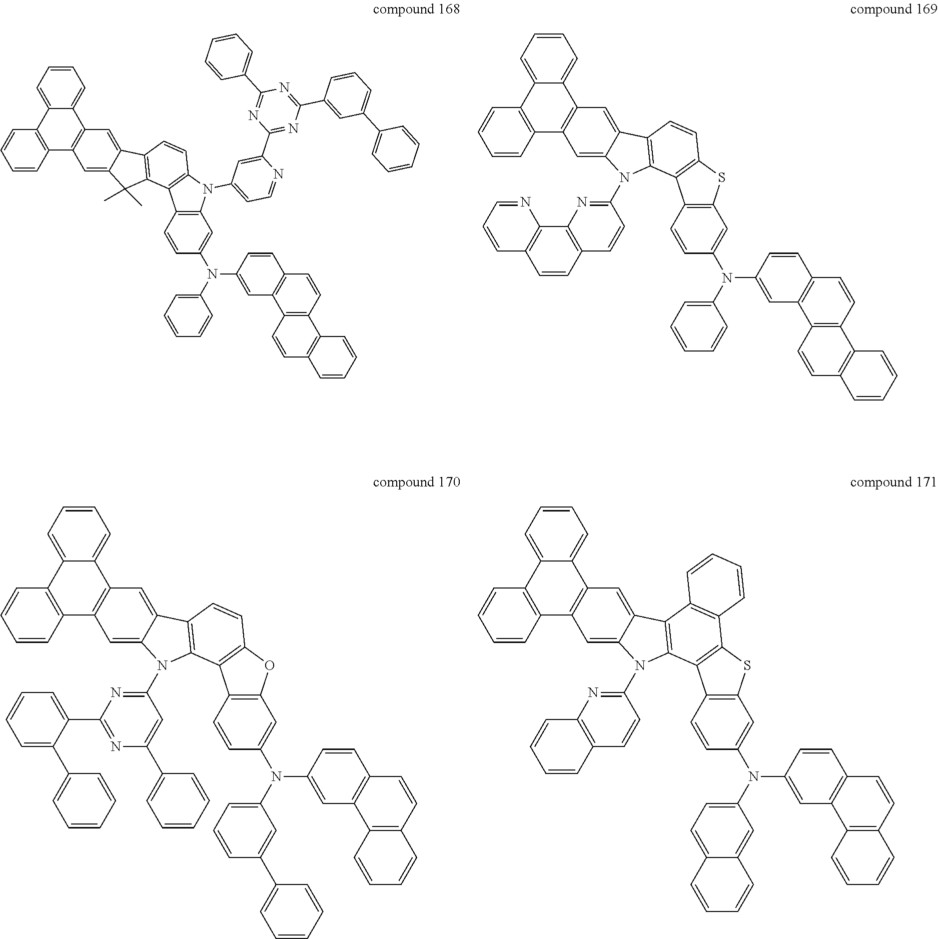

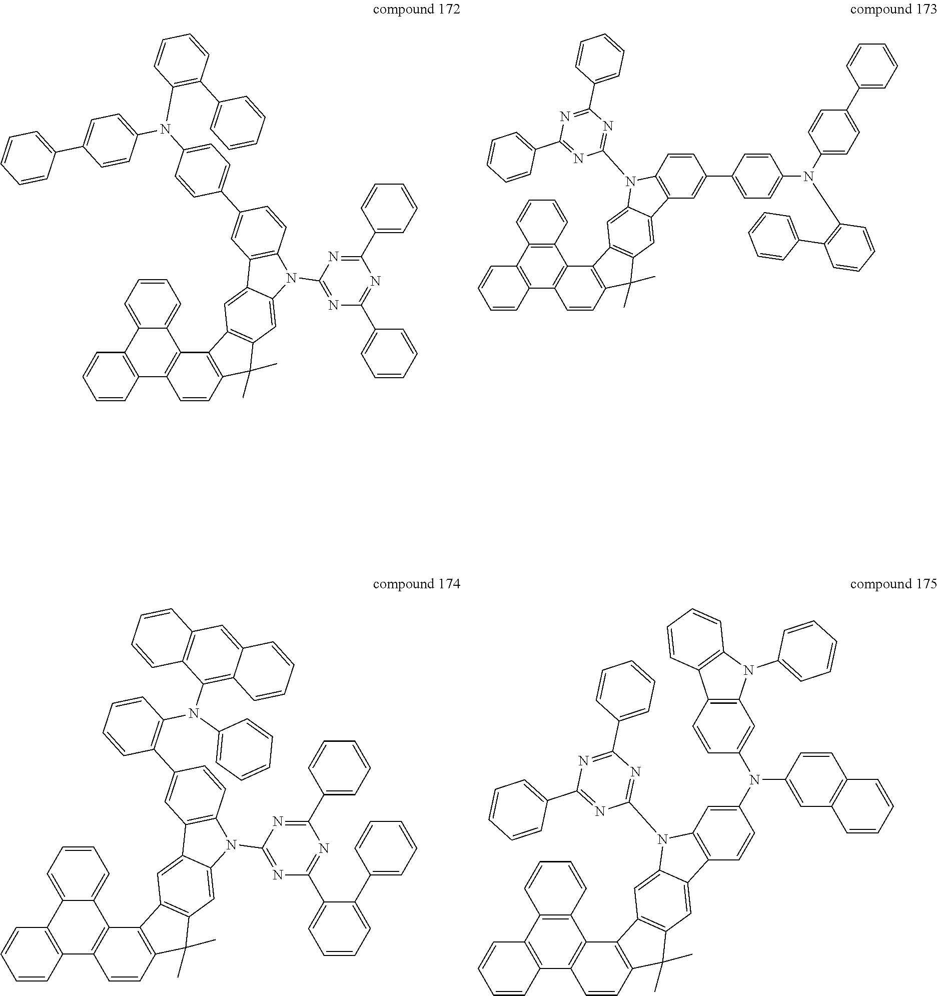

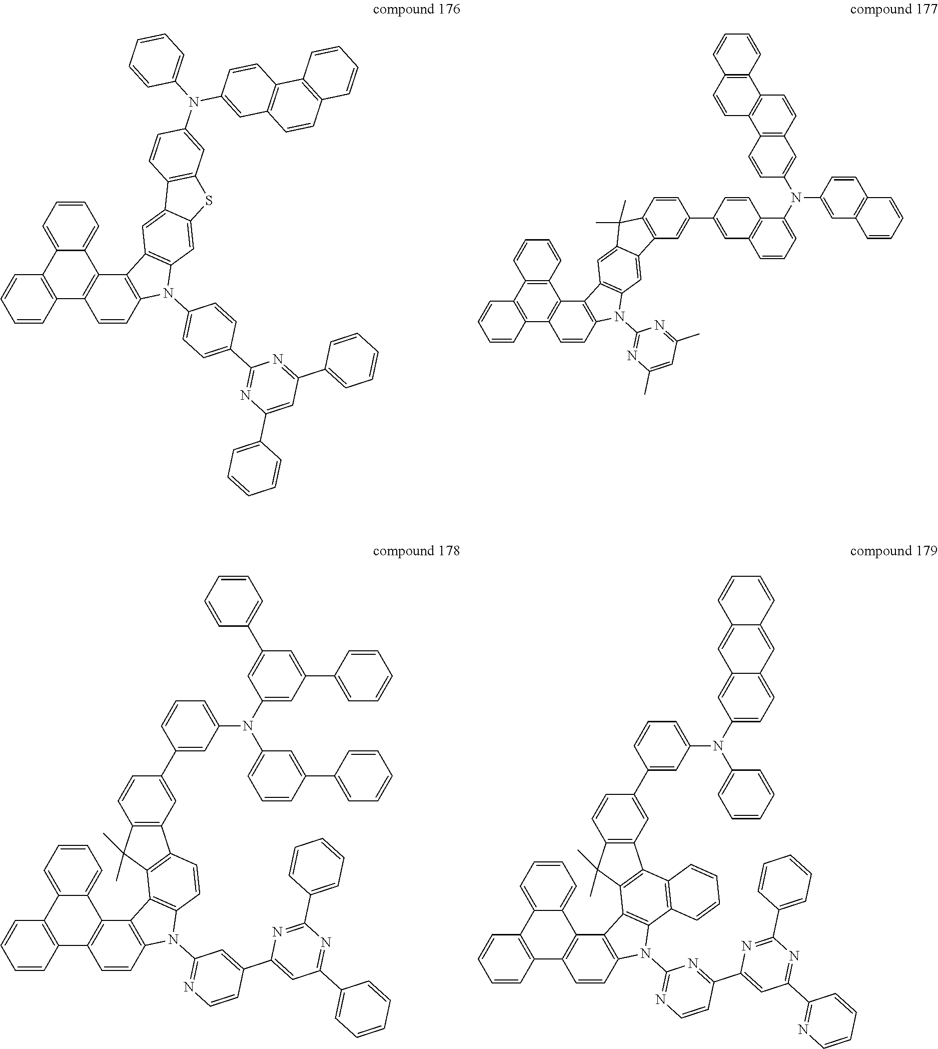

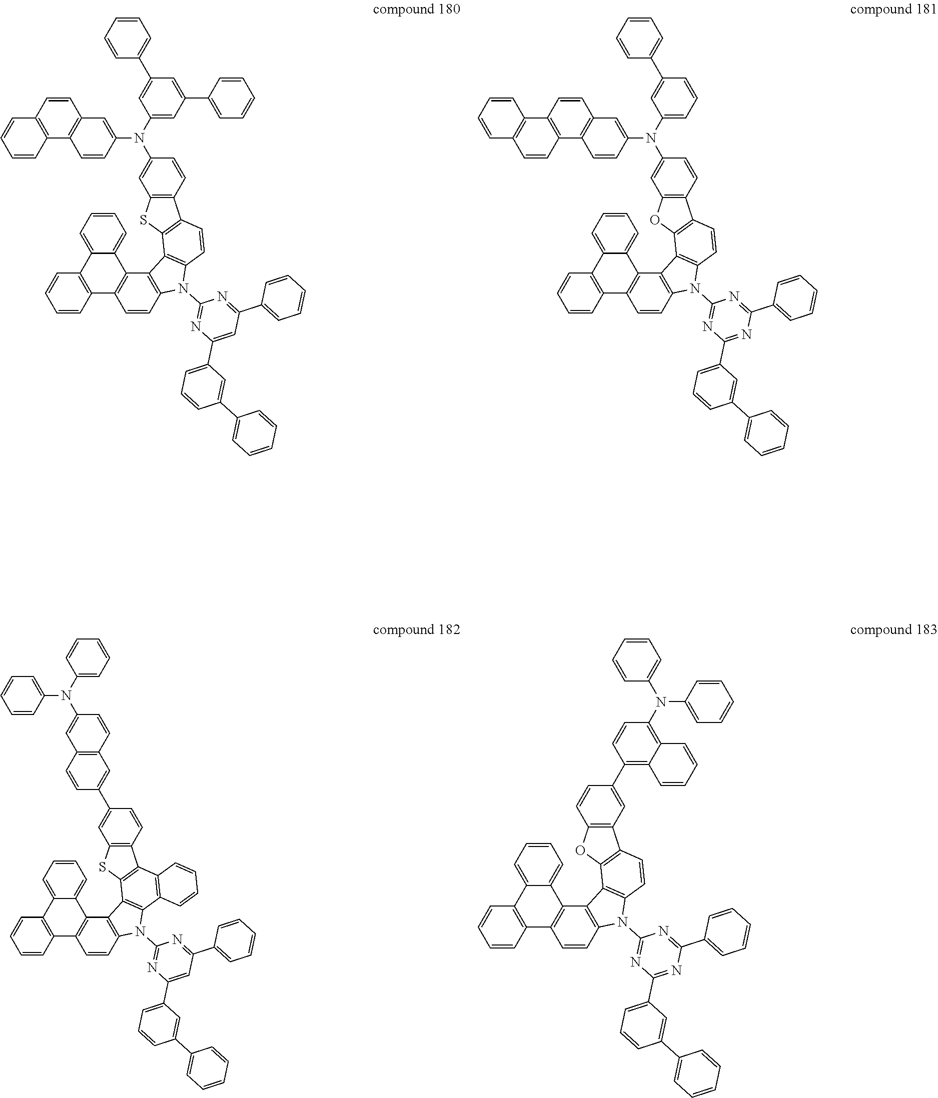

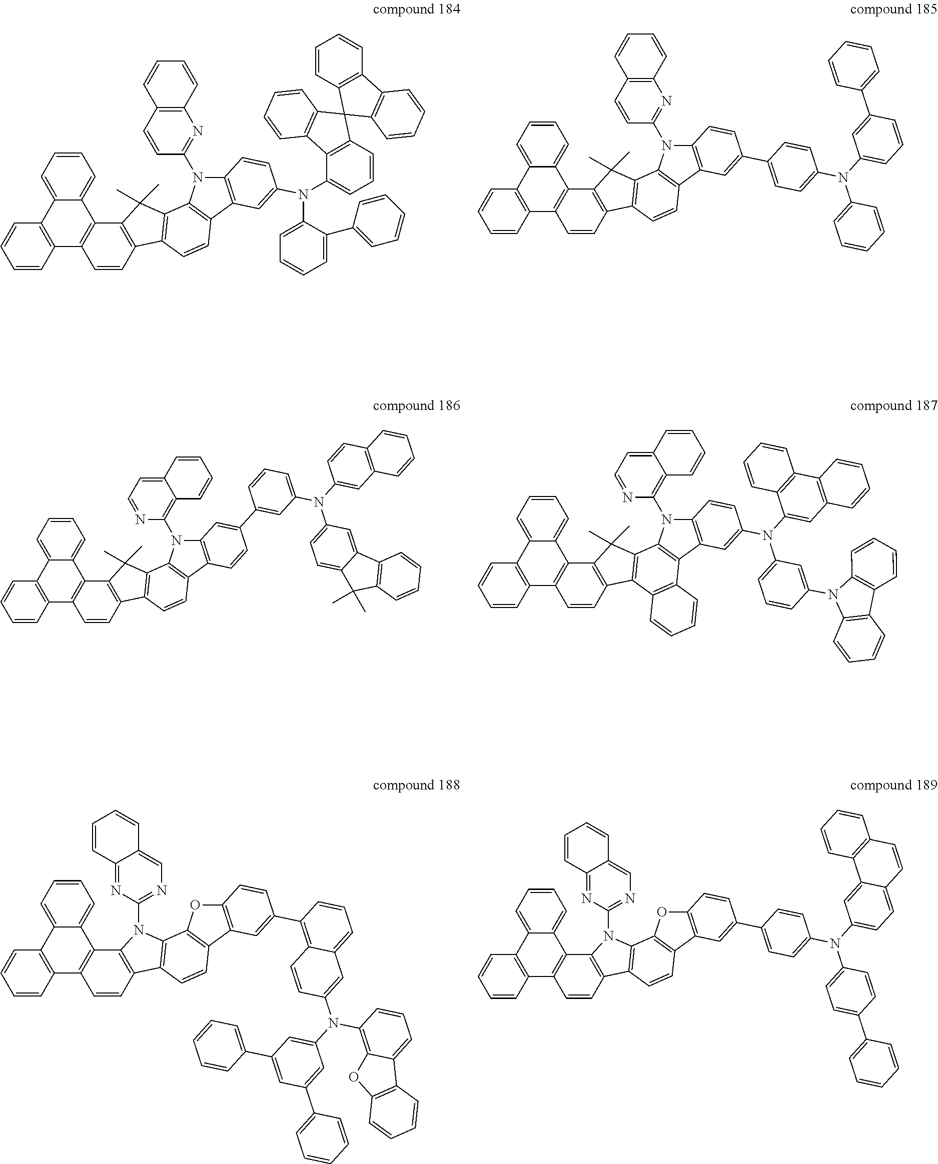

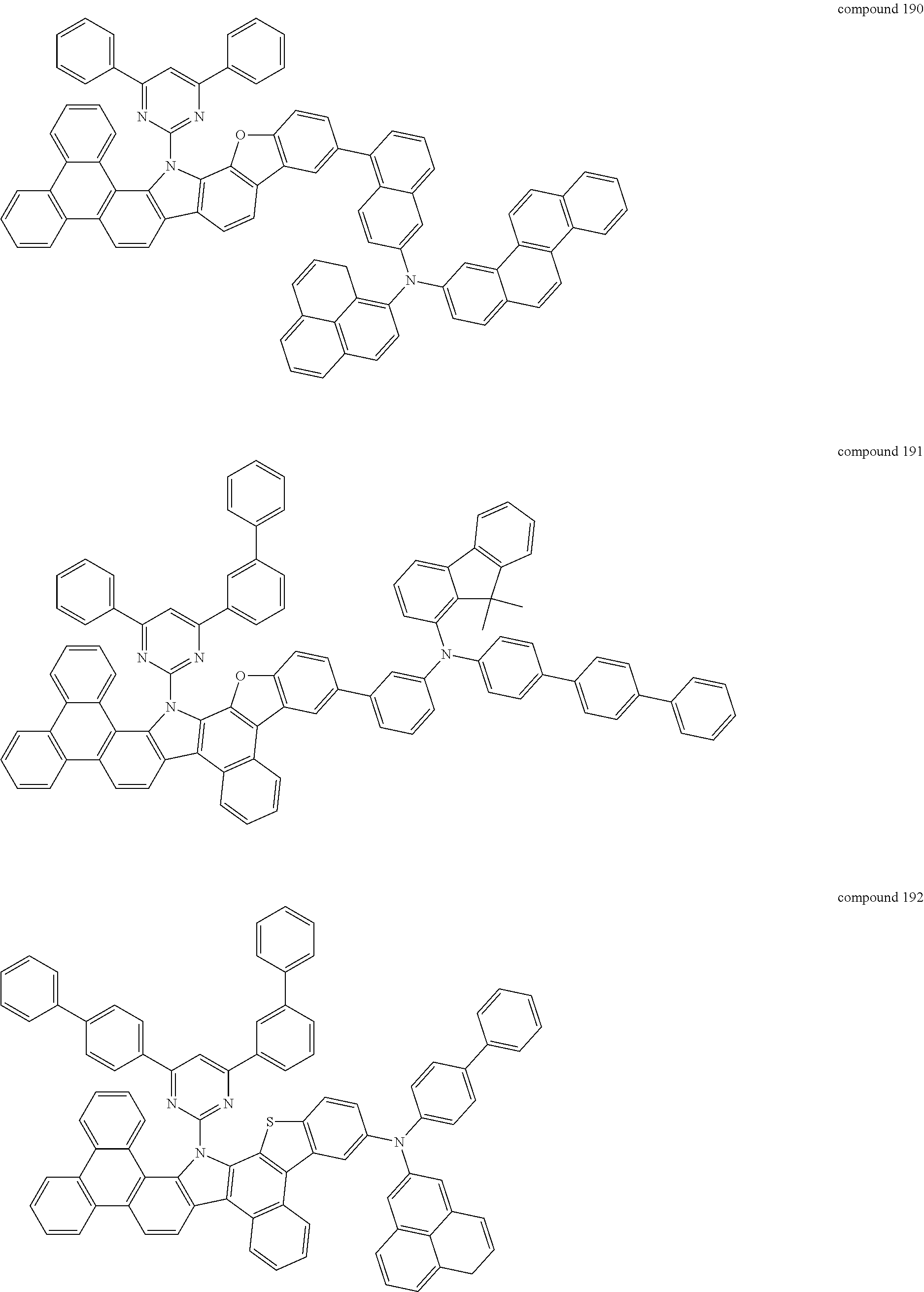

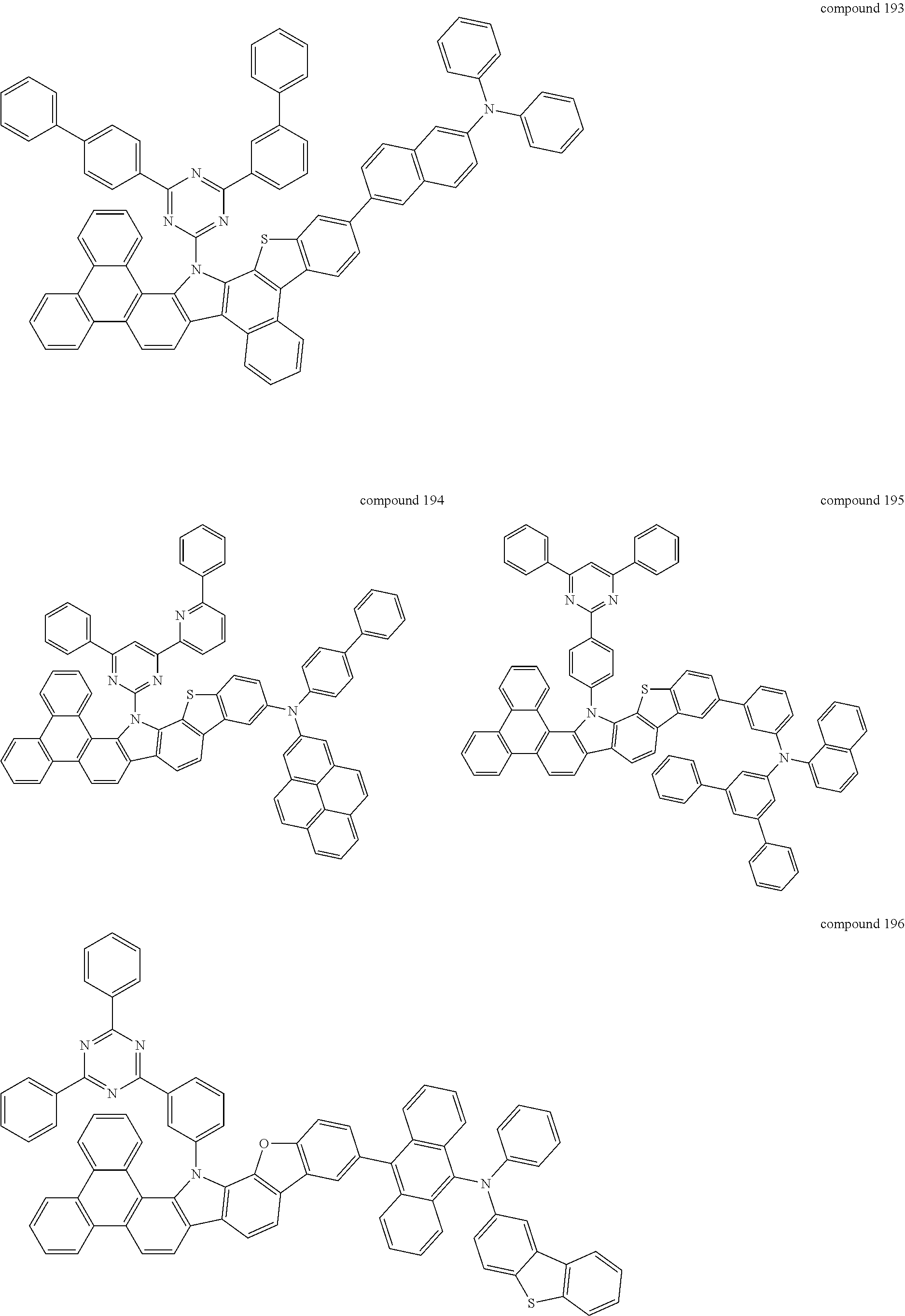

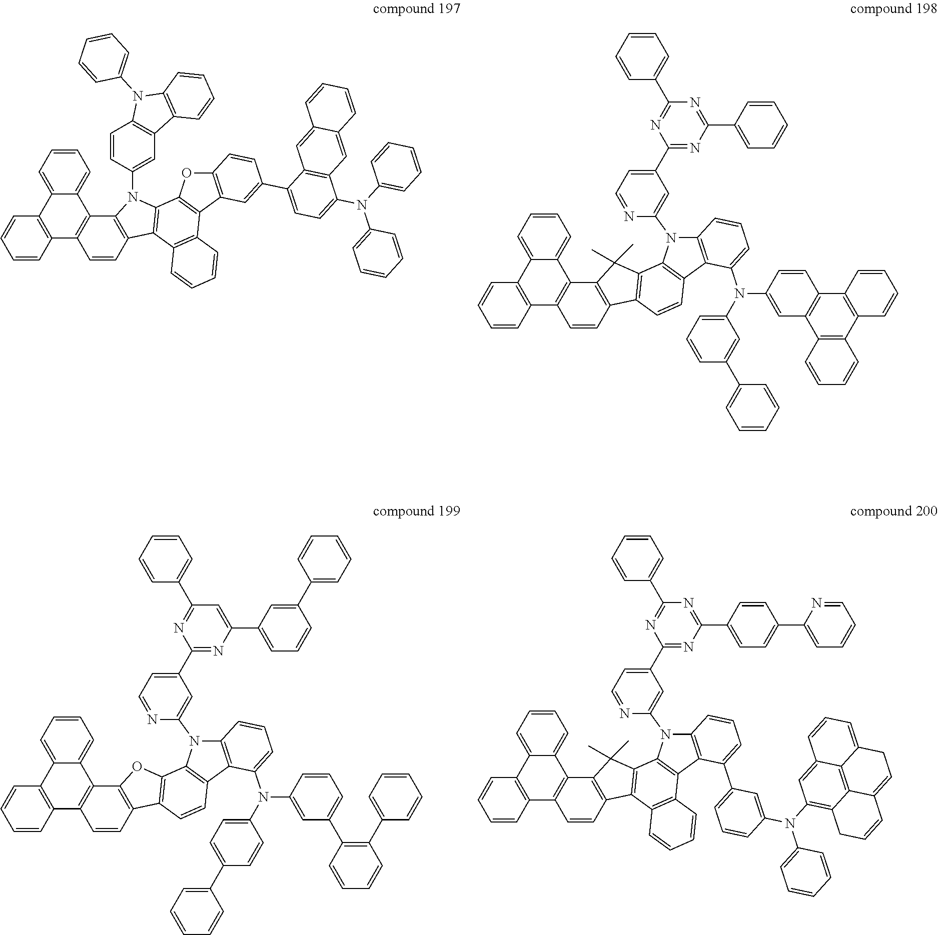

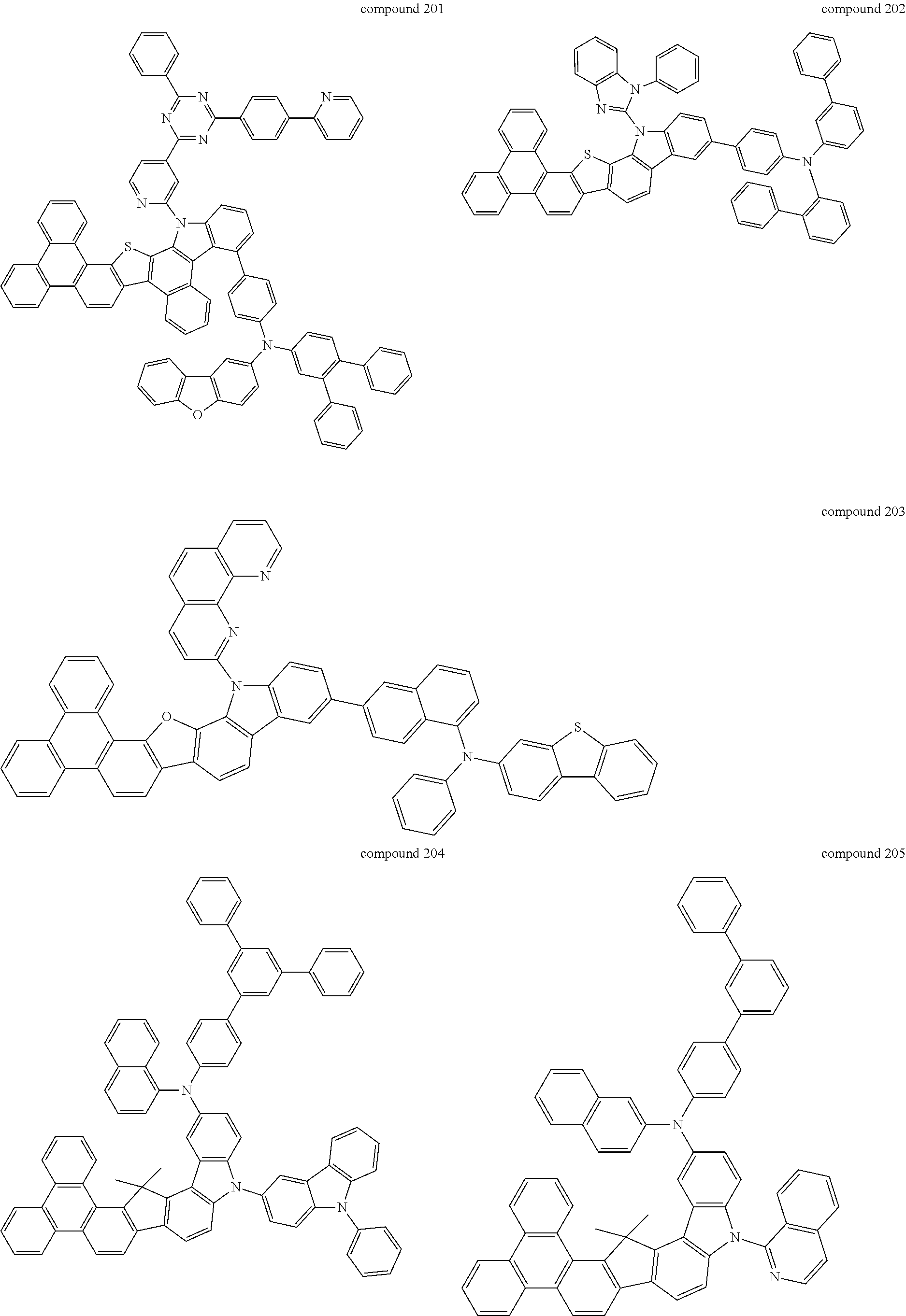

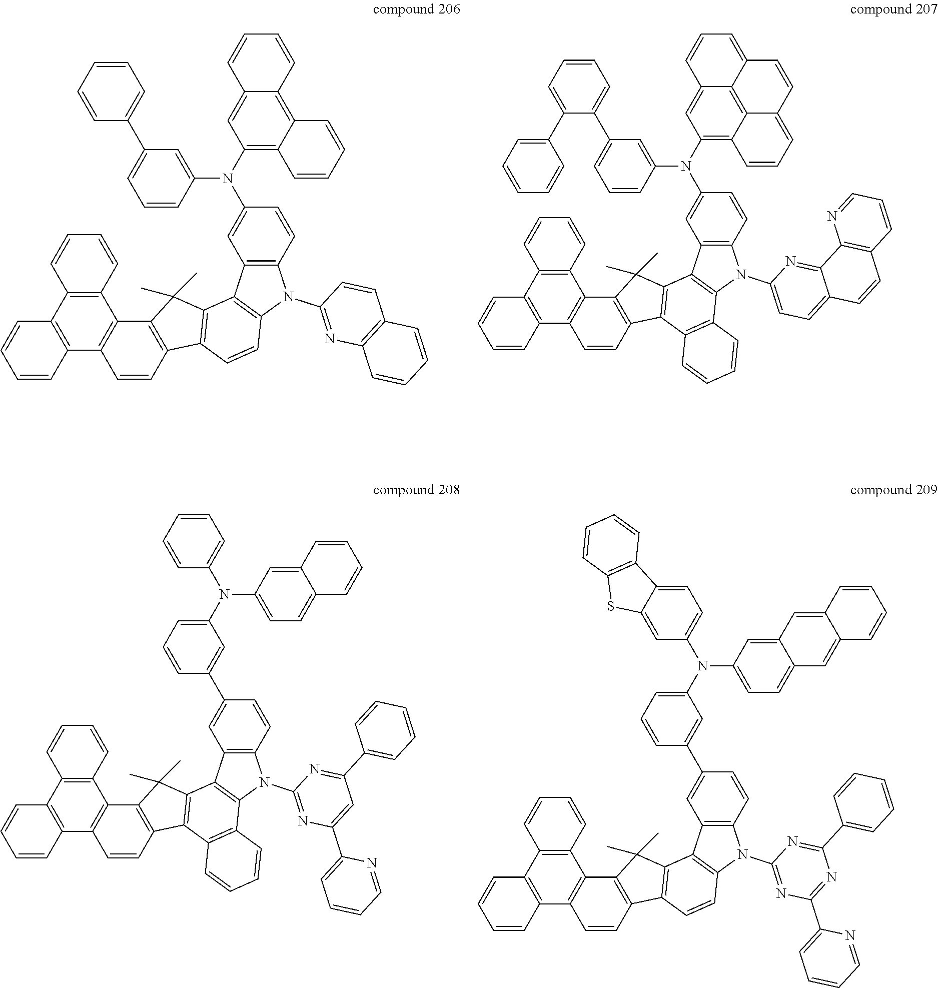

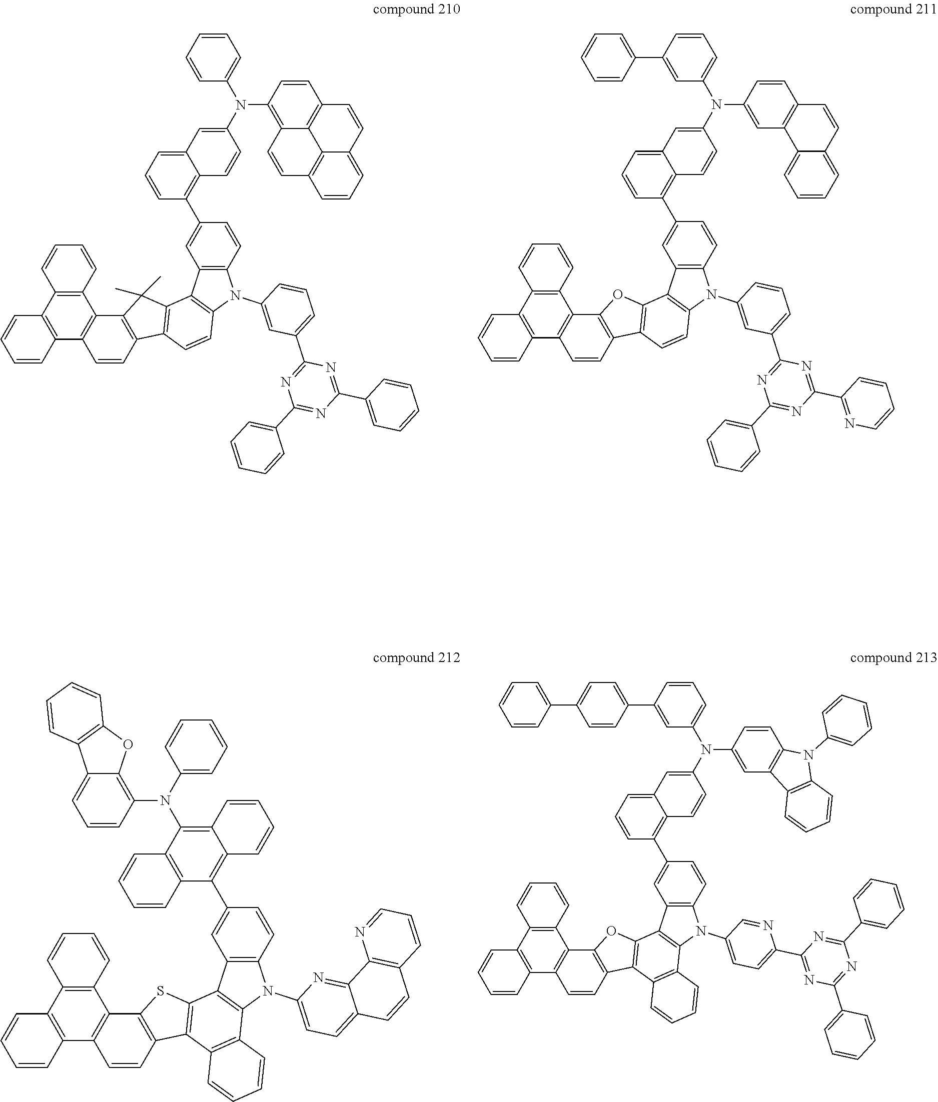

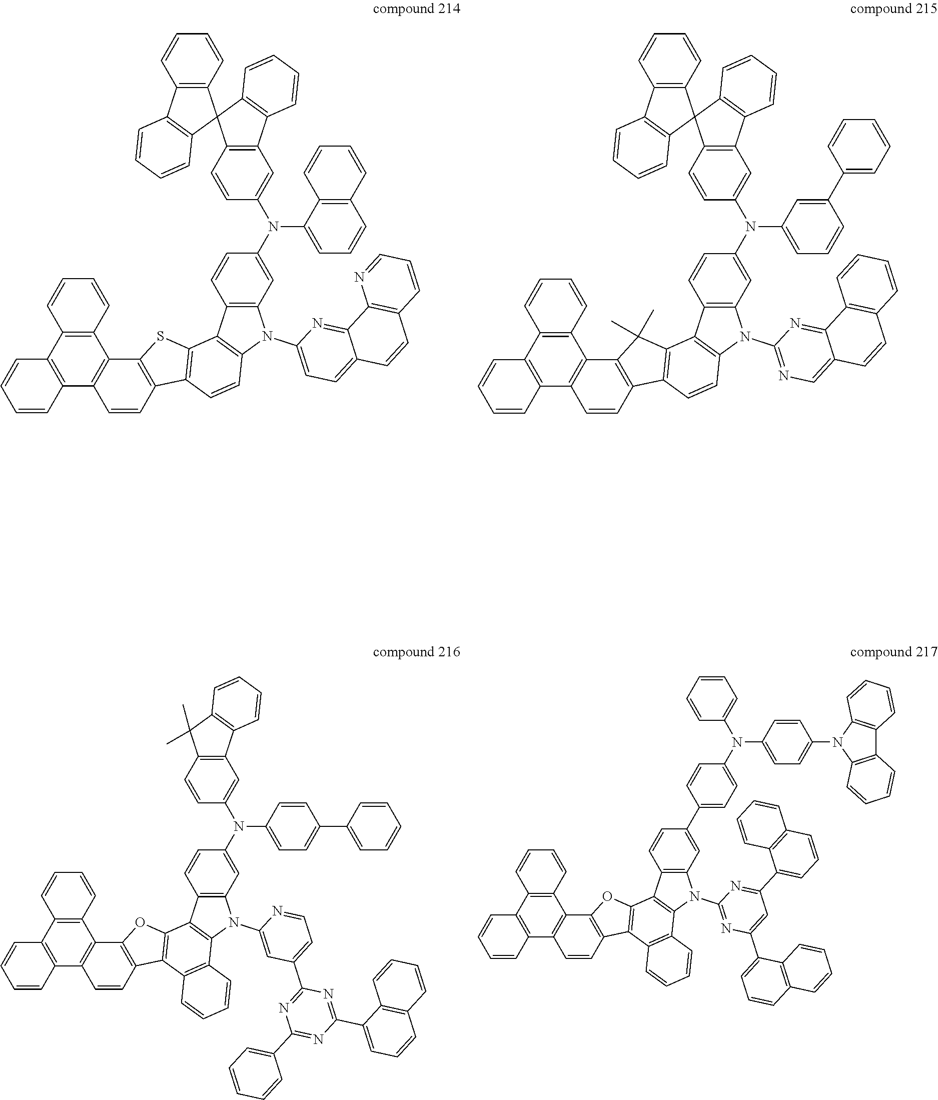

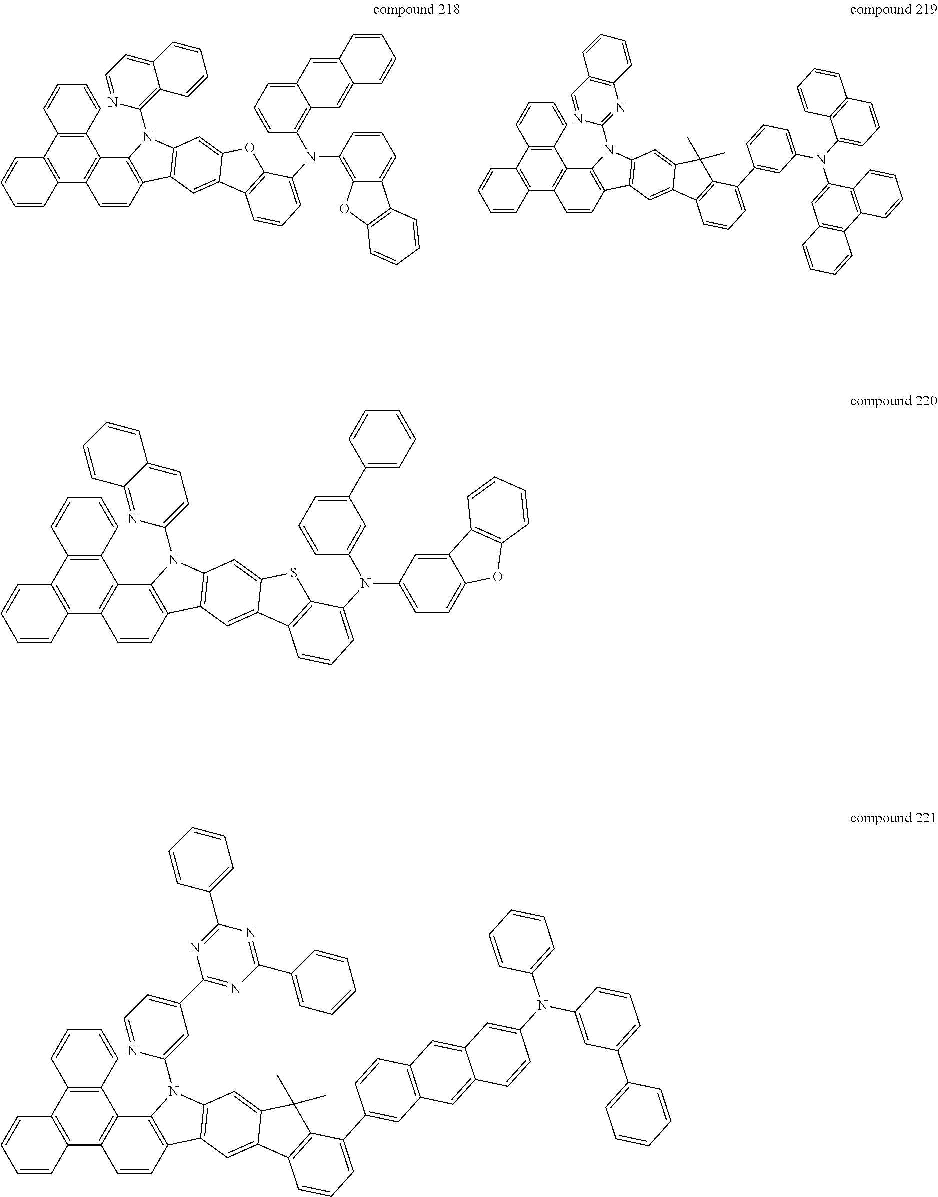 [SMILES: c1ccc2cc3c(N(c4cccc5c4oc4ccccc45)c4cccc5c4oc4cc6c(cc45)c4ccc5c7ccccc7c7ccccc7c5c4n6-c4nccc5ccccc45)cccc3cc2c1 ; CC1(C)c2cc3c(cc2-c2cccc(-c4cccc(N(c5cccc6ccccc56)c5cc6ccccc6c6ccccc56)c4)c21)c1ccc2c4ccccc4c4ccccc4c2c1n3-c1ncc2ccccc2n1 ; c1ccc(-c2cccc(N(c3ccc4oc5ccccc5c4c3)c3cccc4c3sc3cc5c(cc34)c3ccc4c6ccccc6c6ccccc6c4c3n5-c3ccc4ccccc4n3)c2)cc1 ; CC1(C)c2cc3c(cc2-c2cccc(-c4ccc5cc6cc(N(c7ccccc7)c7cccc(-c8ccccc8)c7)ccc6cc5c4)c21)c1ccc2c4ccccc4c4ccccc4c2c1n3-c1cc(-c2nc(-c3ccccc3)nc(-c3ccccc3)n2)ccn1]

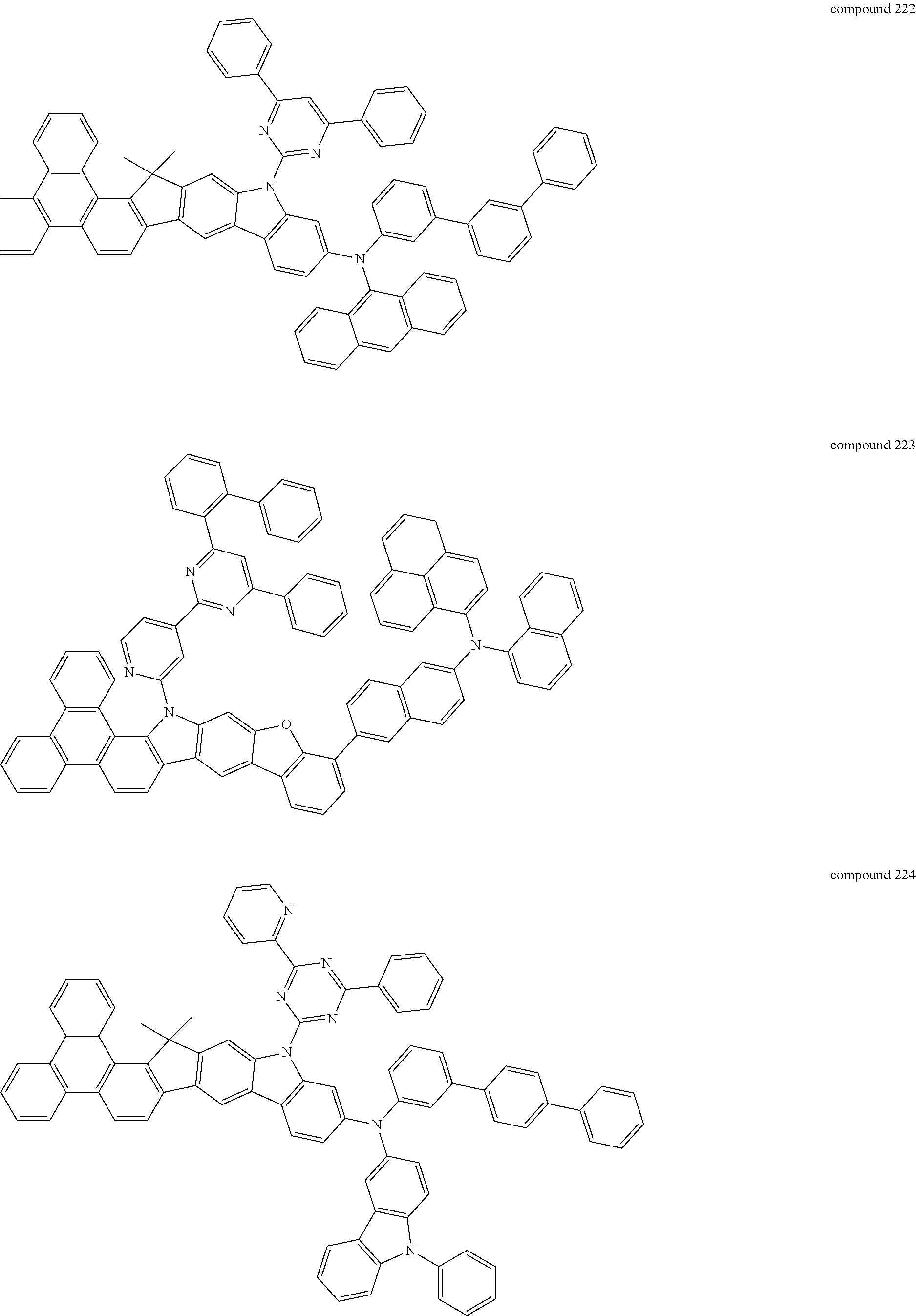 [SMILES: C=Cc1c(C)c2ccccc2c2c3c(ccc12)-c1cc2c4ccc(N(c5cccc(-c6cccc(-c7ccccc7)c6)c5)c5c6ccccc6cc6ccccc56)cc4n(-c4nc(-c5ccccc5)cc(-c5ccccc5)n4)c2cc1C3(C)C ; C1=Cc2cccc3c(N(c4ccc5cc(-c6cccc7c6oc6cc8c(cc67)c6ccc7c9ccccc9c9ccccc9c7c6n8-c6cc(-c7nc(-c8ccccc8)cc(-c8ccccc8-c8ccccc8)n7)ccn6)ccc5c4)c4cccc5ccccc45)ccc(c23)C1 ; CC1(C)c2cc3c(cc2-c2ccc4c5ccccc5c5ccccc5c4c21)c1ccc(N(c2cccc(-c4ccc(-c5ccccc5)cc4)c2)c2ccc4c(c2)c2ccccc2n4-c2ccccc2)cc1n3-c1nc(-c2ccccc2)nc(-c2ccccn2)n1]

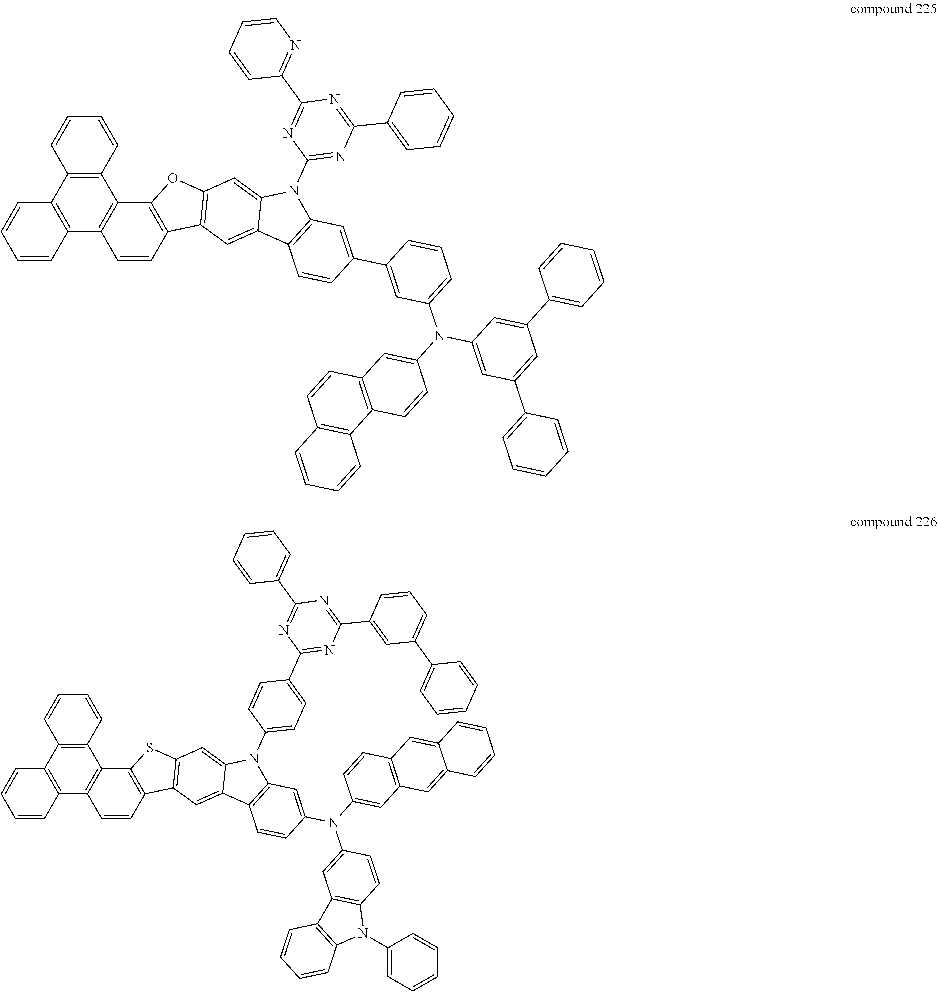 [SMILES: c1ccc(-c2cc(-c3ccccc3)cc(N(c3cccc(-c4ccc5c6cc7c(cc6n(-c6nc(-c8ccccc8)nc(-c8ccccn8)n6)c5c4)oc4c7ccc5c6ccccc6c6ccccc6c54)c3)c3ccc4c(ccc5ccccc54)c3)c2)cc1 ; c1ccc(-c2cccc(-c3nc(-c4ccccc4)nc(-c4ccc(-n5c6cc(N(c7ccc8cc9ccccc9cc8c7)c7ccc8c(c7)c7ccccc7n8-c7ccccc7)ccc6c6cc7c(cc65)sc5c7ccc6c7ccccc7c7ccccc7c65)cc4)n3)c2)cc1]

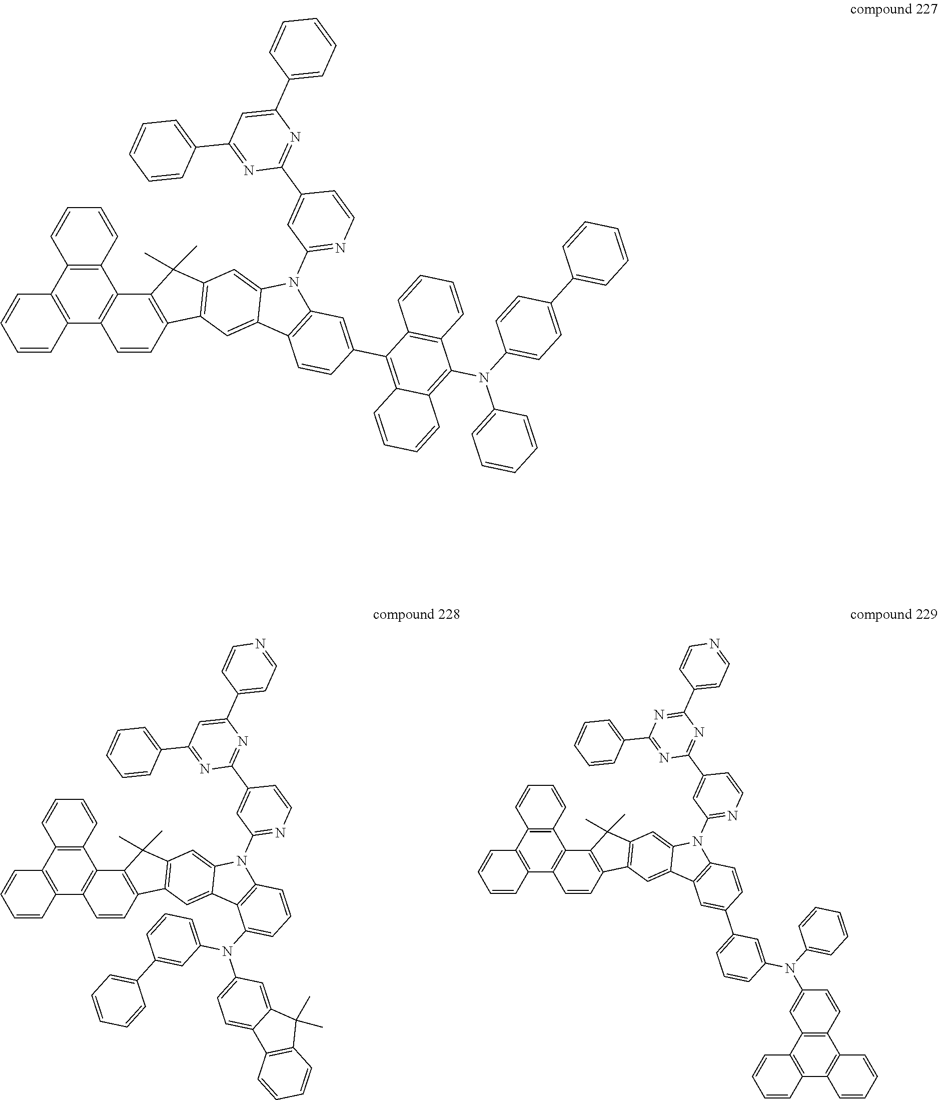 [SMILES: CC1(C)c2cc3c(cc2-c2ccc4c5ccccc5c5ccccc5c4c21)c1ccc(-c2c4ccccc4c(N(c4ccccc4)c4ccc(-c5ccccc5)cc4)c4ccccc24)cc1n3-c1cc(-c2nc(-c3ccccc3)cc(-c3ccccc3)n2)ccn1 ; CC1(C)c2ccccc2-c2ccc(N(c3cccc(-c4ccccc4)c3)c3cccc4c3c3cc5c(cc3n4-c3cc(-c4nc(-c6ccccc6)cc(-c6ccncc6)n4)ccn3)C(C)(C)c3c-5ccc4c5ccccc5c5ccccc5c34)cc21 ; CC1(C)c2cc3c(cc2-c2ccc4c5ccccc5c5ccccc5c4c21)c1cc(-c2cccc(N(c4ccccc4)c4ccc5c6ccccc6c6ccccc6c5c4)c2)ccc1n3-c1cc(-c2nc(-c3ccccc3)nc(-c3ccncc3)n2)ccn1]

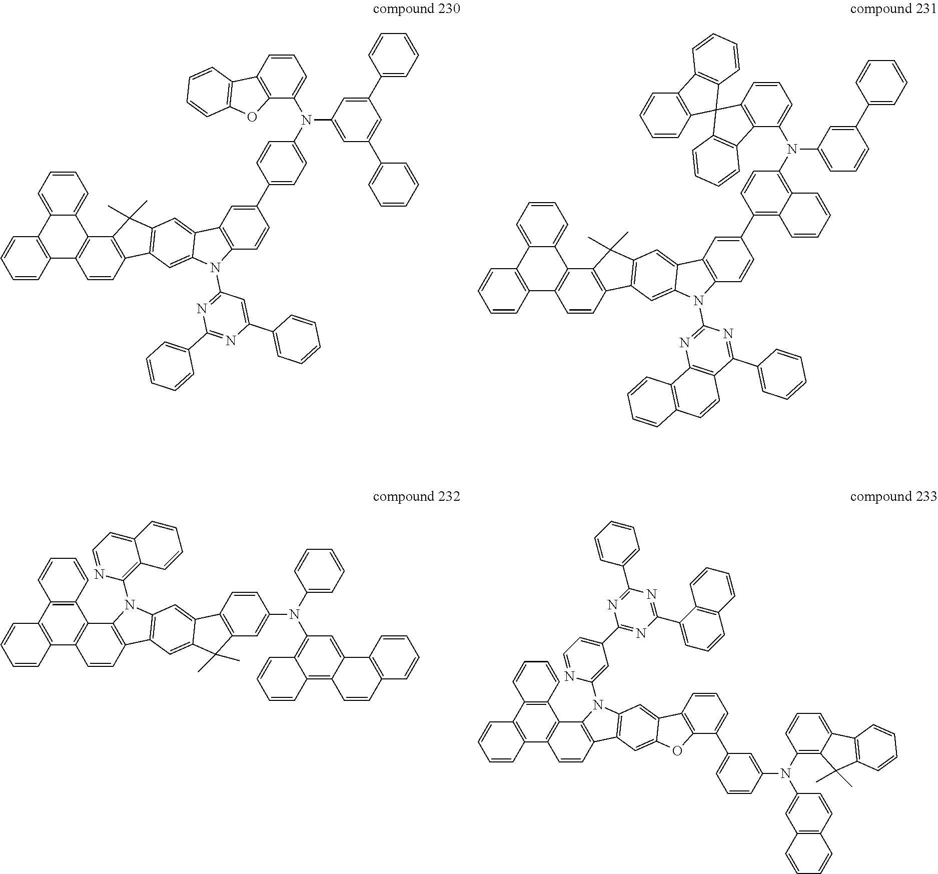

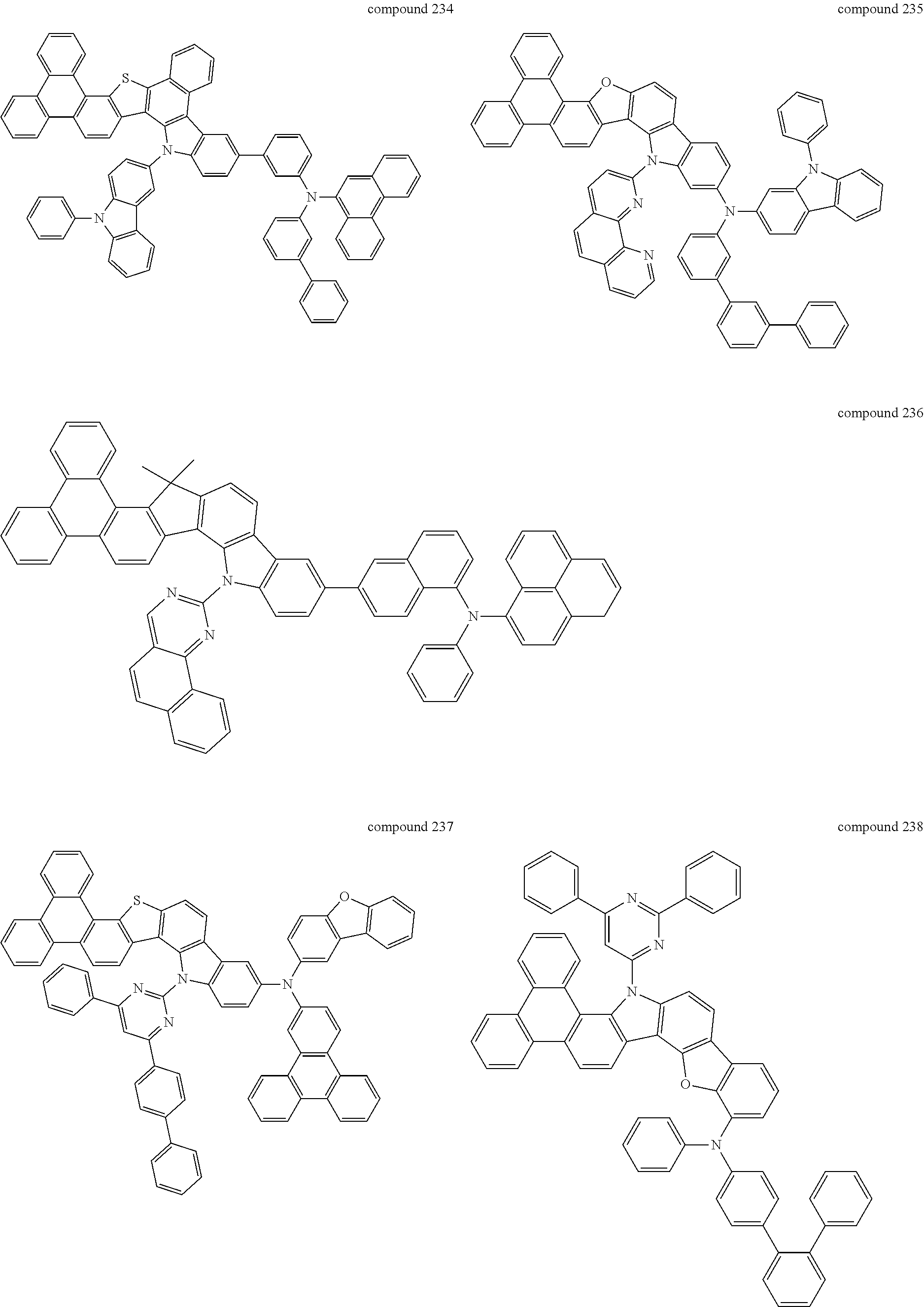

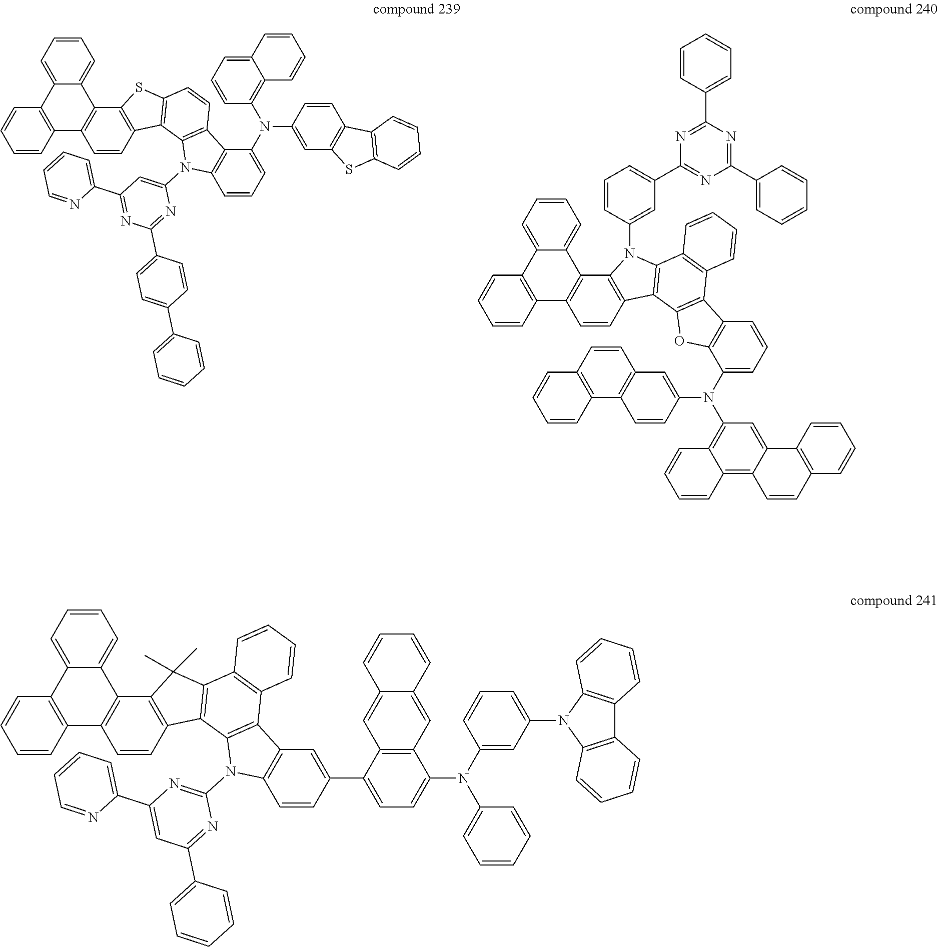 [SMILES: c1ccc(-c2ccc(-c3nc(-c4ccccn4)cc(-n4c5cccc(N(c6ccc7c(c6)sc6ccccc67)c6cccc7ccccc67)c5c5ccc6sc7c(ccc8c9ccccc9c9ccccc9c87)c6c54)n3)cc2)cc1 ; c1ccc(-c2nc(-c3ccccc3)nc(-c3cccc(-n4c5c(ccc6c7ccccc7c7ccccc7c65)c5c6oc7c(N(c8ccc9c(ccc%10ccccc%109)c8)c8cc9c%10ccccc%10ccc9c9ccccc89)cccc7c6c6ccccc6c54)c3)n2)cc1 ; CC1(C)c2c(c3c(c4ccccc24)c2cc(-c4ccc(N(c5ccccc5)c5cccc(-n6c7ccccc7c7ccccc76)c5)c5cc6ccccc6cc45)ccc2n3-c2nc(-c3ccccc3)cc(-c3ccccn3)n2)-c2ccc3c4ccccc4c4ccccc4c3c21]

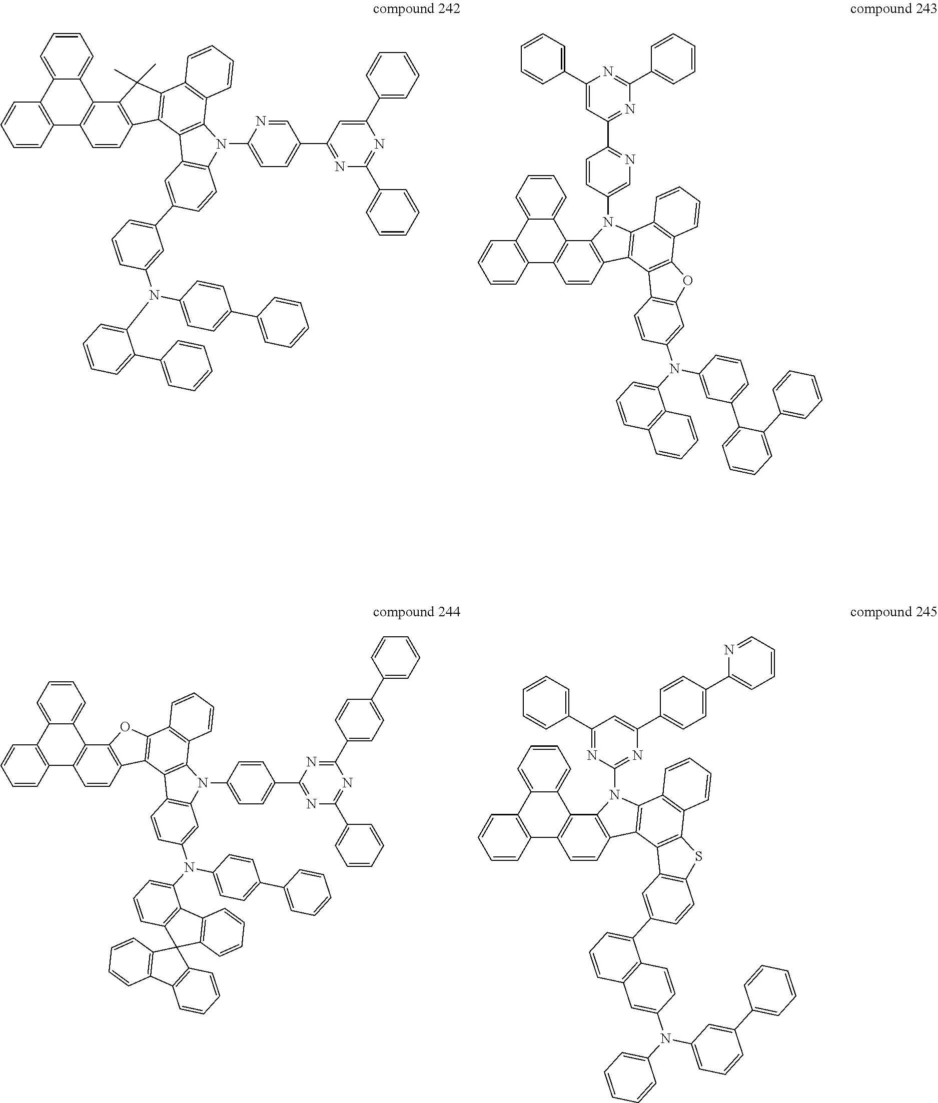

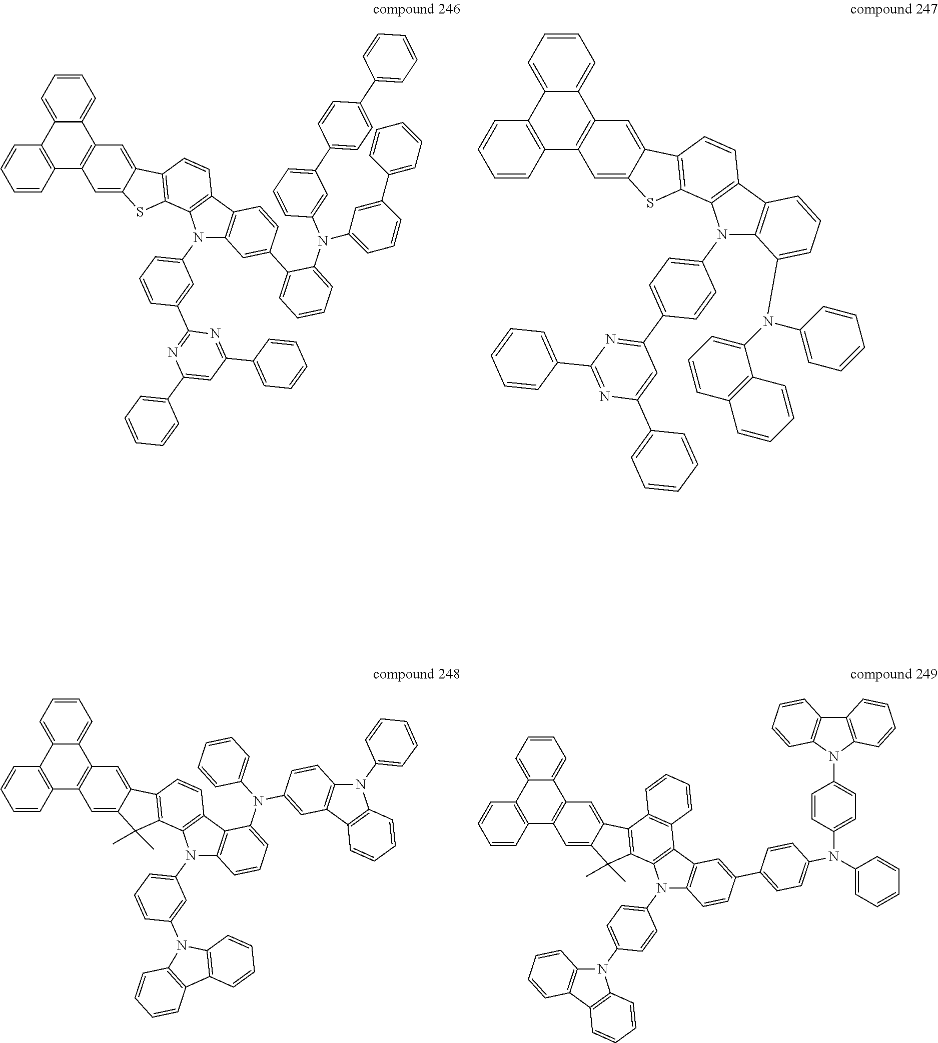

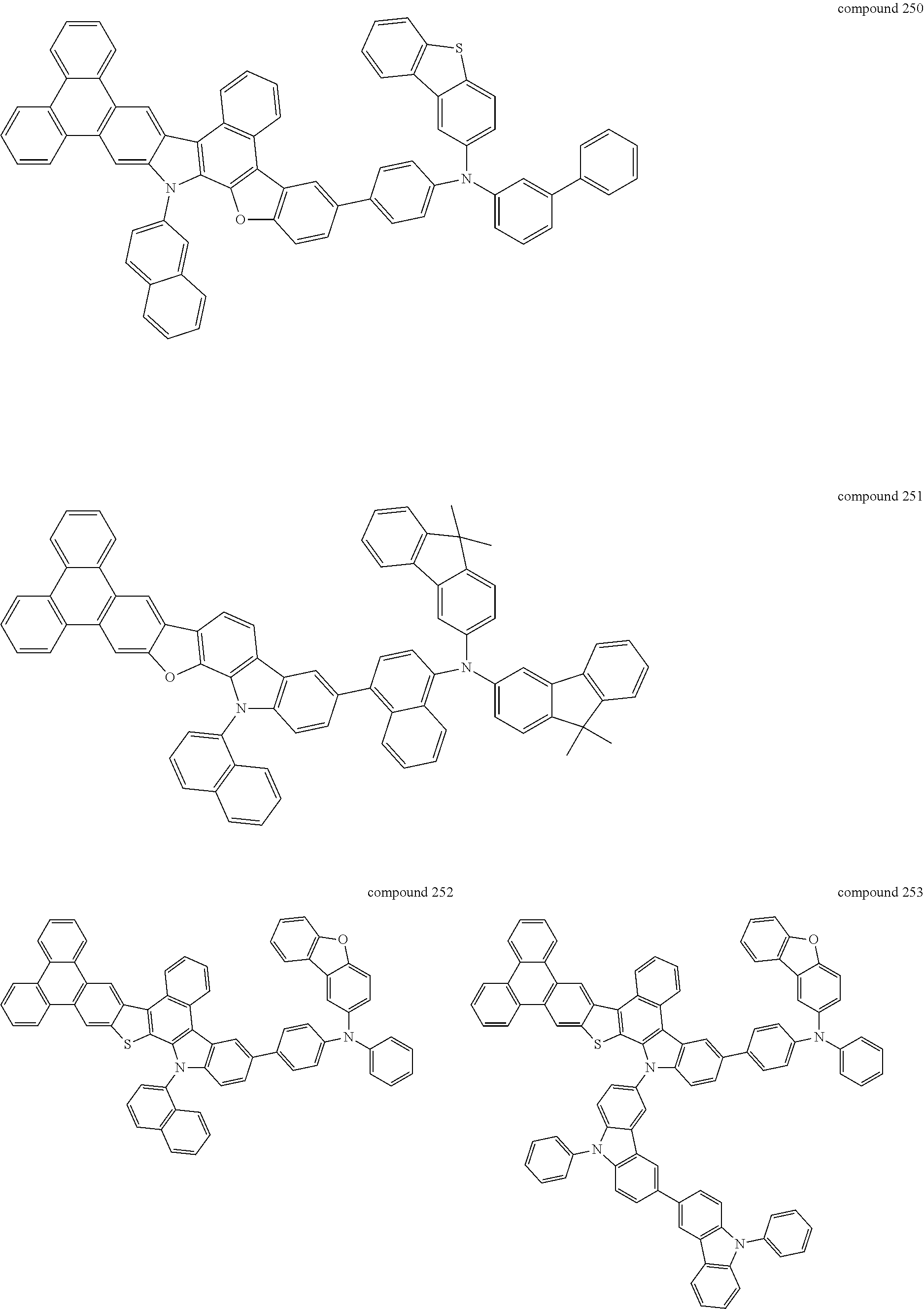

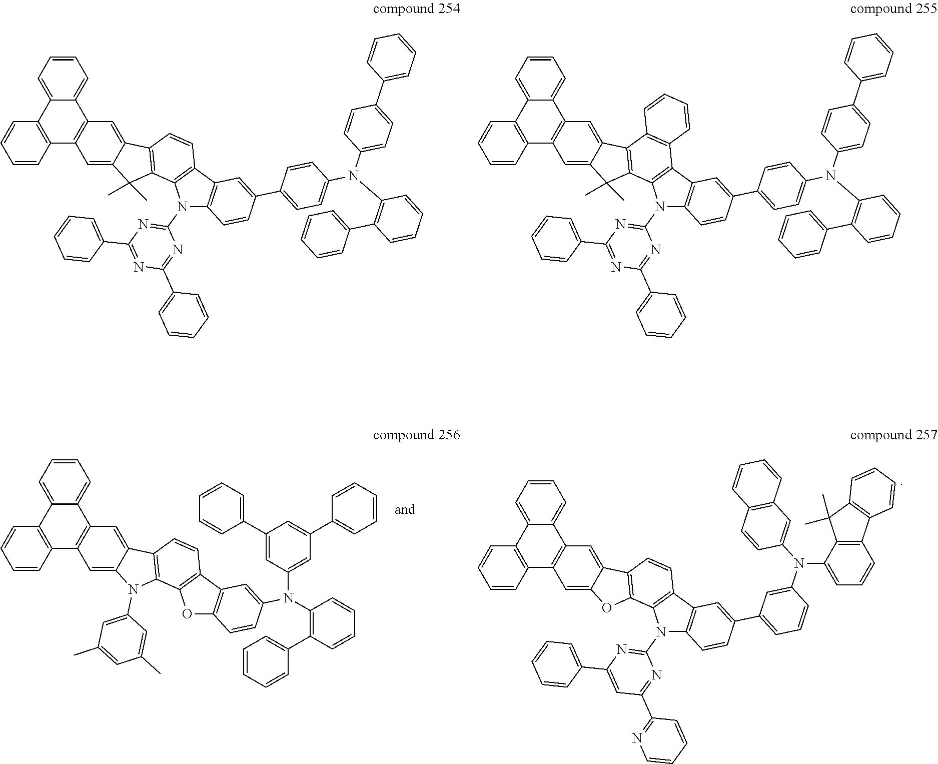

Referring to FIG. 1, the first organic EL device 510 may comprise an anode 310, a cathode 380 and one or more organic layers 320, 330, 340, 350, 360, 370 formed between the anode 310 and the cathode 380. From the bottom to the top, the one or more organic layers may comprise a hole injection layer 320, a hole transport layer 330, an emissive layer 340, a hole blocking layer 350, an electron transport layer 360E and an electron injection layer 370. The emissive layer 340 may comprise a 15% dopant D1 and the H1 doped with the dopant D1. The dopant D1 may be a red guest material for tuning the wavelength at which the emissive layer 340 emits light, so that the color of emitted light may be red.

The electron transport layer (ETL) 360E of FIG. 1 may be applied as an electron transporting material of formula (1) to co-deposit with 40% 8-hydroxyquinolato-lithium (LiQ).

FIG. 2 is a cross-sectional view of an organic EL device without the organic compound of formula (1). Referring to FIG. 2, the organic EL device 400 may comprise an anode 310, a cathode 380 and one or more organic layers 320, 330, 340, 350, 360, 370 formed between the anode 310 and the cathode 380. From the bottom to the top, the one or more organic layers may comprise a hole injection layer 320, a hole transport layer 330, an emissive layer 340, a hole blocking layer 350, an electron transport layer 360 and an electron injection layer 370. The emissive layer 340 may comprise a 15% dopant D1 and an organic compound H1 doped with the dopant D1. The dopant D1 may be a red guest material. The organic compound H1 is a host of the emissive layer 340. The electron transport layer (ETL) 360 of FIG. 2 may be applied as a compound ET2 to co-deposit with 40% 8-hydroxyquinolato-lithium (LiQ).

To those organic EL devices of FIG. 1 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 1 and FIG. 2 may be summarized in Table 1 below. The half-life is defined as the time that the initial luminance of 1000 cd/m² has dropped to half.

TABLE 1

| | Driving | Current | | |
|---|---|---|---|---|
| Material for ETL | Voltage | Efficiency | | Half-life |
| 360 or 360E | (V) | (cd/A) | color | (hours) |
| ET2 | 4.5 | 18 | red | 420 |
| Comp. 8 | 4.3 | 20 | red | 470 |
| Comp. 13 | 4.2 | 20 | red | 450 |
| Comp. 21 | 3.9 | 19 | red | 440 |
| Comp. 29 | 4.3 | 22 | red | 460 |
| Comp. 34 | 3.9 | 19 | red | 430 |
| Comp. 41 | 4.1 | 21 | red | 500 |
| Comp. 47 | 3.9 | 19 | red | 470 |
| Comp. 67 | 4.2 | 23 | red | 460 |
| Comp. 74 | 3.7 | 22 | red | 490 |
| Comp. 85 | 3.5 | 27 | red | 530 |
| Comp. 89 | 4.4 | 25 | red | 500 |
| Comp. 103 | 3.8 | 18 | red | 480 |
| Comp. 112 | 4.1 | 19 | red | 450 |
| Comp. 154 | 3.7 | 20 | red | 450 |
| Comp. 157 | 4.2 | 23 | red | 440 |
| Comp. 173 | 4.3 | 21 | red | 460 |
| Comp. 184 | 3.8 | 20 | red | 500 |
| Comp. 235 | 4.5 | 24 | red | 480 |
| Comp. 248 | 4.1 | 19 | red | 440 |
| Comp. 254 | 3.9 | 22 | red | 430 |

(The “Comp.” is short for “Compound”)

According to Table 1, in the first organic EL device 510, the organic compound of formula (1) comprised as an electron transport material 360C of FIG. 1 exhibits performance better than a prior art organic EL material (ET2).

A method of producing the first organic EL device 510 of FIG. 1 and the organic EL device 400 of FIG. 2 is described. ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water).

Before vapor deposition of the organic layers, cleaned ITO substrates may be further treated by UV and ozone. All pre-treatment processes for ITO substrate are under clean room (class 100), so that an anode 310 may be formed.

One or more organic layers 320, 330, 340 (FIG. 2), 350, 360, 360E (FIG. 1), 370 are applied onto the anode 310 in order by vapor deposition in a high-vacuum unit (10⁻⁷ Torr), such as resistively heated quartz boats. The thickness of the respective layer and the vapor deposition rate (0.1-0.3 nm/sec) are precisely monitored or set with the aid of a quartz-crystal monitor. It is also possible, as described above, each of the organic layers may comprise more than one organic compound. For example, an emissive layer 340 may be formed of a dopant and a host doped with the dopant. An emissive layer 340 may also be formed of a co-host and a host co-deposited with the co-host. This may be successfully achieved by co-vaporization from two or more sources. Accordingly, the compounds for the organic layers of the present invention are thermally stable.

Referring to FIG. 1 and FIG. 2, onto the anode 310, Dipyrazino[2,3-f:2,3-]quinoxaline-2,3,6,7,10,11-hexacarbonitrile (HAT-CN) may be applied to form a hole injection layer 320 having a thickness of about 25 nm. N,N-Bis(naphthalene-1-yl)-N,N-bis(phenyl)-benzidine (NPB) may be applied to form a hole transporting layer 330 having a thickness of about 110 nm. 12-(4,6-diphenyl-1,3,5-triazin-2-yl)-10,10-dimethyl-10,12-dihydrophenanthro[9′,10′:5,6]indeno[2,1-b]carbazole may be applied to form a host H1. The emissive layer 340 may further comprise bis[2-(4-n-hexylphenyl)quinoline](acetylacetonate)iridium(III) as a dopant D1 of an emissive layer 340. The emissive layer 340 may further comprise, also a red guest of the emissive layer 340 having a thickness of about 35 nm.

On the emissive layer 340, a compound HB3 may be a hole blocking material (HBM) to form a hole blocking layer 350 having a thickness of about 10 nm. 2-(naphthalen-1-yl)-9-(4-(1-(4-(10-(naphthalene-2-yl)anthracen-9-yl)-phenyl)-1H-benzo[d]imidazol-2-yl)phenyl)-1,10-phenanthroline (ET2) may be applied as an electron transporting material to co-deposit with 40% 8-hydroxyquinolato-lithium (LiQ), thereby forming an electron transporting layer (ETL) 360 of the organic EL device 510 or 400. The electron transporting layer (ETL) 360 may have a thickness of about 35 nm.

Table 2 shows the layer thickness and materials of the organic EL device 510 (FIG. 1) or 400 (FIG. 2).

TABLE 2

| Ref. No. in | | | Thickness |
|---|---|---|---|
| FIG. 1 or FIG. 2 | Layer | Material | (nm) |
| 380 | Cathode | Al | 160 |
| 370 | EIL | LiQ | 1 |
| 360E (FIG. 1) | ETL | 60% 360C:40% LiQ | 30 |
| or | | or | |
| 360 (FIG. 2) | | 60% ET2:40% LiQ | |
| 350 | HBL | HB3 | 10 |
| 340 | EML | 85% H1:15% D1 | 35 |
| 330 | HTL | NPB | 110 |
| 320 | HIL | HAT-CN | 25 |
| 310 | Anode | ITO substrate | 120~160 |

The organic compounds ET2, HB3, D1, NPB and HAT-CN for producing the organic EL device 400 or 510 in this invention may have the formulas as follows:

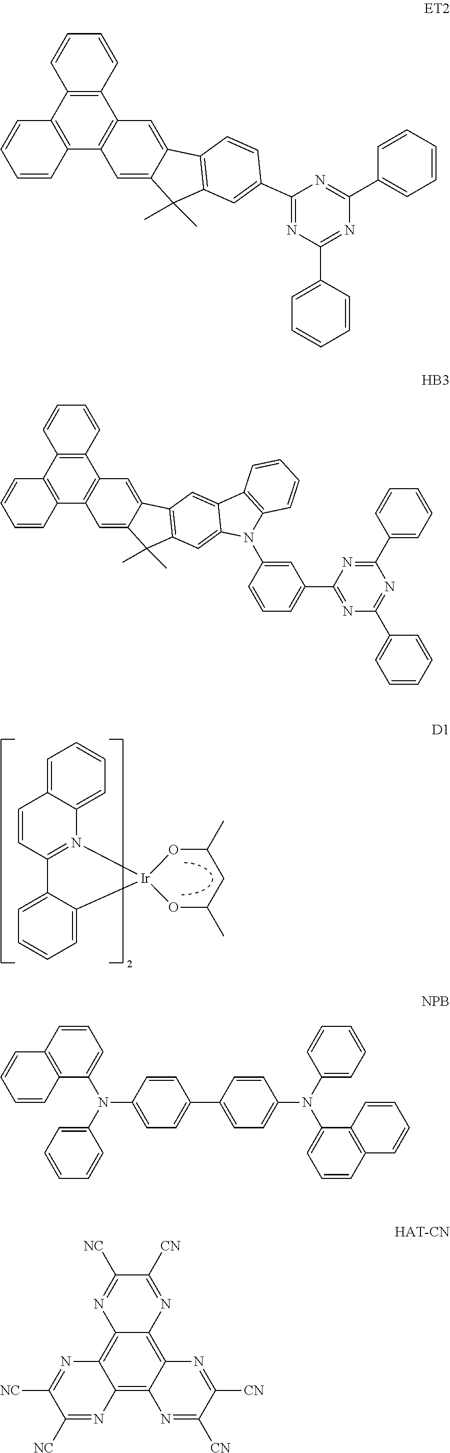

Referring to FIG. 1 and FIG. 2, the organic EL device 510 or 400 may further comprise a low work function metal, such as Al, Mg, Ca, Li or K, as a cathode 380 by thermal evaporation. A low work function metal may help electrons injecting the electron transporting layer 360 from cathode 380. Between the cathode 380 and the electron transporting layer 360, a thin electron injecting layer 370 of LiQ is introduced, to reduce the electron injection barrier and to improve the performance of the organic EL device 510 or 400. The material of the electron injecting layer 370 may alternatively be metal halide or metal oxide with low work function, such as LiF, MgO, or Li₂O.

In any above-mentioned compounds used in each layer of an organic EL device, the hydrogen atoms can be partially or fully deuterated. Thus, any specifically listed substituent, such as, without limitation, methyl, phenyl, pyridyl, etc. may be undeuterated, partially deuterated, and fully deuterated versions thereof. Similarly, classes of substituents such as, without limitation, alkyl, aryl, cycloalkyl, heteroaryl, etc. also may be undeuterated, partially deuterated, and fully deuterated versions thereof.

In one embodiment, a second organic EL device using the organic compound of formula (1) is disclosed. The method of producing the second organic EL device 520 of FIG. 3 is substantially the same as the method of producing the organic EL device 400 of FIG. 2. The difference is that the hole blocking layer (HBL) 350C of FIG. 3 is made by using the organic compound of formula (1), rather than HB3.

Table 3 shows the layer thickness and materials of the organic EL device 520 (FIG. 3) or 400 (FIG. 2).

TABLE 3

| Ref. No. in | | | Thickness |
|---|---|---|---|
| FIG. 3 or FIG. 2 | Layer | Material | (nm) |
| 380 | Cathode | Al | 160 |
| 370 | EIL | LiQ | 1 |
| 360 | ETL | 60% ET2:40% LiQ | 30 |
| 350C or 350 | HBL | 350C or HB3 | 10 |
| 340 | EML | 85% H1:15% D1 | 35 |
| 330 | HTL | NPB | 110 |
| 320 | HIL | HAT-CN | 25 |
| 310 | Anode | ITO substrate | 100 |

To those organic EL devices of FIG. 3 and FIG. 2, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

The I-V-B (at 1000 nits) test reports of those organic EL devices of FIG. 3 and FIG. 2 may be summarized in Table 4 below. The half-life of the fluorescent red-emitting organic EL device 520 or 400 is defined as the time that the initial luminance of 1000 cd/m² has dropped to half.

TABLE 4

| | Driving | Current | | |
|---|---|---|---|---|
| Material for | Voltage | Efficiency | | Half-life |
| HBL 350 or 350C | (V) | (cd/A) | color | (hours) |
| HB3 | 4.5 | 18 | red | 420 |
| Comp. 14 | 3.9 | 26 | red | 510 |
| Comp. 24 | 4.5 | 19 | red | 440 |
| Comp. 35 | 4.3 | 22 | red | 400 |
| Comp. 42 | 4.5 | 21 | red | 510 |
| Comp. 48 | 4.3 | 21 | red | 430 |
| Comp. 55 | 4.4 | 23 | red | 420 |
| Comp. 69 | 4.1 | 20 | red | 490 |
| Comp. 76 | 4.4 | 19 | red | 430 |
| Comp. 82 | 4.7 | 21 | red | 400 |
| Comp. 107 | 4.4 | 23 | red | 480 |
| Comp. 119 | 4.5 | 18 | red | 410 |
| Comp. 141 | 4.8 | 22 | red | 380 |
| Comp. 147 | 4.9 | 20 | red | 380 |
| Comp. 156 | 4.8 | 21 | red | 390 |
| Comp. 190 | 4.6 | 23 | red | 430 |
| Comp. 231 | 4.1 | 19 | red | 500 |

(The “Comp.” is short for “Compound”)

According to Table 4, in the second organic EL device 520, the organic compound of formula (1) comprised as a hole blocking layer 350C of FIG. 3 may exhibit performance substantially better than a prior art hole blocking material (HB3 as a HBL 350 of FIG. 2).

Referring to FIG. 1 or FIG. 3, the organic EL device 510 or 520 of the present invention may alternatively be a lighting panel or a backlight panel.

Detailed preparation of the organic compounds of the present invention will be clarified by exemplary embodiments below, but the present invention is not limited thereto. EXAMPLES 1 to 15 show the preparation of the organic compounds of the present invention.

### Example 1

### Synthesis of Compound 8

### Synthesis of 5-bromo-9-iodo-7,7-dimethyl-7H-benzo[c]fluorine

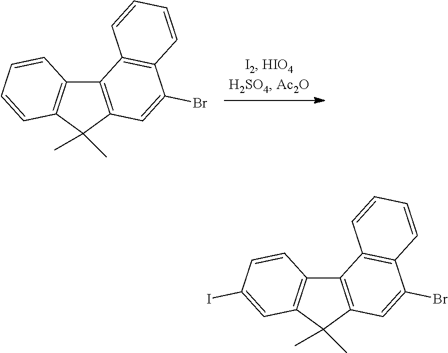 [SMILES: CC1(C)c2ccccc2-c2c1cc(Br)c1ccccc21 ; CC1(C)c2cc(I)ccc2-c2c1cc(Br)c1ccccc21]

At 0° C., 2M aqueous solution of sulfuric acid (105 ml, 210.3 mmol) was dropped into acetic anhydride (580 ml), and then the mixture was stirred at this temperature for 1 hr. 5-bromo-7,7-dimethyl-7H-benzo[c]fluorine (40 g, 123.7 mmol), periodic acid (9.3 g, 40.8 mmol) and iodine (19.8 g, 77.9 mmol) were add into the mixture slowly. Keeping the mixture was stirred at 0° C. for 1 hr. The mixture was allowed to warm to room temperature for 1 hr. After that, the mixture was heated to 90° C. slowly and stirred overnight. After the reaction finished, the mixture was poured into ice water and extracted with ethyl acetate, and the organic layer was removed under reduced pressure and the crude product was purified by column chromatography, yielding 28 g of 5-bromo-9-iodo-7,7-dimethyl-7H-benzo[c]fluorene as pale yellow syrup (50.9%).

### Synthesis of 9-([1,1′-biphenyl]-2-yl)-5-bromo-7,7-dimethyl-7H-benzo[c]fluorine

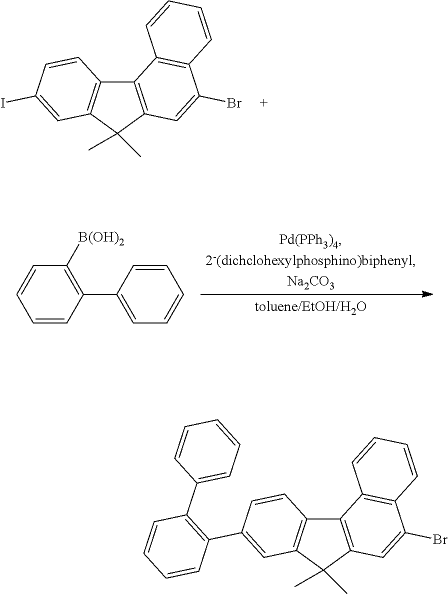 [SMILES: CC1(C)c2cc(I)ccc2-c2c1cc(Br)c1ccccc21 ; OB(O)c1ccccc1-c1ccccc1 ; CC1(C)c2cc(-c3ccccc3-c3ccccc3)ccc2-c2c1cc(Br)c1ccccc21]

A mixture of 5-bromo-9-iodo-7,7-dimethyl-7H-benzo[c]fluorene (28 g, 62.3 mmol), biphenyl-2-ylboronicacid (9.8 g, 49.8 mmol), 2M aqueous solution of sodium carbonate (65 ml), ethanol (140 ml), and toluene (420 ml) was degassed by bubbling nitrogen gas for 15 minutes followed by addition of palladium tetrakis (1.4 g, 1.22 mmol) and 2-(dicyclohexylphosphino)biphenyl (0.9 g, 2.44 mmol). The mixture was heated to reflux overnight. Upon cooling to room temperature, the mixture was extracted with ethyl acetate (3 times) and washed with water. The organic layer was dried with anhydrous magnesium sulfate and evaporated under reduced pressure. The residue was purified by column chromatography on silica, yielding 15 g of 9-([1,1′-biphenyl]-2-yl)-5-bromo-7,7-dimethyl-7H-benzo[c]fluorine as colorless syrup (51%).

### Synthesis of 14-bromo-16,16-dimethyl-16H-benzo[6,7]indeno[1,2-b]triphenylene

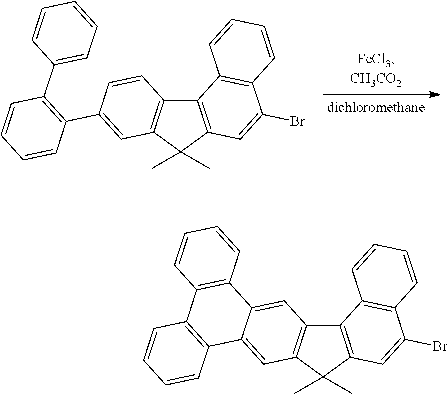 [SMILES: CC1(C)c2cc(-c3ccccc3-c3ccccc3)ccc2-c2c1cc(Br)c1ccccc21 ; CC1(C)c2cc3c4ccccc4c4ccccc4c3cc2-c2c1cc(Br)c1ccccc21]

A mixture of 9-([1,1′-biphenyl]-2-yl)-5-bromo-7,7-dimethyl-7H-benzo[c] fluorene (15 g, 31.5 mmol) and dichlromethane (630 ml) was cooled to −15° C. and followed by addition of iron chloride (25 g, 157.5 mmol) dissolving in nitromethane (30 ml). After the reaction was finished, the mixture quenched with methanol and washed with water. The organic layer was dried with anhydrous magnesium sulfate and evaporated under reduced pressure. The residue was purified by column chromatography on silica, yielding 13 g of 14-bromo-16,16-dimethyl-16H-benzo[6,7]indeno[1,2-b]triphenylene as pale yellow solid (87%).

### Synthesis of 2-(16,16-dimethyl-16H-benzo[6,7]indeno[1,2-b]triphenylen-14-yl)-4,4,5,5-tetramethyl-1,3,2-dioxaborolane

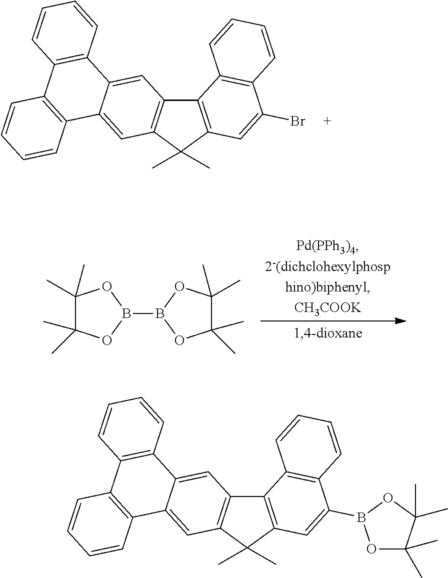

A mixture of 14-bromo-16,16-dimethyl-16H-benzo[6,7]indeno [1,2-b]triphenylene (11.6 g, 24.6 mmol), bis(pinacolato)diboron (11.2 g, 44.4 mmol), potassium acetate (7.3 g, 73.8 mmol), and 1,4-dioxane (61 ml) was degassed by bubbling nitrogen gas for 15 minutes followed by addition of palladium tetrakis (0.6 g, 0.5 mmol) and 2-(dicyclohexylphosphino)biphenyl (0.4 g, 1 mmol). The mixture was heated to reflux for 4 hr. Upon cooling to room temperature, the mixture was filtered with Celite. The filtrate was evaporated under reduced pressure and added with ethyl acetate and hexane to obtain solid. The precipitated solid was filtered with suction, yielding 7.9 g of 2-(16,16-dimethyl-16H-benzo[6,7]indeno[1,2-b]triphenylen-14-yl)-4,4,5,5-tetramethyl-1,3,2-dioxaborolane as pale yellow solid (62%).

### Synthesis of 14-(5-bromo-2-nitrophenyl)-16,16-dimethyl-16H-benzo[6,7]indeno[1,2-b]trip henylene

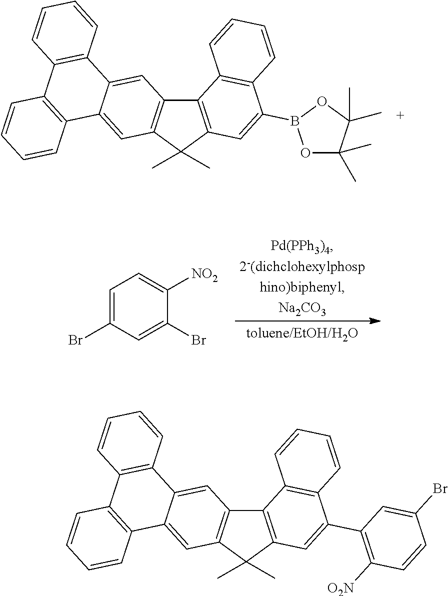 [SMILES: CC1(C)c2cc3c4ccccc4c4ccccc4c3cc2-c2c1cc(B1OC(C)(C)C(C)(C)O1)c1ccccc21 ; O=[N+]([O-])c1ccc(Br)cc1Br ; CC1(C)c2cc3c4ccccc4c4ccccc4c3cc2-c2c1cc(-c1cc(Br)ccc1[N+](=O)[O-])c1ccccc21]

A mixture of 2-(16,16-dimethyl-16H-benzo[6,7]indeno[1,2-b]triphenylen-14-yl)-4,4,5,5-tetramethyl-1,3,2-dioxaborolane (7.9 g, 15.2 mmol), 2,4-dibromo-1-nitrobenzene (4.6 g, 16.7 mmol), 2M aqueous solution of sodium carbonate (16 ml), ethanol (35 ml), and toluene (105 ml) was degassed by bubbling nitrogen gas for 15 minutes followed by addition of palladium tetrakis (1.4 g, 1.22 mmol) and 2-(dicyclohexylphosphino)biphenyl (0.9 g, 2.44 mmol). The mixture was heated to reflux overnight. Upon cooling to room temperature, the mixture was extracted with ethyl acetate (3 times) and washed with water. The organic layer was dried with anhydrous magnesium sulfate and evaporated under reduced pressure. The residue was purified by column chromatography on silica, yielding 4.9 g of 14-(5-bromo-2-nitrophenyl)-16,16-dimethyl-16H-benzo[6,7]indeno[1,2-b]triphenylene as yellow solid (54%).

### Synthesis of 4-bromo-20,20-dimethyl-1,20-dihydrobenzo[c]phenanthro[9′,10′:5,6]indeno[2, 1-a]carbazole

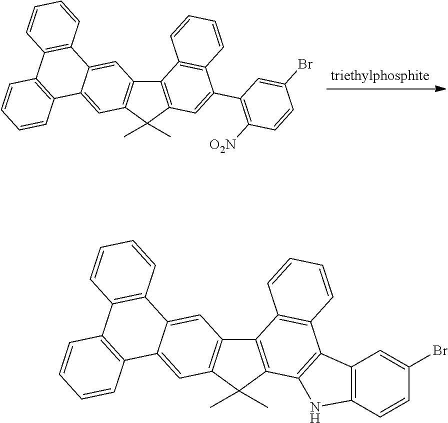 [SMILES: CC1(C)c2cc3c4ccccc4c4ccccc4c3cc2-c2c1cc(-c1cc(Br)ccc1[N+](=O)[O-])c1ccccc21 ; CC1(C)c2cc3c4ccccc4c4ccccc4c3cc2-c2c1c1[nH]c3ccc(Br)cc3c1c1ccccc21]

A mixture of 14-(5-bromo-2-nitrophenyl)-16,16-dimethyl-16H-benzo [6,7]indeno[1,2-b]triphenylene(4.9 g, 8.2 mmol) and triethylphosphite (25 ml) heated to reflux for 12 hrs. After the reaction finished, triethylphosphite was removed under reduced pressure, and the crude product was purified by column chromatography, yielding 2.2 g of 4-bromo-20,20-dimethyl-1,20-dihydrobenzo[c]phenanthro[9′,10′:5,6]indeno[2,1-a]carbazole as pale yellow solid (49%).

### Synthesis of 4-bromo-20,20-dimethyl-1,20-dihydrobenzo[c]phenanthro[9′,10′:5,6]indeno[2,1-a]carbazole

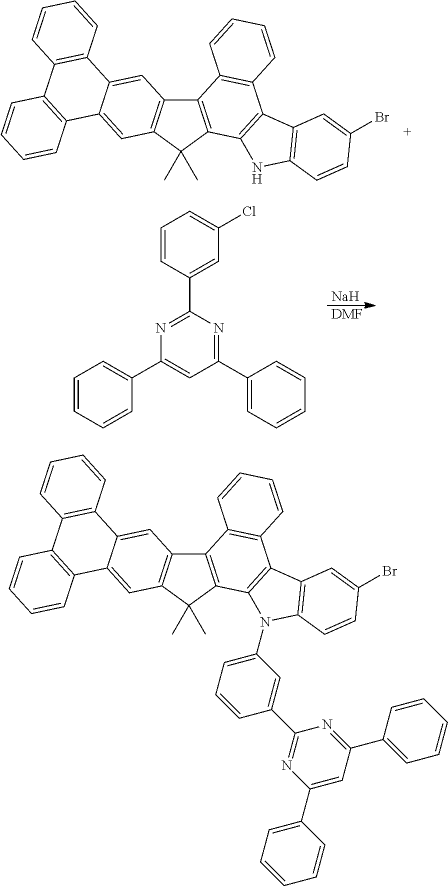

At 0° C., a mixture of sodium hydride (0.1 g, 4.4 mmol) and dimethyl formamide (2 ml) was added the 20,20-dimethyl-1,20-dihydrobenzo[c]phenanthro[9′,10′:5,6]indeno[2,1-a]carb azole (2.2 g, 4 mmol) dissolved in dimethylformamide (6 ml) slowly. After addition is completed, the mixture was warmed to room temperature and kept stirring 30 minutes. The mixture of 2-(3-chlorophenyl)-4,6-diphenylpyrimidine (1.5 g, 4.4 mmol) and dimethylformamide (5 ml) was added and stirred overnight. After the reaction finished, the mixture was quenched with ice water slowly and filtered to get the crude. The crude was purified by column chromatography, yielding 1.8 g of 4-bromo-1-(3-(4,6-diphenylpyrimidin-2-yl)phenyl)-20,20-dimethyl-1,20-dihy drobenzo[c]phenanthro[9′,10′:5,6]indeno[2,1-a]carbazole as pale yellow solid (51%).

Synthesis of N-([1,1′-biphenyl]-2-yl)-1-(3-(4,6-diphenylpyrimidin-2-yl) phenyl)-20,20-dimethyl-N-(naphthalen-1-yl)-1,20-dihydrobenzo[c]phenanthro[9′,10′:5,6]indeno[2,1-a]carbazol-4-amine (Compound 8)

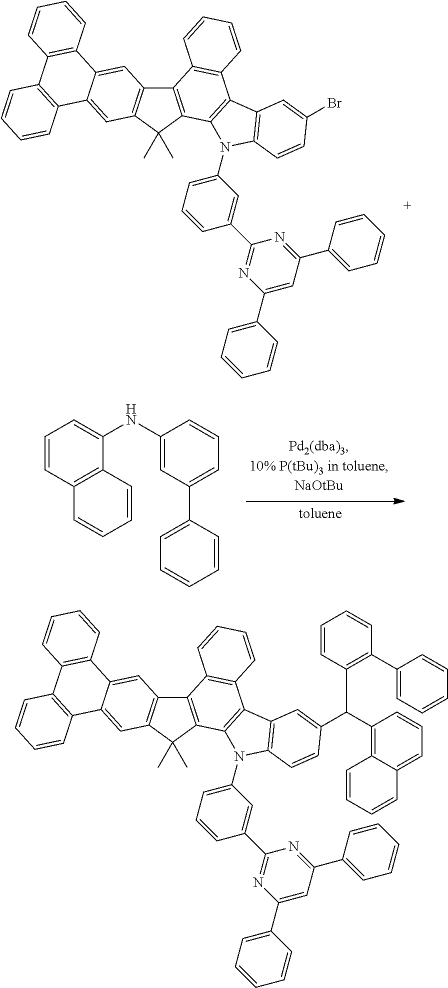 [SMILES: CC1(C)c2cc3c4ccccc4c4ccccc4c3cc2-c2c1c1c(c3ccccc23)c2cc(Br)ccc2n1-c1cccc(-c2nc(-c3ccccc3)cc(-c3ccccc3)n2)c1 ; c1ccc(-c2cccc(Nc3cccc4ccccc34)c2)cc1 ; CC1(C)c2cc3c4ccccc4c4ccccc4c3cc2-c2c1c1c(c3ccccc23)c2cc(C(c3ccccc3-c3ccccc3)c3cccc4ccccc34)ccc2n1-c1cccc(-c2nc(-c3ccccc3)cc(-c3ccccc3)n2)c1]

A mixture of 4-bromo-1-(3-(4,6-diphenylpyrimidin-2-yl)phenyl)-20,20-dimethyl-1,20-dihy drobenzo[c]phenanthro[9′,10′:5,6]indeno[2,1-a]carbazole (1.8 g, 2 mmol), N-([1,1′-biphenyl]-3-yl)naphthalen-1-amine (0.7 g, 2.2 mmol), Pd₂(dba)₃ (0.03 g, 0.04 mmol), 10% P(tBu) in toluene (0.1 ml), NaOtBu (0.3 g, 3 mmol) and toluene (10 ml) was degassed and placed under nitrogen, and then heated to reflux for 12 hrs. After the reaction finished, the mixture was allowed to cool to room temperature and was filtered with Celite. The filtrate was evaporated under reduced pressure and purified by column chromatography, yielding 1.1 g of N-([1,1′-biphenyl]-2-yl)-1-(3-(4,6-diphenylpyrimidin-2-yl)phenyl)-20,20-dimethyl-N-(naphthalen-1-yl)-1,20-dihydrobenzo[c]phenanthro[9′,10′:5,6]indeno[2,1-a]carbazol-4-amine (compound 8) as pale yellow solid (51%).

### Example 2

### Synthesis of Compound 34

### Synthesis of N-(4-(1-(4-([1,1′-biphenyl]-3-yl)-6-phenylpyrimidin-2-yl)-20,20-dimethyl-1,20-dihydrobenzo[c]phenanthro[9′,10′:5,6]indeno[2,1-a]carbazol-4-yl)phenyl)-N-([1,1′-biphenyl]-4-yl)dibenzo[b,d]furan-4-amine (Compound 34)

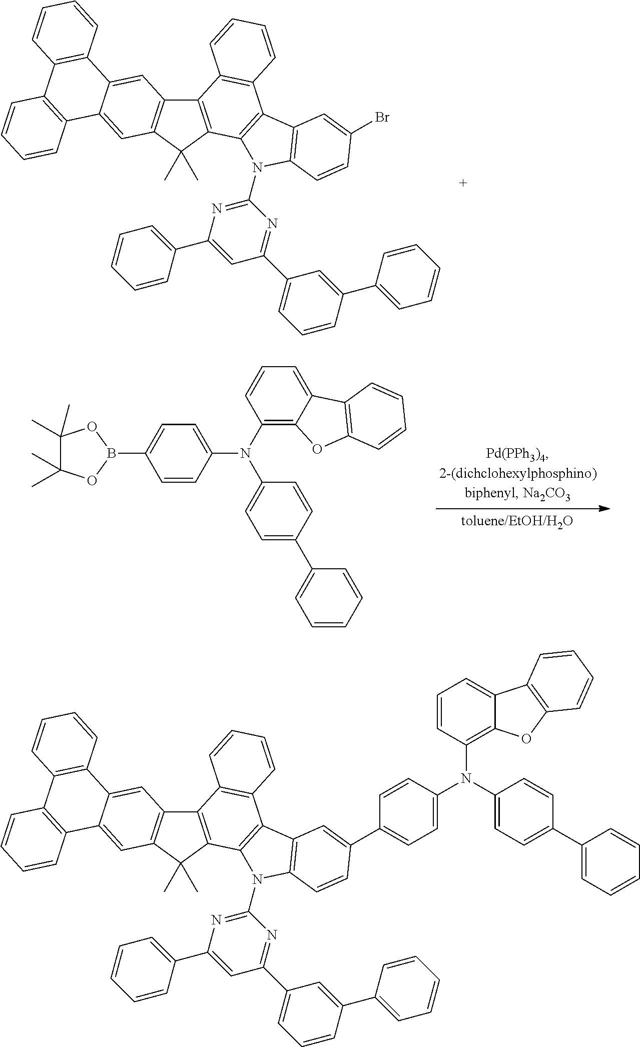

A mixture of 1-(4-([1,1′-biphenyl]-3-yl)-6-phenylpyrimidin-2-yl)-4-bromo-20,20-dimethyl-1,20-dihydrobenzo[c]phenanthro[9′,10′:5,6]indeno[2,1-a]carbazole (2.7 g, 3.2 mmol), N-([1,1′-biphenyl]-4-yl)-N-(4-(4,4,5,5-tetramethyl-1,3,2-dioxaborolan-2-yl)phenyl) dibenzo[b,d]furan-4-amine (g, 3.6 mmol), 2M aqueous solution of sodium carbonate (3 ml), ethanol (6 ml), and toluene (18 ml) was degassed by bubbling nitrogen gas for 15 minutes followed by addition of palladium tetrakis (0.07 g, 0.06 mmol) and 2-(dicyclohexylphosphino)biphenyl (0.04 g, 0.12 mmol). The mixture was heated to reflux overnight. Upon cooling to room temperature, the mixture was filtered to obtain solid. The solid was washed with water and methanol. The residue was purified by column chromatography on silica, yielding 2.3 g of N-(4-(1-(4-([1,1′-biphenyl]-3-yl)-6-phenylpyrimidin-2-yl)-20,20-dimethyl-1,20-dihydrobenzo[c]phenanthro[9′,10′:5,6]indeno[2,1-a]carbazol-4-yl)phenyl)-N-([1,1′-biphenyl]-4-yl)dibenzo[b,d]furan-4-amine (compound 34) as pale yellow solid (61%).

### Example 3

### Synthesis of Compound 85

### Synthesis of N-(2-chlorophenyl)-14,14-dimethyl-14H-indeno[1,2-b]triphenylen-12-amine

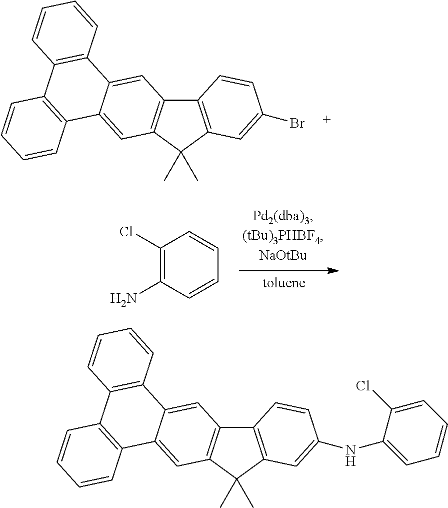

A mixture of 12-bromo-14,14-dimethyl-14H-indeno[1,2-b]triphenylene (10 g, 23.7 mmol), 2-chloroaniline (3.3 g, 26 mmol), Pd₂(dba)₃ (0.2 g, 0.24 mmol), (tBu)₃PHBF₄ (0.3 g, 0.95 mmol), NaOtBu (3.5 g, 35.6 mmol) and toluene (100 ml) was degassed and placed under nitrogen, and then heated to reflux. After the reaction finished, the mixture was filtered with Celite. The filtrate was evaporated under reduced pressure. The residue was washed with ethyl acetate and hexane, yielding 7.8 g of N-(2-chlorophenyl)-14,14-dimethyl-14H-indeno[1,2-b]triphenylen-12-amine as brown solid (70%).

### Synthesis of 10,10-dimethyl-10,12-dihydrophenanthro[9′,10′:5,6]indeno[2,1-b]carbazole

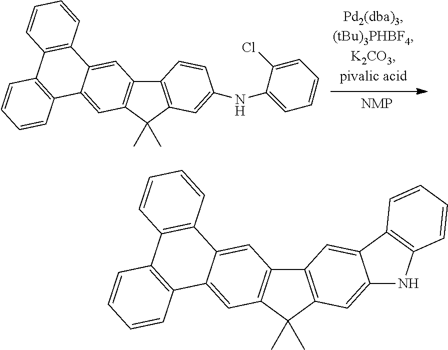

A mixture of N-(2-chlorophenyl)-14,14-dimethyl-14H-indeno[1,2-b]triphenylen-12-amine (38.7 g, 82.3 mmol), Pd₂(dba)₃ (7.5 g, 8.2 mmol), (tBu)₃PHBF₄ (4.7 g, 16.5 mmol) potassium carbonate (28.4 g, 205.8 mmol), pivalic acid (2.5 g, 24.7 mmol) and 1-Methyl-2-pyrrolidone (230 ml) was heated to 130° C. After the reaction finished, the mixture filtered with Celite and removed the 1-Methyl-2-pyrrolidone under reduced pressure. The residue was washed with dichloromethane and ethyl acetate to obtain brown solid, yielding 29.1 g of 10,10-dimethyl-10,12-dihydrophenanthro[9′,10′:5,6]indeno[2,1-b]carbazole as grey solid (72%)

### Synthesis of 15-bromo-10,10-dimethyl-10,12-dihydrophenanthro[9′,10′:5,6]indeno[2,1-b]carbazole

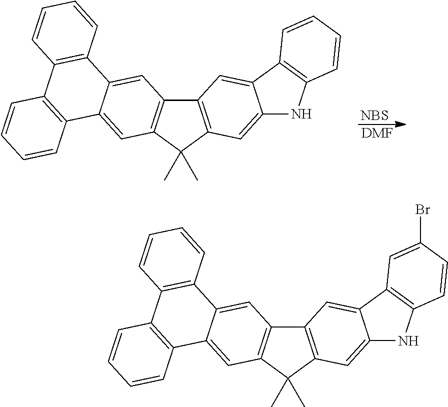

At ice bath, a mixture of 10,10-dimethyl-10,12-dihydrophenanthro[9′,10′:5,6]indeno[2,1-b]carbazole (1 g, 2.3 mmol) and dimethylformamide (10 ml) was added N-bromosuccinimide (0.4 g, 2.3 mmol) portion wise. After addition is completed, the mixture was warmed to room temperature and stirred overnight. Water was added into mixture after the reaction was finished. The mixture was filtered to obtain solid. The solid was washed with methanol, yielding 0.8 g of 15-bromo-10,10-dimethyl-10,12-dihydrophenanthro[9′,10′:5,6]indeno[2,1-b]carbazole as grey solid (67%)

### Synthesis of 15-bromo-12-(4,6-diphenyl-1,3,5-triazin-2-yl)-10,10-dimethyl-10,12-dihydro phenanthro[9′,10′:5,6]indeno[2,1-b]carbazole

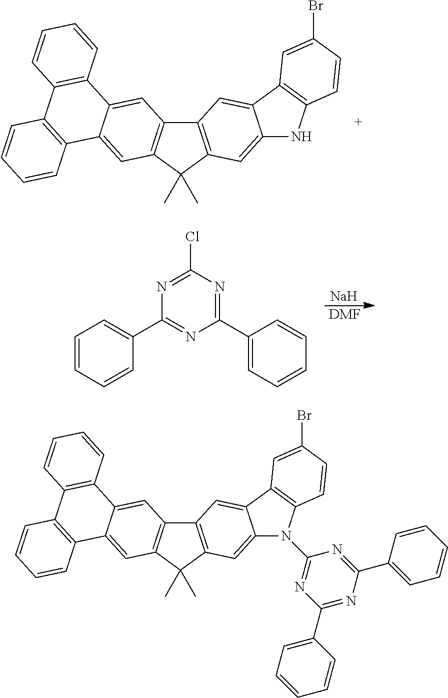

At 0° C., a mixture of sodium hydride (0.5 g, 22 mmol) and dimethyl formamide (10 ml) was added the 15-bromo-10,10-dimethyl-10,12-dihydrophenanthro[9′,10′:5,6]indeno[2,1-b]carbazole (10.2 g, 20 mmol) dissolved in dimethylformamide (30 ml) slowly. After addition is completed, the mixture was warmed to room temperature and kept stirring 30 minutes. The mixture of 2-chloro-4,6-diphenyl-1,3,5-triazine (5.9 g, 22 mmol) and dimethylformamide (25 ml) was added and stirred overnight. After the reaction finished, the mixture was quenched with ice water slowly and filtered to get the crude. The crude was purified by column chromatography, yielding 8.9 g of 15-bromo-12-(4,6-diphenyl-1,3,5-triazin-2-yl)-10,10-dimethyl-10,12-dihydro phenanthro[9′,10′:5,6]indeno[2,1-b]carbazole as yellow solid (60%).

### Synthesis of N-([1,1′-biphenyl]-4-yl)-N-(4-(12-(4,6-diphenyl-1,3,5-triazin-2-yl)-10,10-dimethyl-10,12-dihydrophenanthro[9′,10′:5,6]indeno[2,1-b]carbazol-15-yl)phenyl)-[1,1′-biphenyl]-2-amine (Compound 85)

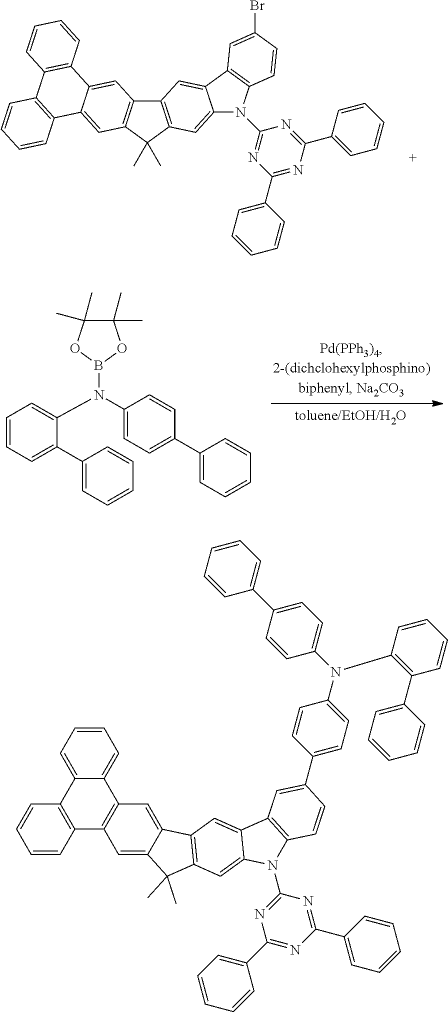 [SMILES: CC1(C)c2cc3c4ccccc4c4ccccc4c3cc2-c2cc3c4cc(Br)ccc4n(-c4nc(-c5ccccc5)nc(-c5ccccc5)n4)c3cc21 ; CC1(C)OB(N(c2ccc(-c3ccccc3)cc2)c2ccccc2-c2ccccc2)OC1(C)C ; CC1(C)c2cc3c4ccccc4c4ccccc4c3cc2-c2cc3c4cc(-c5ccc(N(c6ccc(-c7ccccc7)cc6)c6ccccc6-c6ccccc6)cc5)ccc4n(-c4nc(-c5ccccc5)nc(-c5ccccc5)n4)c3cc21]

A mixture of 15-bromo-12-(4,6-diphenyl-1,3,5-triazin-2-yl)-10,10-dimethyl-10,12-dihydro phenanthro[9′,10′:5,6]indeno[2,1-b]carbazole (4 g, 5.3 mmol), N-([1,1′-biphenyl]-4-yl)-N-(4-(4,4,5,5-tetramethyl-1,3,2-dioxaborolan-2-yl)phenyl)dibenzo[b,d]furan-4-amine (3.1 g, 5.9 mmol), 2M aqueous solution of sodium carbonate (6 ml), ethanol (12 ml), and toluene (36 ml) was degassed by bubbling nitrogen gas for 15 minutes followed by addition of palladium tetrakis (0.1 g, 0.1 mmol) and 2-(dicyclohexylphosphino)biphenyl (0.07 g, 0.2 mmol). The mixture was heated to reflux overnight. Upon cooling to room temperature, the mixture was filtered to obtain solid. The solid was washed with water and methanol. The residue was purified by column chromatography on silica, yielding 2.9 g N-([1,1′-biphenyl]-4-yl)-N-(4-(12-(4,6-diphenyl-1,3,5-triazin-2-yl)-10,10-dimethyl-10,12-dihydrophenanthro[9′,10′:5,6]indeno[2,1-b]carbazol-15-yl)phenyl)-[1,1′-biphenyl]-2-amine (compound 85) as yellow solid (53%).

### Example 4

### Synthesis of Compound 74

### Synthesis of 3-([1,1′-biphenyl]-2-yl)-7-bromodibenzo[b,d]thiophene

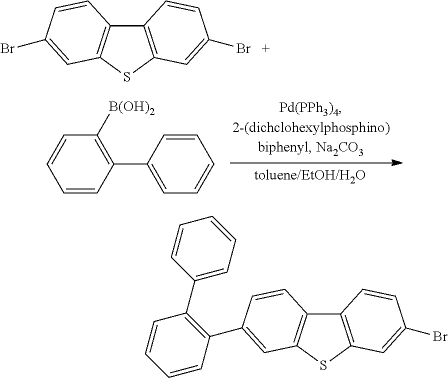

A mixture of 3,7-dibromodibenzo[b,d]thiophene (20.9 g, 61.3 mmol),biphenyl-2-ylboronic acid (12.2 g, 61.3 mmol), sodium carbonate (13 g, 122.6 mmol), water (60 ml), ethanol (60 ml) and toluene (120 ml) was degassed by bubbling nitrogen gas for 15 minutes followed by addition of palladium tetrakis (1.4 g, 1.22 mmol) and 2-(dicyclohexylphosphino)biphenyl (0.9 g, 2.44 mmol). The mixture was heated to reflux overnight. Upon cooling to room temperature, the mixture was extracted with ethyl acetate (3 times) and washed with water. The organic layer was dried with anhydrous magnesium sulfate and evaporated under reduced pressure. The residue was purified by column chromatography on silica, yielding 12.4 g of 3-([1,1′-biphenyl]-2-yl)-7-bromodibenzo[b,d]thiophene as yellow solid (43%).

### Synthesis of 12-bromobenzo[b]triphenyleno[2,3-d]thiophene

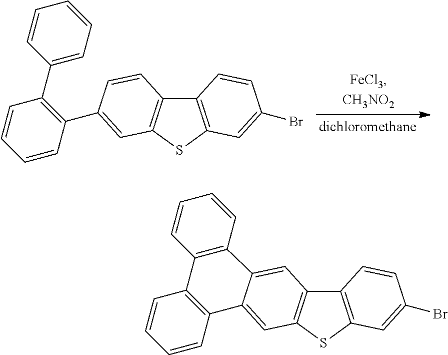 [SMILES: Brc1ccc2c(c1)sc1cc(-c3ccccc3-c3ccccc3)ccc12 ; Brc1ccc2c(c1)sc1cc3c4ccccc4c4ccccc4c3cc12]

A mixture of 3-([1,1′-biphenyl]-2-yl)-7-bromodibenzo[b,d]thiophene (12.4 g, 26.3 mmol) and dichlromethane (525 ml) was cooled to −30° C. and followed by addition of iron chloride (21.3 g, 131.5 mmol) dissolving in nitromethane (26 ml). After the reaction was finished, the mixture quenched with methanol and washed with water. The organic layer was dried with anhydrous magnesium sulfate and evaporated under reduced pressure. The residue was purified by column chromatography on silica, yielding 3.1 g of 12-bromobenzo[b]triphenyleno[2,3-d]thiophene as dark-green solid (29%).

### Synthesis of 2-(benzo[b]triphenyleno[2,3-d]thiophen-12-yl)-4,4,5,5-tetramethyl-1,3,2-dioxaborolane

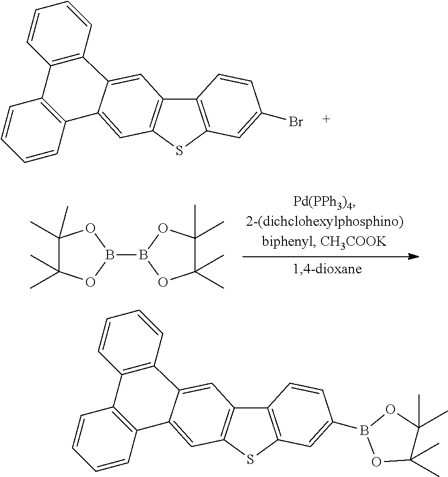

A mixture of 12-bromobenzo[b]triphenyleno[2,3-d]thiophene (3.1 g, 7.5 mmol), bis(pinacolato)diboron (3.4 g, 13.5 mmol), potassium acetate (2.2 g, 22.5 mmol), and 1,4-dioxane (18 ml) was degassed by bubbling nitrogen gas for 15 minutes followed by addition of palladium tetrakis (0.3 g, 0.3 mmol) and 2-(dicyclohexylphosphino)biphenyl (0.05 g, 0.15 mmol). The mixture was heated to reflux for 4 hr. Upon cooling to room temperature, the mixture was filtered with Celite. The filtrate was evaporated under reduced pressure and added with ethyl acetate and hexane to obtain solid. The precipitated solid was filtered with suction, yielding 2 g of 2-(benzo[b]triphenyleno[2,3-d]thiophen-12-yl)-4,4,5,5-tetramethyl-1,3,2-dioxaborolane as pale yellow solid (59%).

### Synthesis of 12-(4-bromo-2-nitrophenyl)benzo[b]triphenyleno[2,3-d]thiophene

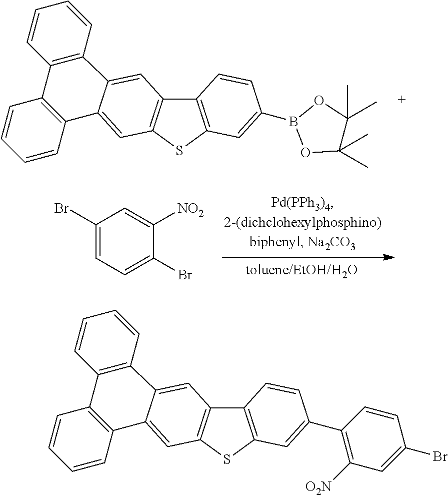

A mixture of 2-(benzo[b]triphenyleno[2,3-d]thiophen-12-yl)-4,4,5,5-tetramethyl-1,3,2-dioxaborolane (6.9 g, 15.2 mmol), 2,4-dibromo-1-nitrobenzene (4.6 g, 16.7 mmol), 2M aqueous solution of sodium carbonate (16 ml), ethanol (35 ml), and toluene (105 ml) was degassed by bubbling nitrogen gas for 15 minutes followed by addition of palladium tetrakis (1.4 g, 1.22 mmol) and 2-(dicyclohexylphosphino)biphenyl (0.9 g, 2.44 mmol). The mixture was heated to reflux overnight. Upon cooling to room temperature, the mixture was extracted with ethyl acetate (3 times) and washed with water. The organic layer was dried with anhydrous magnesium sulfate and evaporated under reduced pressure. The residue was purified by column chromatography on silica, yielding 5 g of 12-(4-bromo-2-nitrophenyl)benzo[b]triphenyleno[2,3-d]thiophene as yellow solid (62%).

### Synthesis of 14-bromo-16H-triphenyleno[2′,3′:4,5]thieno[3,2-b]carbazole

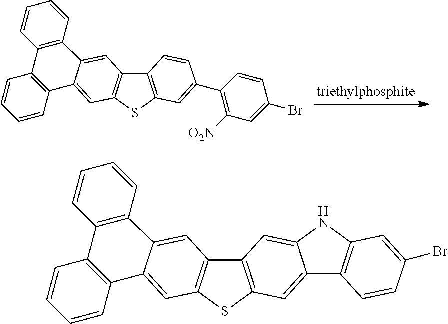 [SMILES: O=[N+]([O-])c1cc(Br)ccc1-c1ccc2c(c1)sc1cc3c4ccccc4c4ccccc4c3cc12 ; Brc1ccc2c(c1)[nH]c1cc3c(cc12)sc1cc2c4ccccc4c4ccccc4c2cc13]

A mixture of 12-(4-bromo-2-nitrophenyl)benzo[b]triphenyleno[2,3-d]thiophene (4.3 g, 8.2 mmol) and triethylphosphite (25 ml) heated to reflux for 12 hrs. After the reaction finished, triethylphosphite was removed under reduced pressure, and the crude product was purified by column chromatography, yielding 1.9 g of 14-bromo-16H-triphenyleno[2′,3′:4,5]thieno[3,2-b]carbazole as yellow solid (47%).

### Synthesis of 14-bromo-16-(2,6-diphenylpyrimidin-4-yl)-16H-triphenyleno[2′,3′:4,5]thieno[3,2-b]carbazole

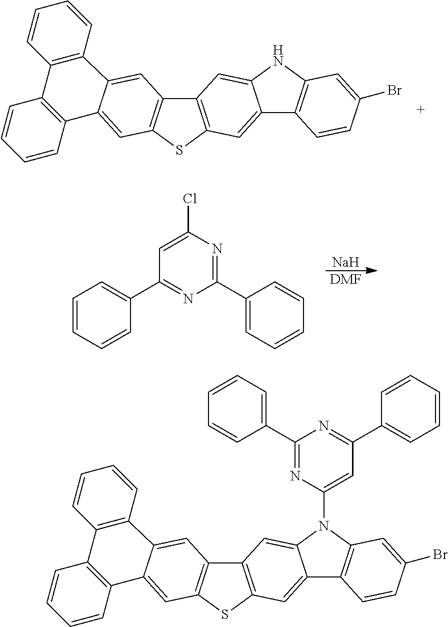

At 0° C., a mixture of sodium hydride (0.5 g, 22 mmol) and dimethylformamide (10 ml) was added the 14-bromo-16H-triphenyleno[2′,3′:4,5]thieno[3,2-b]carbazole (10 g, 20 mmol) dissolved in dimethylformamide (30 ml) slowly. After addition is completed, the mixture was warmed to room temperature and kept stirring 30 minutes. The mixture of 4-chloro-2,6-diphenylpyrimidine (5.8 g, 22 mmol) and dimethylformamide (25 ml) was added and stirred overnight. After the reaction finished, the mixture was quenched with ice water slowly and filtered to get the crude. The crude was purified by column chromatography, yielding 7.8 g of 14-bromo-16-(2,6-diphenylpyrimidin-4-yl)-16H-triphenyleno[2′,3′:4,5]thieno[3,2-b]carbazole as pale yellow solid (49%).

### Synthesis of 16-(2,6-diphenylpyrimidin-4-yl)-N-(4-methoxyphenyl)-N-(1H-phenalen-5-yl)-1 6H-triphenyleno[2′,3′:4,5]thieno[3,2-b]carbazol-14-amine (Compound 74)

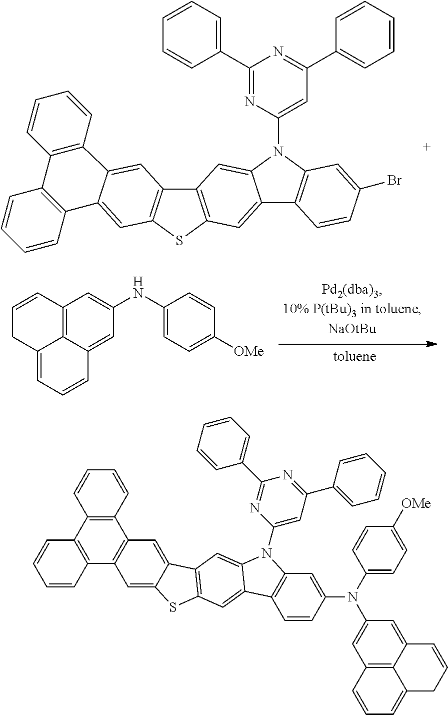 [SMILES: Brc1ccc2c3cc4sc5cc6c7ccccc7c7ccccc7c6cc5c4cc3n(-c3cc(-c4ccccc4)nc(-c4ccccc4)n3)c2c1 ; COc1ccc(Nc2cc3c4c(cccc4c2)CC=C3)cc1 ; COc1ccc(N(c2cc3c4c(cccc4c2)CC=C3)c2ccc3c4cc5sc6cc7c8ccccc8c8ccccc8c7cc6c5cc4n(-c4cc(-c5ccccc5)nc(-c5ccccc5)n4)c3c2)cc1]

A mixture of 14-bromo-16-(2,6-diphenylpyrimidin-4-yl)-16H-triphenyleno[2′,3′:4,5]thieno[3,2-b]carbazole (2.3 g, 3.2 mmol), N-(4-methoxyphenyl)-1H-phenalen-5-amine (1 g, 3.6 mmol), 2M aqueous solution of sodium carbonate (3 ml), ethanol (6 ml), and toluene (18 ml) was degassed by bubbling nitrogen gas for 15 minutes followed by addition of palladium tetrakis (0.07 g, 0.06 mmol) and 2-(dicyclohexylphosphino)biphenyl (0.04 g, 0.12 mmol). The mixture was heated to reflux overnight. Upon cooling to room temperature, the mixture was filtered to obtain solid. The solid was washed with water and methanol. The residue was purified by column chromatography on silica, yielding 1.8 g of 16-(2,6-diphenylpyrimidin-4-yl)-N-(4-methoxyphenyl)-N-(1H-phenalen-5-yl)-1 6H-triphenyleno[2,3′:4,5]thieno[3,2-b]carbazol-14-amine (compound 74) as pale yellow solid (62%).

### Examples 5-15

According to the previous synthesis methods, series of intermediates and the product compounds are synthesized analogously, as follows.

| Ex. | Intermediate 1 | Intermediate 2 |
|---|---|---|
| 5 | 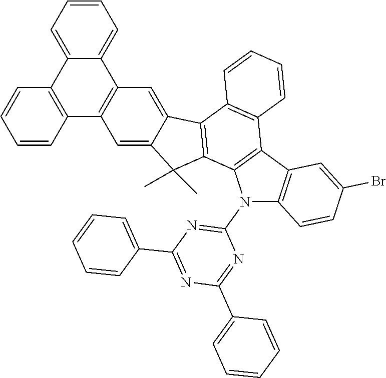 [SMILES: CC1(C)c2cc3c4ccccc4c4ccccc4c3cc2-c2c1c1c(c3ccccc23)c2cc(Br)ccc2n1-c1nc(-c2ccccc2)nc(-c2ccccc2)n1] | 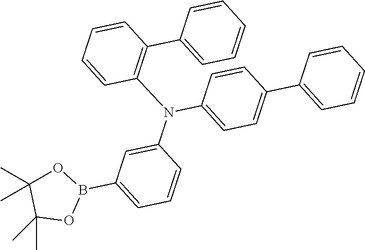 [SMILES: CC1(C)OB(c2cccc(N(c3ccc(-c4ccccc4)cc3)c3ccccc3-c3ccccc3)c2)OC1(C)C] |
| 6 | 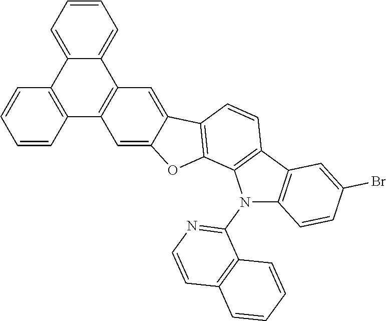 [SMILES: Brc1ccc2c(c1)c1ccc3c4cc5c6ccccc6c6ccccc6c5cc4oc3c1n2-c1nccc2ccccc12] | 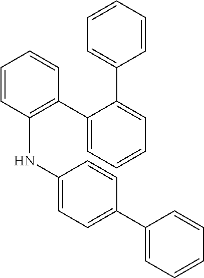 [SMILES: c1ccc(-c2ccc(Nc3ccccc3-c3ccccc3-c3ccccc3)cc2)cc1] |
| 7 | 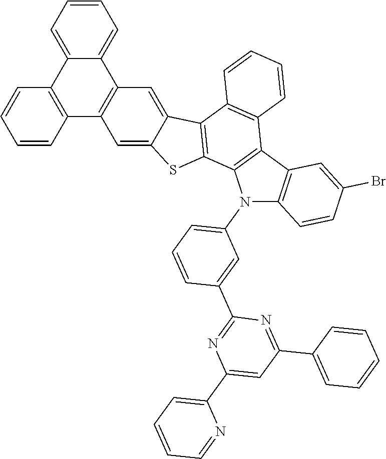 [SMILES: Brc1ccc2c(c1)c1c3ccccc3c3c4cc5c6ccccc6c6ccccc6c5cc4sc3c1n2-c1cccc(-c2nc(-c3ccccc3)cc(-c3ccccn3)n2)c1] | 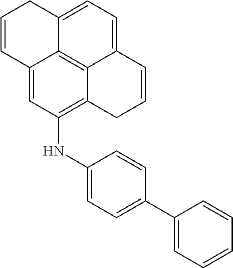 [SMILES: C1=Cc2cc(Nc3ccc(-c4ccccc4)cc3)c3c4c(ccc(c24)C1)C=CC3] |
| 8 | 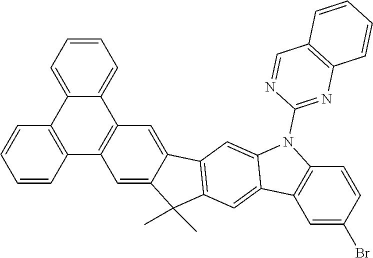 [SMILES: CC1(C)c2cc3c4ccccc4c4ccccc4c3cc2-c2cc3c(cc21)c1cc(Br)ccc1n3-c1ncc2ccccc2n1] | 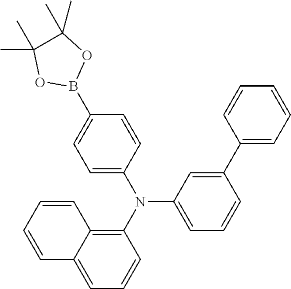 [SMILES: CC1(C)OB(c2ccc(N(c3cccc(-c4ccccc4)c3)c3cccc4ccccc34)cc2)OC1(C)C] |
| 9 | 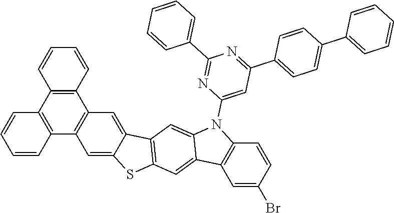 [SMILES: Brc1ccc2c(c1)c1cc3sc4cc5c6ccccc6c6ccccc6c5cc4c3cc1n2-c1cc(-c2ccc(-c3ccccc3)cc2)nc(-c2ccccc2)n1] | 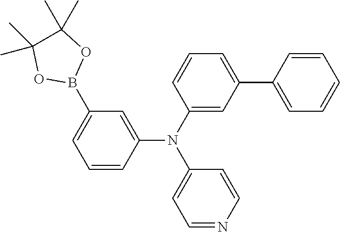 [SMILES: CC1(C)OB(c2cccc(N(c3ccncc3)c3cccc(-c4ccccc4)c3)c2)OC1(C)C] |
| 10 | 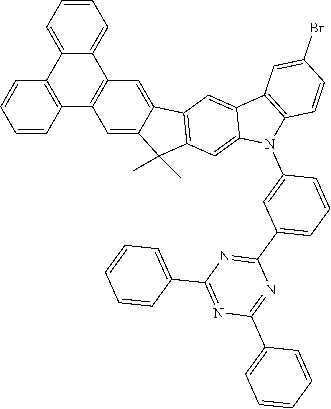 [SMILES: CC1(C)c2cc3c4ccccc4c4ccccc4c3cc2-c2cc3c4cc(Br)ccc4n(-c4cccc(-c5nc(-c6ccccc6)nc(-c6ccccc6)n5)c4)c3cc21] | 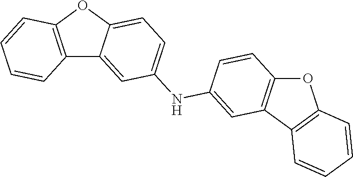 [SMILES: c1ccc2c(c1)oc1ccc(Nc3ccc4oc5ccccc5c4c3)cc12] |
| 11 | 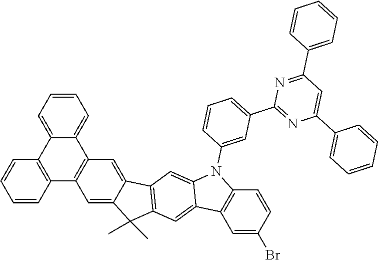 [SMILES: CC1(C)c2cc3c4ccccc4c4ccccc4c3cc2-c2cc3c(cc21)c1cc(Br)ccc1n3-c1cccc(-c2nc(-c3ccccc3)cc(-c3ccccc3)n2)c1] | 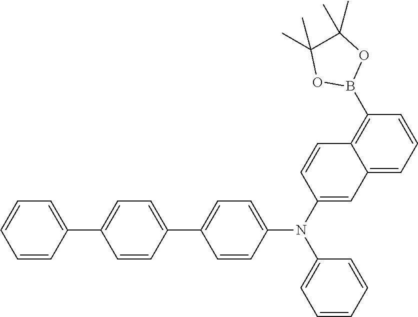 [SMILES: CC1(C)OB(c2cccc3cc(N(c4ccccc4)c4ccc(-c5ccc(-c6ccccc6)cc5)cc4)ccc23)OC1(C)C] |
| 12 | 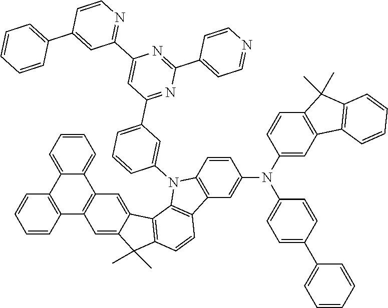 [SMILES: CC1(C)c2ccccc2-c2cc(N(c3ccc(-c4ccccc4)cc3)c3ccc4c(c3)c3ccc5c(c3n4-c3cccc(-c4cc(-c6cc(-c7ccccc7)ccn6)nc(-c6ccncc6)n4)c3)-c3cc4c6ccccc6c6ccccc6c4cc3C5(C)C)ccc21] | 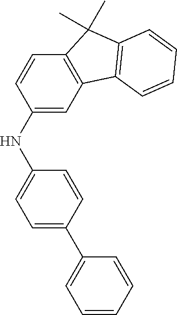 [SMILES: CC1(C)c2ccccc2-c2cc(Nc3ccc(-c4ccccc4)cc3)ccc21] |
| 13 | 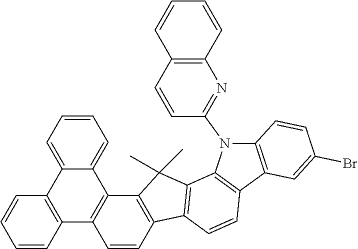 [SMILES: CC1(C)c2c(ccc3c4ccccc4c4ccccc4c23)-c2ccc3c4cc(Br)ccc4n(-c4ccc5ccccc5n4)c3c21] | 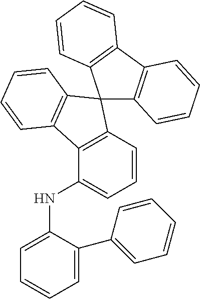 [SMILES: c1ccc(-c2ccccc2Nc2cccc3c2-c2ccccc2C32c3ccccc3-c3ccccc32)cc1] |
| 14 | 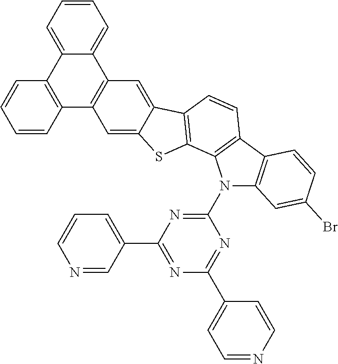 [SMILES: Brc1ccc2c3ccc4c5cc6c7ccccc7c7ccccc7c6cc5sc4c3n(-c3nc(-c4ccncc4)nc(-c4cccnc4)n3)c2c1] | 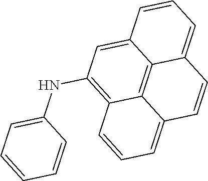 [SMILES: c1ccc(Nc2cc3cccc4ccc5cccc2c5c43)cc1] |
| 15 | 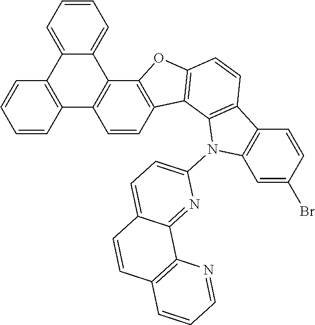 [SMILES: Brc1ccc2c3ccc4oc5c(ccc6c7ccccc7c7ccccc7c65)c4c3n(-c3ccc4ccc5cccnc5c4n3)c2c1] | 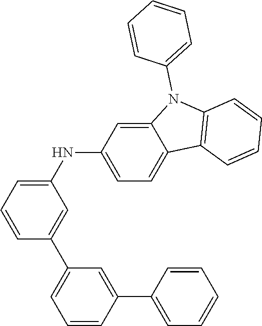 [SMILES: c1ccc(-c2cccc(-c3cccc(Nc4ccc5c6ccccc6n(-c6ccccc6)c5c4)c3)c2)cc1] |

| Ex. | product |
|---|---|
| 5 | 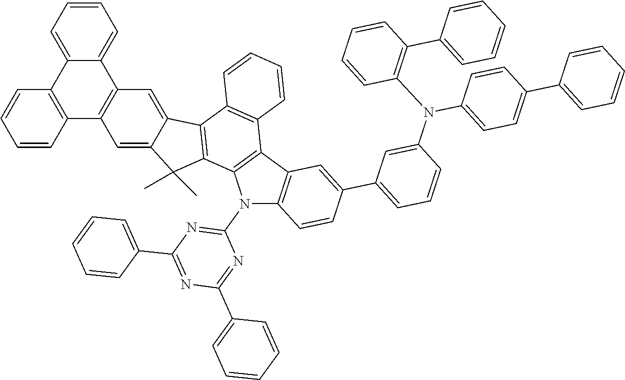 [SMILES: CC1(C)c2cc3c4ccccc4c4ccccc4c3cc2-c2c1c1c(c3ccccc23)c2cc(-c3cccc(N(c4ccc(-c5ccccc5)cc4)c4ccccc4-c4ccccc4)c3)ccc2n1-c1nc(-c2ccccc2)nc(-c2ccccc2)n1] |
| 6 | 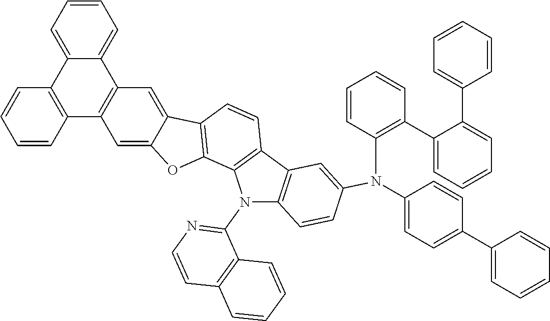 [SMILES: c1ccc(-c2ccc(N(c3ccc4c(c3)c3ccc5c6cc7c8ccccc8c8ccccc8c7cc6oc5c3n4-c3nccc4ccccc34)c3ccccc3-c3ccccc3-c3ccccc3)cc2)cc1] |
| 7 | 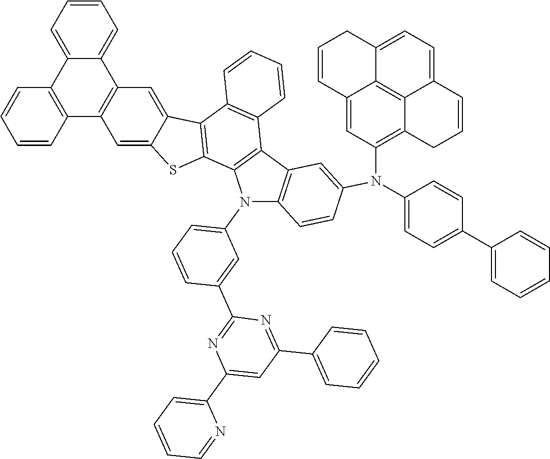 [SMILES: C1=Cc2cc(N(c3ccc(-c4ccccc4)cc3)c3ccc4c(c3)c3c5ccccc5c5c6cc7c8ccccc8c8ccccc8c7cc6sc5c3n4-c3cccc(-c4nc(-c5ccccc5)cc(-c5ccccn5)n4)c3)c3c4c(ccc(c24)C1)C=CC3] |
| 8 | 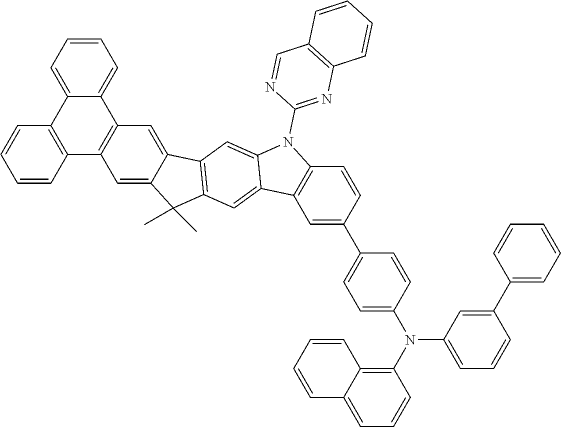 [SMILES: CC1(C)c2cc3c4ccccc4c4ccccc4c3cc2-c2cc3c(cc21)c1cc(-c2ccc(N(c4cccc(-c5ccccc5)c4)c4cccc5ccccc45)cc2)ccc1n3-c1ncc2ccccc2n1] |
| 9 | 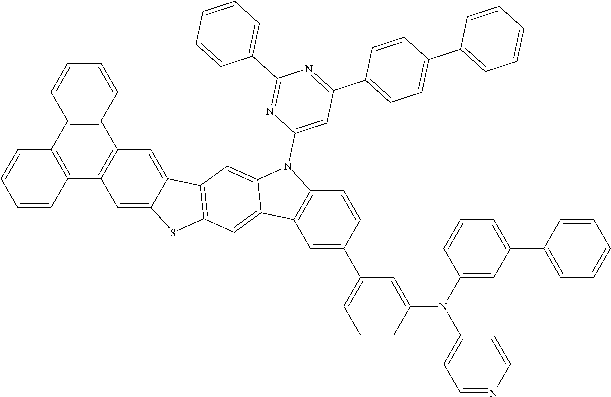 [SMILES: c1ccc(-c2ccc(-c3cc(-n4c5ccc(-c6cccc(N(c7ccncc7)c7cccc(-c8ccccc8)c7)c6)cc5c5cc6sc7cc8c9ccccc9c9ccccc9c8cc7c6cc54)nc(-c4ccccc4)n3)cc2)cc1] |
| 10 | 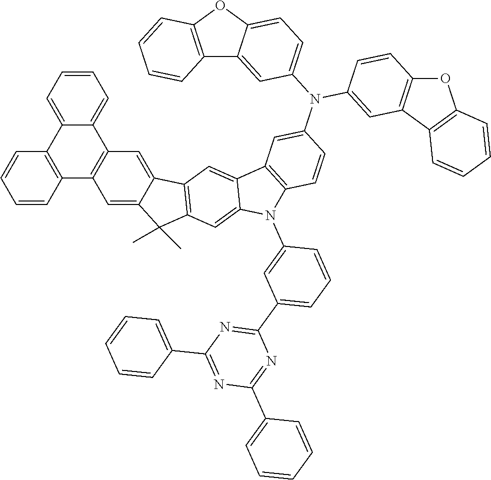 [SMILES: CC1(C)c2cc3c4ccccc4c4ccccc4c3cc2-c2cc3c4cc(N(c5ccc6oc7ccccc7c6c5)c5ccc6oc7ccccc7c6c5)ccc4n(-c4cccc(-c5nc(-c6ccccc6)nc(-c6ccccc6)n5)c4)c3cc21] |
| 11 | 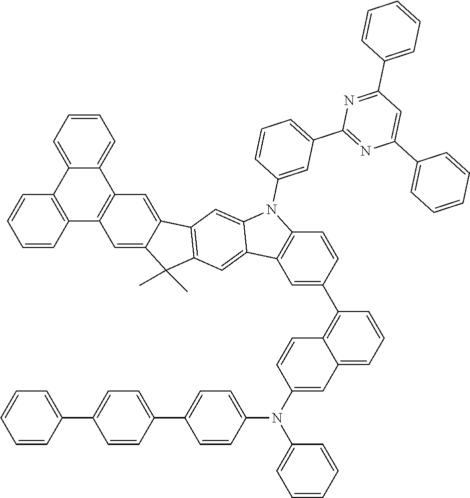 [SMILES: CC1(C)c2cc3c4ccccc4c4ccccc4c3cc2-c2cc3c(cc21)c1cc(-c2cccc4cc(N(c5ccccc5)c5ccc(-c6ccc(-c7ccccc7)cc6)cc5)ccc24)ccc1n3-c1cccc(-c2nc(-c3ccccc3)cc(-c3ccccc3)n2)c1] |
| 12 | 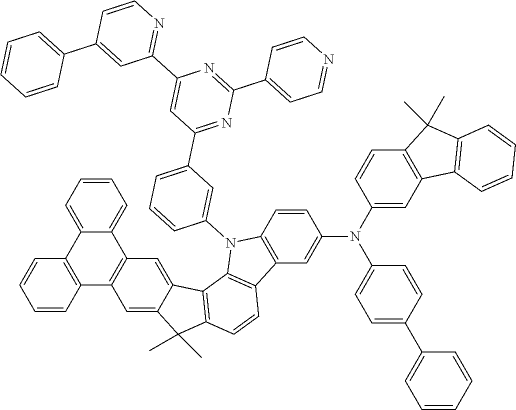 [SMILES: CC1(C)c2ccccc2-c2cc(N(c3ccc(-c4ccccc4)cc3)c3ccc4c(c3)c3ccc5c(c3n4-c3cccc(-c4cc(-c6cc(-c7ccccc7)ccn6)nc(-c6ccncc6)n4)c3)-c3cc4c6ccccc6c6ccccc6c4cc3C5(C)C)ccc21] |
| 13 | 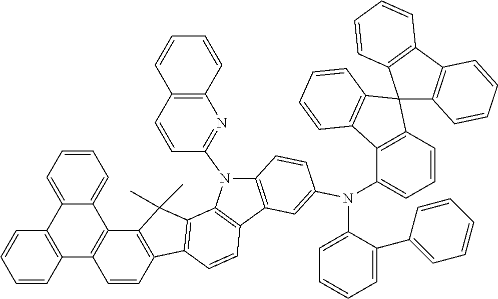 [SMILES: CC1(C)c2c(ccc3c4ccccc4c4ccccc4c23)-c2ccc3c4cc(N(c5ccccc5-c5ccccc5)c5cccc6c5-c5ccccc5C65c6ccccc6-c6ccccc65)ccc4n(-c4ccc5ccccc5n4)c3c21] |
| 14 | 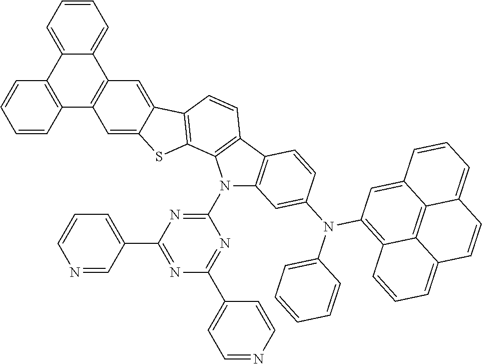 [SMILES: c1ccc(N(c2ccc3c4ccc5c6cc7c8ccccc8c8ccccc8c7cc6sc5c4n(-c4nc(-c5ccncc5)nc(-c5cccnc5)n4)c3c2)c2cc3cccc4ccc5cccc2c5c43)cc1] |
| 15 | 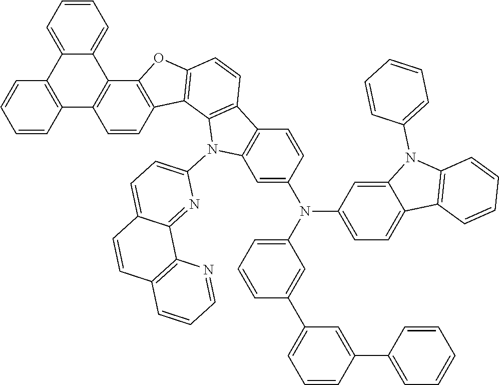 [SMILES: c1ccc(-c2cccc(-c3cccc(N(c4ccc5c6ccccc6n(-c6ccccc6)c5c4)c4ccc5c6ccc7oc8c(ccc9c%10ccccc%10c%10ccccc%10c98)c7c6n(-c6ccc7ccc8cccnc8c7n6)c5c4)c3)c2)cc1] |

What is claimed is:

1. An organic compound represented by the following formula (1): 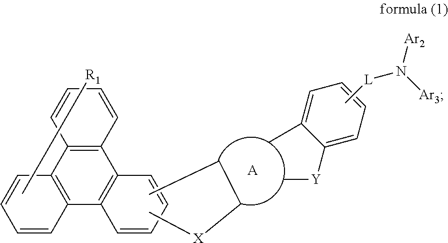
wherein X and Y independently represent a divalent bridge selected from the group consisting of O, S, NAr₁ and CR₂R₃;
wherein ring A represents a monocyclic aromatic group or a polycyclic aromatic group having at least two fused rings;
wherein L represents a single bond or an aromatic linker;
wherein R₁ to R₃ independently represent no substitution or mono to the maximum allowable substitution, each of the substituents is selected from the group consisting of alkyl, aryl, aralkyl, heteroaryl, and combinations thereof; and
wherein each of Ar₁, Ar₂ and Ar₃ is selected from the group consisting of aryl, heteroaryl, and combinations thereof.

2. The organic compound according to claim 1, wherein the organic compound is represented by one of the following formulae:

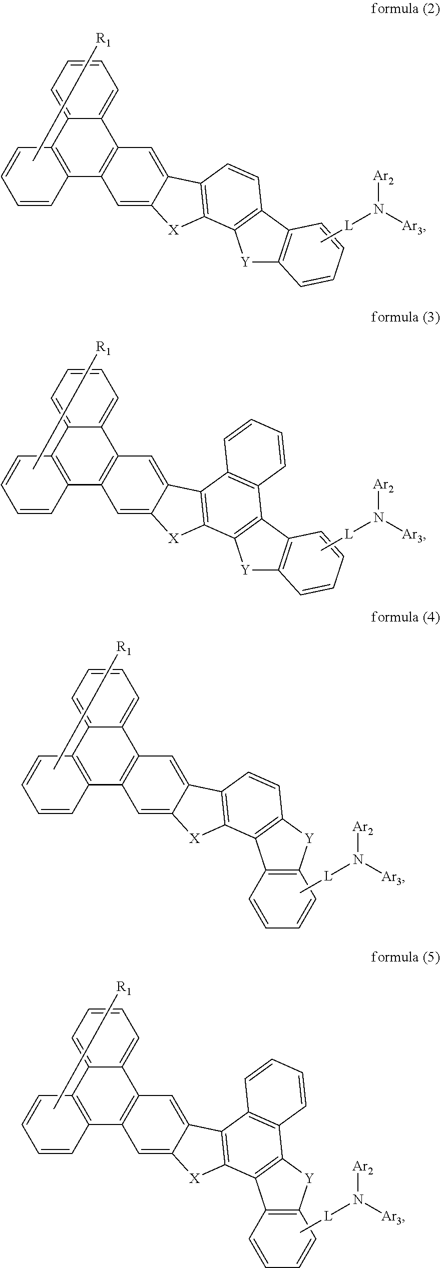

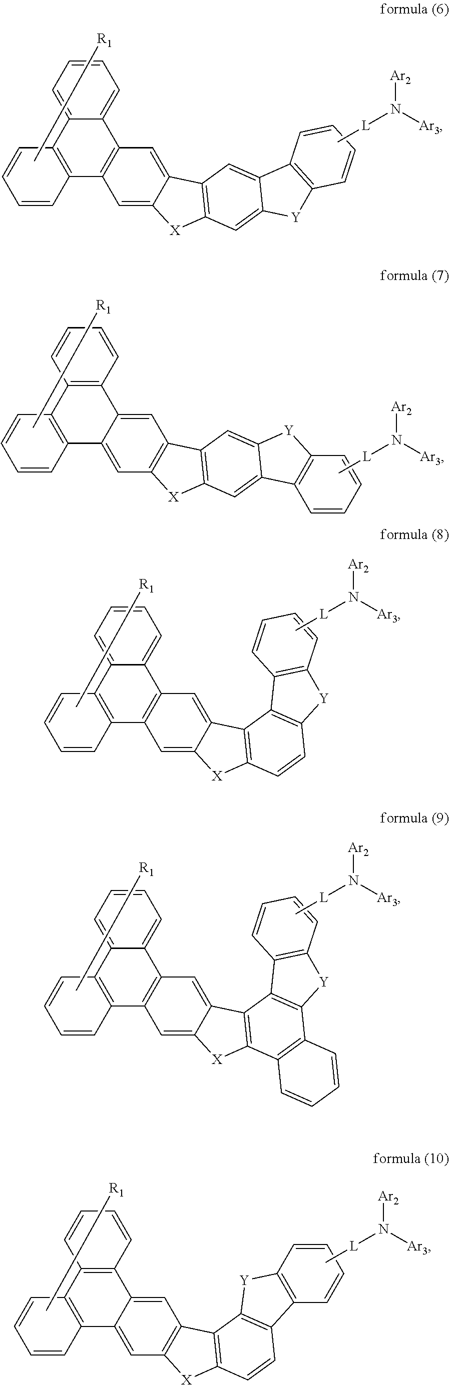

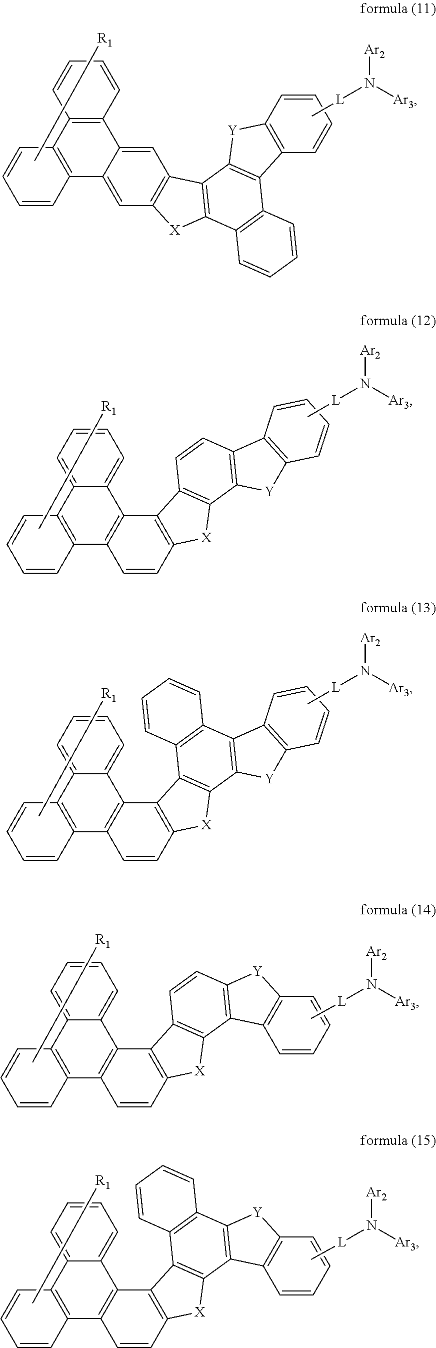

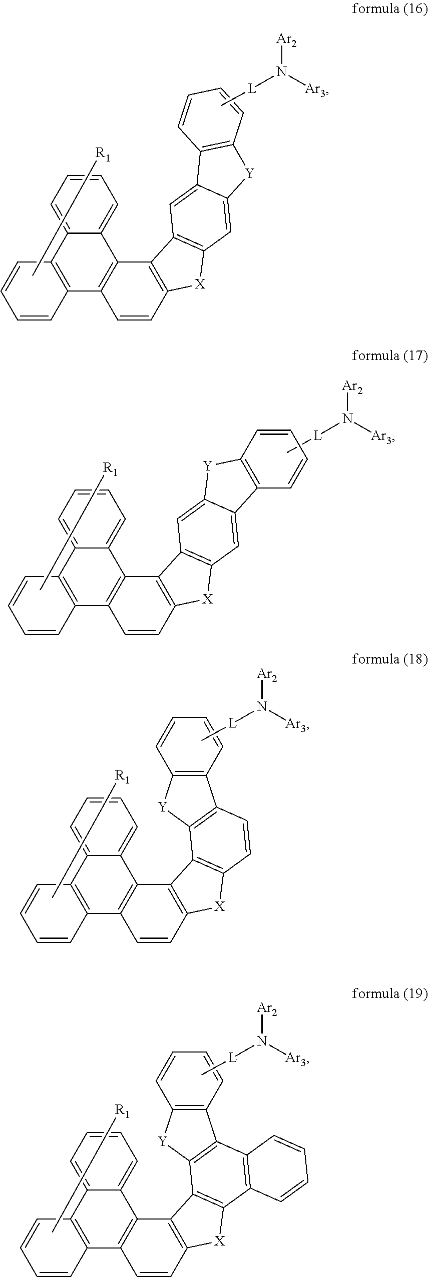

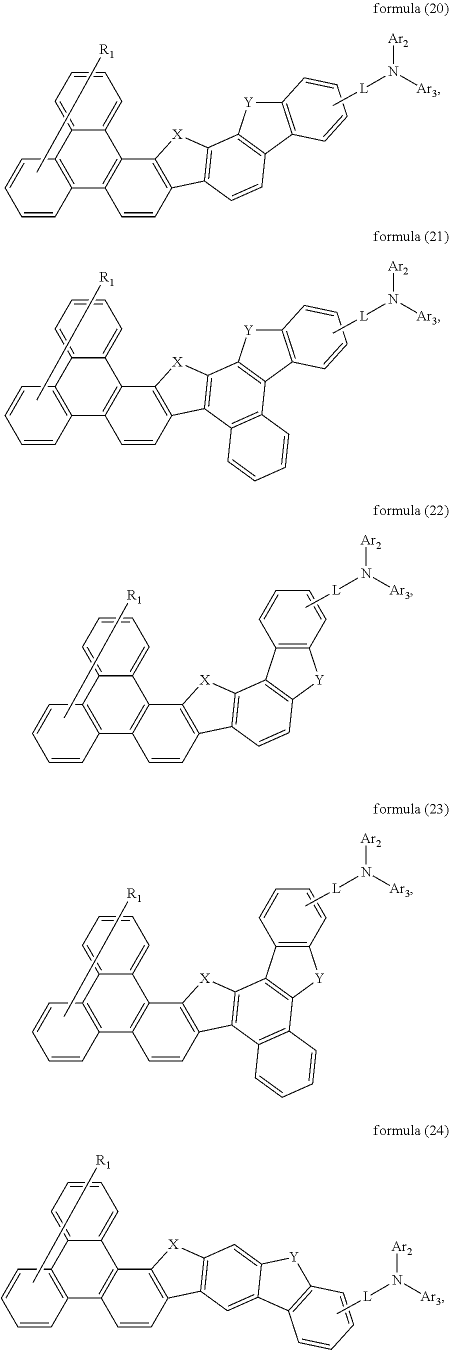

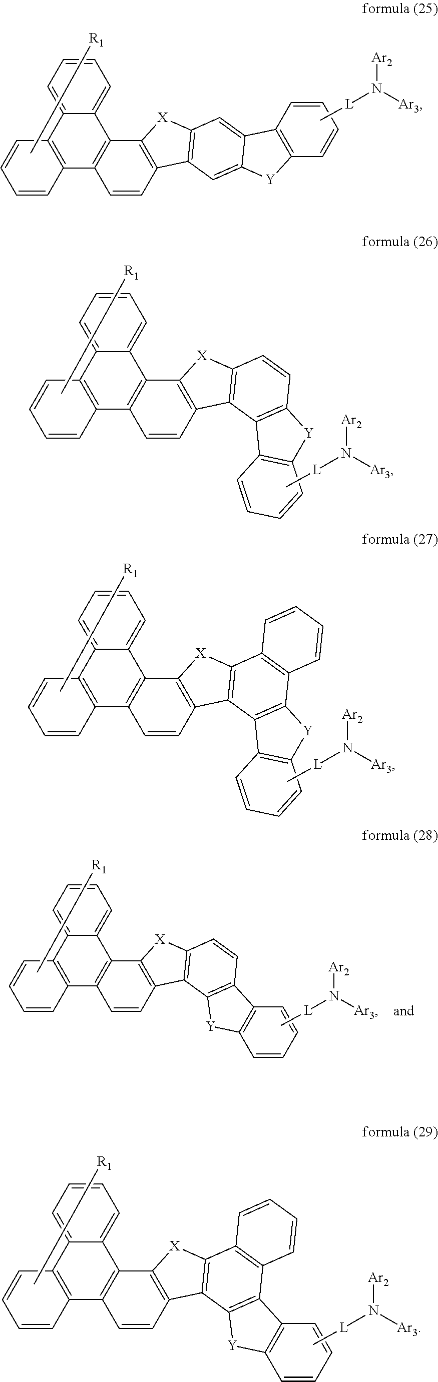

3. The organic compound according to claim 1, wherein Ar₂ and Ar₃ are independently selected from the group consisting of biphenyl, terphenyl, quaterphenyl, fluorenyl, carbazolyl, dibenzofuranyl, dibenzothiophenyl, naphtyl, anthracyl, phenanthryl, chrysenyl, triphenylenyl, pyrenyl, and combinations thereof.

4. The organic compound according to claim 1, wherein Ar₂ and Ar₃ are independently selected from the group consisting of: 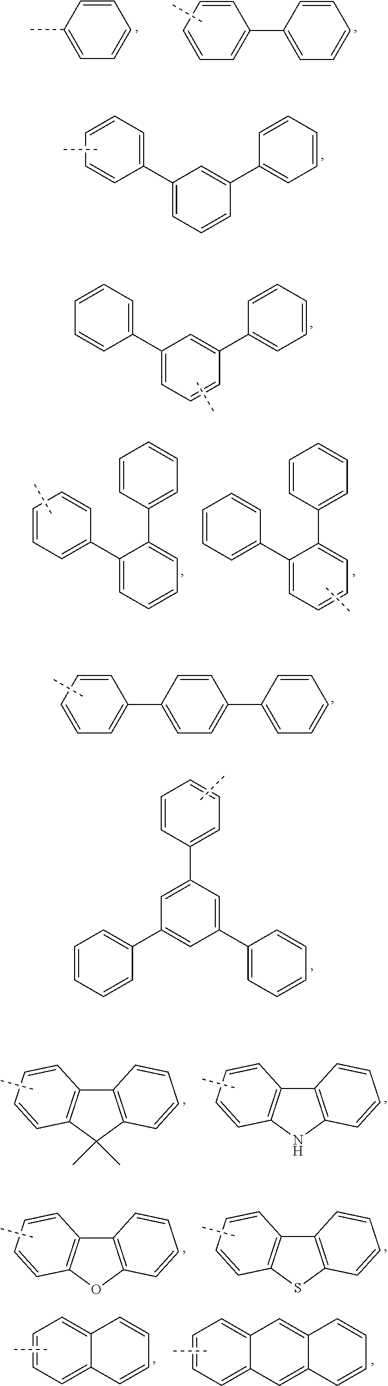 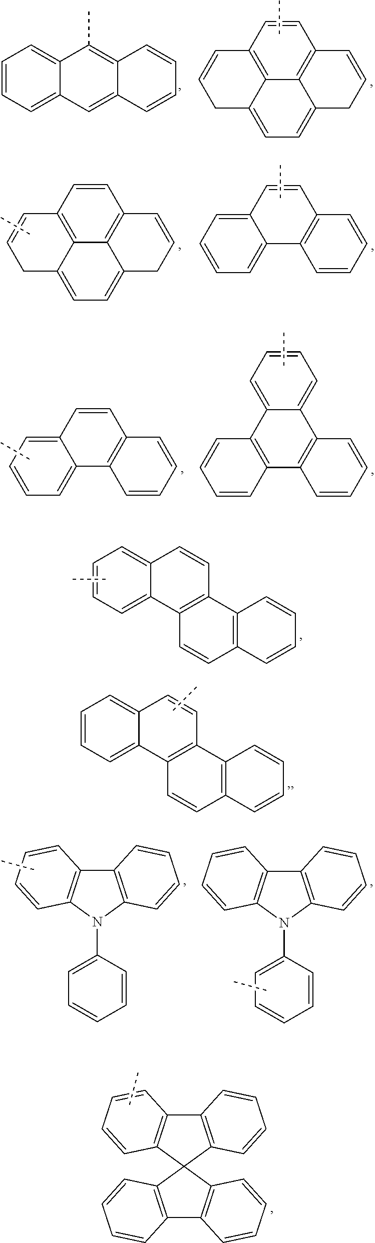
and combinations thereof.

5. The organic compound according to claim 1, wherein each of the R₁ to R₃ represents no substitution or is a substituent selected from the group consisting of alkyl having 20 or fewer carbon atoms, aryl having 30 or fewer carbon atoms, aralkyl having 30 or fewer carbon atoms, heteroaryl having 30 or fewer carbon atoms, and combinations thereof.

6. The organic compound according to claim 1, wherein R₁ represents no substitution or mono, di, tri, tetra, penta, hexa, hepta, or octa substitutions.

7. The organic compound according to claim 1, wherein L represents a single bond or an aromatic linker selected from the group consisting of alkyl, aryl, aralkyl, heteroaryl, and combinations thereof.

8. The organic compound according to claim 1, wherein L represents a single bond or an aromatic linker of arylene.

9. The organic compound according to claim 1, wherein L represents a substituted or unsubstituted divalent arylene having 6 to 30 ring carbon atoms.

10. The organic compound of claim 1, wherein the organic compound is selected from the group consisting of:

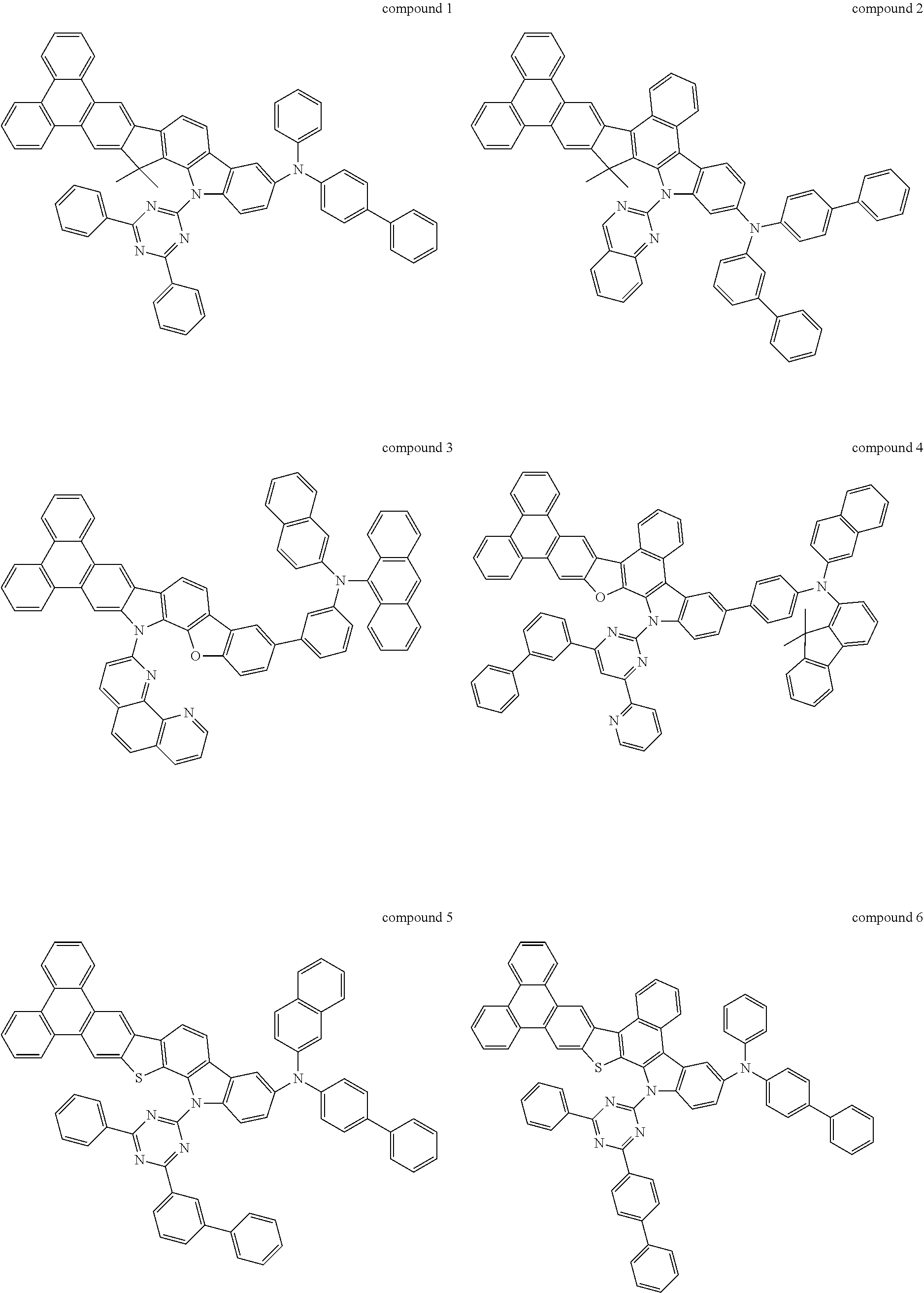

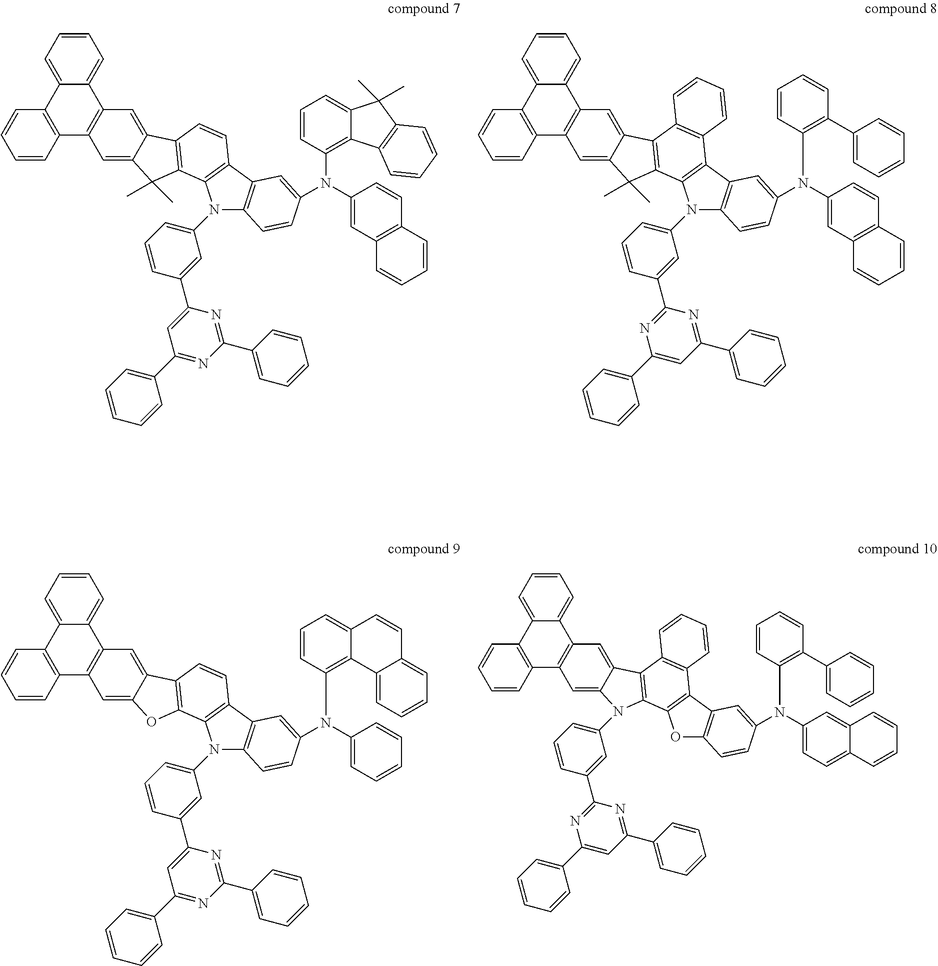

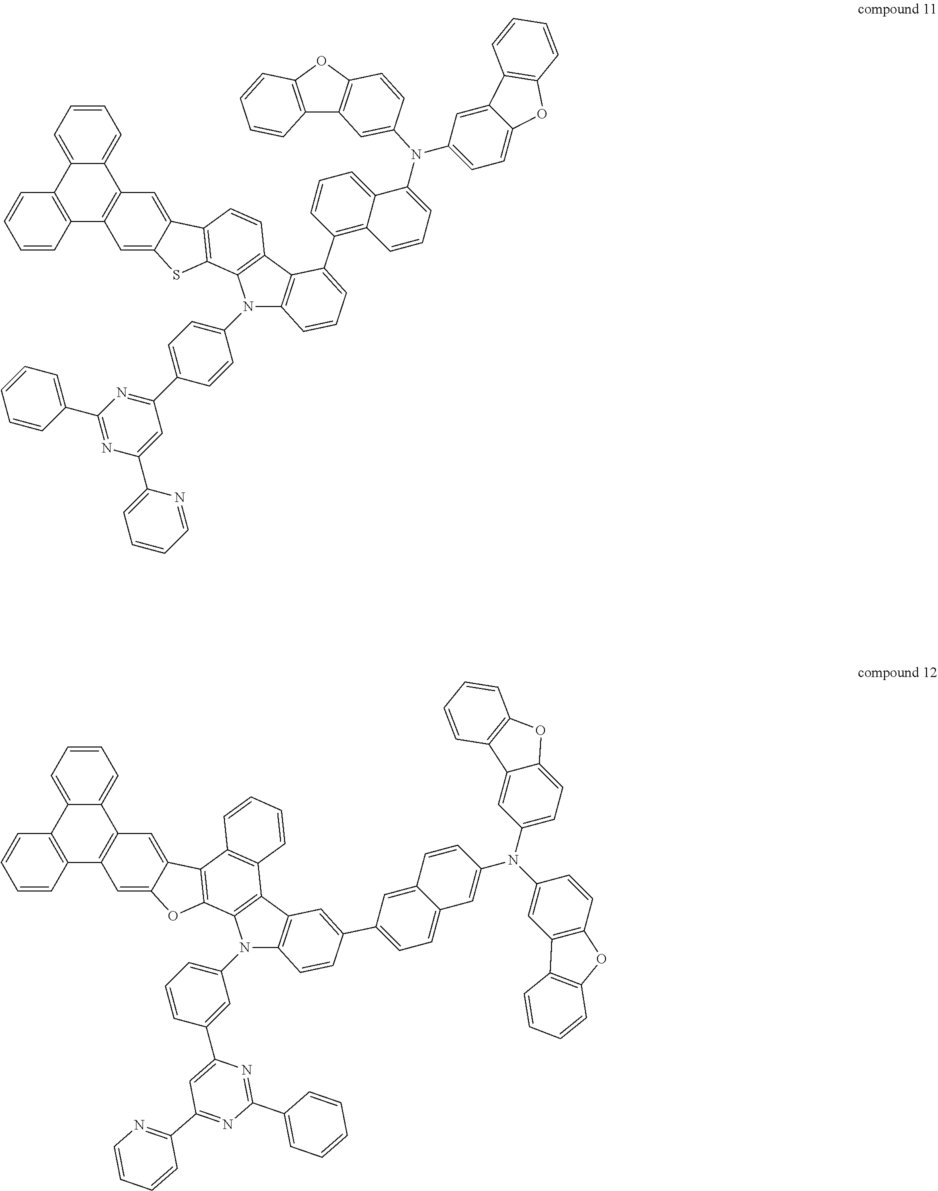 [SMILES: c1ccc(-c2nc(-c3ccc(-n4c5cccc(-c6cccc7c(N(c8ccc9oc%10ccccc%10c9c8)c8ccc9oc%10ccccc%10c9c8)cccc67)c5c5ccc6c7cc8c9ccccc9c9ccccc9c8cc7sc6c54)cc3)cc(-c3ccccn3)n2)cc1 ; c1ccc(-c2nc(-c3cccc(-n4c5ccc(-c6ccc7cc(N(c8ccc9oc%10ccccc%10c9c8)c8ccc9oc%10ccccc%10c9c8)ccc7c6)cc5c5c6ccccc6c6c7cc8c9ccccc9c9ccccc9c8cc7oc6c54)c3)cc(-c3ccccn3)n2)cc1]

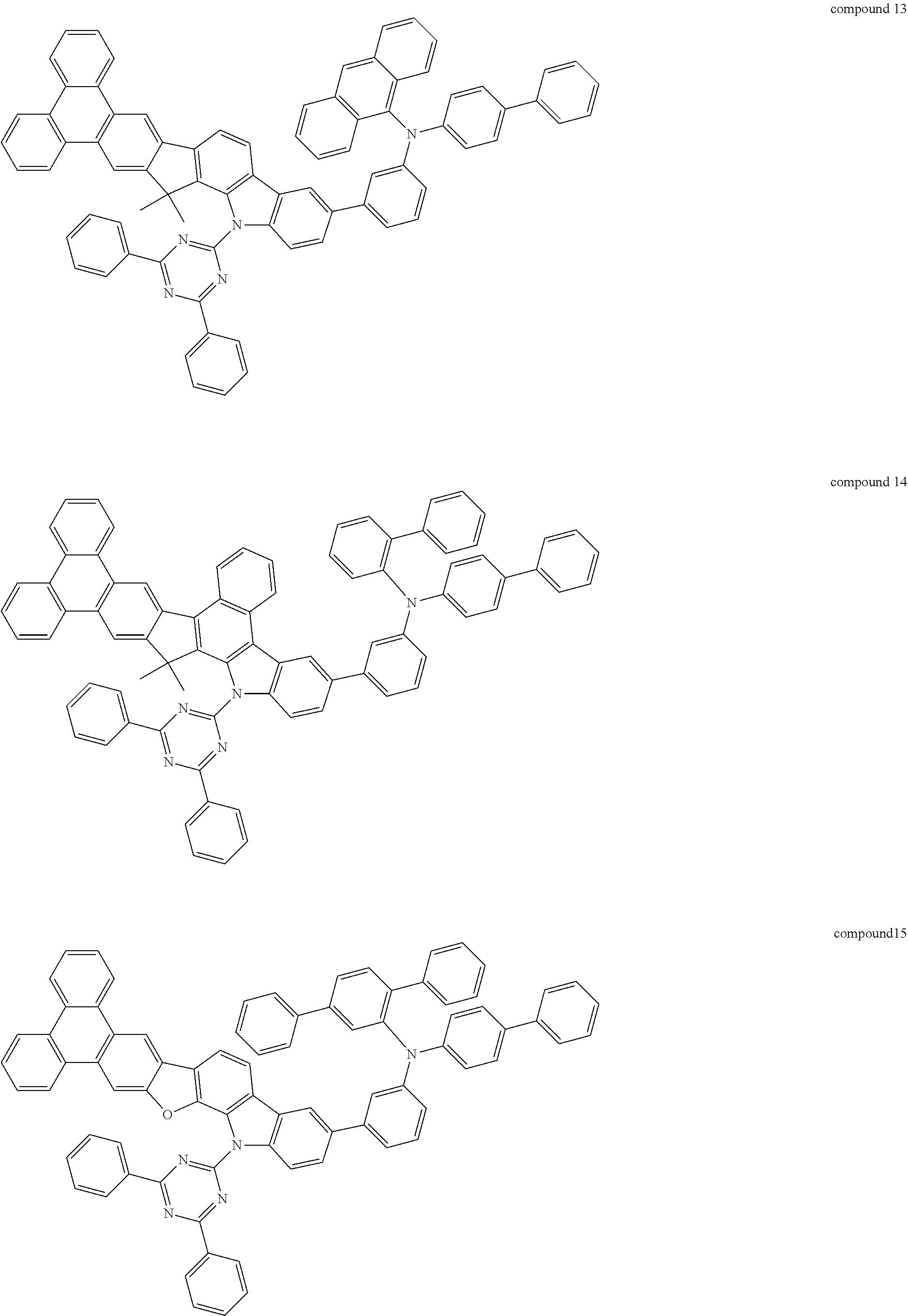 [SMILES: CC1(C)c2cc3c4ccccc4c4ccccc4c3cc2-c2ccc3c4cc(-c5cccc(N(c6ccc(-c7ccccc7)cc6)c6c7ccccc7cc7ccccc67)c5)ccc4n(-c4nc(-c5ccccc5)nc(-c5ccccc5)n4)c3c21 ; CC1(C)c2cc3c4ccccc4c4ccccc4c3cc2-c2c1c1c(c3ccccc23)c2cc(-c3cccc(N(c4ccc(-c5ccccc5)cc4)c4ccccc4-c4ccccc4)c3)ccc2n1-c1nc(-c2ccccc2)nc(-c2ccccc2)n1 ; c1ccc(-c2ccc(N(c3cccc(-c4ccc5c(c4)c4ccc6c7cc8c9ccccc9c9ccccc9c8cc7oc6c4n5-c4nc(-c5ccccc5)nc(-c5ccccc5)n4)c3)c3cc(-c4ccccc4)ccc3-c3ccccc3)cc2)cc1]

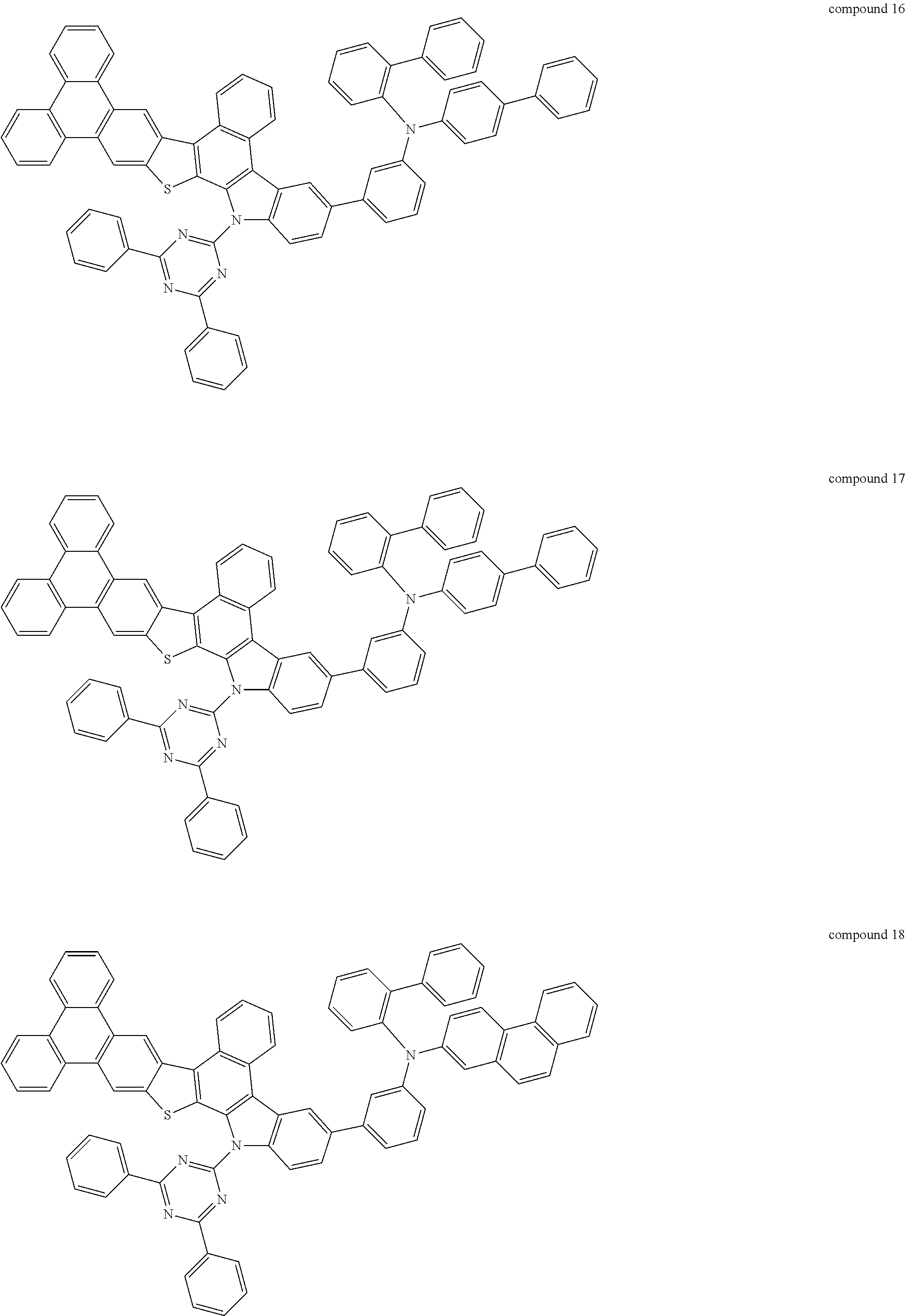 [SMILES: c1ccc(-c2ccc(N(c3cccc(-c4ccc5c(c4)c4c6ccccc6c6c7cc8c9ccccc9c9ccccc9c8cc7sc6c4n5-c4nc(-c5ccccc5)nc(-c5ccccc5)n4)c3)c3ccccc3-c3ccccc3)cc2)cc1 ; c1ccc(-c2ccc(N(c3cccc(-c4ccc5c(c4)c4c6ccccc6c6c7cc8c9ccccc9c9ccccc9c8cc7sc6c4n5-c4nc(-c5ccccc5)nc(-c5ccccc5)n4)c3)c3ccccc3-c3ccccc3)cc2)cc1 ; c1ccc(-c2nc(-c3ccccc3)nc(-n3c4ccc(-c5cccc(N(c6ccc7c(ccc8ccccc87)c6)c6ccccc6-c6ccccc6)c5)cc4c4c5ccccc5c5c6cc7c8ccccc8c8ccccc8c7cc6sc5c43)n2)cc1]

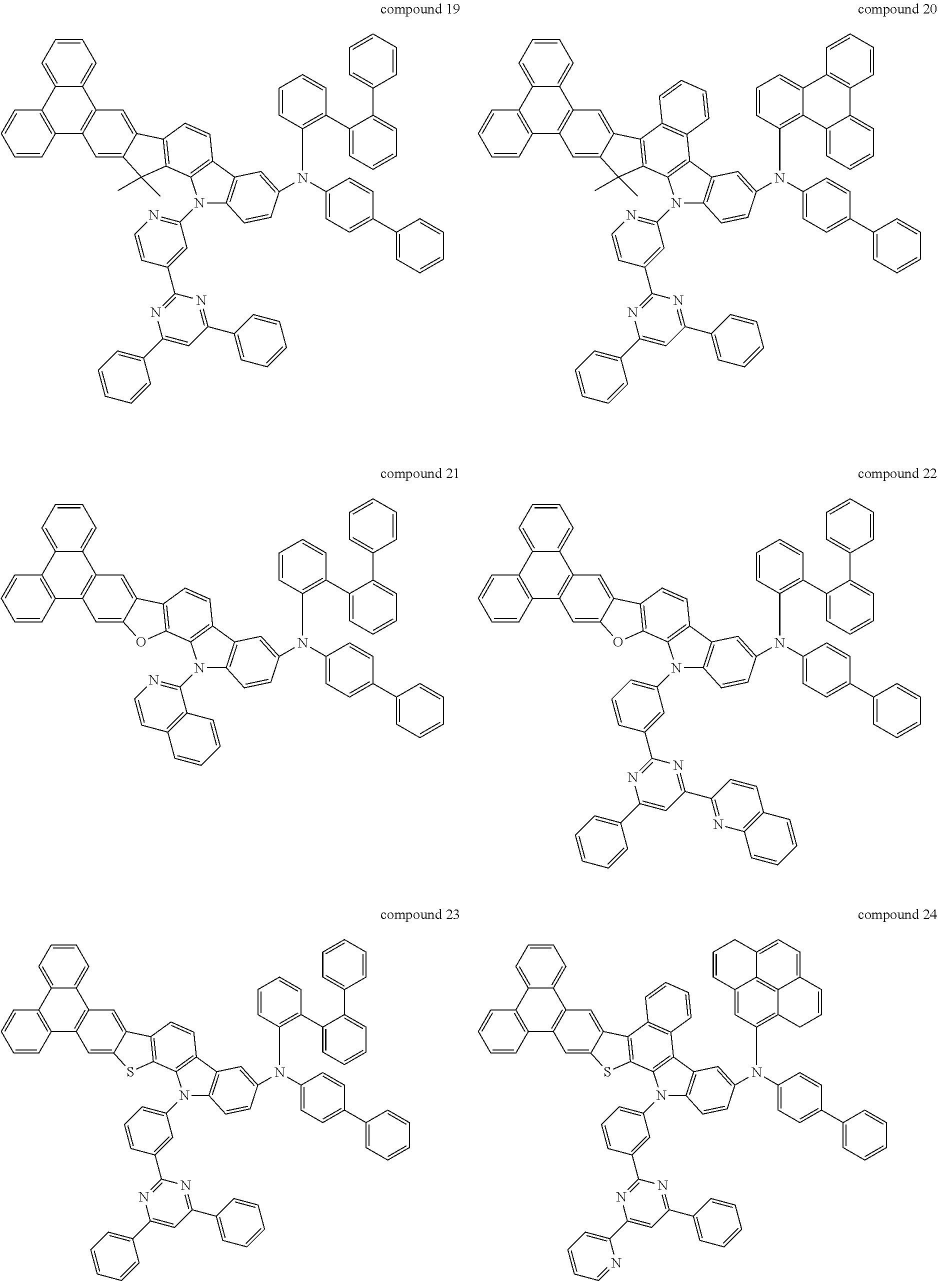

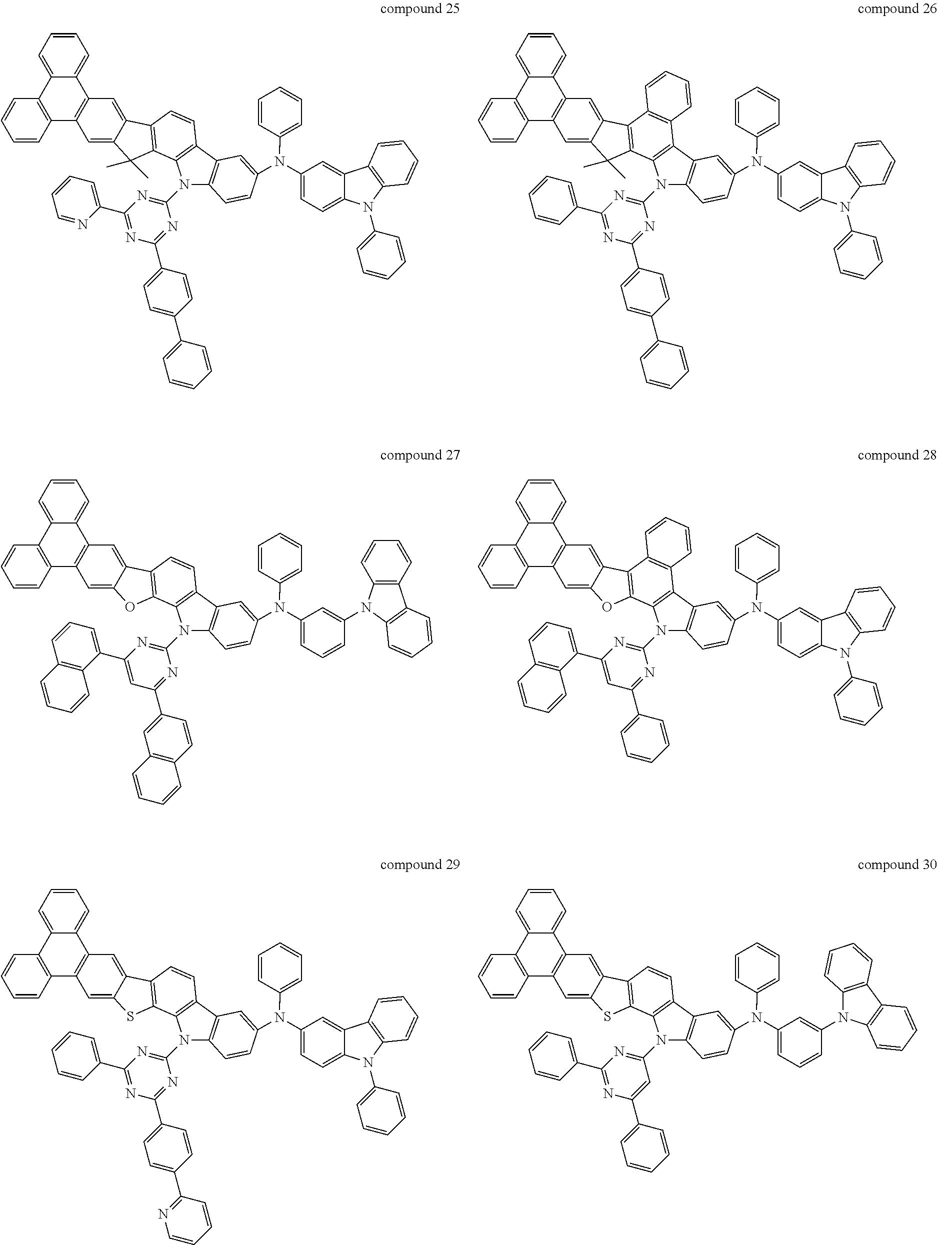

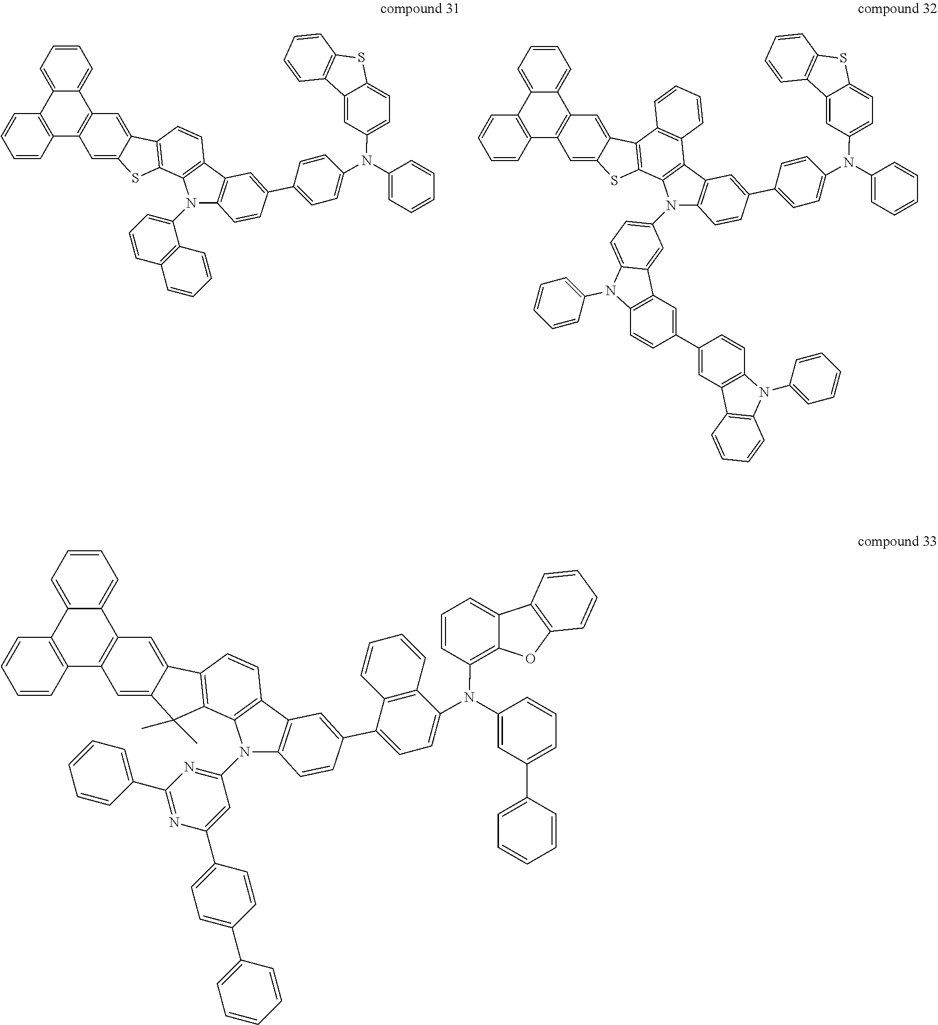 [SMILES: c1ccc(N(c2ccc(-c3ccc4c(c3)c3ccc5c6cc7c8ccccc8c8ccccc8c7cc6sc5c3n4-c3cccc4ccccc34)cc2)c2ccc3sc4ccccc4c3c2)cc1 ; c1ccc(N(c2ccc(-c3ccc4c(c3)c3c5ccccc5c5c6cc7c8ccccc8c8ccccc8c7cc6sc5c3n4-c3ccc4c(c3)c3cc(-c5ccc6c(c5)c5ccccc5n6-c5ccccc5)ccc3n4-c3ccccc3)cc2)c2ccc3sc4ccccc4c3c2)cc1 ; CC1(C)c2cc3c4ccccc4c4ccccc4c3cc2-c2ccc3c4cc(-c5ccc(N(c6cccc(-c7ccccc7)c6)c6cccc7c6oc6ccccc67)c6ccccc56)ccc4n(-c4cc(-c5ccc(-c6ccccc6)cc5)nc(-c5ccccc5)n4)c3c21]

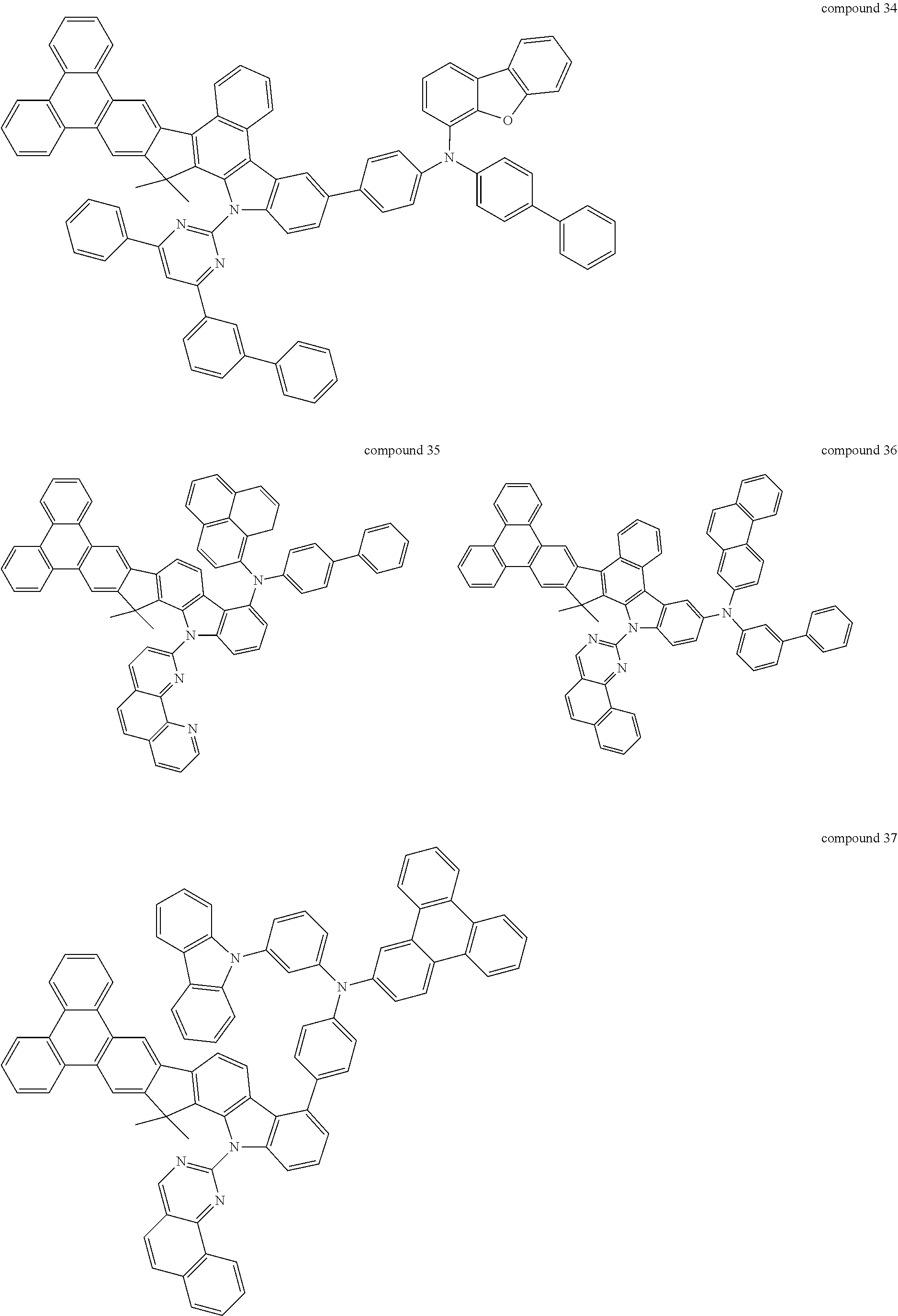

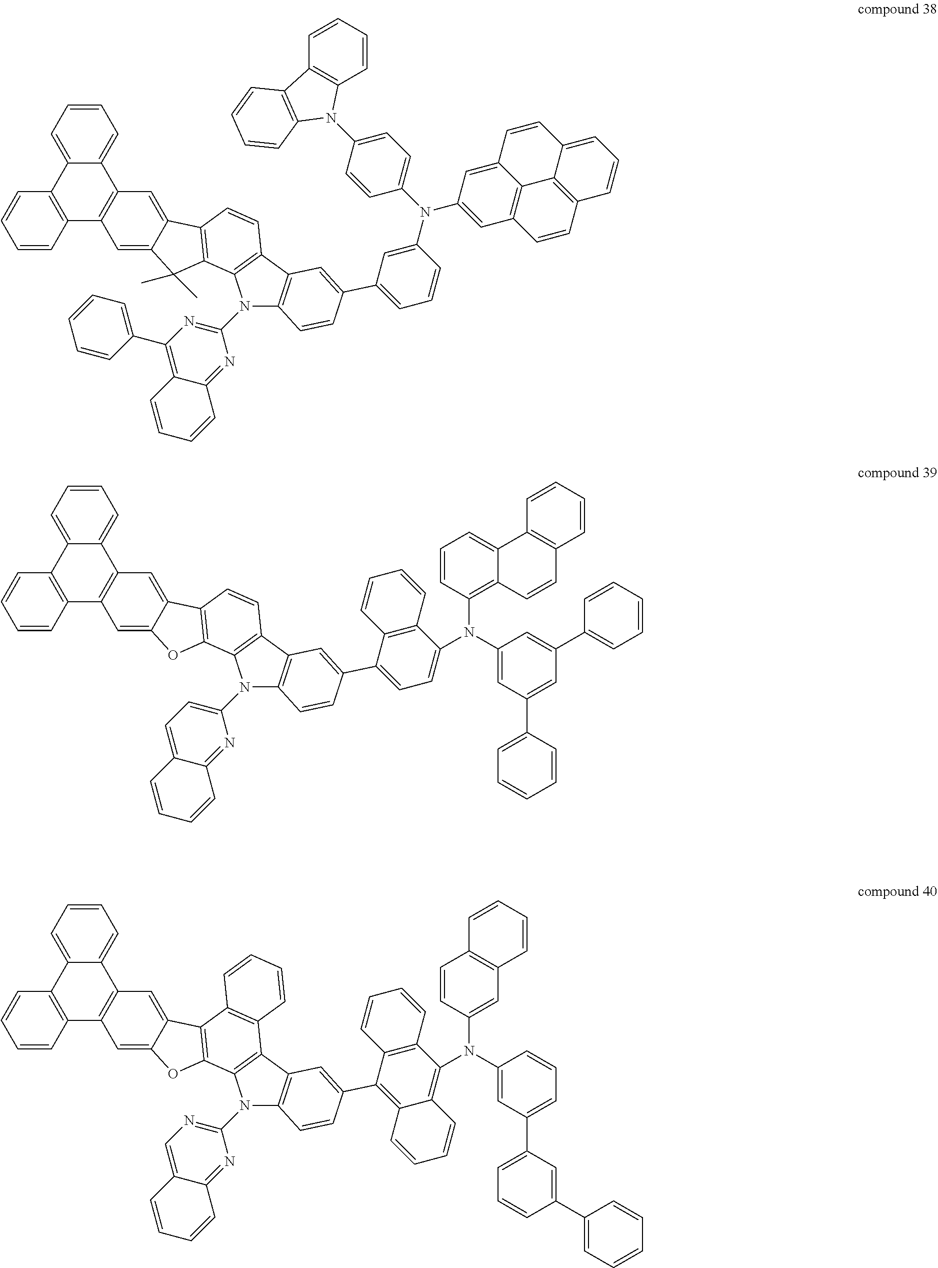 [SMILES: CC1(C)c2cc3c4ccccc4c4ccccc4c3cc2-c2ccc3c4cc(-c5cccc(N(c6ccc(-n7c8ccccc8c8ccccc87)cc6)c6cc7ccc8cccc9ccc(c6)c7c89)c5)ccc4n(-c4nc(-c5ccccc5)c5ccccc5n4)c3c21 ; c1ccc(-c2cc(-c3ccccc3)cc(N(c3ccc(-c4ccc5c(c4)c4ccc6c7cc8c9ccccc9c9ccccc9c8cc7oc6c4n5-c4ccc5ccccc5n4)c4ccccc34)c3cccc4c3ccc3ccccc34)c2)cc1 ; c1ccc(-c2cccc(-c3cccc(N(c4ccc5ccccc5c4)c4c5ccccc5c(-c5ccc6c(c5)c5c7ccccc7c7c8cc9c%10ccccc%10c%10ccccc%10c9cc8oc7c5n6-c5ncc6ccccc6n5)c5ccccc45)c3)c2)cc1]

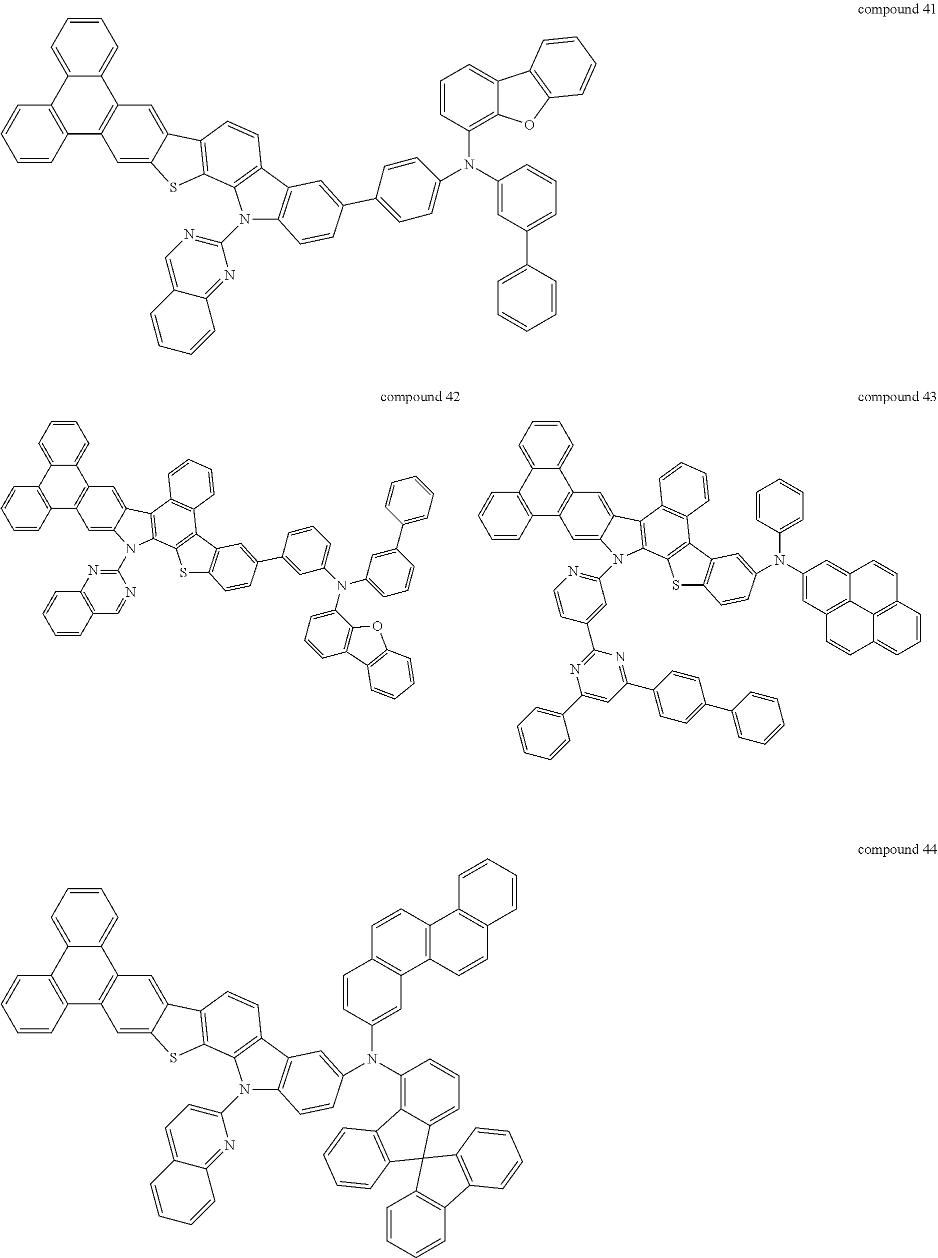

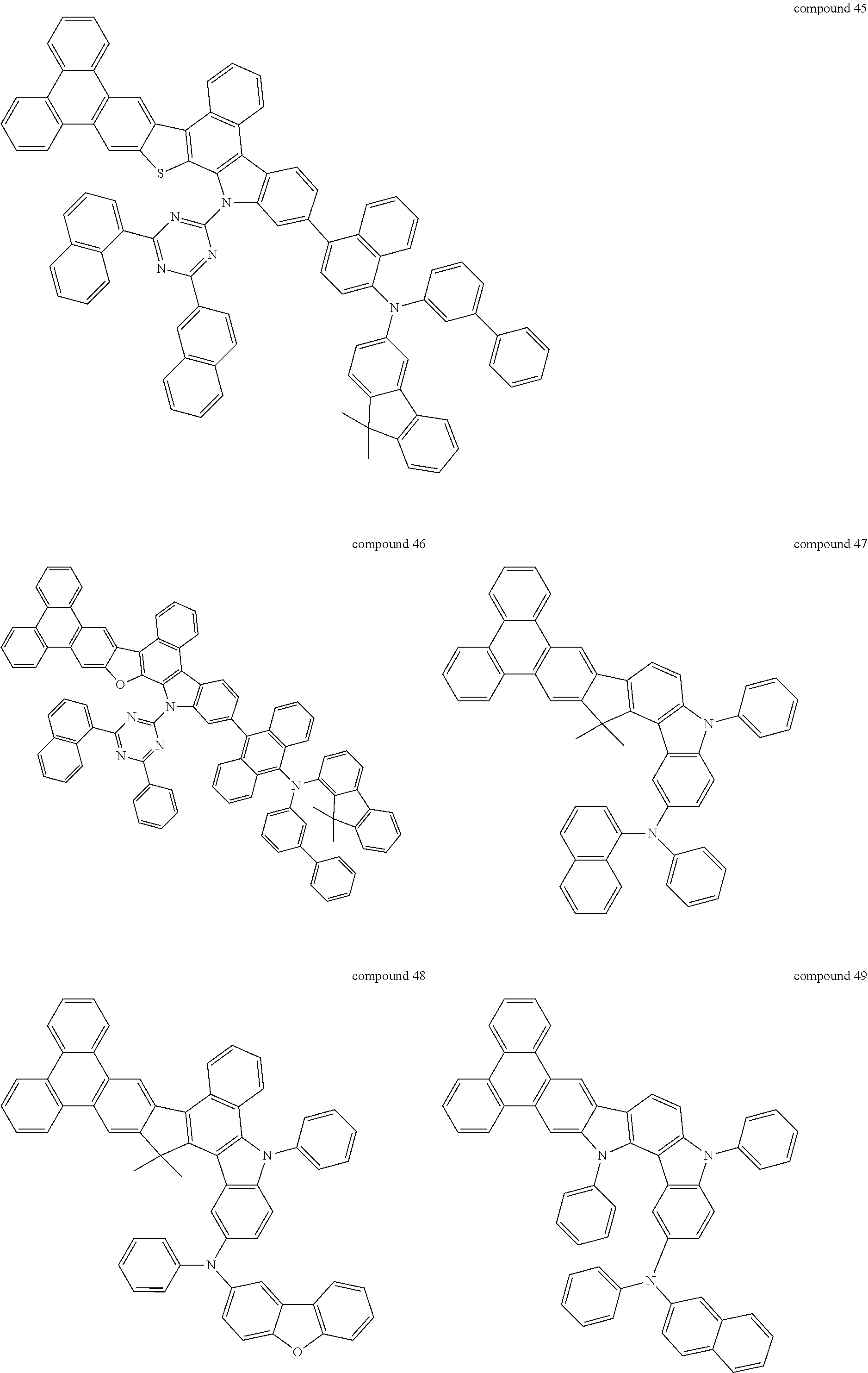

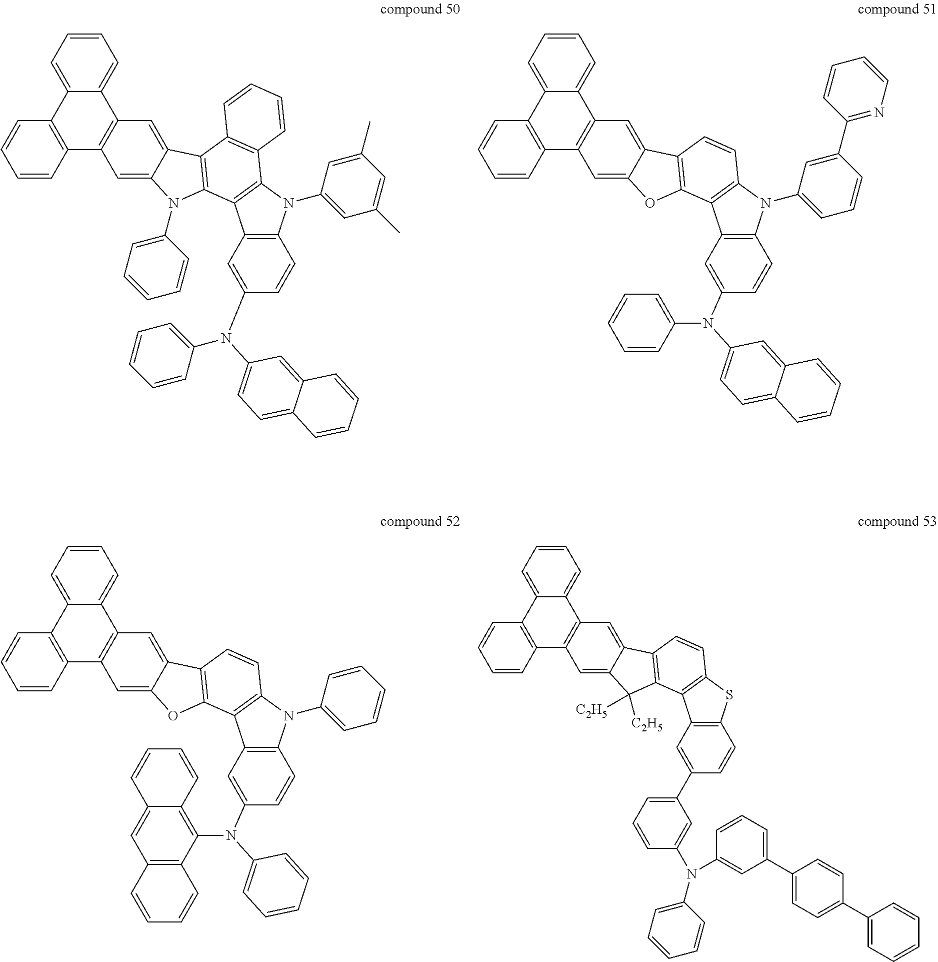 [SMILES: Cc1cc(C)cc(-n2c3ccc(N(c4ccccc4)c4ccc5ccccc5c4)cc3c3c2c2ccccc2c2c4cc5c6ccccc6c6ccccc6c5cc4n(-c4ccccc4)c23)c1 ; c1ccc(N(c2ccc3ccccc3c2)c2ccc3c(c2)c2c4oc5cc6c7ccccc7c7ccccc7c6cc5c4ccc2n3-c2cccc(-c3ccccn3)c2)cc1 ; c1ccc(N(c2ccc3c(c2)c2c4oc5cc6c7ccccc7c7ccccc7c6cc5c4ccc2n3-c2ccccc2)c2c3ccccc3cc3ccccc23)cc1 ; CCC1(CC)c2cc3c4ccccc4c4ccccc4c3cc2-c2ccc3sc4ccc(-c5cccc(N(c6ccccc6)c6cccc(-c7ccc(-c8ccccc8)cc7)c6)c5)cc4c3c21]

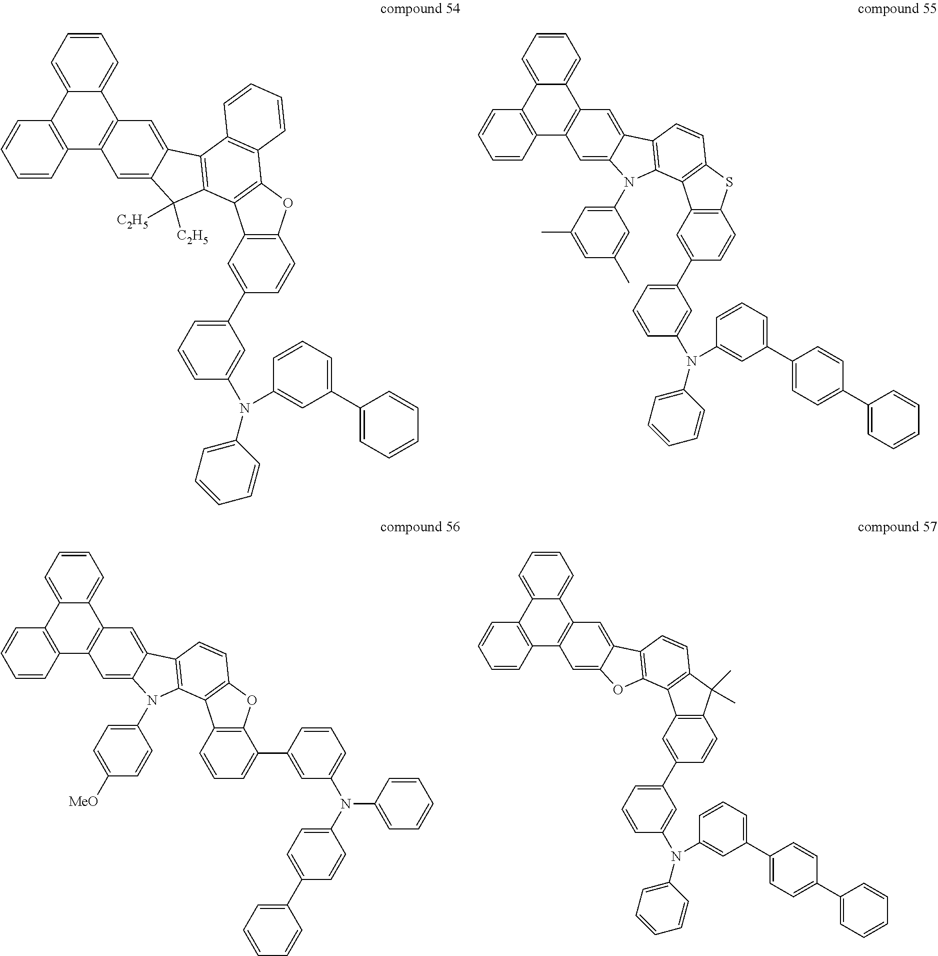 [SMILES: CCC1(CC)c2cc3c4ccccc4c4ccccc4c3cc2-c2c1c1c3cc(-c4cccc(N(c5ccccc5)c5cccc(-c6ccccc6)c5)c4)ccc3oc1c1ccccc21 ; Cc1cc(C)cc(-n2c3cc4c5ccccc5c5ccccc5c4cc3c3ccc4sc5ccc(-c6cccc(N(c7ccccc7)c7cccc(-c8ccc(-c9ccccc9)cc8)c7)c6)cc5c4c32)c1 ; COc1ccc(-n2c3cc4c5ccccc5c5ccccc5c4cc3c3ccc4oc5c(-c6cccc(N(c7ccccc7)c7ccc(-c8ccccc8)cc7)c6)cccc5c4c32)cc1 ; CC1(C)c2ccc(-c3cccc(N(c4ccccc4)c4cccc(-c5ccc(-c6ccccc6)cc5)c4)c3)cc2-c2c1ccc1c2oc2cc3c4ccccc4c4ccccc4c3cc21]

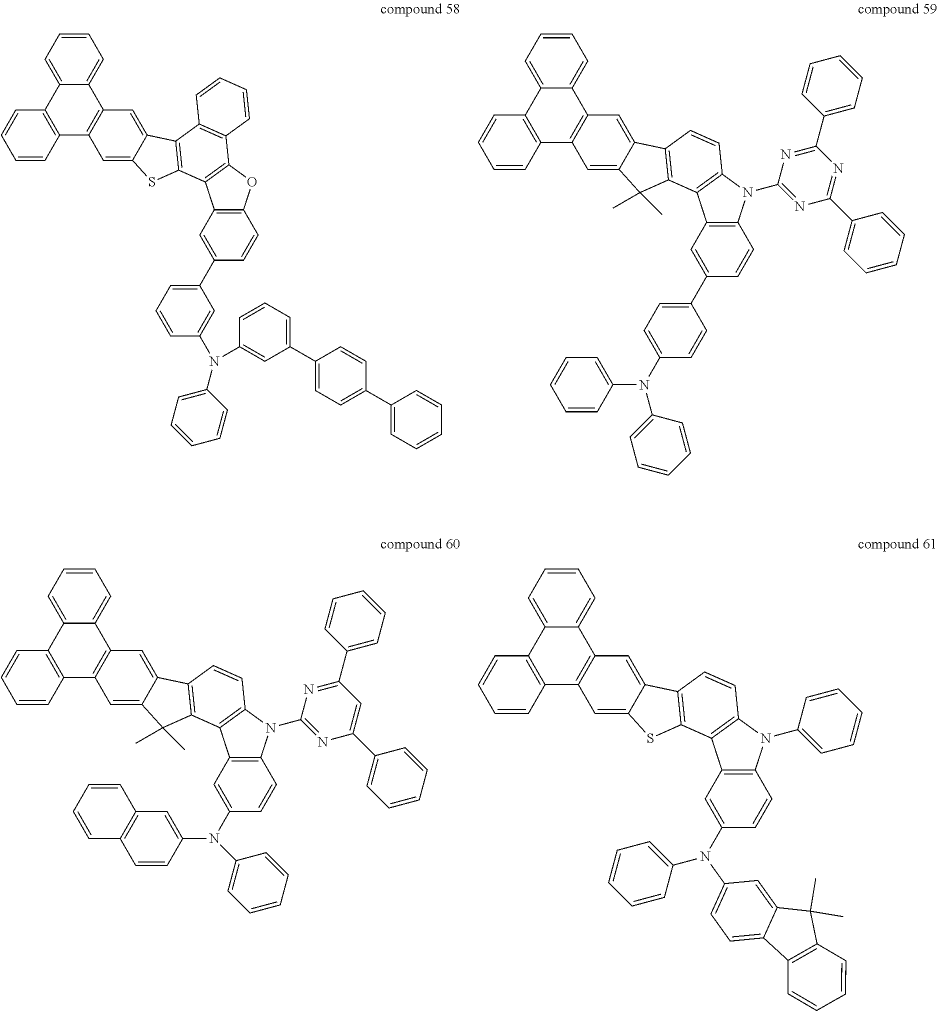 [SMILES: c1ccc(-c2ccc(-c3cccc(N(c4ccccc4)c4cccc(-c5ccc6oc7c8ccccc8c8c9cc%10c%11ccccc%11c%11ccccc%11c%10cc9sc8c7c6c5)c4)c3)cc2)cc1 ; CC1(C)c2cc3c4ccccc4c4ccccc4c3cc2-c2ccc3c(c21)c1cc(-c2ccc(N(c4ccccc4)c4ccccc4)cc2)ccc1n3-c1nc(-c2ccccc2)nc(-c2ccccc2)n1 ; CC1(C)c2cc3c4ccccc4c4ccccc4c3cc2-c2ccc3c(c21)c1cc(N(c2ccccc2)c2ccc4ccccc4c2)ccc1n3-c1nc(-c2ccccc2)cc(-c2ccccc2)n1 ; CC1(C)c2ccccc2-c2ccc(N(c3ccccc3)c3ccc4c(c3)c3c5sc6cc7c8ccccc8c8ccccc8c7cc6c5ccc3n4-c3ccccc3)cc21]

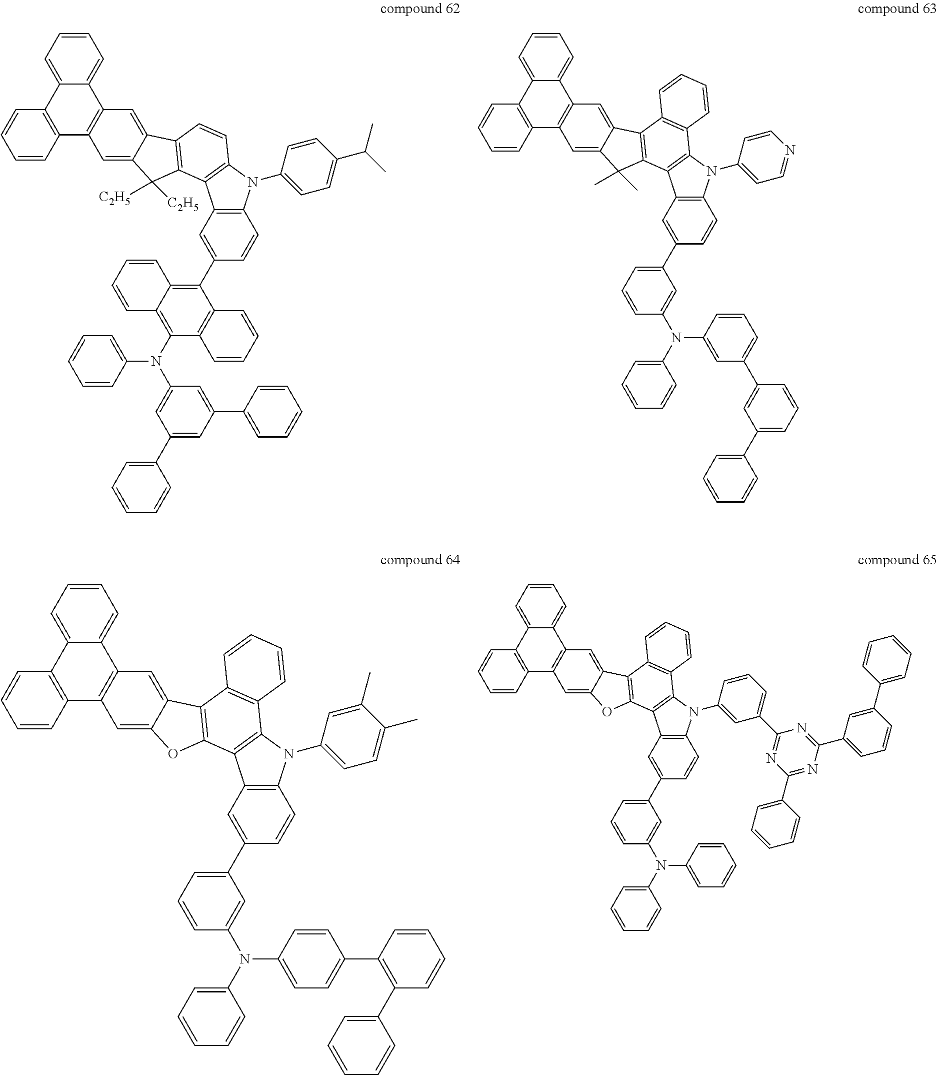

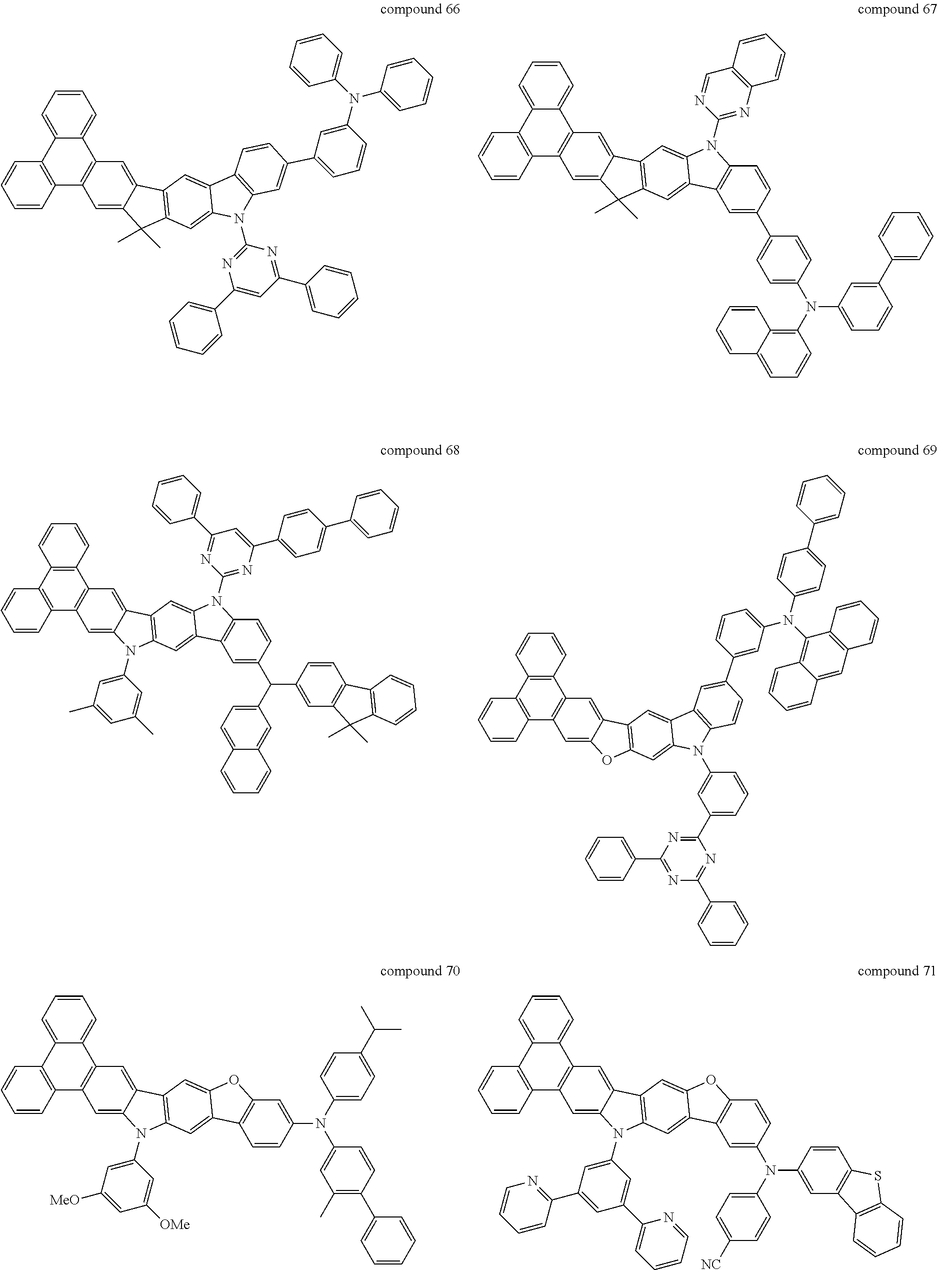

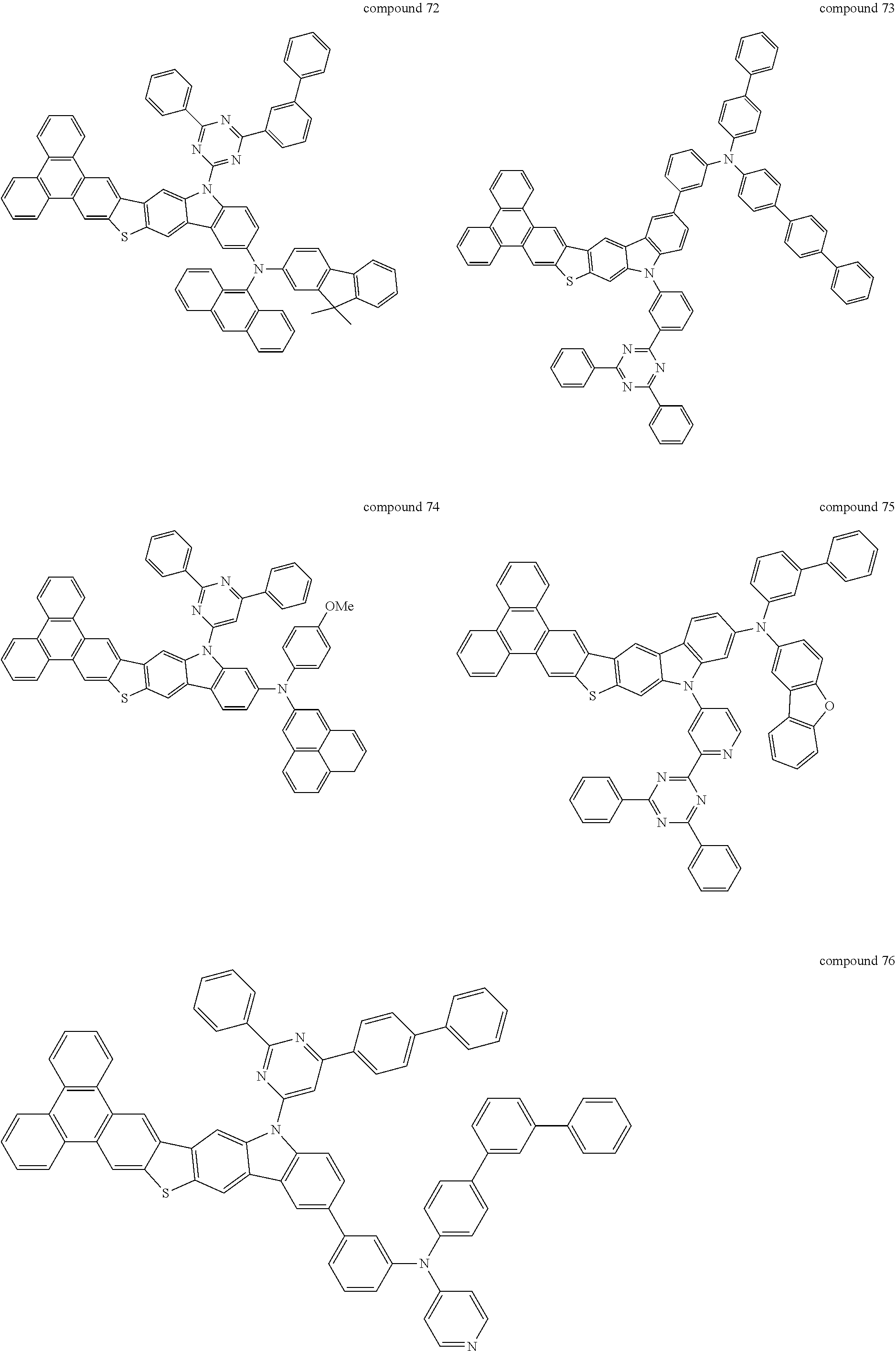

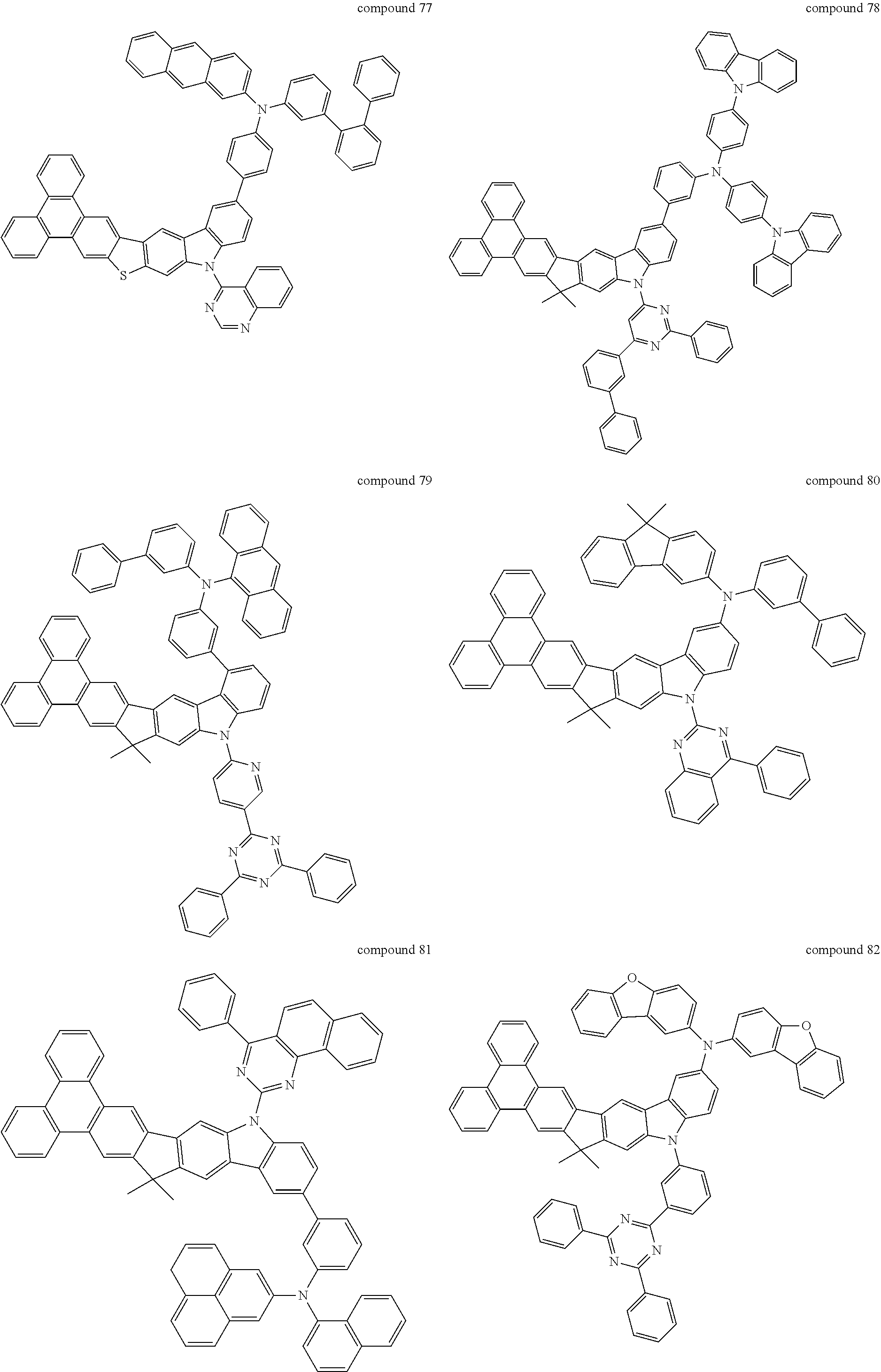

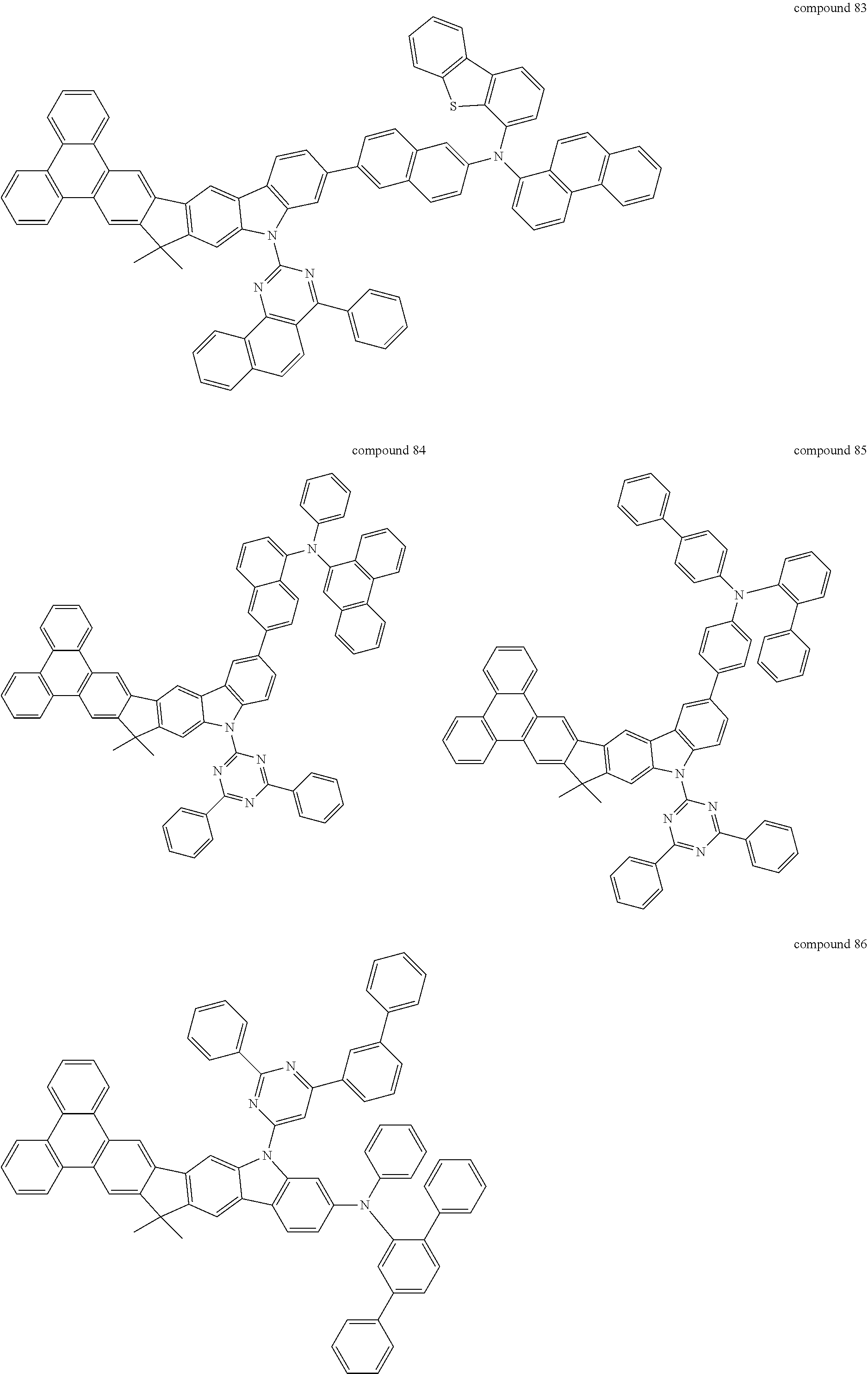

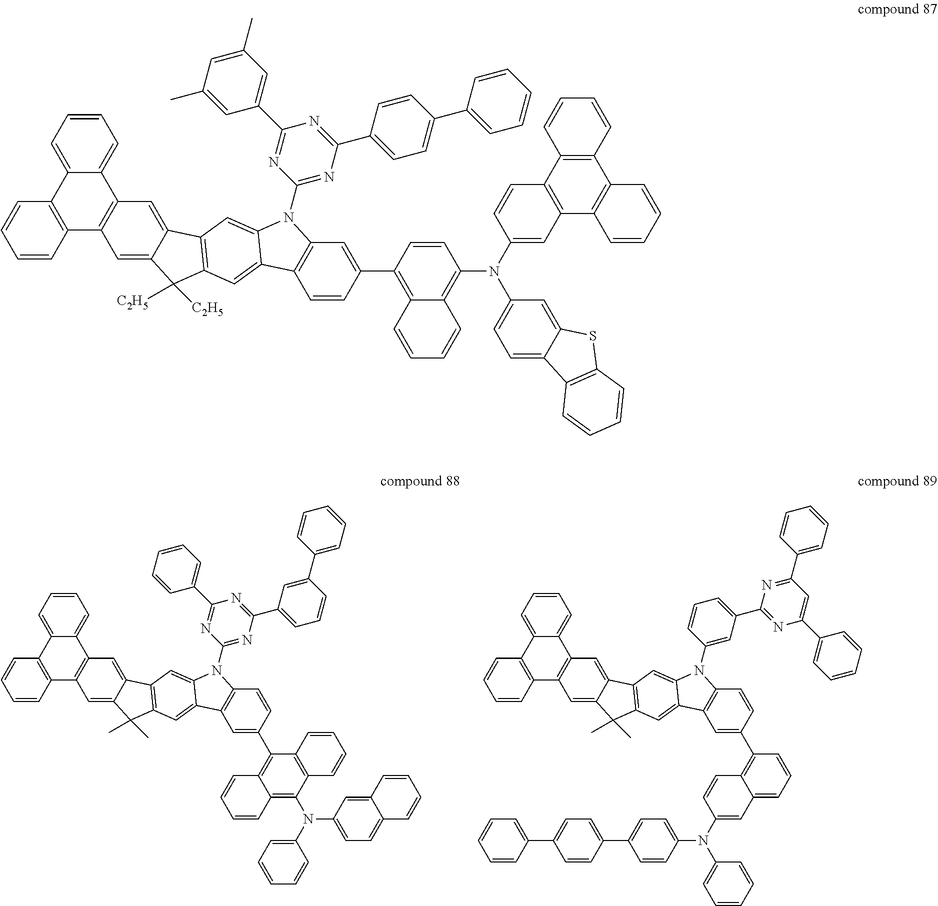 [SMILES: CCC1(CC)c2cc3c4ccccc4c4ccccc4c3cc2-c2cc3c(cc21)c1ccc(-c2ccc(N(c4ccc5c(c4)sc4ccccc45)c4ccc5c6ccccc6c6ccccc6c5c4)c4ccccc24)cc1n3-c1nc(-c2ccc(-c3ccccc3)cc2)nc(-c2cc(C)cc(C)c2)n1 ; CC1(C)c2cc3c4ccccc4c4ccccc4c3cc2-c2cc3c(cc21)c1cc(-c2c4ccccc4c(N(c4ccccc4)c4ccc5ccccc5c4)c4ccccc24)ccc1n3-c1nc(-c2ccccc2)nc(-c2cccc(-c3ccccc3)c2)n1 ; CC1(C)c2cc3c4ccccc4c4ccccc4c3cc2-c2cc3c(cc21)c1cc(-c2cccc4cc(N(c5ccccc5)c5ccc(-c6ccc(-c7ccccc7)cc6)cc5)ccc24)ccc1n3-c1cccc(-c2nc(-c3ccccc3)cc(-c3ccccc3)n2)c1]

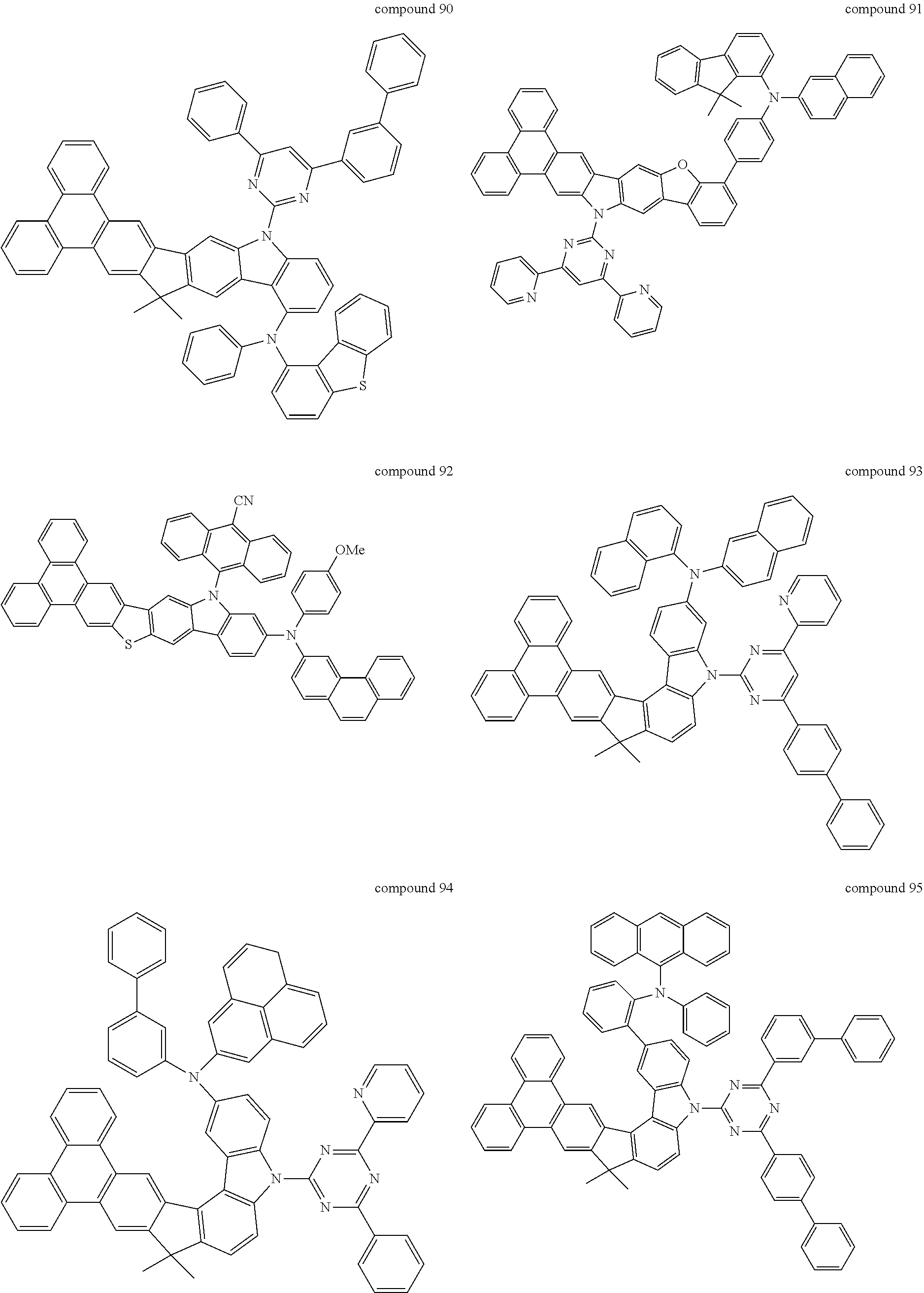

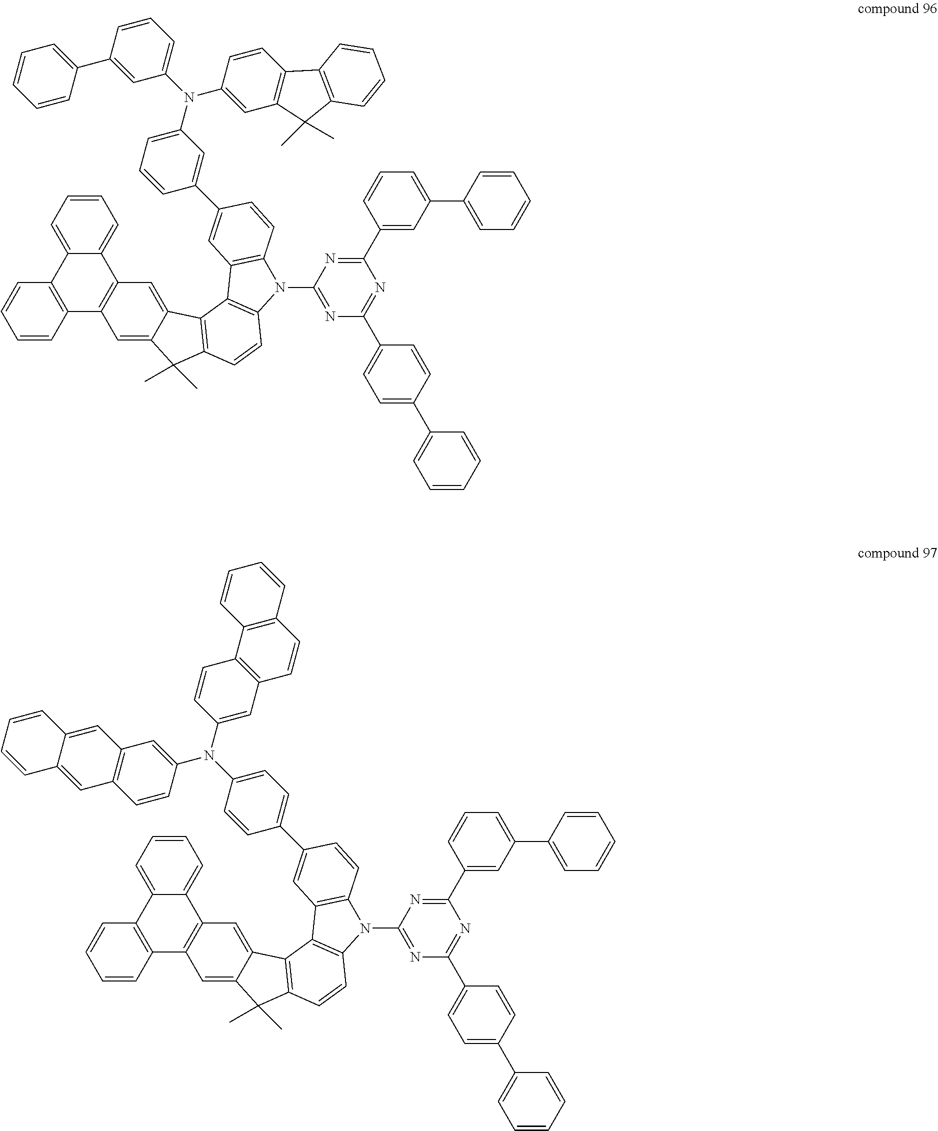 [SMILES: CC1(C)c2ccccc2-c2ccc(N(c3cccc(-c4ccccc4)c3)c3cccc(-c4ccc5c(c4)c4c6c(ccc4n5-c4nc(-c5ccc(-c7ccccc7)cc5)nc(-c5cccc(-c7ccccc7)c5)n4)C(C)(C)c4cc5c7ccccc7c7ccccc7c5cc4-6)c3)cc21 ; CC1(C)c2cc3c4ccccc4c4ccccc4c3cc2-c2c1ccc1c2c2cc(-c3ccc(N(c4ccc5cc6ccccc6cc5c4)c4ccc5c(ccc6ccccc65)c4)cc3)ccc2n1-c1nc(-c2ccc(-c3ccccc3)cc2)nc(-c2cccc(-c3ccccc3)c2)n1]

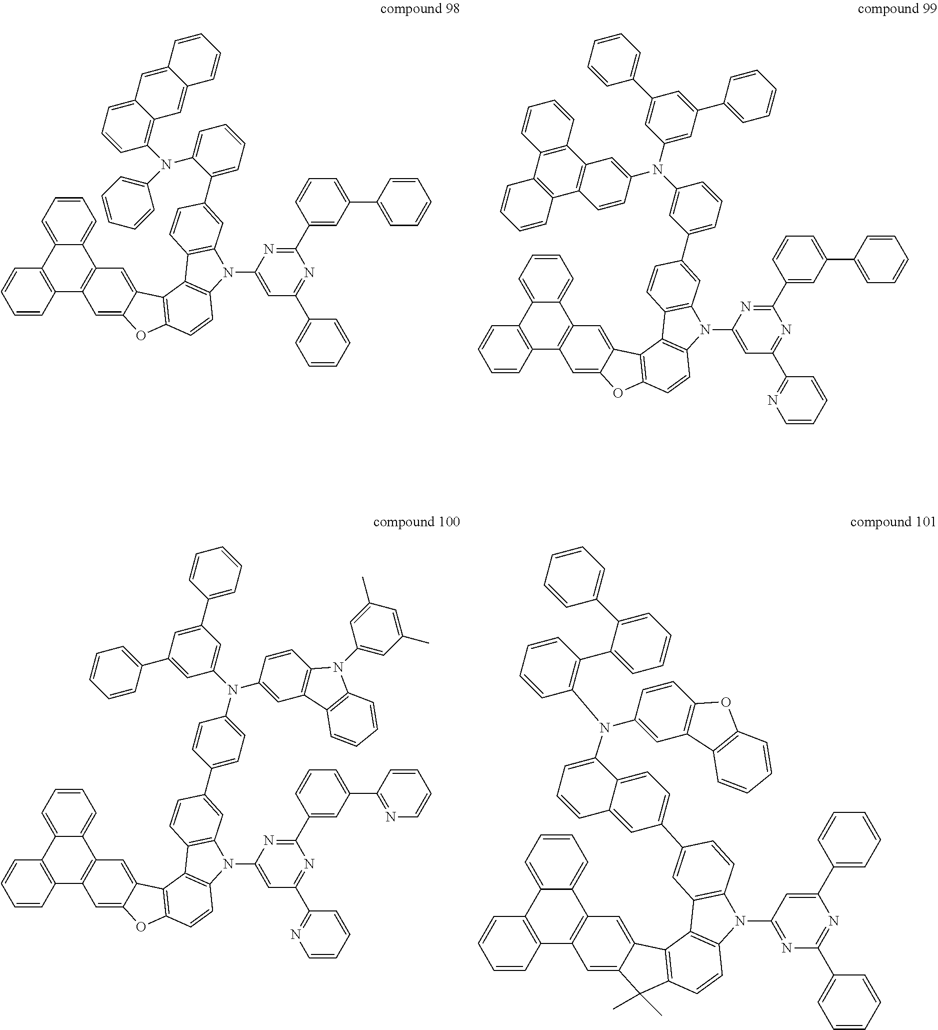

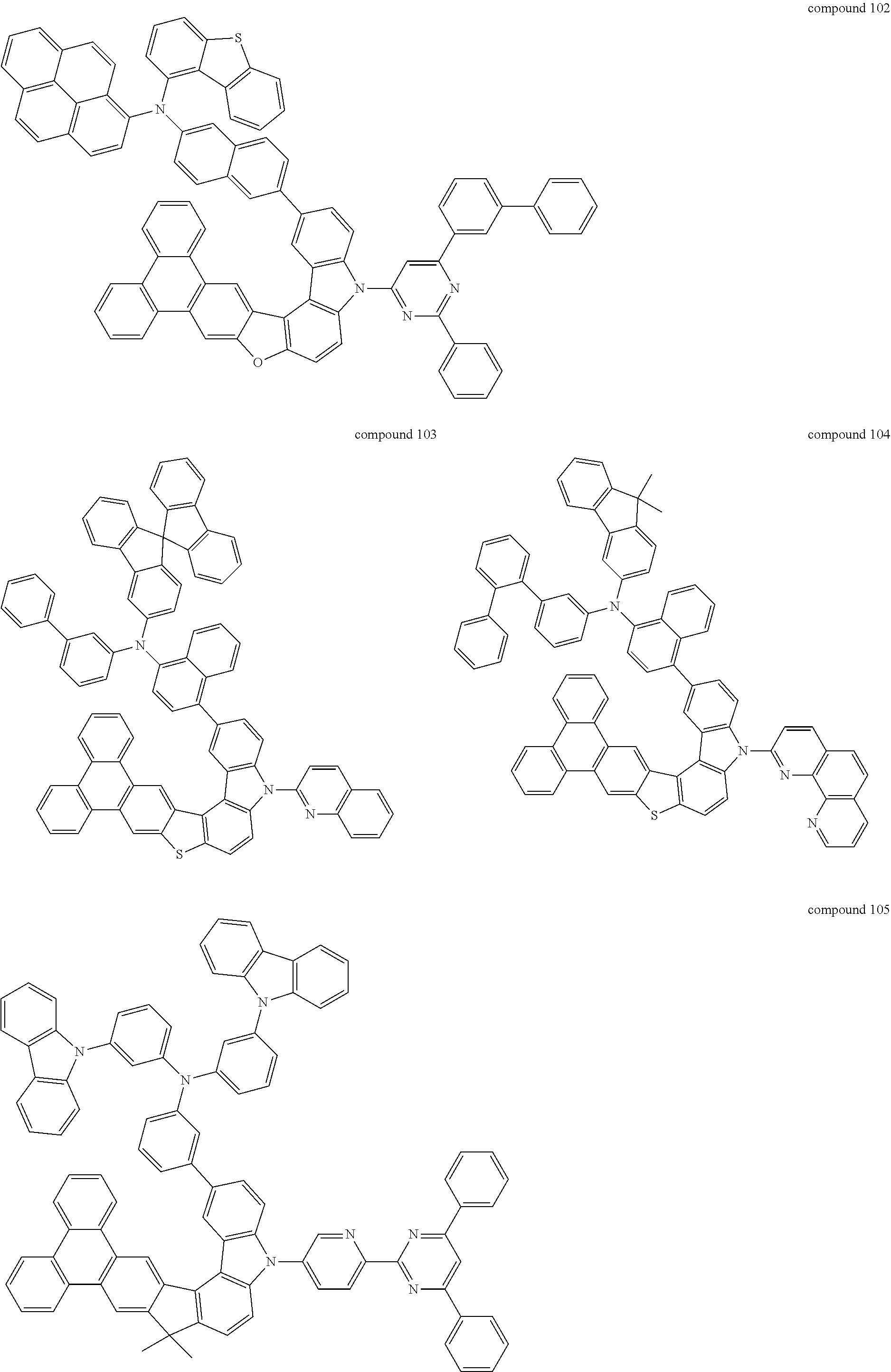

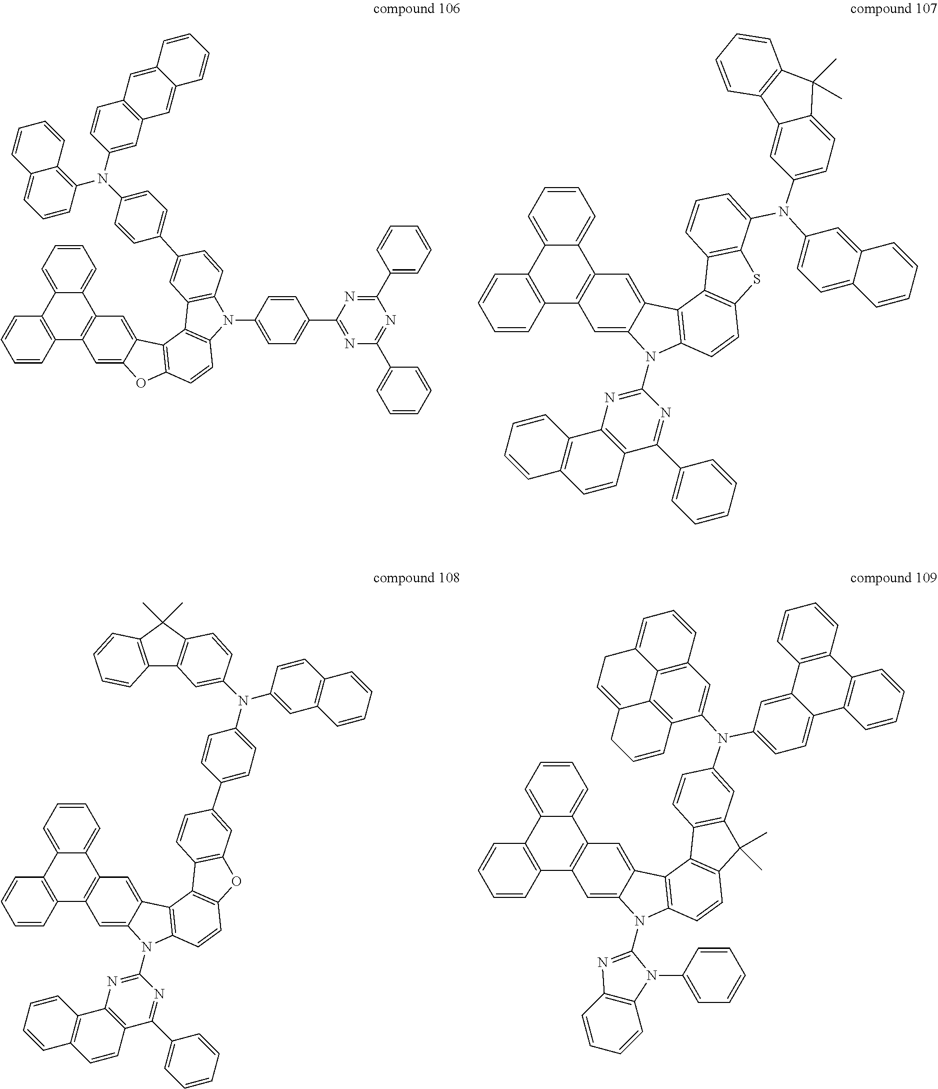

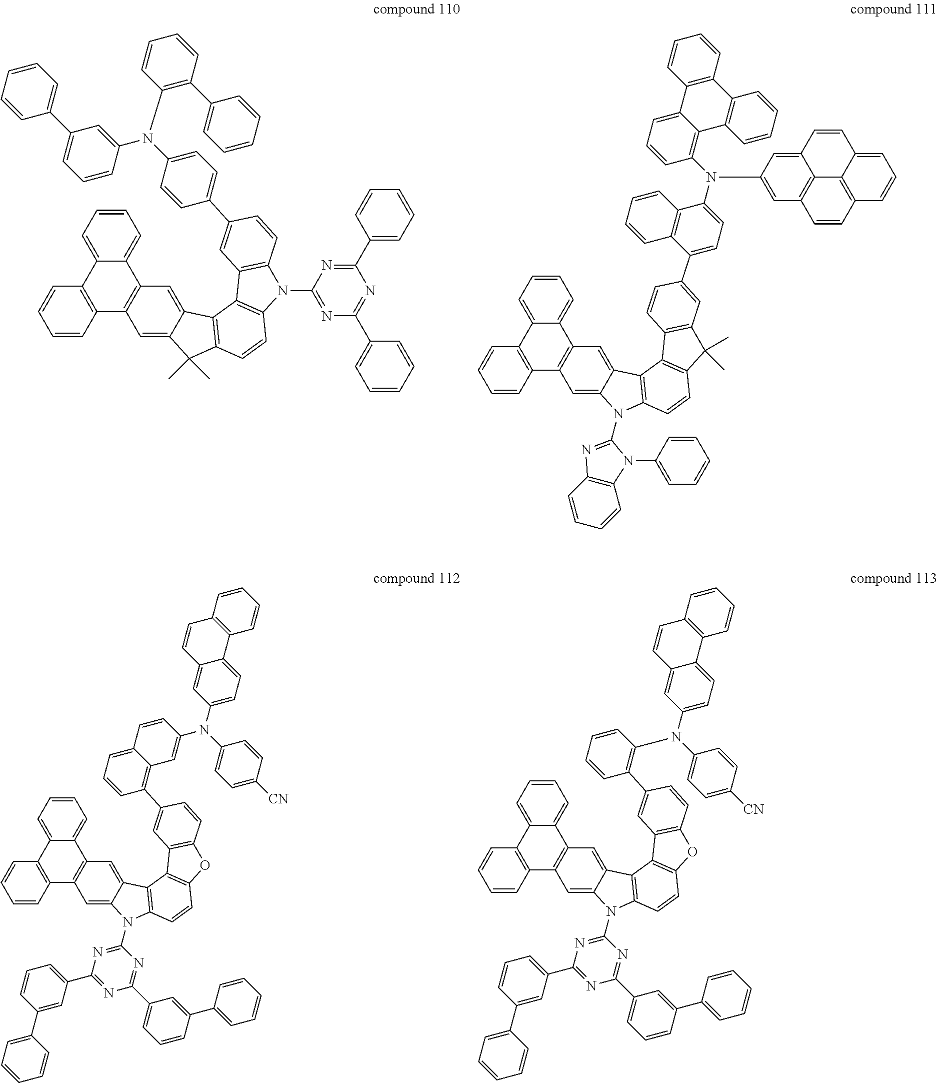

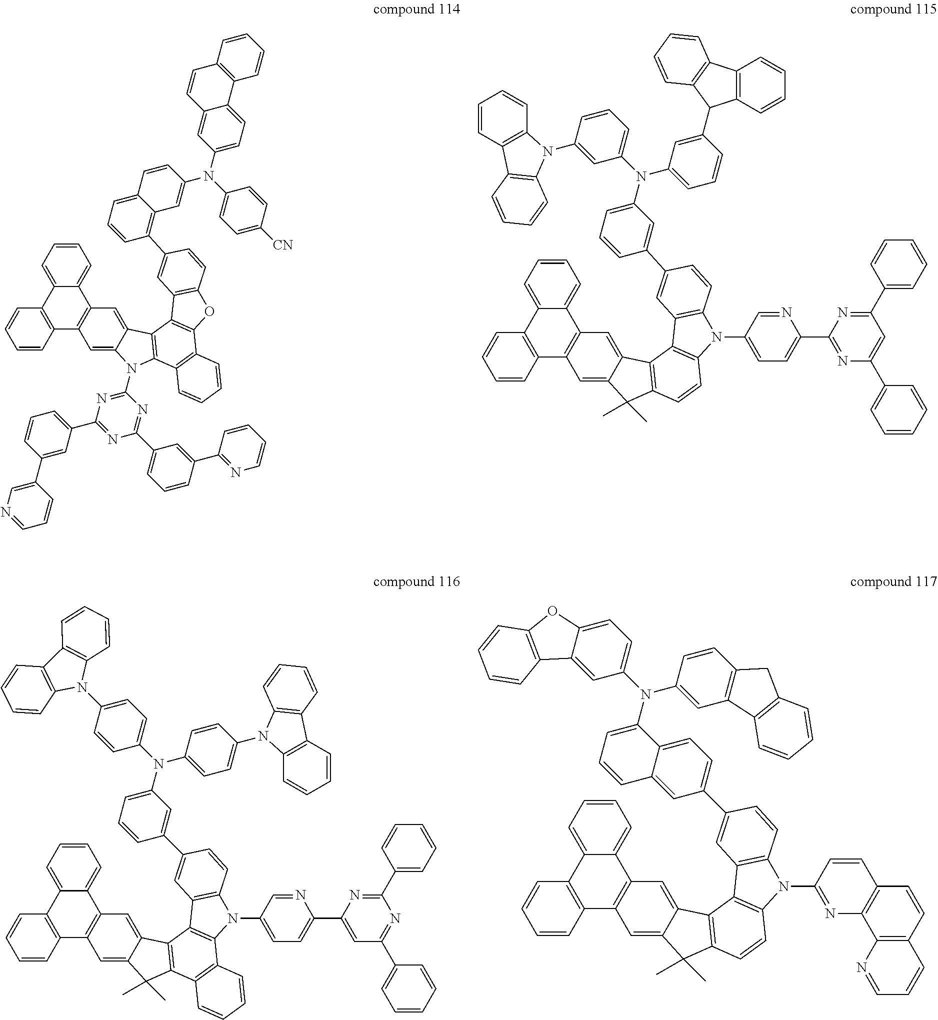

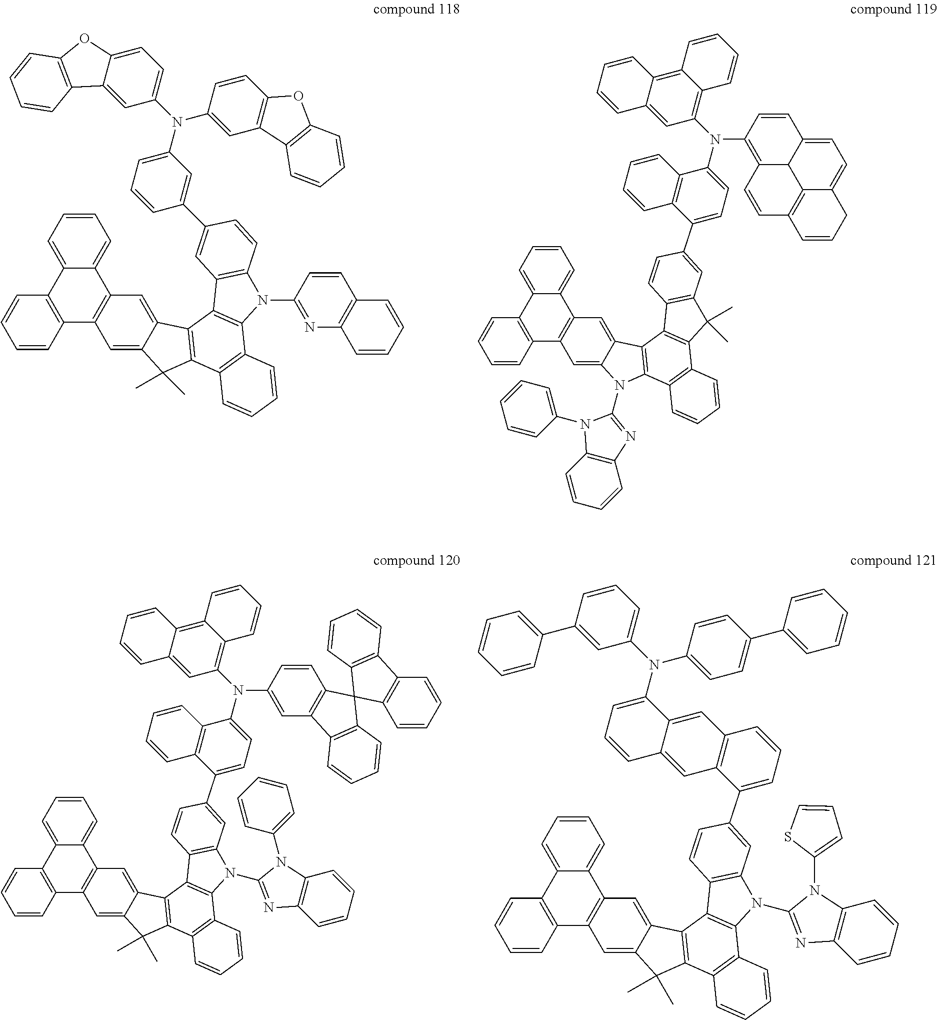

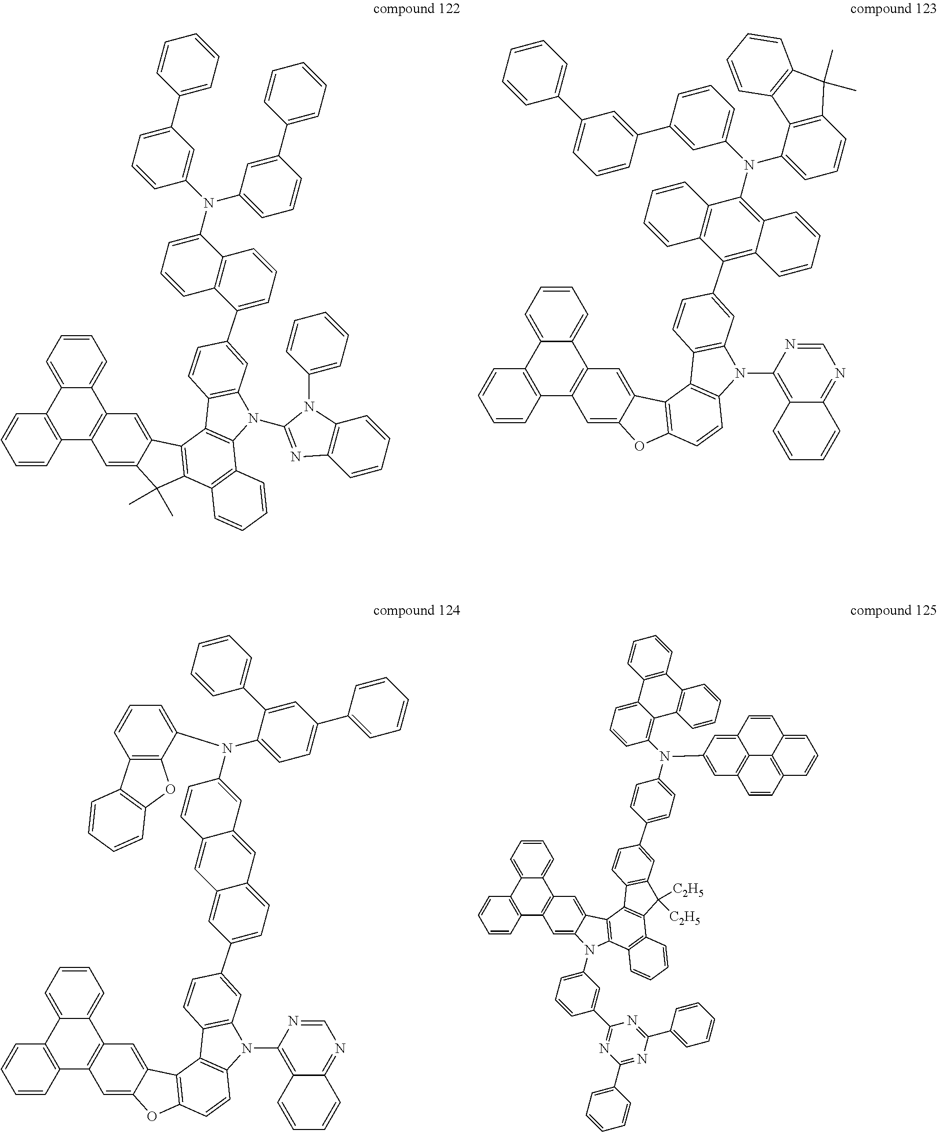

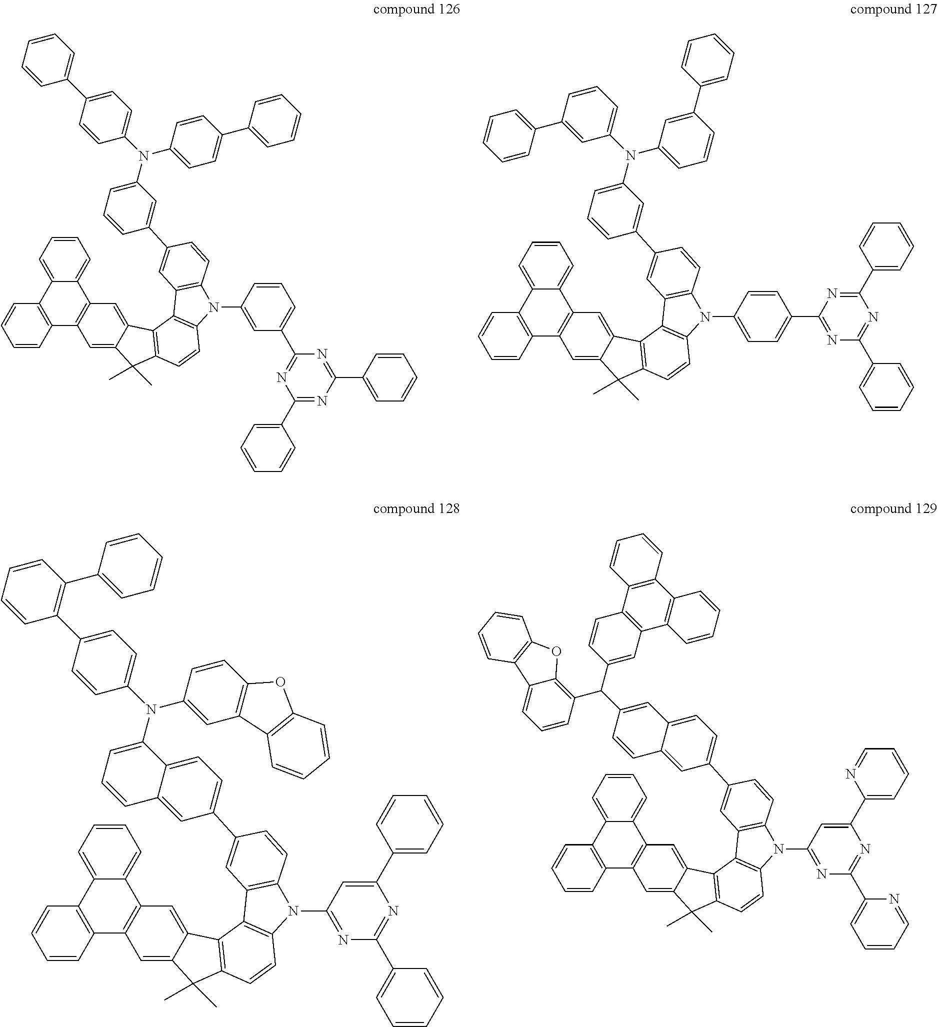

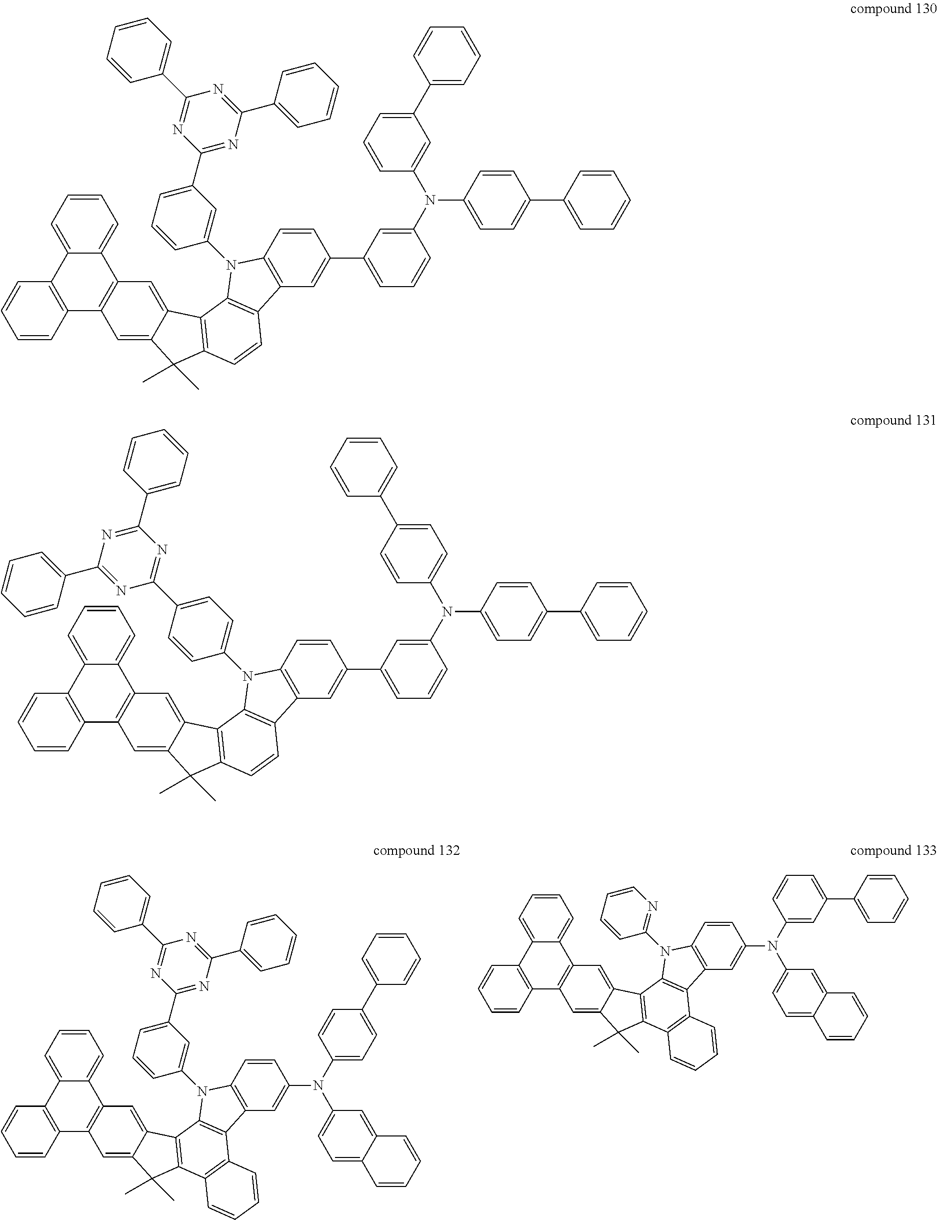

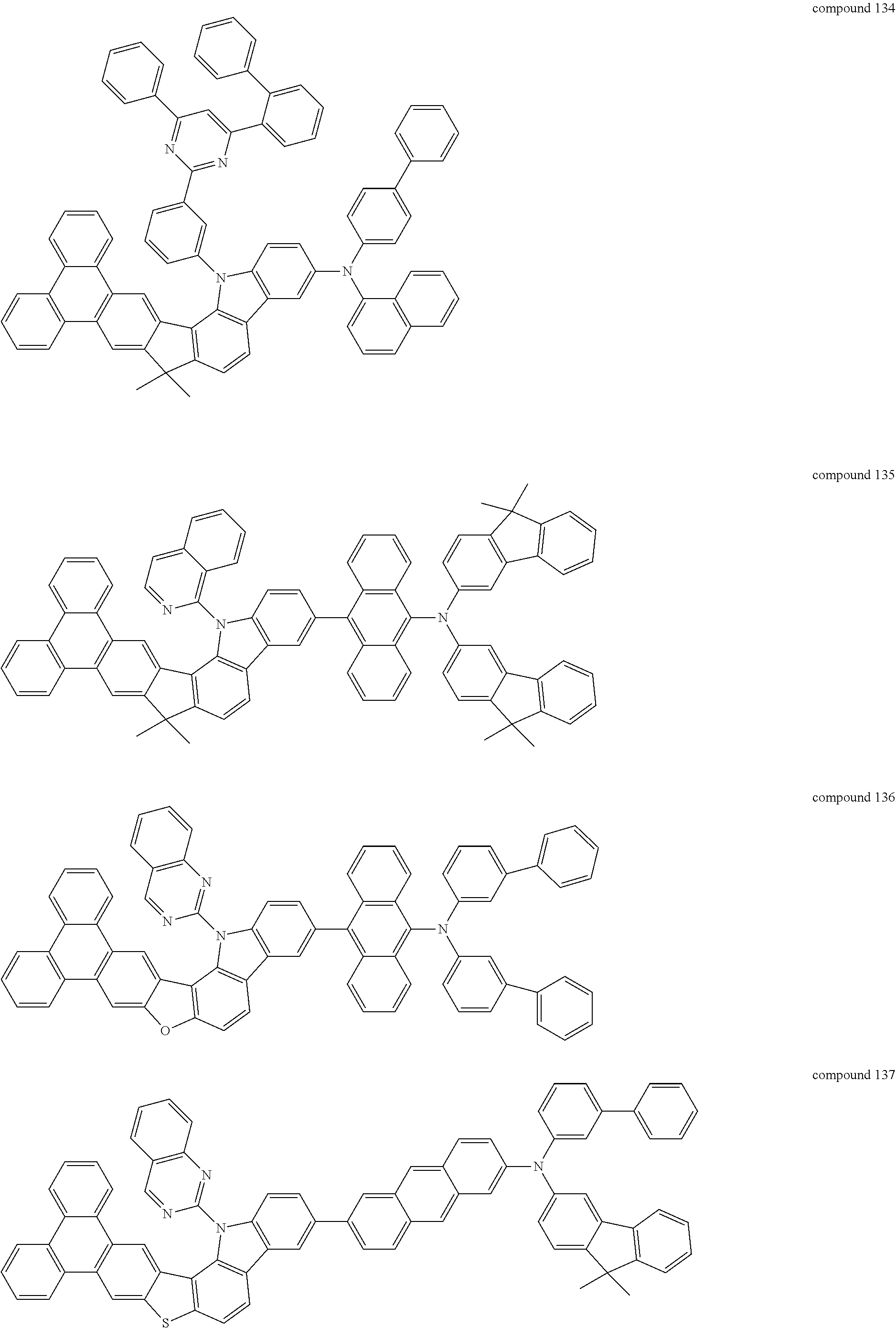

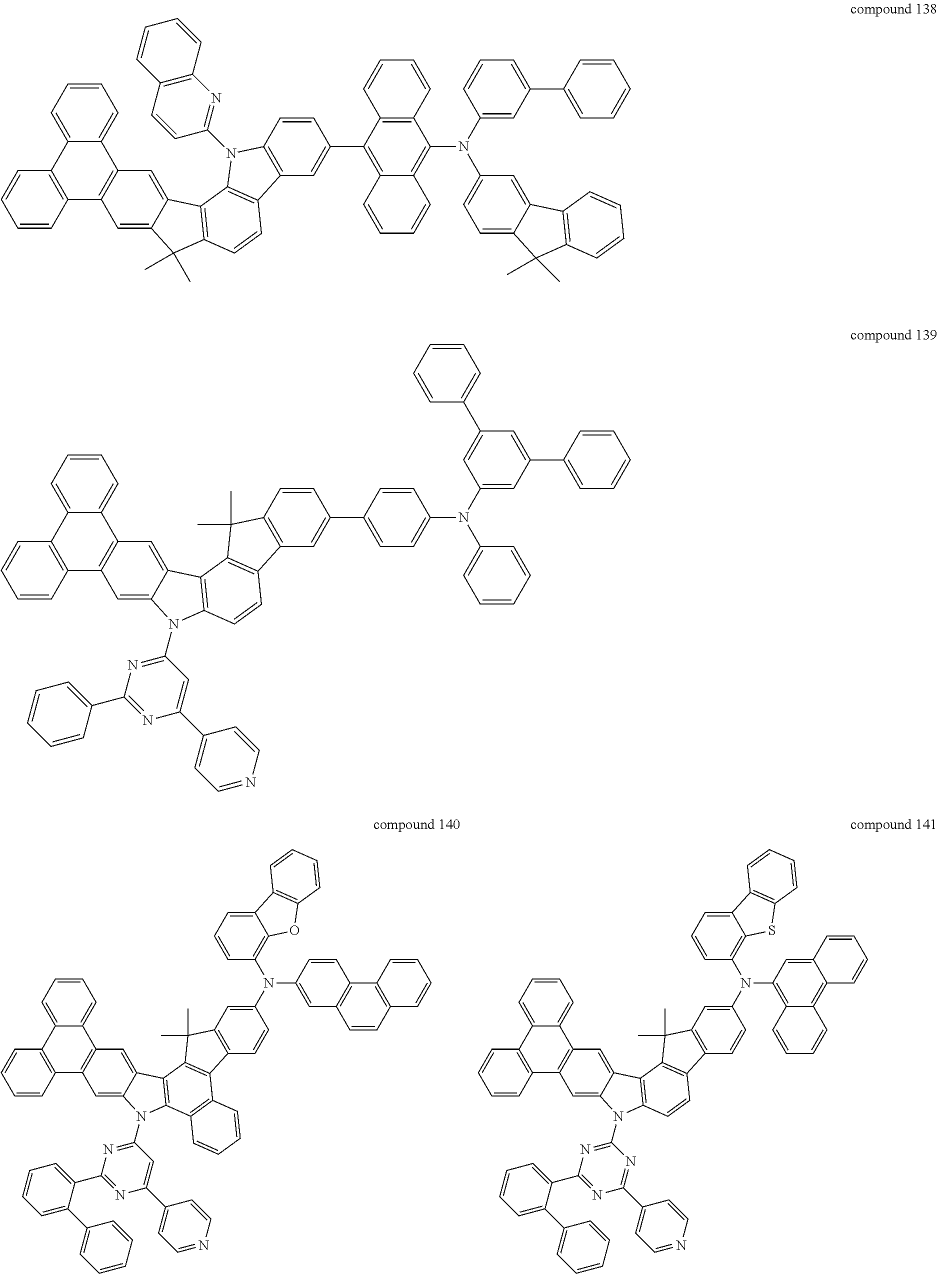

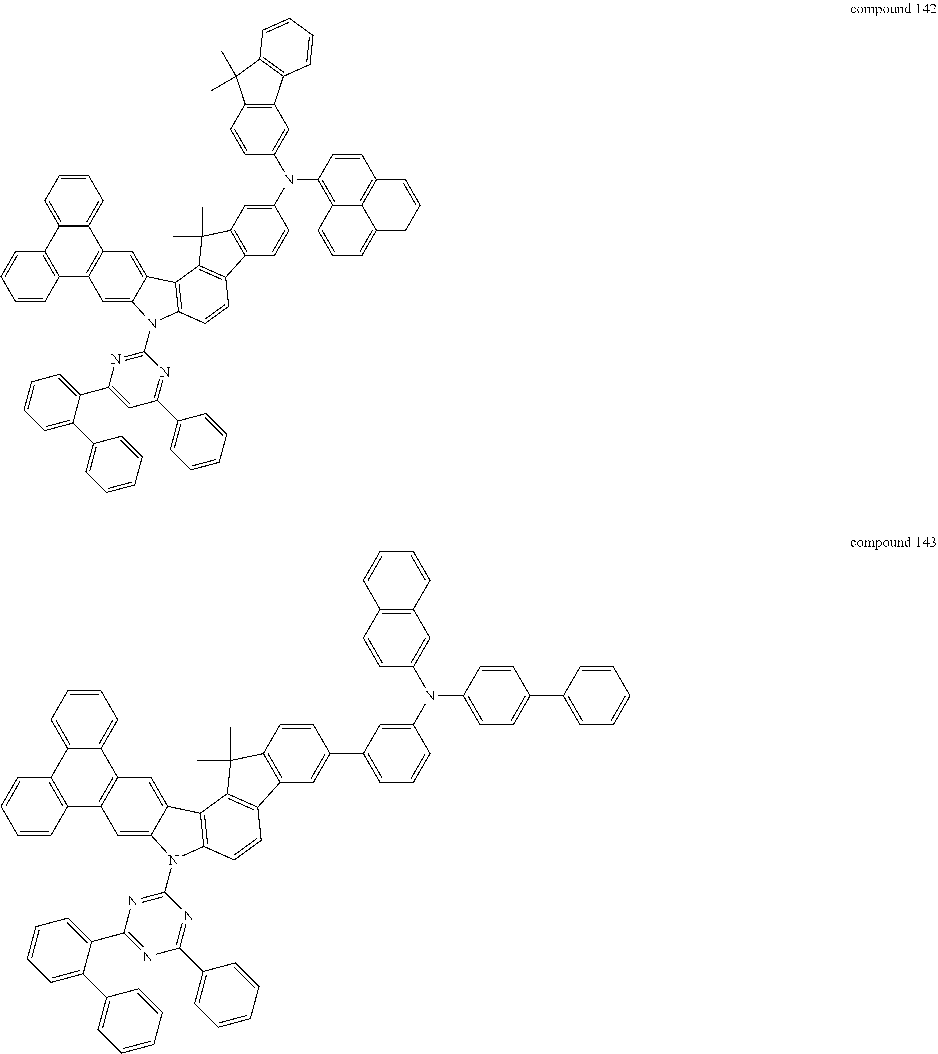 [SMILES: CC1(C)c2ccccc2-c2cc(N(c3ccc4c(c3)C(C)(C)c3c-4ccc4c3c3cc5c6ccccc6c6ccccc6c5cc3n4-c3nc(-c4ccccc4)cc(-c4ccccc4-c4ccccc4)n3)c3ccc4c5c(cccc35)CC=C4)ccc21 ; CC1(C)c2ccc(-c3cccc(N(c4ccc(-c5ccccc5)cc4)c4ccc5ccccc5c4)c3)cc2-c2ccc3c(c21)c1cc2c4ccccc4c4ccccc4c2cc1n3-c1nc(-c2ccccc2)nc(-c2ccccc2-c2ccccc2)n1]

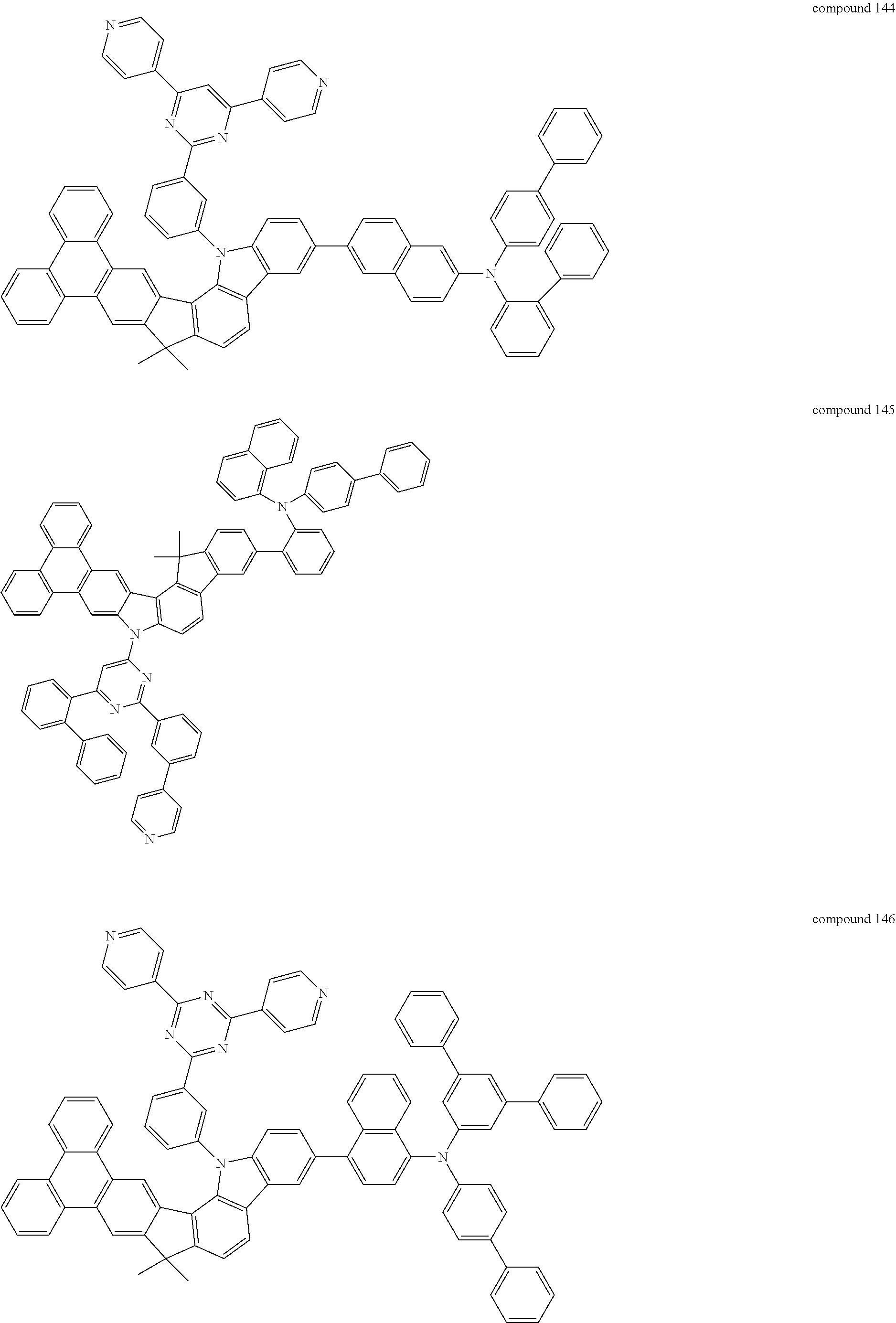 [SMILES: CC1(C)c2cc3c4ccccc4c4ccccc4c3cc2-c2c1ccc1c3cc(-c4ccc5cc(N(c6ccc(-c7ccccc7)cc6)c6ccccc6-c6ccccc6)ccc5c4)ccc3n(-c3cccc(-c4nc(-c5ccncc5)cc(-c5ccncc5)n4)c3)c21 ; CC1(C)c2ccc(-c3ccccc3N(c3ccc(-c4ccccc4)cc3)c3cccc4ccccc34)cc2-c2ccc3c(c21)c1cc2c4ccccc4c4ccccc4c2cc1n3-c1cc(-c2ccccc2-c2ccccc2)nc(-c2cccc(-c3ccncc3)c2)n1 ; CC1(C)c2cc3c4ccccc4c4ccccc4c3cc2-c2c1ccc1c3cc(-c4ccc(N(c5ccc(-c6ccccc6)cc5)c5cc(-c6ccccc6)cc(-c6ccccc6)c5)c5ccccc45)ccc3n(-c3cccc(-c4nc(-c5ccncc5)nc(-c5ccncc5)n4)c3)c21]

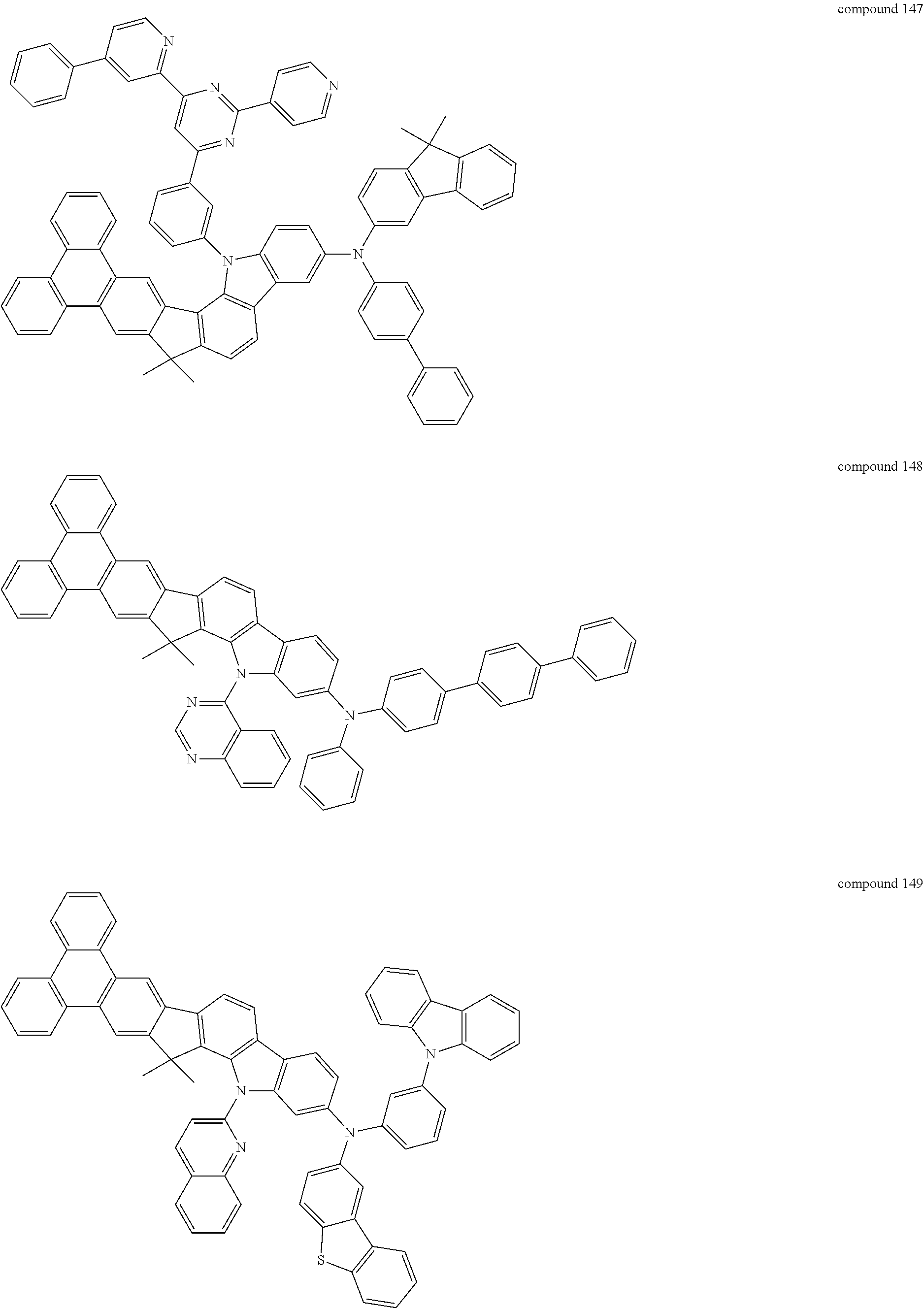 [SMILES: CC1(C)c2ccccc2-c2cc(N(c3ccc(-c4ccccc4)cc3)c3ccc4c(c3)c3ccc5c(c3n4-c3cccc(-c4cc(-c6cc(-c7ccccc7)ccn6)nc(-c6ccncc6)n4)c3)-c3cc4c6ccccc6c6ccccc6c4cc3C5(C)C)ccc21 ; CC1(C)c2cc3c4ccccc4c4ccccc4c3cc2-c2ccc3c4ccc(N(c5ccccc5)c5ccc(-c6ccc(-c7ccccc7)cc6)cc5)cc4n(-c4ncnc5ccccc45)c3c21 ; CC1(C)c2cc3c4ccccc4c4ccccc4c3cc2-c2ccc3c4ccc(N(c5cccc(-n6c7ccccc7c7ccccc76)c5)c5ccc6sc7ccccc7c6c5)cc4n(-c4ccc5ccccc5n4)c3c21]

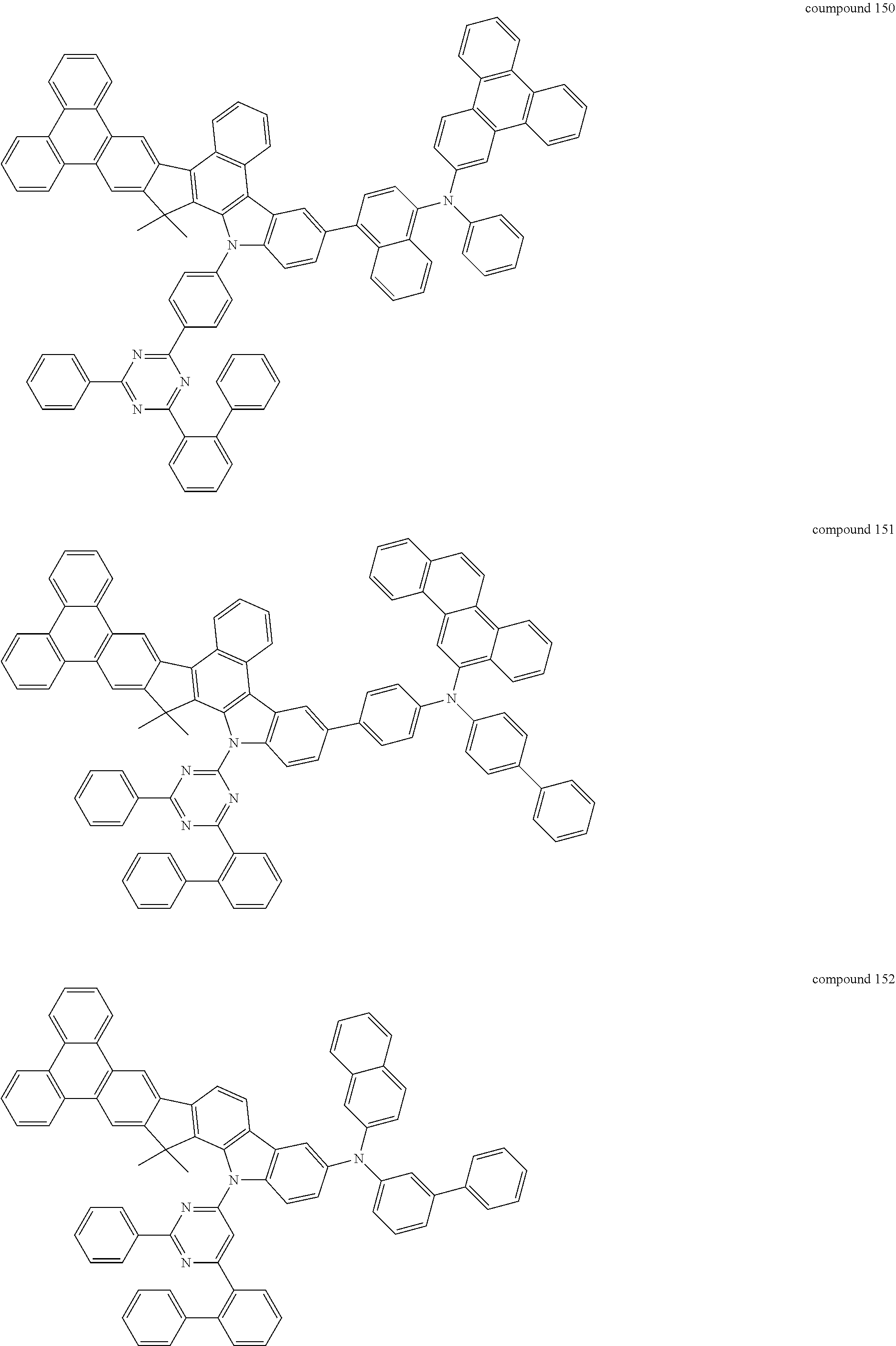 [SMILES: CC1(C)c2cc3c4ccccc4c4ccccc4c3cc2-c2c1c1c(c3ccccc23)c2cc(-c3ccc(N(c4ccccc4)c4ccc5c6ccccc6c6ccccc6c5c4)c4ccccc34)ccc2n1-c1ccc(-c2nc(-c3ccccc3)nc(-c3ccccc3-c3ccccc3)n2)cc1 ; CC1(C)c2cc3c4ccccc4c4ccccc4c3cc2-c2c1c1c(c3ccccc23)c2cc(-c3ccc(N(c4ccc(-c5ccccc5)cc4)c4cc5c6ccccc6ccc5c5ccccc45)cc3)ccc2n1-c1nc(-c2ccccc2)nc(-c2ccccc2-c2ccccc2)n1 ; CC1(C)c2cc3c4ccccc4c4ccccc4c3cc2-c2ccc3c4cc(N(c5cccc(-c6ccccc6)c5)c5ccc6ccccc6c5)ccc4n(-c4cc(-c5ccccc5-c5ccccc5)nc(-c5ccccc5)n4)c3c21]

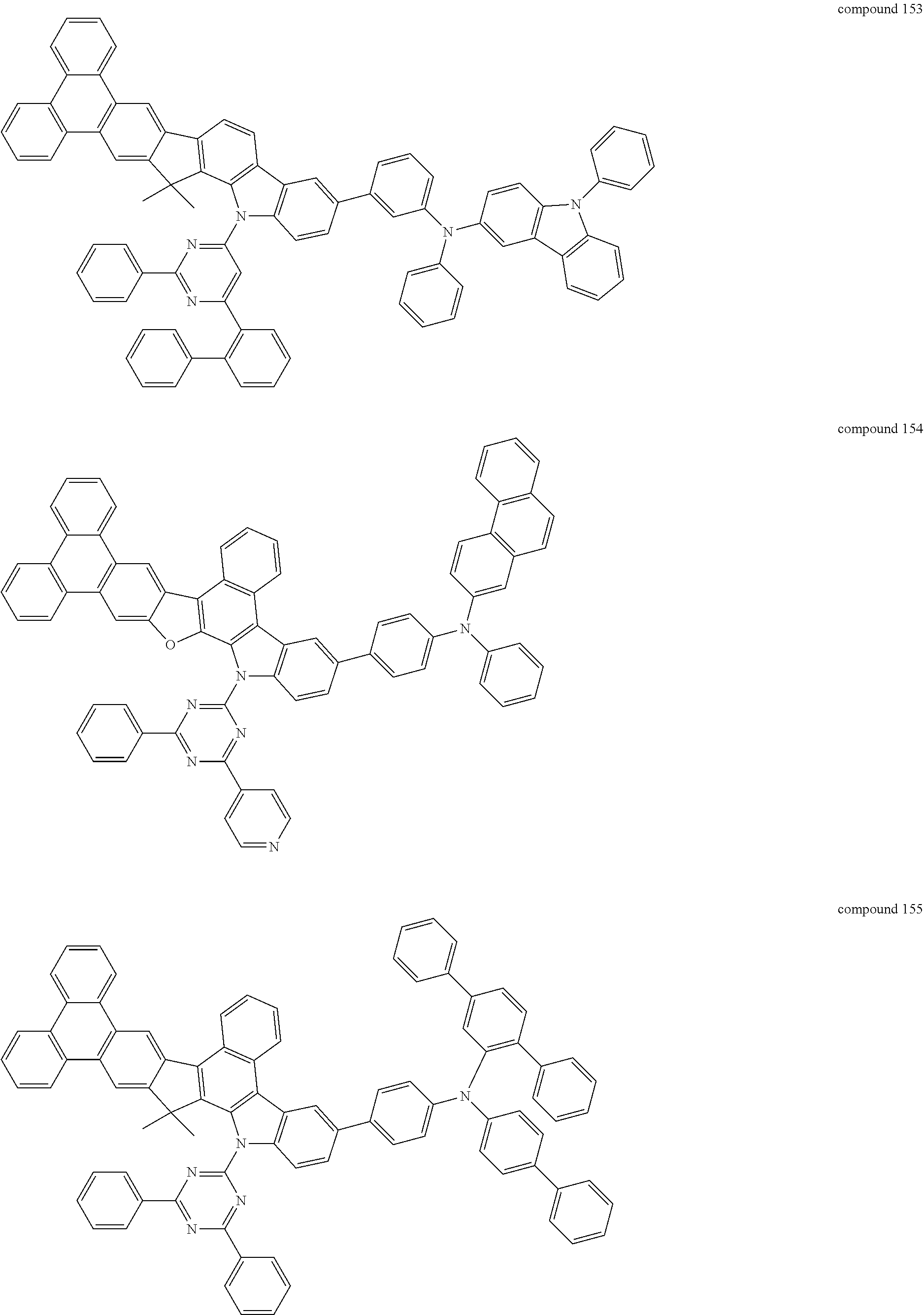 [SMILES: CC1(C)c2cc3c4ccccc4c4ccccc4c3cc2-c2ccc3c4cc(-c5cccc(N(c6ccccc6)c6ccc7c(c6)c6ccccc6n7-c6ccccc6)c5)ccc4n(-c4cc(-c5ccccc5-c5ccccc5)nc(-c5ccccc5)n4)c3c21 ; c1ccc(-c2nc(-c3ccncc3)nc(-n3c4ccc(-c5ccc(N(c6ccccc6)c6ccc7c(ccc8ccccc87)c6)cc5)cc4c4c5ccccc5c5c6cc7c8ccccc8c8ccccc8c7cc6oc5c43)n2)cc1 ; CC1(C)c2cc3c4ccccc4c4ccccc4c3cc2-c2c1c1c(c3ccccc23)c2cc(-c3ccc(N(c4ccc(-c5ccccc5)cc4)c4cc(-c5ccccc5)ccc4-c4ccccc4)cc3)ccc2n1-c1nc(-c2ccccc2)nc(-c2ccccc2)n1]

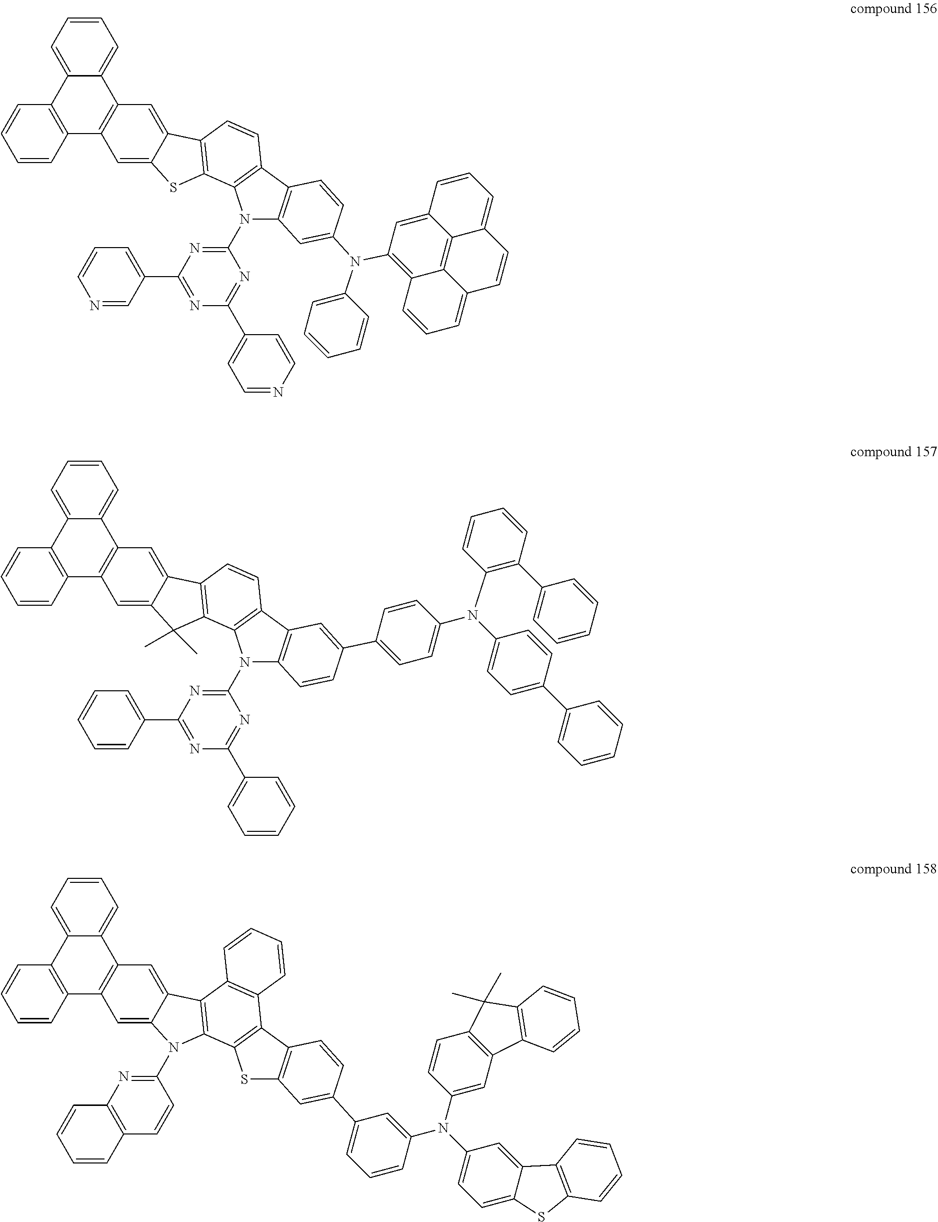 [SMILES: c1ccc(N(c2ccc3c4ccc5c6cc7c8ccccc8c8ccccc8c7cc6sc5c4n(-c4nc(-c5ccncc5)nc(-c5cccnc5)n4)c3c2)c2cc3cccc4ccc5cccc2c5c43)cc1 ; CC1(C)c2cc3c4ccccc4c4ccccc4c3cc2-c2ccc3c4cc(-c5ccc(N(c6ccc(-c7ccccc7)cc6)c6ccccc6-c6ccccc6)cc5)ccc4n(-c4nc(-c5ccccc5)nc(-c5ccccc5)n4)c3c21 ; CC1(C)c2ccccc2-c2cc(N(c3cccc(-c4ccc5c(c4)sc4c5c5ccccc5c5c6cc7c8ccccc8c8ccccc8c7cc6n(-c6ccc7ccccc7n6)c45)c3)c3ccc4sc5ccccc5c4c3)ccc21]

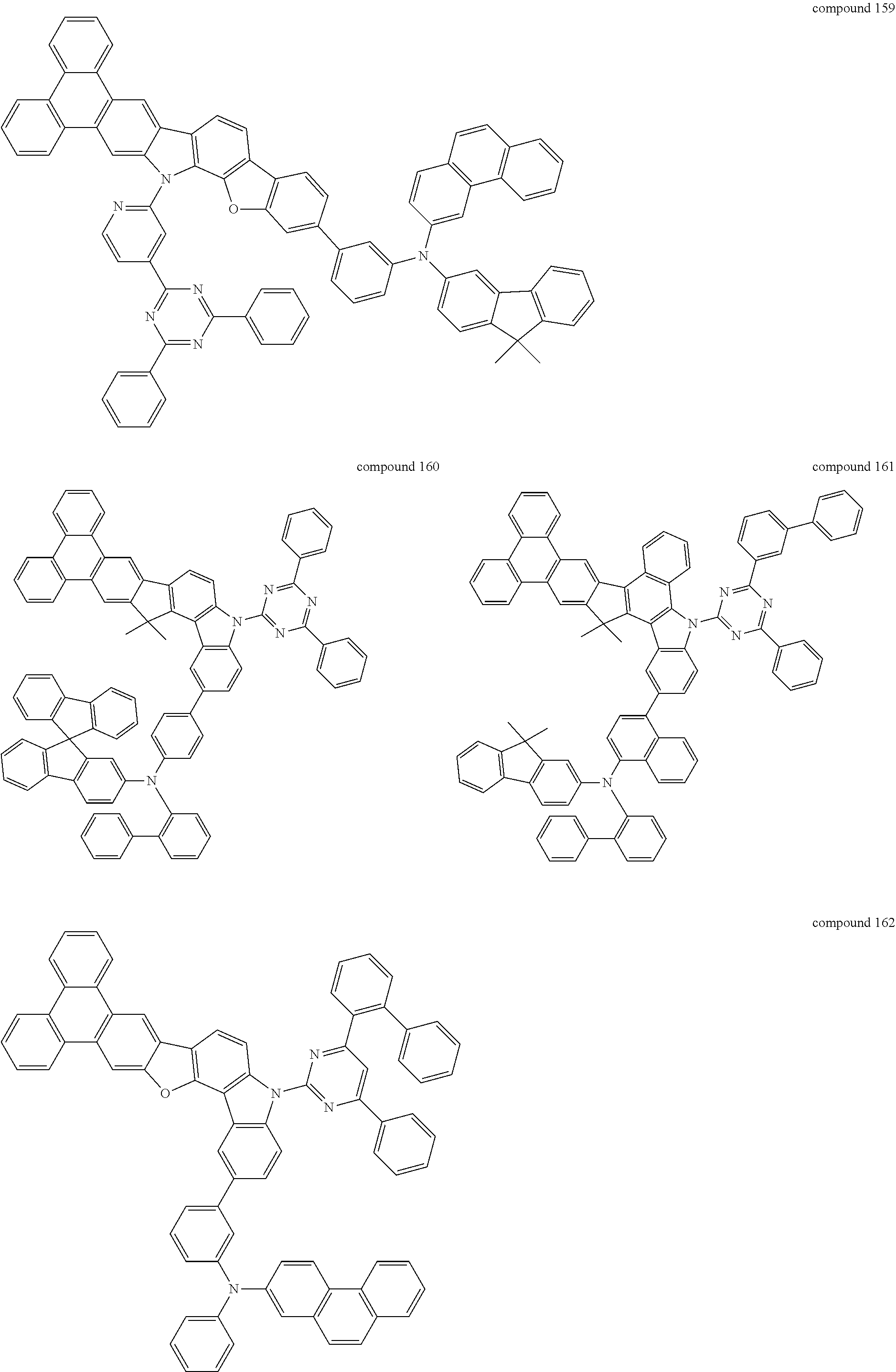

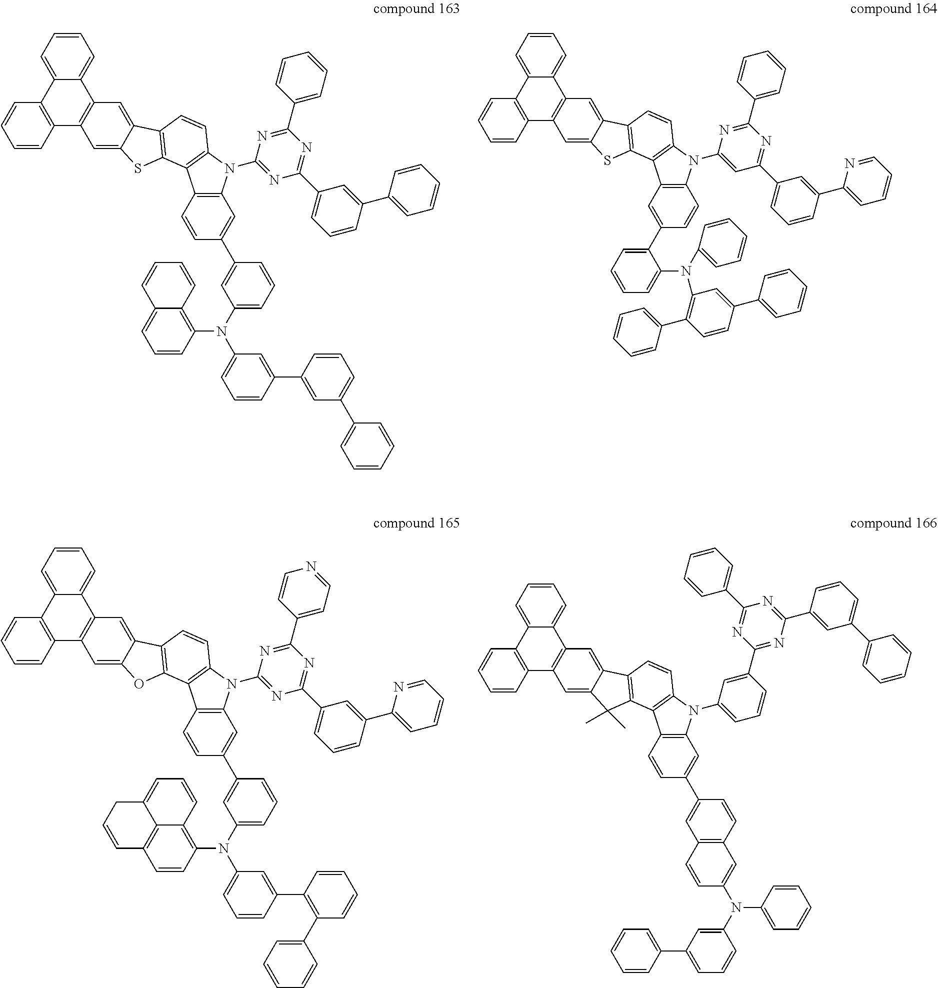

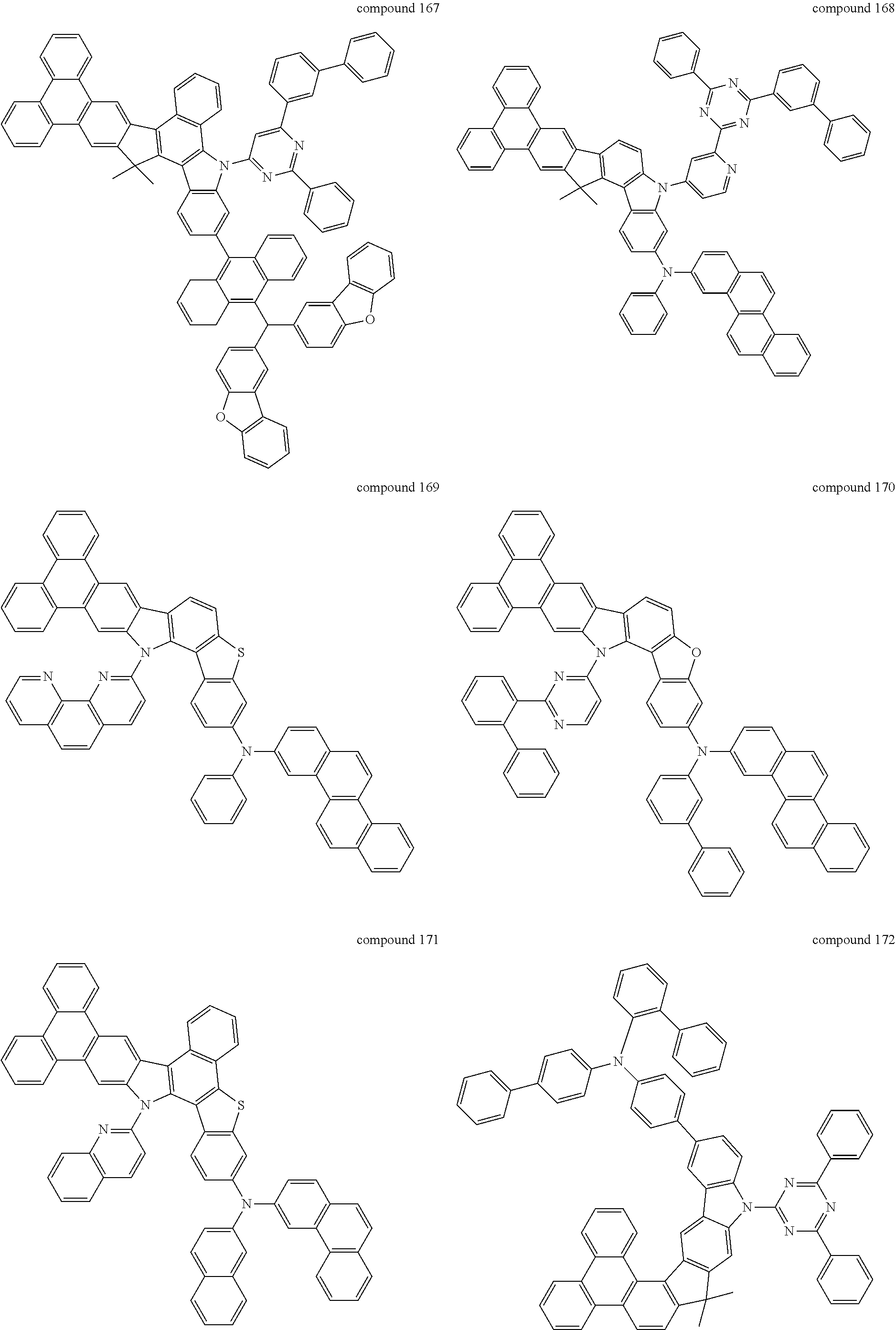

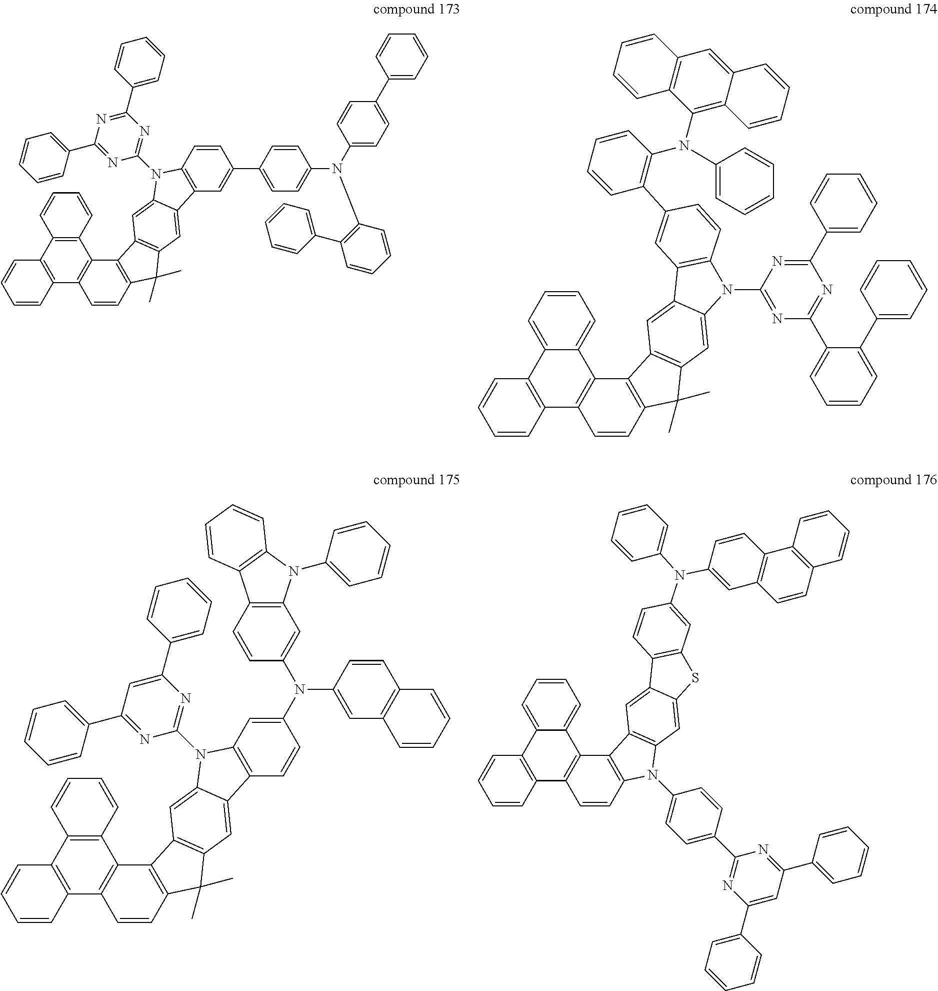

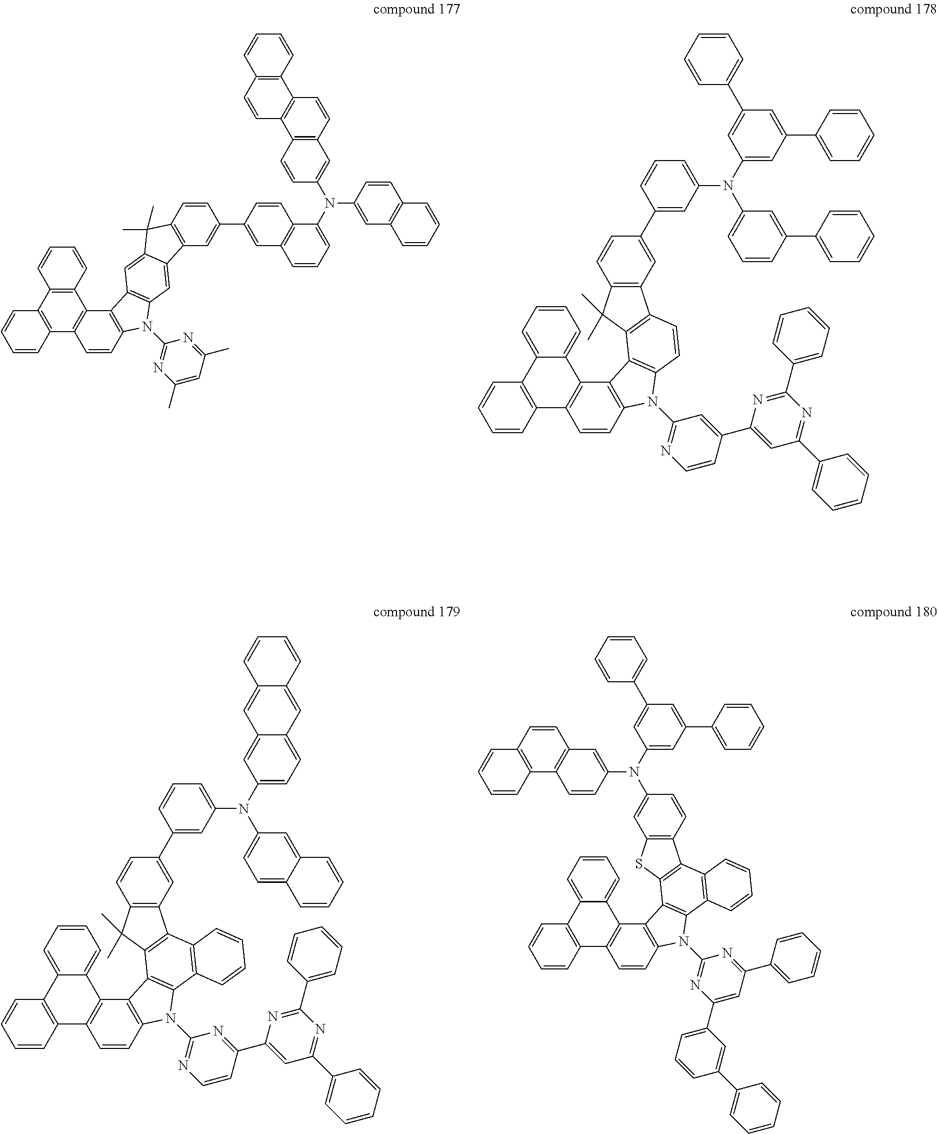

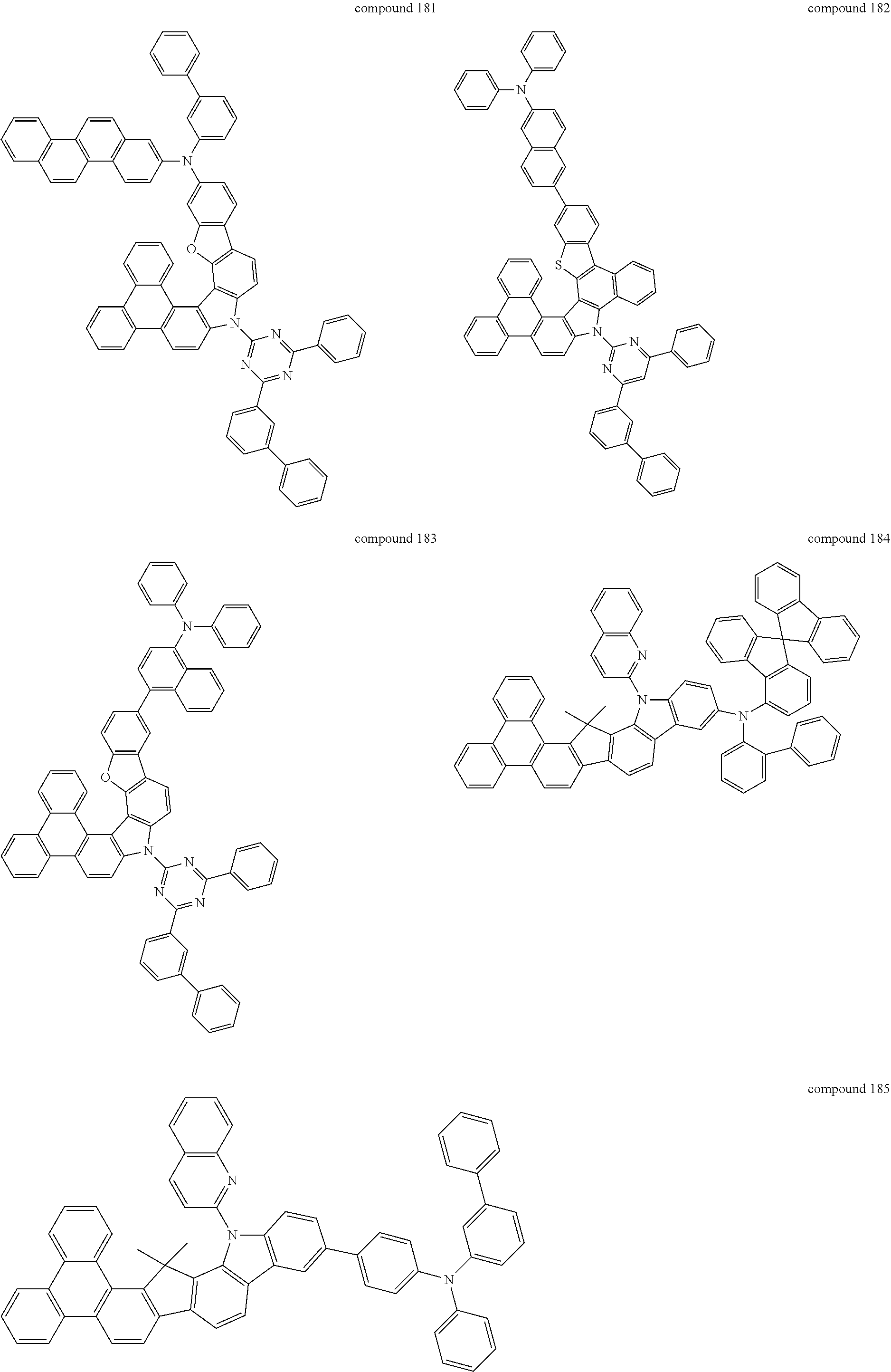

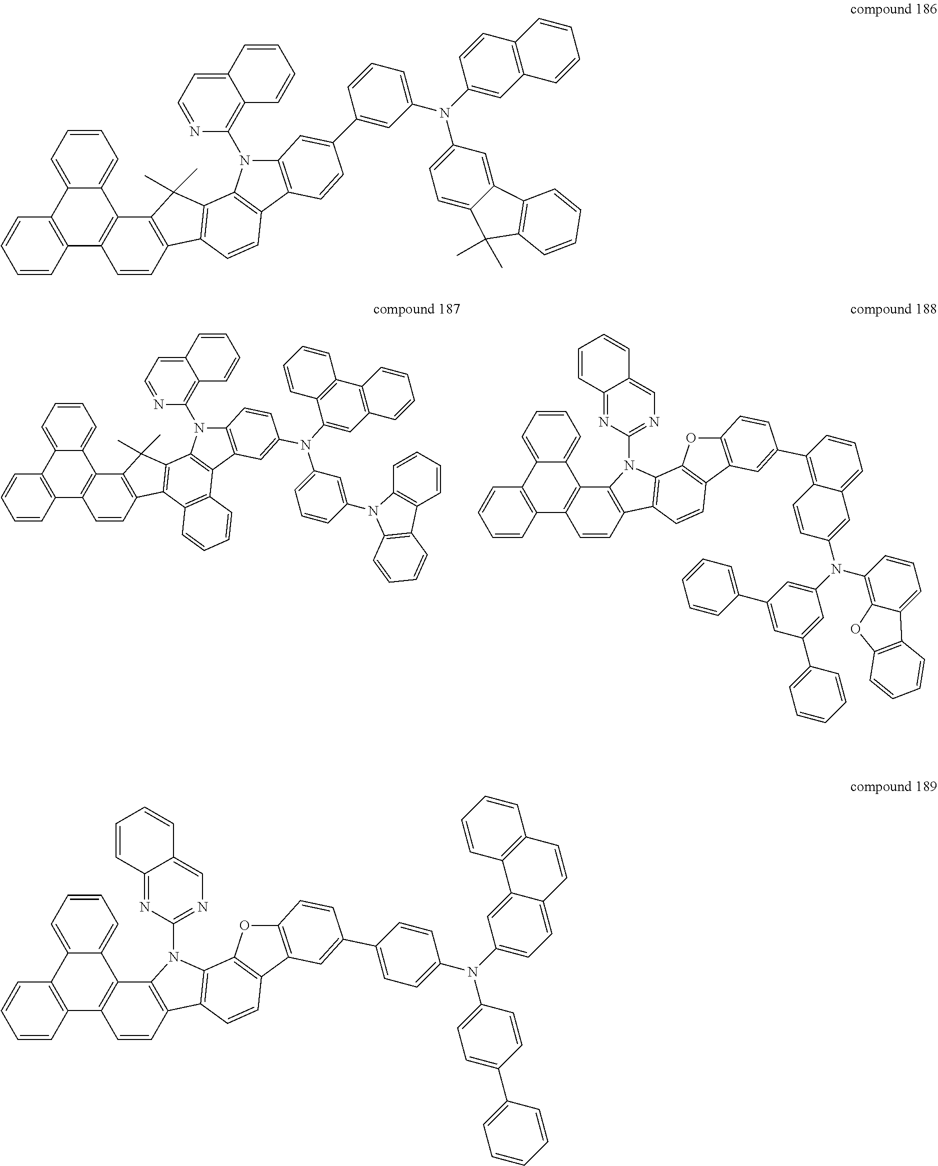

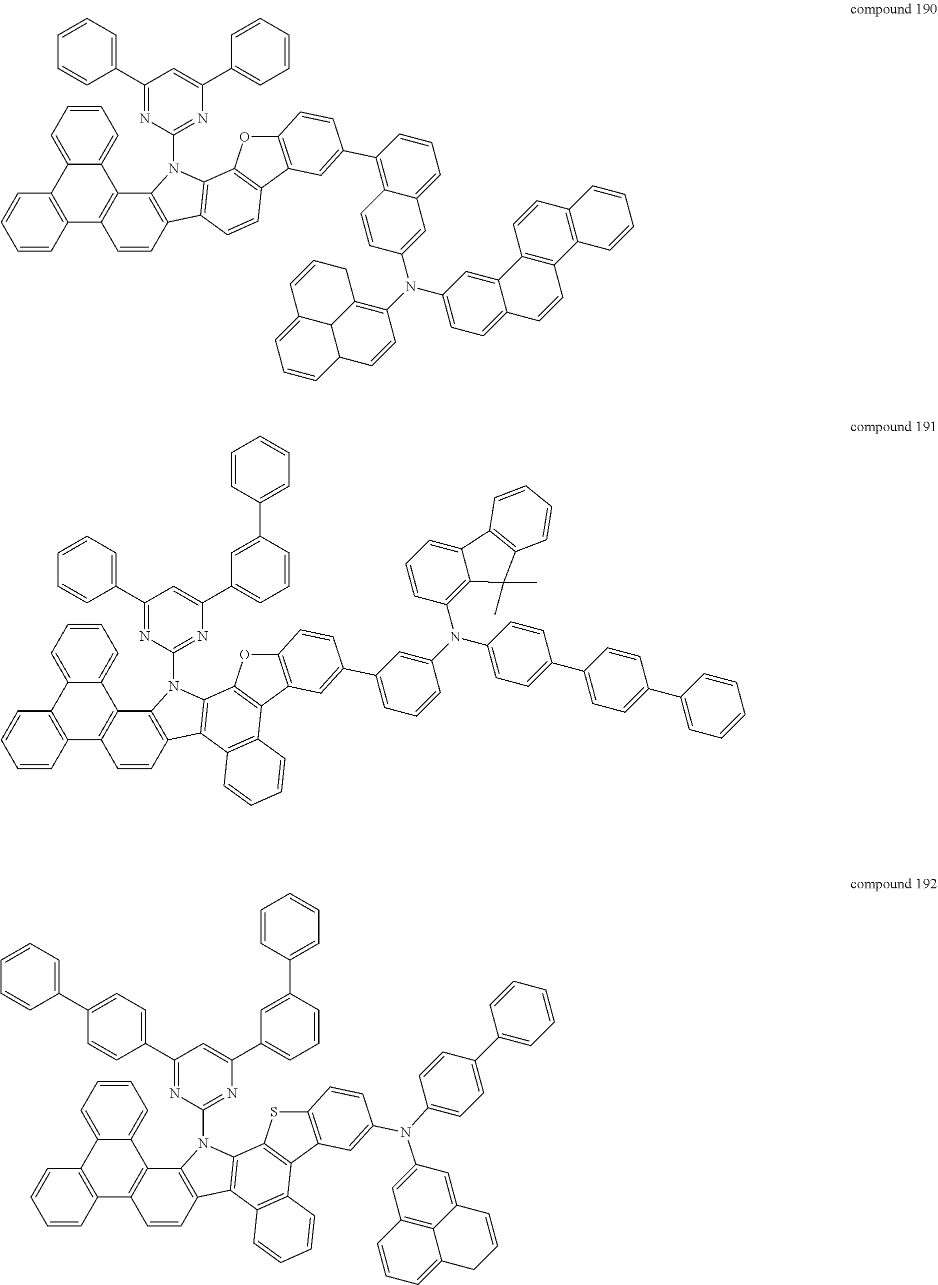

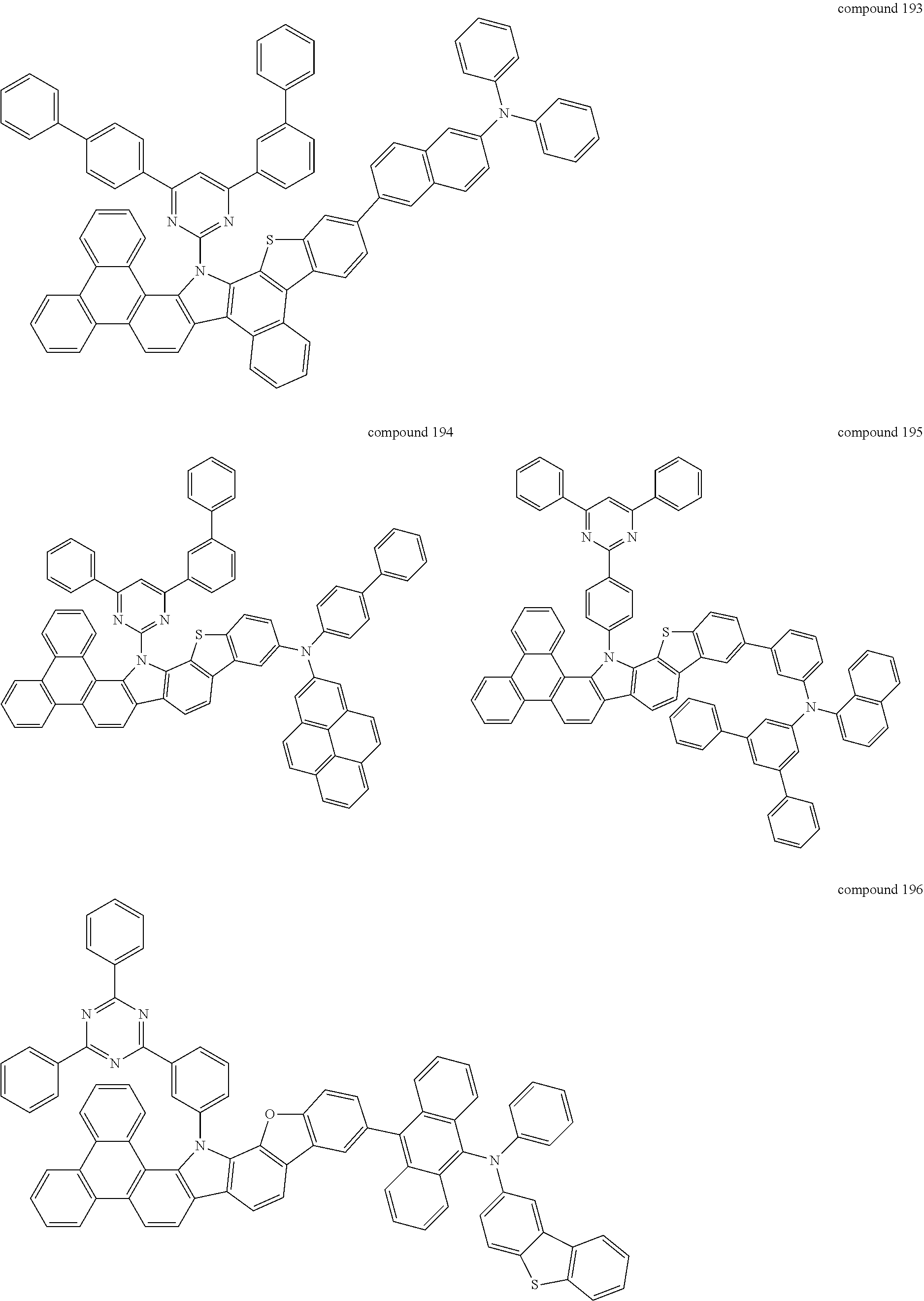

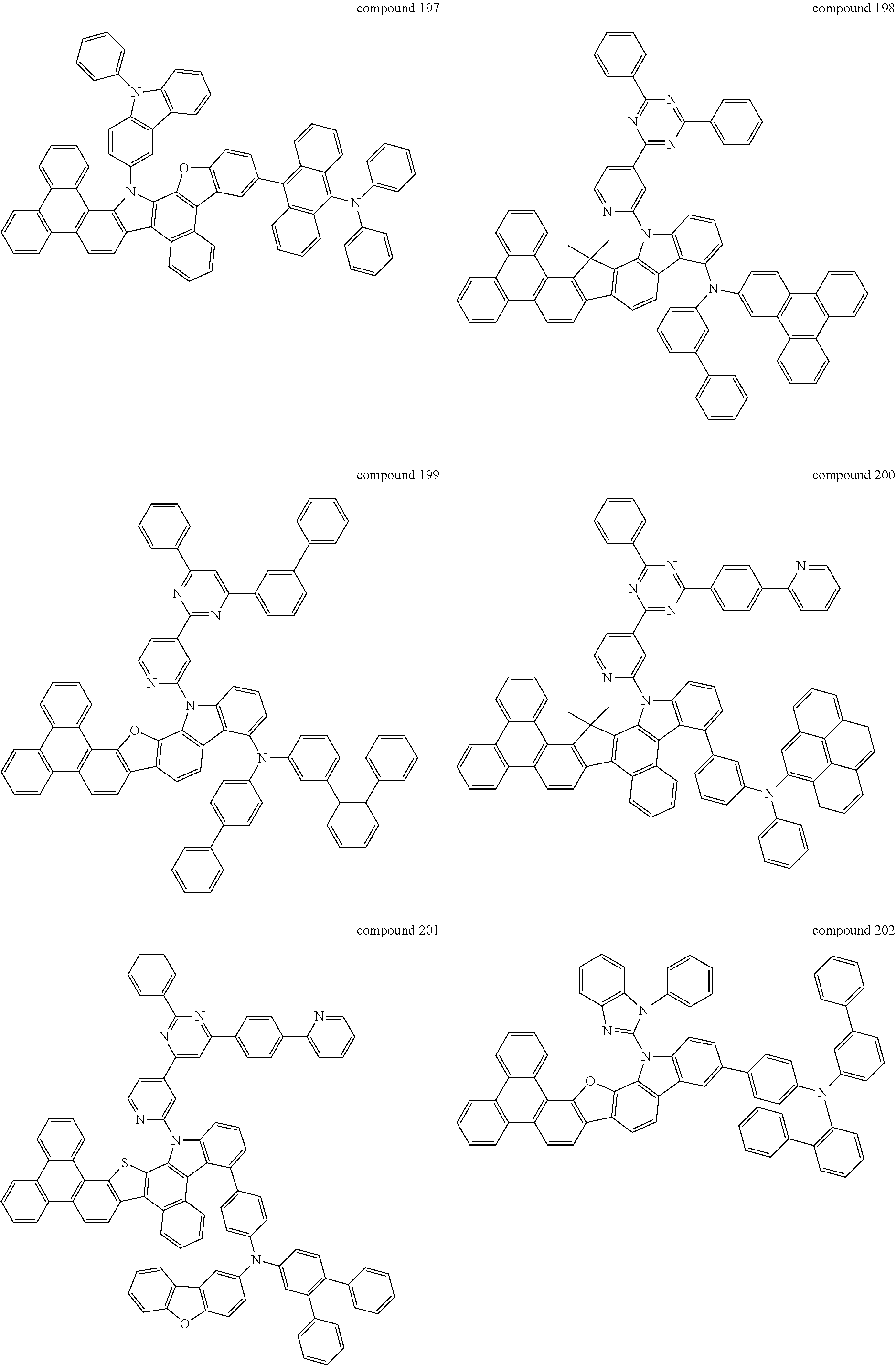

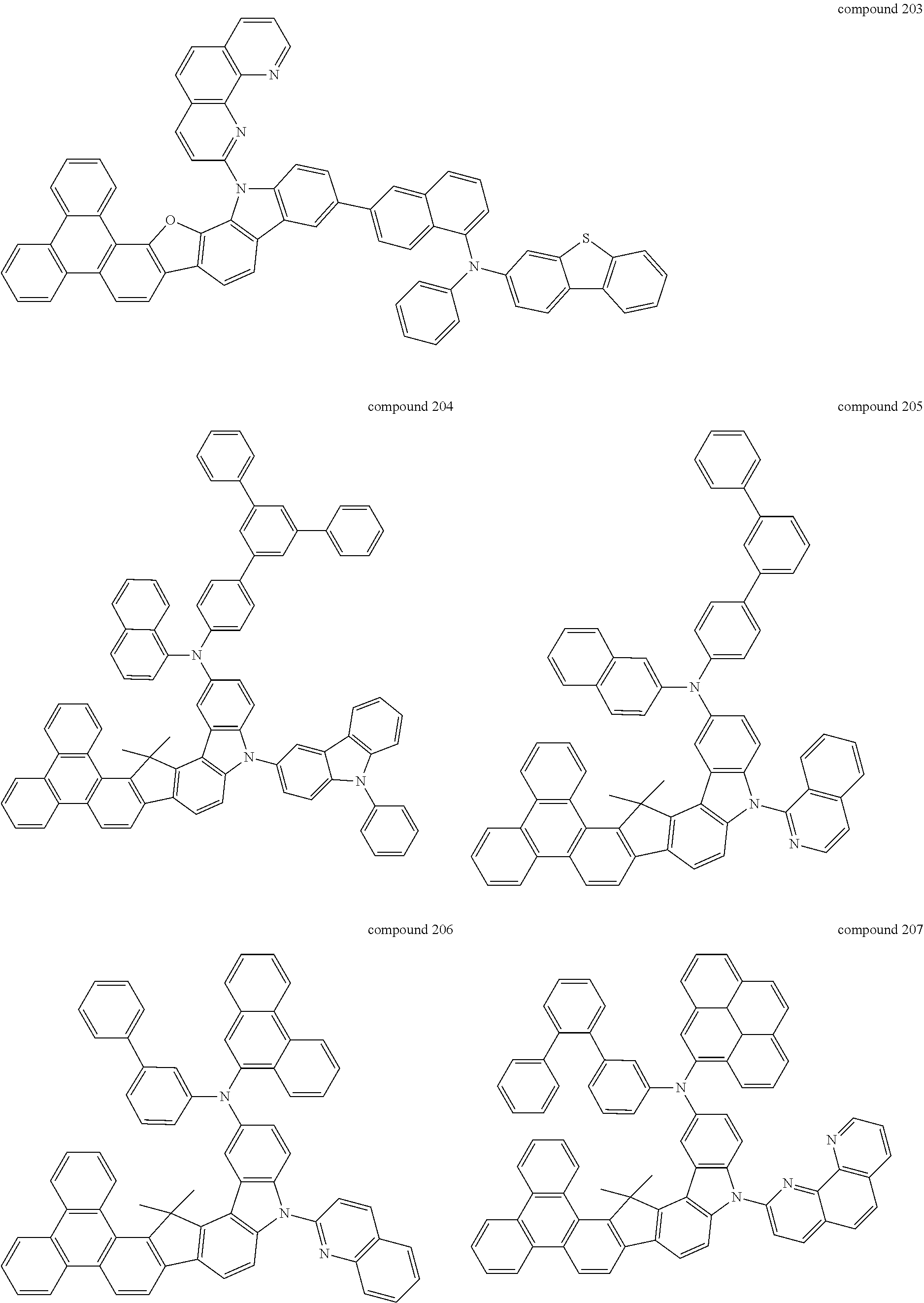

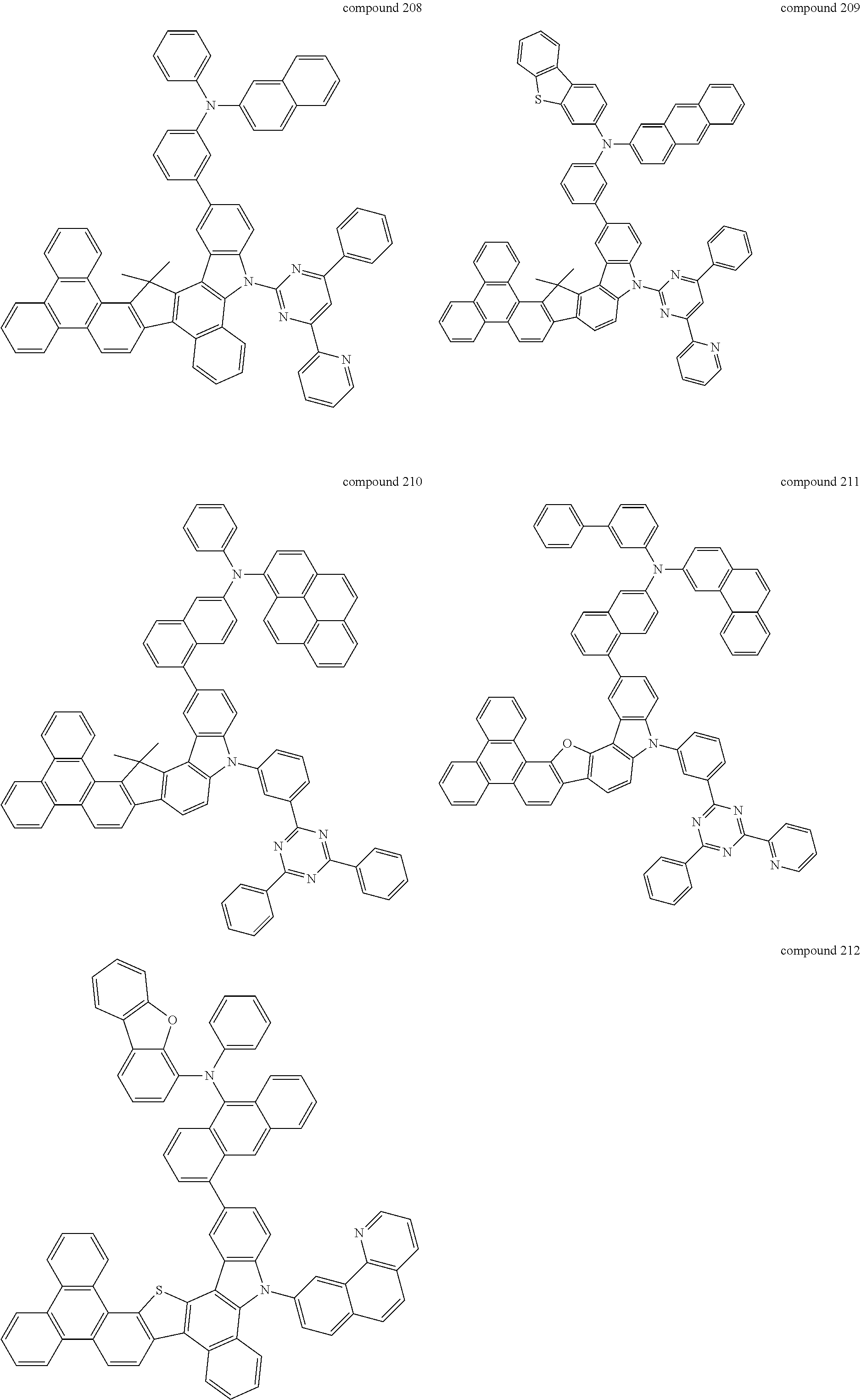

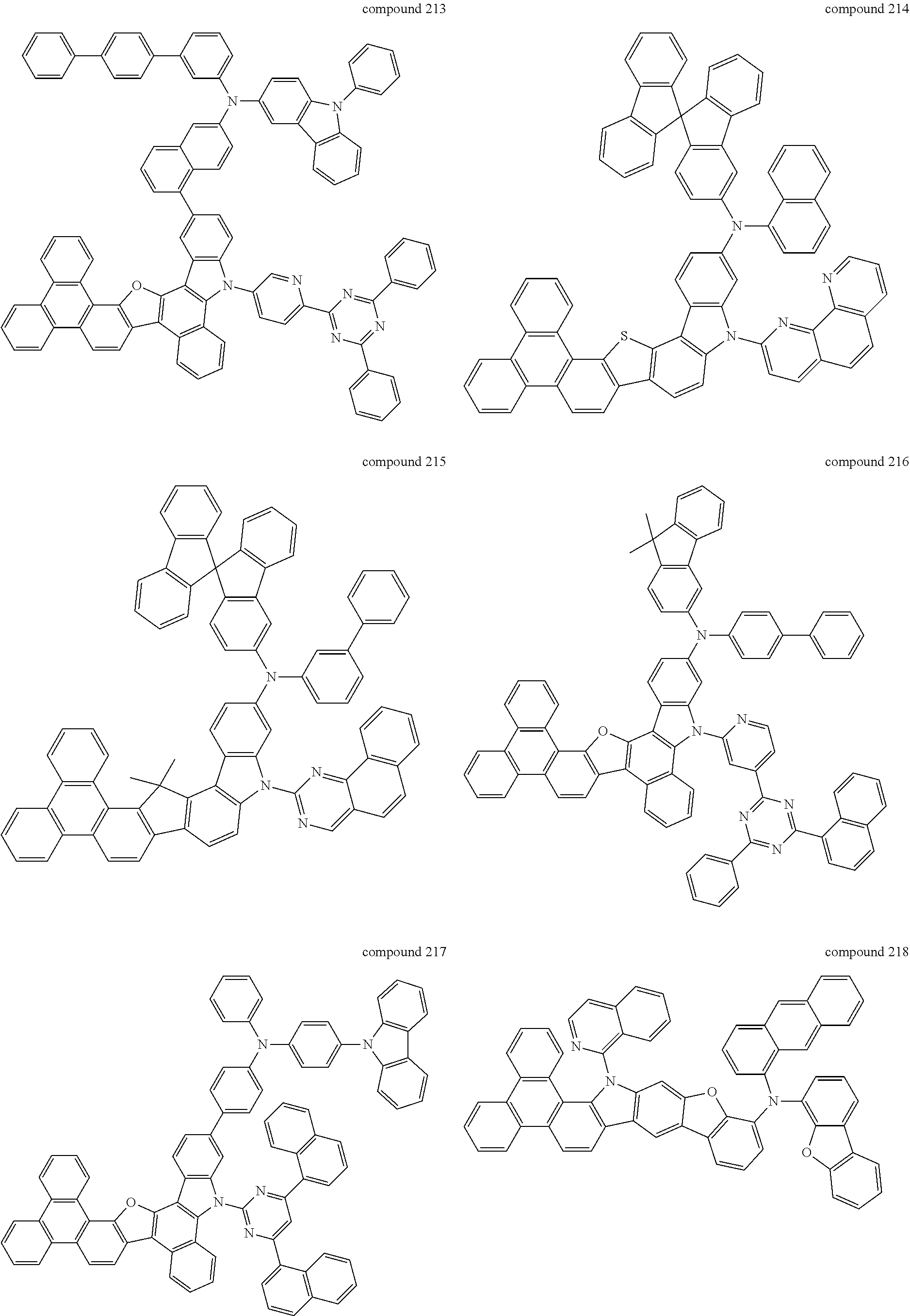

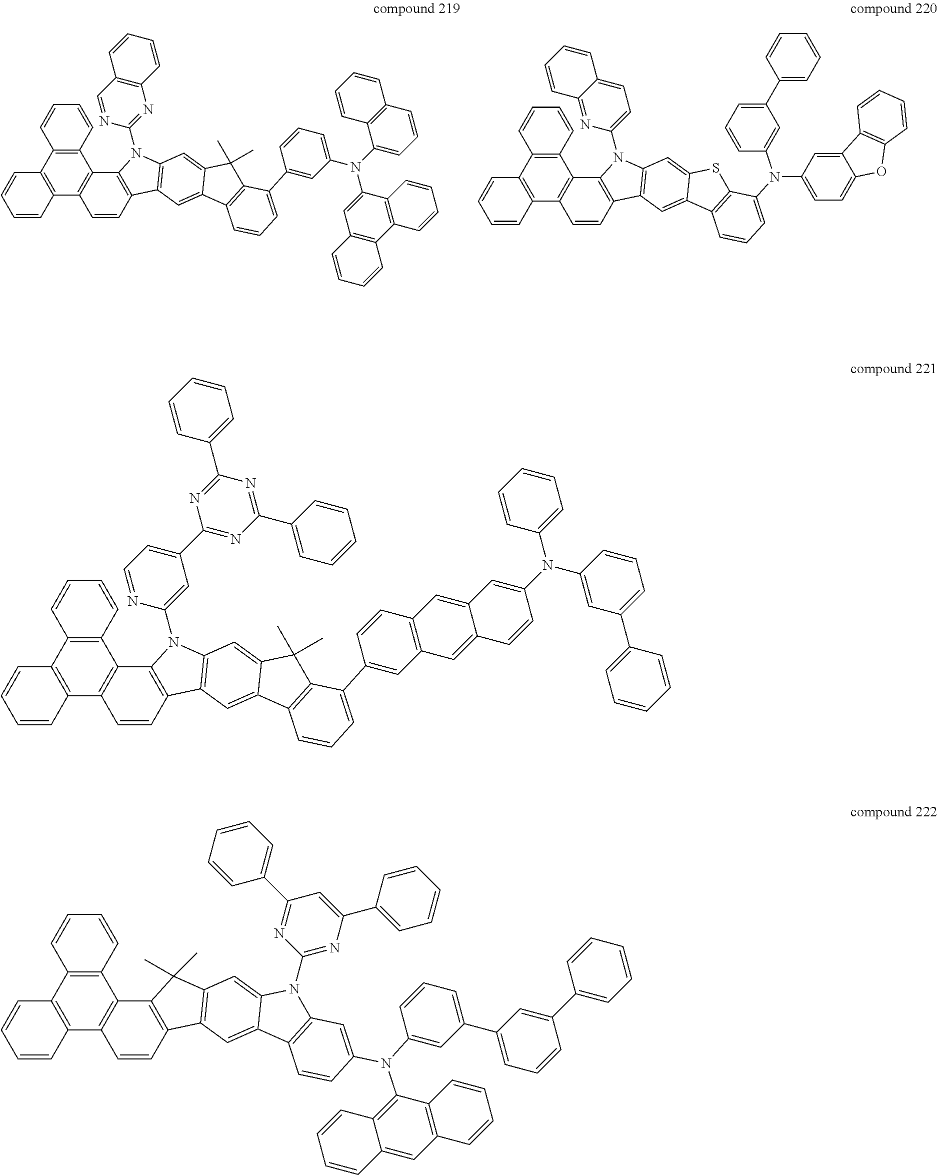

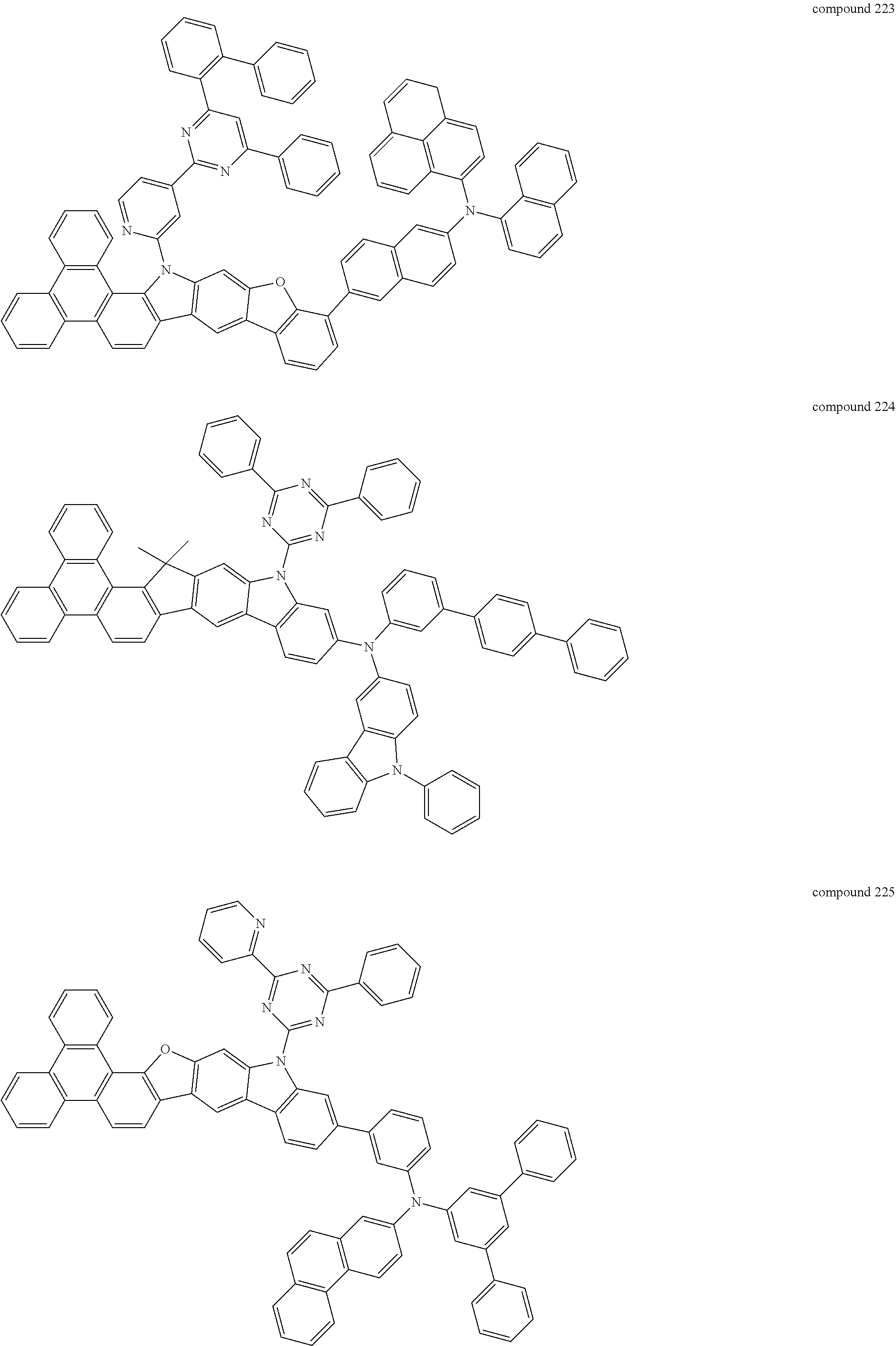 [SMILES: C1=Cc2cccc3c(N(c4ccc5cc(-c6cccc7c6oc6cc8c(cc67)c6ccc7c9ccccc9c9ccccc9c7c6n8-c6cc(-c7nc(-c8ccccc8)cc(-c8ccccc8-c8ccccc8)n7)ccn6)ccc5c4)c4cccc5ccccc45)ccc(c23)C1 ; CC1(C)c2cc3c(cc2-c2ccc4c5ccccc5c5ccccc5c4c21)c1ccc(N(c2cccc(-c4ccc(-c5ccccc5)cc4)c2)c2ccc4c(c2)c2ccccc2n4-c2ccccc2)cc1n3-c1nc(-c2ccccc2)nc(-c2ccccc2)n1 ; c1ccc(-c2cc(-c3ccccc3)cc(N(c3cccc(-c4ccc5c6cc7c(cc6n(-c6nc(-c8ccccc8)nc(-c8ccccn8)n6)c5c4)oc4c7ccc5c6ccccc6c6ccccc6c54)c3)c3ccc4c(ccc5ccccc54)c3)c2)cc1]

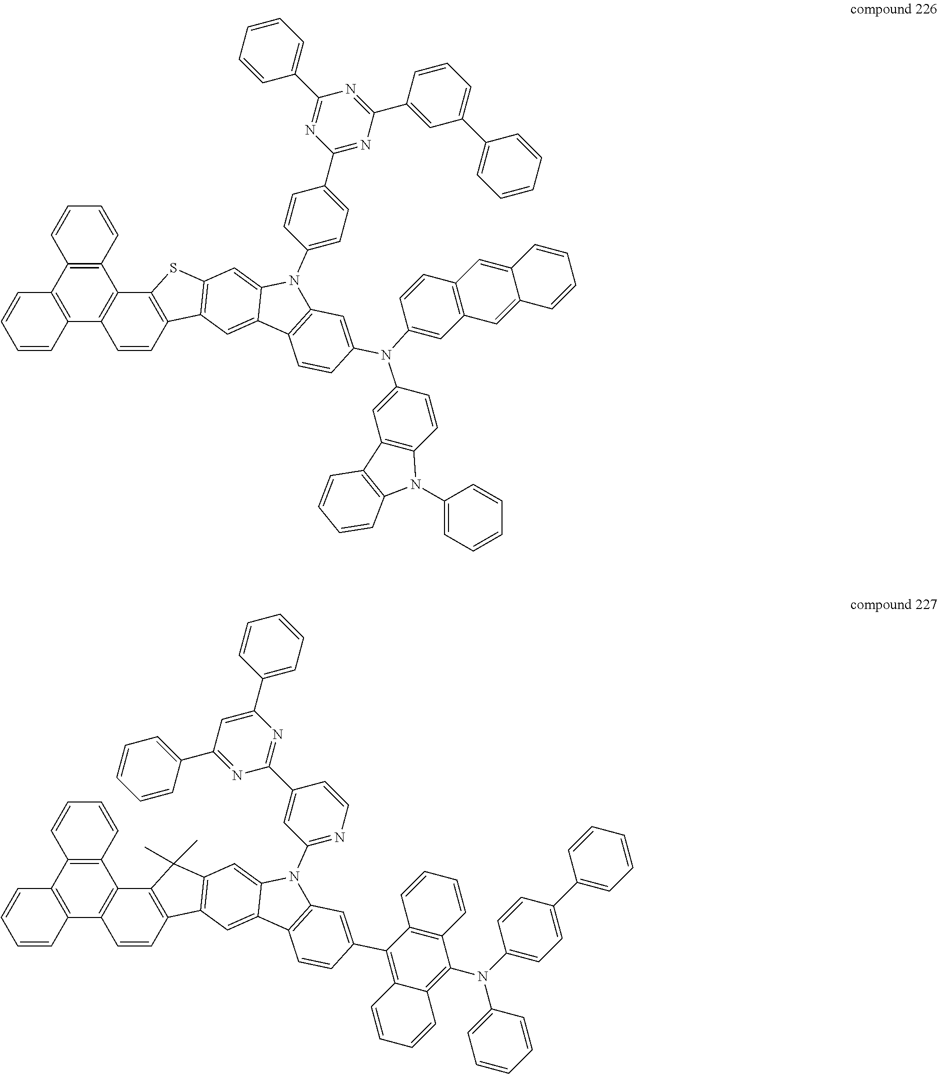 [SMILES: c1ccc(-c2cccc(-c3nc(-c4ccccc4)nc(-c4ccc(-n5c6cc(N(c7ccc8cc9ccccc9cc8c7)c7ccc8c(c7)c7ccccc7n8-c7ccccc7)ccc6c6cc7c(cc65)sc5c7ccc6c7ccccc7c7ccccc7c65)cc4)n3)c2)cc1 ; CC1(C)c2cc3c(cc2-c2ccc4c5ccccc5c5ccccc5c4c21)c1ccc(-c2c4ccccc4c(N(c4ccccc4)c4ccc(-c5ccccc5)cc4)c4ccccc24)cc1n3-c1cc(-c2nc(-c3ccccc3)cc(-c3ccccc3)n2)ccn1]

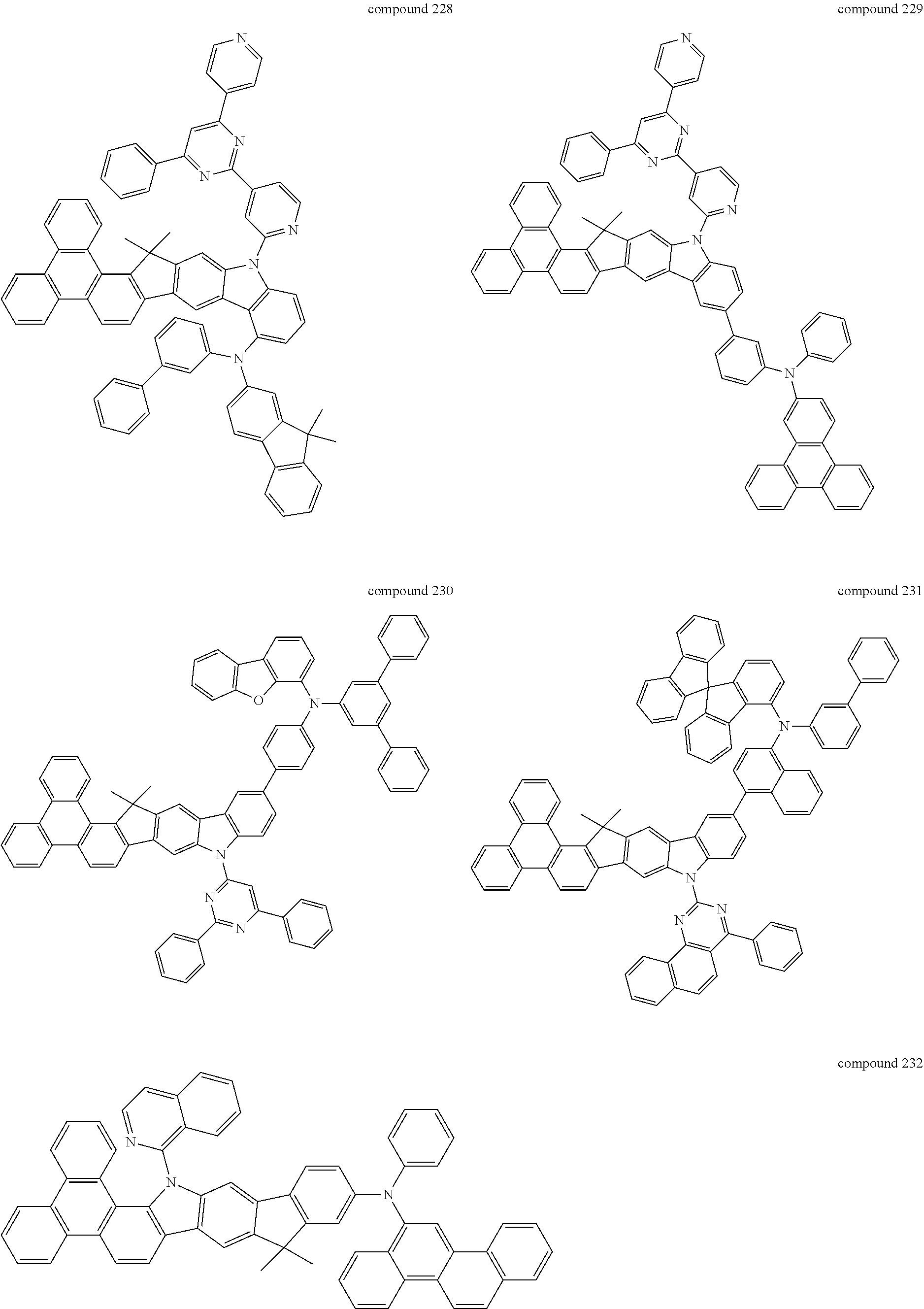

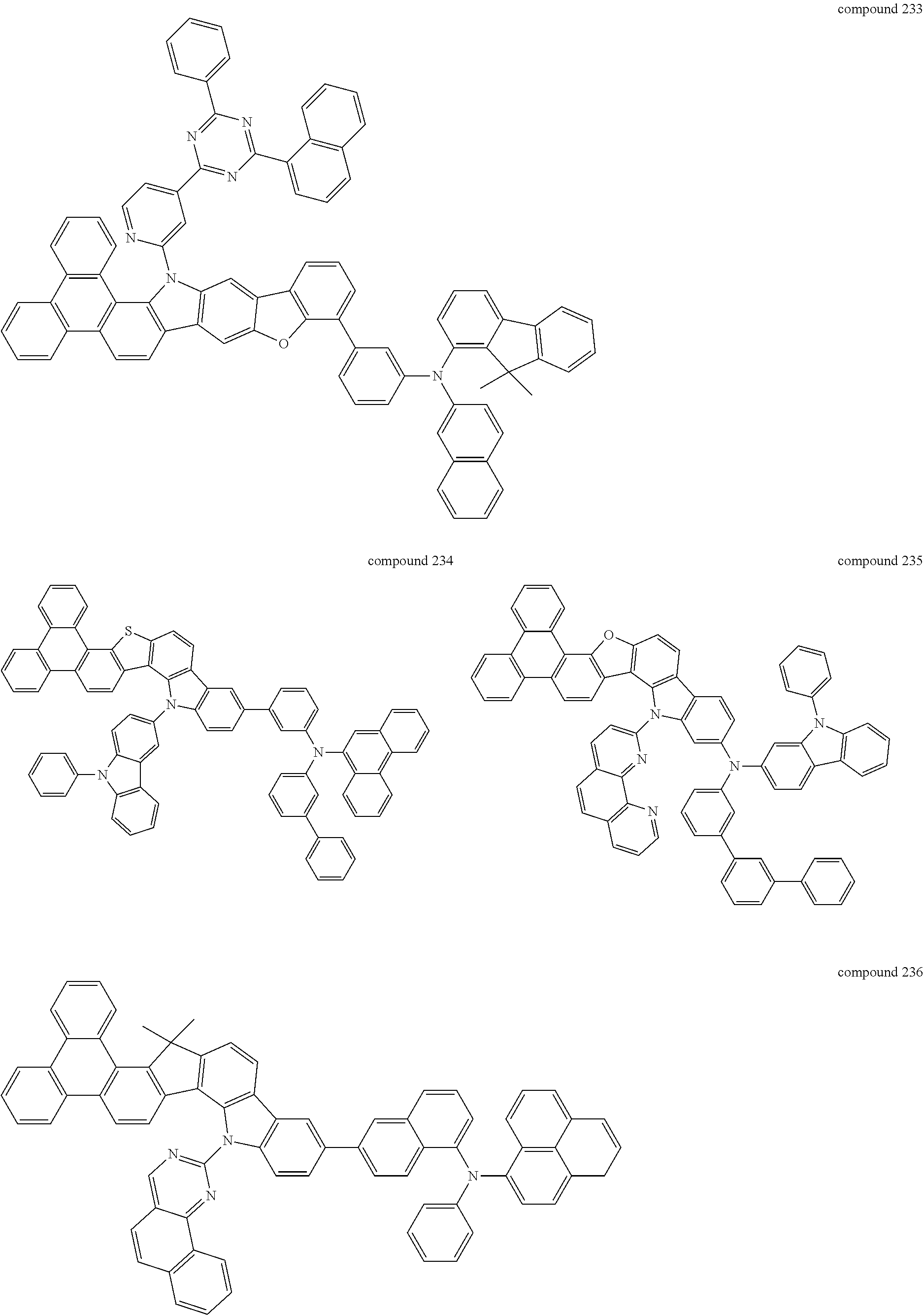

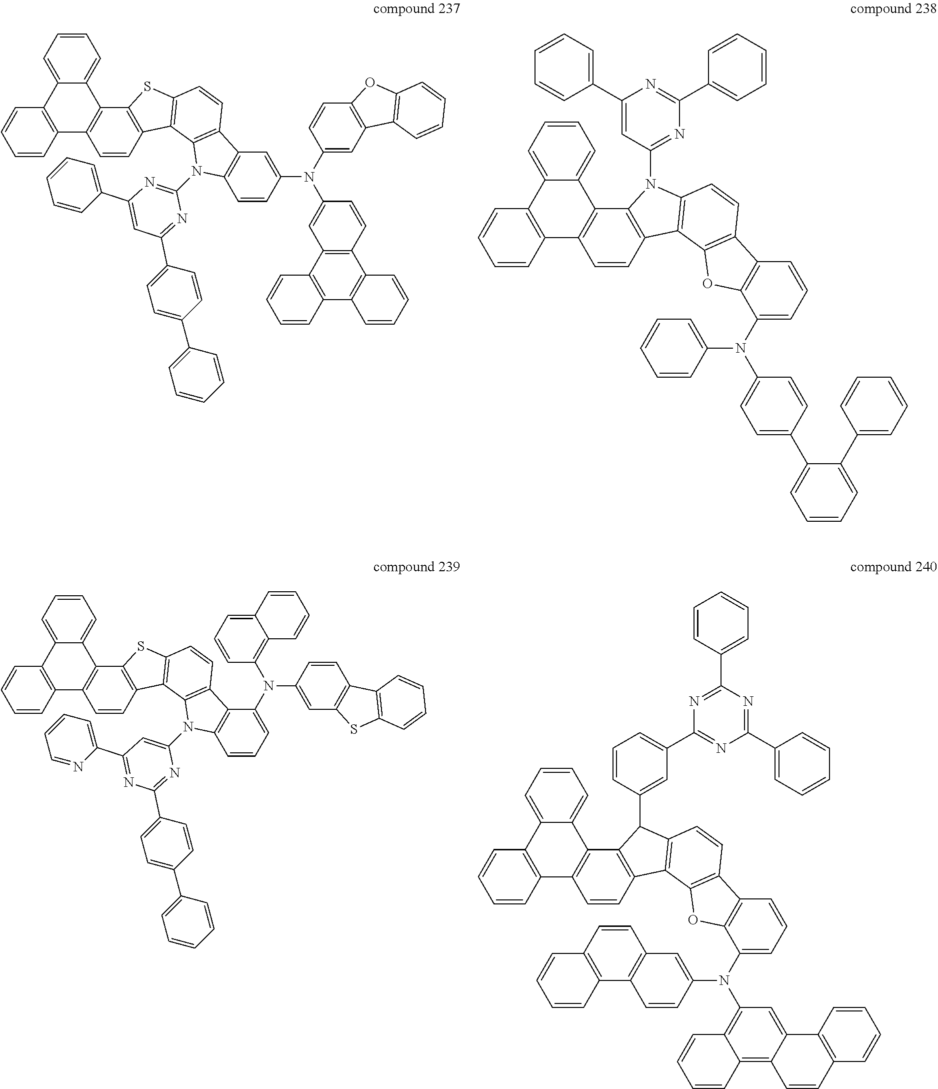

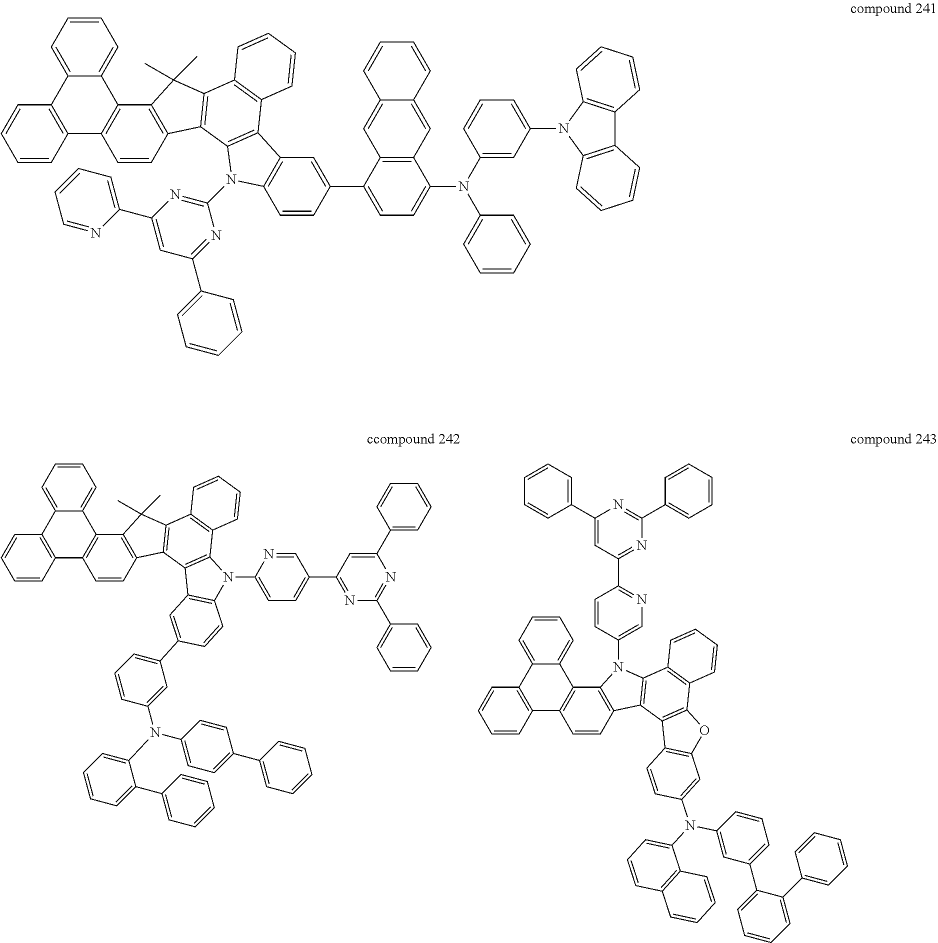 [SMILES: CC1(C)c2c(c3c(c4ccccc24)c2cc(-c4ccc(N(c5ccccc5)c5cccc(-n6c7ccccc7c7ccccc76)c5)c5cc6ccccc6cc45)ccc2n3-c2nc(-c3ccccc3)cc(-c3ccccn3)n2)-c2ccc3c4ccccc4c4ccccc4c3c21 ; CC1(C)c2c(c3c4cc(-c5cccc(N(c6ccc(-c7ccccc7)cc6)c6ccccc6-c6ccccc6)c5)ccc4n(-c4ccc(-c5cc(-c6ccccc6)nc(-c6ccccc6)n5)cn4)c3c3ccccc23)-c2ccc3c4ccccc4c4ccccc4c3c21 ; c1ccc(-c2cc(-c3ccc(-n4c5c(ccc6c7ccccc7c7ccccc7c65)c5c6c7ccc(N(c8cccc(-c9ccccc9-c9ccccc9)c8)c8cccc9ccccc89)cc7oc6c6ccccc6c54)cn3)nc(-c3ccccc3)n2)cc1]

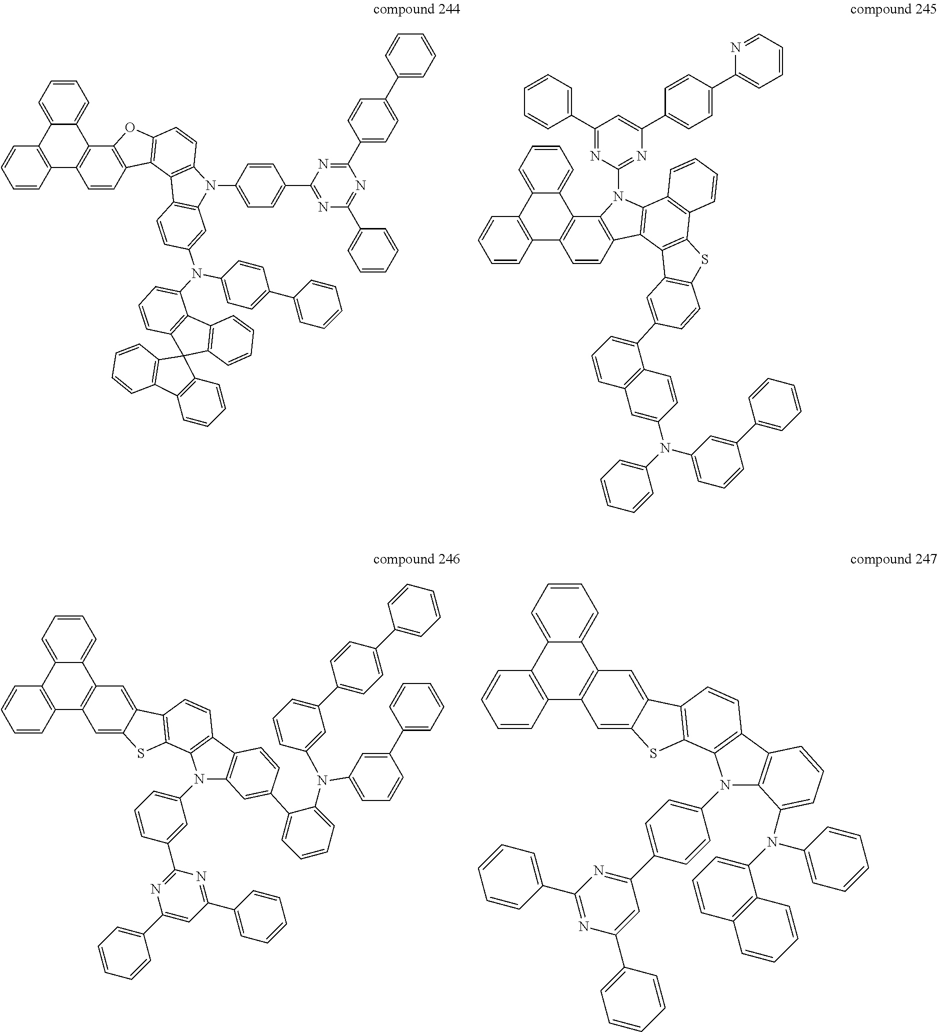

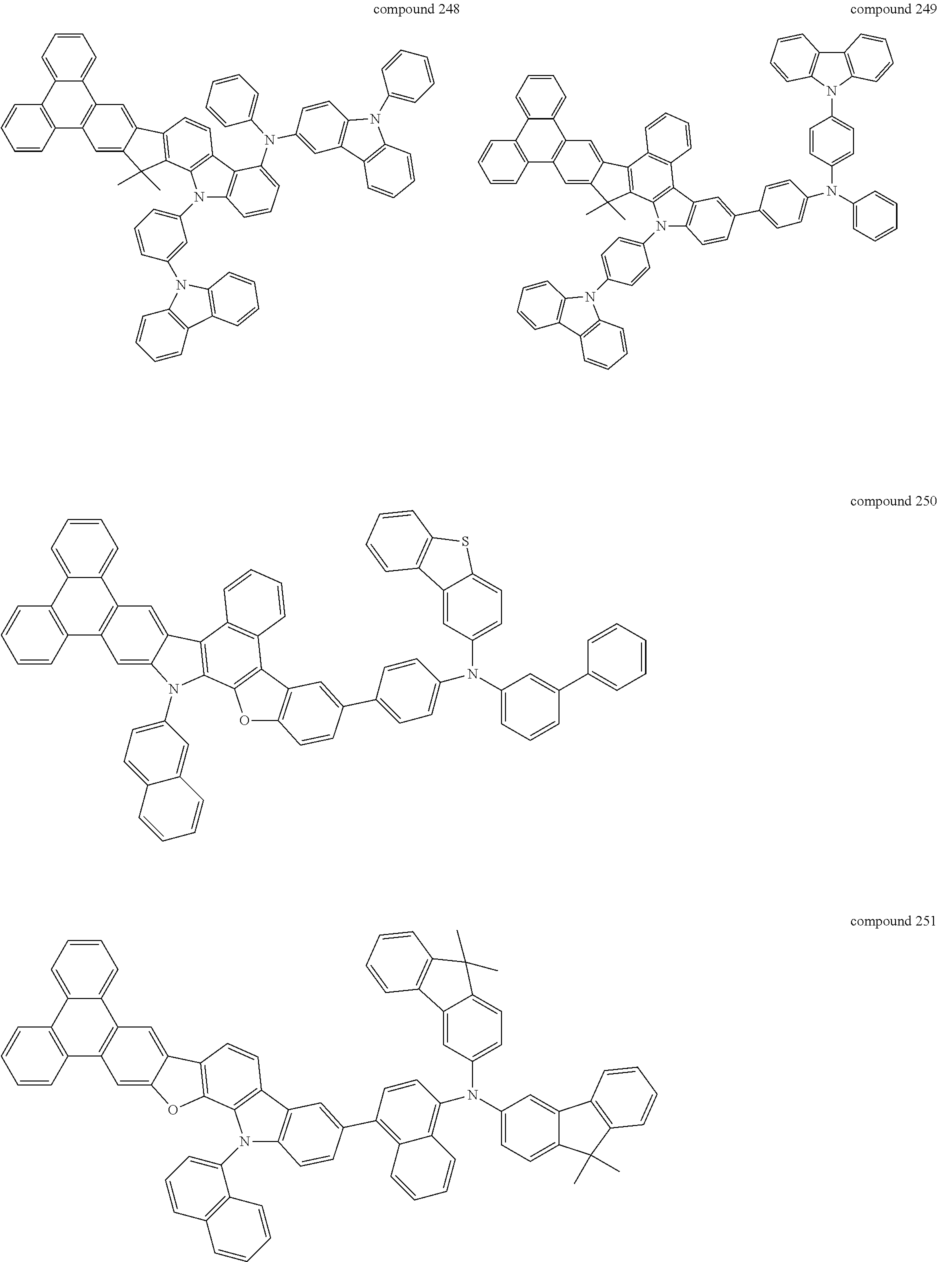

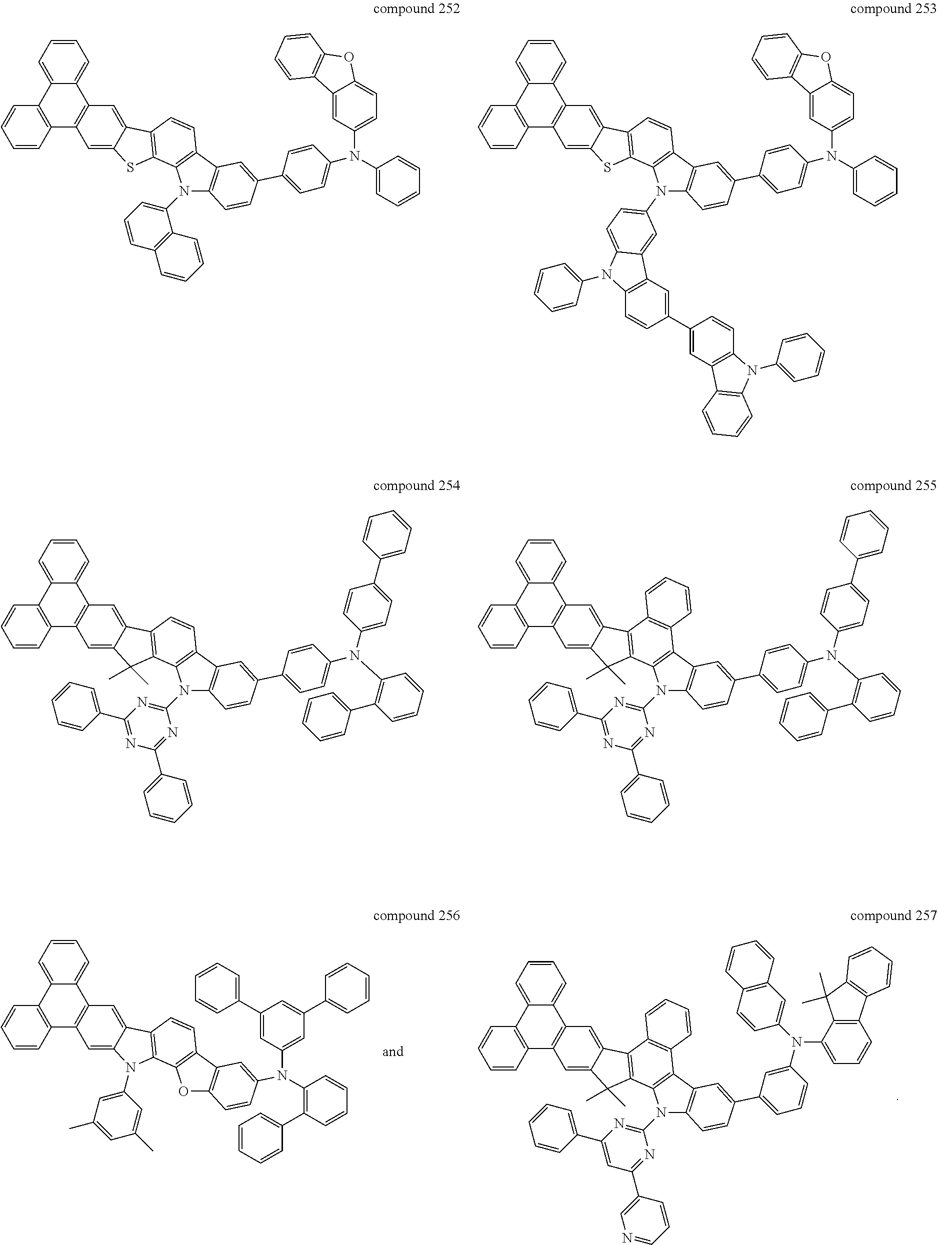

11. An organic electroluminescence device comprising an anode, a cathode and one or more organic layers between the anode and the cathode, wherein at least one of the organic layers comprises the organic compound of claim 1.

12. The organic electroluminescence device of claim 11, wherein the organic layers comprise an electron transporting layer, and wherein the organic compound is comprised as the electron transporting layer.

13. The organic electroluminescence device of claim 11, wherein the organic layers comprise a hole blocking layer, and wherein the organic compound is comprised as the hole blocking layer.

14. The organic electroluminescence device of claim 11, wherein the organic electroluminescence device is a lighting panel.

15. The organic electroluminescence device of claim 11, wherein the organic electroluminescence device is a backlight panel.
